SEQUENCE LISTING (240 sequences)

SEQ ID NO: 1 - DNA, 2178 bp, synthetic construct
  source 1..2178: mol_type = other DNA
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtga

SEQ ID NO: 2 - protein, 725 aa, synthetic construct
  source 1..725: note = CAR Construct 2
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 2277 bp, synthetic construct
  source 1..2277: mol_type = other DNA; note = CAR Construct 3
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccactacaactcctgccccacggccacctacacctgcgcctacaattgccagccagccactgtccttacgaccagaggcttgccgccccgctgcgggaggcgccgtccatacccggggccttgacttcgcatgcgatatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtga

SEQ ID NO: 4 - protein, 758 aa, synthetic construct
  source 1..758: note = CAR Construct 4
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIASQPLSLRPEACRPAAGGAVHTRGLDFACDMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 2259 bp, synthetic construct
  source 1..2259: mol_type = other DNA; note = CAR Construct 5
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccattgaagttatgtatcctcctccttacctagacaatgagaagagcaatggaaccattatccatgtgaaagggaaacacctttgtccaagtcccctatttcccggaccttctaagcccatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtga

SEQ ID NO: 6 - protein, 752 aa, synthetic construct
  source 1..752: note = CAR Construct 6
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITIEVMYPPPYLDNEKSNGTIIHVKGKHLCPSPLFPGPSKPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - DNA, 2286 bp, synthetic construct
  source 1..2286: mol_type = other DNA; note = CAR Construct 7
gctagcgccgccaccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccactacaactcctgccccacggccacctacacctgcgcctacaattgccagccagccactgtccttacgaccagaggcttgccgccccgctgcgggaggcgccgtccatacccggggccttgacttcgcatgcgatatttacatttgggcacccttggccggcacctgcggagttttacttctgagcttggtgataacgctgtactgtaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 8 - protein, 754 aa, synthetic construct
  source 1..754: note = CAR Construct 8
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 2214 bp, synthetic construct
  source 1..2214: mol_type = other DNA; note = CAR Construct 9
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcacctctgggcaagttcttcttgagtctaatatcaaggtgctgcccacctggtctactcctgtccaaccgatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 10 - protein, 735 aa, synthetic construct
  source 1..735: note = CAR Construct 10
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSGQVLLESNIKVLPTWSTPVQPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 2235 bp, synthetic construct
  source 1..2235: mol_type = other DNA; note = CAR Construct 11
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccagccagccactgtccttacgaccagaggcttgccgccccgctgcgggaggcgccgtccatacccggggccttgacttcgcatgcgatatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 12 - protein, 742 aa, synthetic construct
  source 1..742: note = CAR Construct 12
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSQPLSLRPEACRPAAGGAVHTRGLDFACDMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 2202 bp, synthetic construct
  source 1..2202: mol_type = other DNA; note = CAR Construct 13
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcacccgccccgctgcgggaggcgccgtccatacccggggccttgacttcgcatgcgatatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 14 - protein, 731 aa, synthetic construct
  source 1..731: note = CAR Construct 14
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRPAAGGAVHTRGLDFACDMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 2199 bp, synthetic construct
  source 1..2199: mol_type = other DNA; note = CAR Construct 15
gctagcgccgccaccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccgagcctaaaagttgtgataaaacgcatacctgtccaatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 16 - protein, 725 aa, synthetic construct
  source 1..725: note = CAR Construct 16
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITEPKSCDKTHTCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 2199 bp, synthetic construct
  source 1..2199: mol_type = other DNA; note = CAR Construct 17
gctagcgccgccaccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccgagccaaagtcatgtgatacacctcctccctgtcccatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 18 - protein, 725 aa, synthetic construct
  source 1..725: note = CAR Construct 18
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITEPKSCDTPPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 2205 bp, synthetic construct
  source 1..2205: mol_type = other DNA; note = CAR Construct 19
gctagcgccgccaccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccgtaccctgtcgagtaccgcctccgcccccatgctgccacccgatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 20 - protein, 727 aa, synthetic construct
  source 1..727: note = CAR Construct 20
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITVPCRVPPPPPCCHPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 12 aa, synthetic construct
  source 1..12: note = IgG4 hinge
ESKYGPPCPPCP

SEQ ID NO: 22 - protein, 45 aa, synthetic construct
  source 1..45: note = CD8alpha hinge (45 aa)
TTTPAPRPPTPAPTIASQPLSLRPEACRPAAGGAVHTRGLDFACD

SEQ ID NO: 23 - protein, 39 aa, synthetic construct
  source 1..39: note = CD28 hinge
IEVMYPPPYLDNEKSNGTIIHVKGKHLCPSPLFPGPSKP

SEQ ID NO: 24 - protein, 22 aa, synthetic construct
  source 1..22: note = CD4 hinge
SGQVLLESNIKVLPTWSTPVQP

SEQ ID NO: 25 - protein, 29 aa, synthetic construct
  source 1..29: note = CD8alpha hinge (29 aa)
SQPLSLRPEACRPAAGGAVHTRGLDFACD

SEQ ID NO: 26 - protein, 18 aa, synthetic construct
  source 1..18: note = CD8alpha hinge (18 aa)
RPAAGGAVHTRGLDFACD

SEQ ID NO: 27 - protein, 12 aa, synthetic construct
  source 1..12: note = IgG1 hinge
EPKSCDKTHTCP

SEQ ID NO: 28 - protein, 12 aa, synthetic construct
  source 1..12: note = IgG3 hinge
EPKSCDTPPPCP

SEQ ID NO: 29 - protein, 14 aa, synthetic construct
  source 1..14: note = IgA2 hinge
VPCRVPPPPPCCHP

SEQ ID NO: 30 - protein, 27 aa, synthetic construct
  source 1..27: note = CD28 TM
FWVLVVVGGVLACYSLLVTVAFIIFWV

SEQ ID NO: 31 - protein, 24 aa, synthetic construct
  source 1..24: note = CD8alpha TM
IYIWAPLAGTCGVLLLSLVITLYC

SEQ ID NO: 32 - protein, 42 aa, synthetic construct
  source 1..42: note = 4-1BB signaling domain
KRGRKKLLYIFKQPFMRPVQTTQEEDGCSCRFPEEEEGGCEL

SEQ ID NO: 33 - protein, 112 aa, synthetic construct
  source 1..112: note = CD3 zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 106 aa, synthetic construct
  source 1..106: note = Murine anti CD20 Leu16 hybridoma VK sequence
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 35 - protein, 122 aa, synthetic construct
  source 1..122: note = Murine anti CD20 hybridoma VH sequence
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 36 - protein, 120 aa, synthetic construct
  source 1..120: note = Murine anti CD19 VH sequence
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 37 - protein, 107 aa, synthetic construct
  source 1..107: note = Murine anti CD19 VK sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 38 - DNA, 22 bp, synthetic construct
  source 1..22: mol_type = other DNA; note = Forward:Set A: Leu16 scFv (Target)
cttattactgccagcagtggag

SEQ ID NO: 39 - DNA, 20 bp, synthetic construct
  source 1..20: mol_type = other DNA; note = Reverse:Set A: Leu16 scFv (Target)
caggacatcttcactgaggc

SEQ ID NO: 40 - DNA, 23 bp, synthetic construct
  source 1..23: mol_type = other DNA; note = Probe:FAM/Iowa Black FQ
cggagccaccaccgctagtactg

SEQ ID NO: 41 - DNA, 19 bp, synthetic construct
  source 1..19: mol_type = other DNA; note = Forward: Set B: FMC63 scFv (Target)
cgacatccagatgacccag

SEQ ID NO: 42 - DNA, 20 bp, synthetic construct
  source 1..20: mol_type = other DNA; note = Reverse:Set B: FMC63 scFv (Target)
gttccaggttggagatggtc

SEQ ID NO: 43 - DNA, 29 bp, synthetic construct
  source 1..29: mol_type = other DNA; note = Probe:HEX/ZEN/Iowa Black FQ
caccgtcaagctgctgatctaccacacca

SEQ ID NO: 44 - DNA, 19 bp, synthetic construct
  source 1..19: mol_type = other DNA; note = Forward:Set D: SDC4 (Reference)
cagggtctgggagccaagt

SEQ ID NO: 45 - DNA, 21 bp, synthetic construct
  source 1..21: mol_type = other DNA; note = Reverse:Set D: SDC4 (Reference)
gcacagtgctggacattgaca

SEQ ID NO: 46 - DNA, 30 bp, synthetic construct
  source 1..30: mol_type = other DNA; note = Probe:HEX/ZEN/Iowa Black FQ
cccaccgaacccaagaaactagaggagaat

SEQ ID NO: 47 - skipped

SEQ ID NO: 48 - protein, 5 aa, synthetic construct
  source 1..5: note = (G4S)1
GGGGS

SEQ ID NO: 49 - protein, 10 aa, synthetic construct
  source 1..10: note = (G4S)2
GGGGSGGGGS

SEQ ID NO: 50 - protein, 15 aa, synthetic construct
  source 1..15: note = (G4S)3
GGGGSGGGGSGGGGS

SEQ ID NO: 51 - protein, 20 aa, synthetic construct
  source 1..20: note = (G4S)4
GGGGSGGGGSGGGGSGGGGS

SEQ ID NO: 52 - protein, 725 aa, synthetic construct
  source 1..725: note = CAR Construct 21
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDIATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 725 aa, synthetic construct
  source 1..725: note = CAR Construct 22
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDKSTSTVYMELSSLRSEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDIATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 725 aa, synthetic construct; identical to SEQ ID NO: 53
  source 1..725: note = CAR Construct 23

SEQ ID NO: 55 - protein, 725 aa, synthetic construct; identical to SEQ ID NO: 53
  source 1..725: note = CAR Construct 24

SEQ ID NO: 56-59 - skipped (4 entries)

SEQ ID NO: 60 - protein, 725 aa, synthetic construct
  source 1..725: note = CAR Construct 25
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 725 aa, synthetic construct
  source 1..725: note = CAR Construct 26
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 725 aa, synthetic construct
  source 1..725: note = CAR Construct 27
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 725 aa, synthetic construct
  source 1..725: note = CAR Construct 28
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDKSTSTVYMELSSLRSEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 725 aa, synthetic construct
  source 1..725: note = CAR Construct 29
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 725 aa, synthetic construct
  source 1..725: note = CAR Construct 30
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISADNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 725 aa, synthetic construct
  source 1..725: note = CAR Construct 31
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYFCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 725 aa, synthetic construct
  source 1..725: note = CAR Construct 32
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYFCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 725 aa, synthetic construct
  source 1..725: note = CAR Construct 33
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYFCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - skipped

SEQ ID NO: 70 - protein, 725 aa, synthetic construct
  source 1..725: note = CAR Construct 34
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDKSTSTVYMELSSLRSEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYFCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 725 aa, synthetic construct
  source 1..725: note = CAR Construct 35
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYFCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 725 aa, synthetic construct
  source 1..725: note = CAR Construct 36
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISADNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYFCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 10 aa, synthetic construct
  source 1..10: note = Murine anti CD20 Leu16 hybridoma CDRL1
RASSSVNYMD

SEQ ID NO: 74 - protein, 8 aa, synthetic construct
  source 1..8: note = Murine anti CD20 Leu16 hybridoma CDRL2
YATSNLAS

SEQ ID NO: 75 - protein, 9 aa, synthetic construct
  source 1..9: note = Murine anti CD20 Leu16 hybridoma CDRL3
QQWSFNPPT

SEQ ID NO: 76 - protein, 5 aa, synthetic construct
  source 1..5: note = Murine anti CD20 hybridoma CDRH1
SYNMH

SEQ ID NO: 77 - protein, 17 aa, synthetic construct
  source 1..17: note = Murine anti CD20 hybridoma CDRH2
AIYPGNGDTSYNQKFKG

SEQ ID NO: 78 - protein, 13 aa, synthetic construct
  source 1..13: note = Murine anti CD20 hybridoma CDRH3
SNYYGSSYWFFDV

SEQ ID NO: 79 - protein, 122 aa, synthetic construct
  source 1..122: note = Hz. Leu16(a) VH
QVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSNYYGSSYWFFDVWGQGTTVTVSS

SEQ ID NO: 80 - protein, 5 aa, synthetic construct
  source 1..5: note = Hz. Leu16 (a) VH CDRH1
SYNMH

SEQ ID NO: 81 - protein, 16 aa, synthetic construct
  source 1..16: note = Hz. Leu16 (a) VH CDRH2
AIYPGNGDTSYNQKFK

SEQ ID NO: 82 - protein, 14 aa, synthetic construct
  source 1..14: note = Hz. Leu16 (a) VH CDRH3
RSNYYGSSYWFFDV

SEQ ID NO: 83 - protein, 122 aa, synthetic construct
  source 1..122: note = Hz. Leu16 (b) VH
QVQLVQSGAEVKKPGASVKVSCKASGYTFTSYNMHWVRQAPGQGLEWMGAIYPGNGDTSYNQKFKGRVTMTRDKSTSTVYMELSSLRSEDTAVYYCARSNYYGSSYWFFDVWGQGTTVTVSS

SEQ ID NO: 84 - protein, 5 aa, synthetic construct
  source 1..5: note = Hz. Leu16 (b) VH CDRH1
SYNMH

SEQ ID NO: 85 - protein, 17 aa, synthetic construct
  source 1..17: note = Hz. Leu16 (b) VH CDRH2
AIYPGNGDTSYNQKFKG

SEQ ID NO: 86 - protein, 14 aa, synthetic construct
  source 1..14: note = Hz. Leu16 (b) VH CDRH3
RSNYYGSSYWFFDV

SEQ ID NO: 87 - protein, 121 aa, synthetic construct
  source 1..121: note = Hz. Leu16 (c) VH
EVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCARSNYYGSSYWFFDVWGQGTTVTVS

SEQ ID NO: 88 - protein, 5 aa, synthetic construct
  source 1..5: note = Hz. Leu16 (c) VH CDRH1
SYNMH

SEQ ID NO: 89 - protein, 17 aa, synthetic construct
  source 1..17: note = Hz. Leu16 (c) VH CDRH2
AIYPGNGDTSYNQKFKG

SEQ ID NO: 90 - protein, 14 aa, synthetic construct
  source 1..14: note = Hz. Leu16 (c) VH CDRH3
RSNYYGSSYWFFDV

SEQ ID NO: 91 - protein, 122 aa, synthetic construct
  source 1..122: note = Hz. Leu16 (d) VH
EVQLLESGGGLVQPGGSLRLSCAASGFTFTSYNMHWVRQAPGKGLEWVSAIYPGNGDTSYNQKFKGRFTISRDNSKNTLYLQMNSLRAEDTAVYYCARSNYYGSSYWFFDVWGQGTTVTVSS

SEQ ID NO: 92 - protein, 5 aa, synthetic construct
  source 1..5: note = Hz. Leu16 (d) VH CDRH1
SYNMH

SEQ ID NO: 93 - protein, 17 aa, synthetic construct
  source 1..17: note = Hz. Leu16 (d) VH CDRH2
AIYPGNGDTSYNQKFKG

SEQ ID NO: 94 - protein, 14 aa, synthetic construct
  source 1..14: note = Hz. Leu16 (d) VH CDRH3
RSNYYGSSYWFFDV

SEQ ID NO: 95 - protein, 122 aa, synthetic construct
  source 1..122: note = Hz. Leu16 (e) VH
EVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSS

SEQ ID NO: 96 - protein, 5 aa, synthetic construct
  source 1..5: note = Hz. Leu16 (e) VH CDRH1
SYNMH

SEQ ID NO: 97 - protein, 17 aa, synthetic construct
  source 1..17: note = Hz. Leu16 (e) VH CDRH2
AIYPGNGDTSYNQKFKG

SEQ ID NO: 98 - protein, 14 aa, synthetic construct
  source 1..14: note = Hz. Leu16 (e) VH CDRH3
RSNYYGSSYWFFDV

SEQ ID NO: 99 - protein, 122 aa, synthetic construct
  source 1..122: note = Hz. Leu16 (f) VH
EVQLLESGGGLVQPGGSLRLSCAASGFTFTSYNMHWVRQAPGKGLEWVGAIYPGNGDTSYNQKFKGRFTISADNSKNTLYLQMNSLRAEDTAVYYCARSNYYGSSYWFFDVWGQGTTVTVSS

SEQ ID NO: 100 - protein, 5 aa, synthetic construct
  source 1..5: note = Hz. Leu16 (f) VH CDRH1
SYNMH

SEQ ID NO: 101 - protein, 17 aa, synthetic construct
  source 1..17: note = Hz. Leu16 (f) VH CDRH2
AIYPGNGDTSYNQKFKG

SEQ ID NO: 102 - protein, 14 aa, synthetic construct
  source 1..14: note = Hz. Leu16 (f) VH CDRH3
RSNYYGSSYWFFDV

SEQ ID NO: 103 - protein, 106 aa, synthetic construct; identical to SEQ ID NO: 34
  source 1..106: note = Murine anti CD20 Leu16

SEQ ID NO: 104 - protein, 106 aa, synthetic construct
  source 1..106: note = Hz. Leu16(a) VL
DIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIK

SEQ ID NO: 105 - protein, 10 aa, synthetic construct
  source 1..10: note = Hz. Leu16 (a) VL CDRH1
RASSSVNYMD

SEQ ID NO: 106 - protein, 8 aa, synthetic construct
  source 1..8: note = Hz. Leu16 (a) VL CDRH2
YATSNLAS

SEQ ID NO: 107 - protein, 9 aa, synthetic construct
  source 1..9: note = Hz. Leu16 (a) VL CDRH3
QQWSFNPPT

SEQ ID NO: 108 - protein, 106 aa, synthetic construct
  source 1..106: note = Hz. Leu16 (b) VL
EIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIK

SEQ ID NO: 109 - protein, 10 aa, synthetic construct
  source 1..10: note = Hz. Leu16 (b) VL CDRH1
RASSSVNYMD

SEQ ID NO: 110 - protein, 8 aa, synthetic construct
  source 1..8: note = Hz. Leu16 (b) VL CDRH2
YATSNLAS

SEQ ID NO: 111 - protein, 9 aa, synthetic construct
  source 1..9: note = Hz. Leu16 (b) VL CDRH3
QQWSFNPPT

SEQ ID NO: 112 - protein, 60 aa, synthetic construct
  source 1..60: note = Leu16 VH1-46
QVQLVQSGAEVKKPGASVKVSCKASGYTFTSYNMHWVRQAPGQGLEWMGAIYPGNGDTSY

SEQ ID NO: 113 - protein, 60 aa, synthetic construct
  source 1..60: note = Leu16 VH3-23
EVQLLESGGGLVQPGGSLRLSCAASGFTFSSYNMHWVRQAPGKGLEWVSAIYPGNGDTSY

SEQ ID NO: 114 - protein, 60 aa, synthetic construct
  source 1..60: note = Leu16 VH
EVQLQQSGAELVKPGASVKMSCKASGYTFTSYNMHWVKQTPGQGLEWIGAIYPGNGDTSY

SEQ ID NO: 115 - protein, 60 aa, synthetic construct
  source 1..60: note = Leu16 VH1-46
NQKFKGRVTMTRDTSTSTVYMELSSLRSEDTAVYYCARSNYYGSSYWFFDVWGQGTTVTV

SEQ ID NO: 116 - protein, 60 aa, synthetic construct
  source 1..60: note = Leu16 VH3-23
NQKFKGRFTISRDNSKNTLYLQMNSLRAEDTAVYYCARSNYYGSSYWFFDVWGQGTTVTV

SEQ ID NO: 117 - protein, 60 aa, synthetic construct
  source 1..60: note = Leu16 VH
NQKFKGKATLTADKSSSTAYMQLSSLTSEDSADYYCARSNYYGSSYWFFDVWGAGTTVTV

SEQ ID NO: 118 - protein, 60 aa, synthetic construct
  source 1..60: note = Leu16 A11
EIVLTQSPATLSLSPGERATLSCRASSSVNYMDWYQQKPGLAPRLLIYATSNLASGIPDR

SEQ ID NO: 119 - protein, 60 aa, synthetic construct
  source 1..60: note = Leu16 O2
DIQMTQSPSSLSASVGDRVTITCRASSSVNYMDWYQQKPGKAPKLLIYATSNLASGVPSR

SEQ ID NO: 120 - protein, 60 aa, synthetic construct
  source 1..60: note = Leu16 VK
DIVLTQSPAILSASPGEKVTMTCRASSSVNYMDWYQKKPGSSPKPWIYATSNLASGVPAR

SEQ ID NO: 121 - protein, 46 aa, synthetic construct
  source 1..46: note = Leu16 A11
FSGSGSGTDFTLTISRLEPEDFAVYYCQQWSFNPPTFGGGTKVEIK

SEQ ID NO: 122 - protein, 46 aa, synthetic construct
  source 1..46: note = Leu16 O2
FSGSGSGTDFTLTISSLQPEDFATYYCQQWSFNPPTFGGGTKVEIK

SEQ ID NO: 123 - protein, 46 aa, synthetic construct
  source 1..46: note = Leu16 VK
FSGSGSGTSYSLTISRVEAEDAATYYCQQWSFNPPTFGGGTKLEIK

SEQ ID NO: 124 - protein, 677 aa, synthetic construct
  source 1..677: note = CC373 murine Leu16 VL_VH CAR T
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 677 aa, synthetic construct
  source 1..677: note = CC374_Hz.Leu16_ O2/VH1-46 VL_VH CAR T
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSNYYGSSYWFFDVWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 677 aa, synthetic construct
  source 1..677: note = CC375_ Hz.Leu16_ O2_VH1-46(73K) VL_VH CAR T
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDKSTSTVYMELSSLRSEDTAVYYCARSNYYGSSYWFFDVWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 677 aa, synthetic construct
  source 1..677: note = CC376_ Hz.Leu16_ 02_VH3-23 VL_VH CAR T
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCARSNYYGSSYWFFDVWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 677 aa, synthetic construct
  source 1..677: note = CC377_Hz.Leu16_ O2_VH3-23(30T) VL_VH CAR T
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFTSYNMHWVRQAPGKGLEWVSAIYPGNGDTSYNQKFKGRFTISRDNSKNTLYLQMNSLRAEDTAVYYCARSNYYGSSYWFFDVWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 677 aa, synthetic construct
  source 1..677: note = CC378- Hz.Leu16_ 02_VH3-3(30T.49G) VL_VH CAR T
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 677 aa, synthetic construct
  source 1..677: note = CC379_Hz.Leu16_O2_VH3-23(30T.49G.71A) VL_VH CAR T
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISADNSKNTLYLQMNSLRAEDTAVYYCARSNYYGSSYWFFDVWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 677 aa, synthetic construct
  source 1..677: note = CC380_Hz.Leu16_ A11_VH1-46 VL_VH CAR T
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSNYYGSSYWFFDVWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 677 aa, synthetic construct
  source 1..677: note = CC381-Hz.Lue16_ A11/VH1-46(73K) VL_VH CAR T
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDKSTSTVYMELSSLRSEDTAVYYCARSNYYGSSYWFFDVWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 677 aa, synthetic construct
  source 1..677: note = CC382_ Hz.Leu16_A11/VH3-23 VL_VH CAR T
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCARSNYYGSSYWFFDVWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 677 aa, synthetic construct
  source 1..677: note = CC383_HZ.Lue16_A11_VH3-23(30T) VL_VH CAR T
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVSAIYPGNGDTSYNQKFKGRFTISRDNSKNTLYLQMNSLRAEDTAVYYCARSNYYGSSYWFFDVWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 677 aa, synthetic construct
  source 1..677: note = CC384- HZ.Leu16_ A11/VH3-23(30T.49G) VL_VH CAR T
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 677 aa, synthetic construct
  source 1..677: note = CC385-Hz.Lue16_A11/VH3-23(30T.49G.71A) VL_VH CAR T
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISADNSKNTLYLQMNSLRAEDTAVYYCARSNYYGSSYWFFDVWGQGTTVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 20 aa, synthetic construct
  source 1..20: note = mIgK leader Sequence
METDTLLLWVLLLWVPGSTG

SEQ ID NO: 138 - protein, 18 aa, synthetic construct
  source 1..18: note = GS(18) Linker
GSTSGGGSGGGSGGGGSS

SEQ ID NO: 139 - protein, 26 aa, synthetic construct
  source 1..26: note = CD28 TM
FWVLVVVGGVLACYSLLVTVAFIIFW

SEQ ID NO: 140 - protein, 43 aa, synthetic construct
  source 1..43: note = 41BB
VKRGRKKLLYIFKQPFMRPVQTTQEEDGCSCRFPEEEEGGCEL

SEQ ID NO: 141 - protein, 112 aa, synthetic construct; identical to SEQ ID NO: 33
  source 1..112: note = CD3z

SEQ ID NO: 142 - protein, 120 aa, synthetic construct; identical to SEQ ID NO: 36
  source 1..120: note = Murine anti CD19 FMC63VH sequence

SEQ ID NO: 143 - protein, 5 aa, synthetic construct
  source 1..5: note = CDRH1
DYGVS

SEQ ID NO: 144 - protein, 16 aa, synthetic construct
  source 1..16: note = CDRH2
VIWGSETTYYNSALKS

SEQ ID NO: 145 - protein, 12 aa, synthetic construct
  source 1..12: note = CDRH3
HYYYGGSYAMDY

SEQ ID NO: 146 - protein, 60 aa, synthetic construct
  source 1..60: note = VH4-59 (a)
QVQLQESGPGLVKPSETLSLTCTVSGGSISSYYWSWIRQPPGKGLEWIGYIYYSGSTNYN

SEQ ID NO: 147 - protein, 60 aa, synthetic construct
  source 1..60: note = FMC63 VH (a)
EVKLQESGPGLVAPSQSLSVTCTVSGVSLPDYGVSWIRQPPRKGLEWLGVIWGSETTYYN

SEQ ID NO: 148 - protein, 60 aa, synthetic construct
  source 1..60: note = Hz FMC63 VH4-59 (a)
QVQLQESGPGLVKPSETLSLTCTVSGGSISDYGVSWIRQPPGKGLEWIGVIWGSETTYYN

SEQ ID NO: 149 - protein, 37 aa, synthetic construct
  source 1..37: note = VH4-59 (b)
PSLKSRVTISVDTSKNQFSLKLSSVTAADTAVYYCAR

SEQ ID NO: 150 - protein, 60 aa, synthetic construct
  source 1..60: note = FMC63 VH (b)
SALKSRLTIIKDNSKSQVFLKMNSLQTDDTAIYYCAKHYYYGGSYAMDYWGQGTSVTVSS

SEQ ID NO: 151 - protein, 60 aa, synthetic construct
  source 1..60: note = Hz FMC63 VH4-59 (b)
SALKSRVTISVDTSKNQFSLKLSSVTAADTAVYYCAKHYYYGGSYAMDYWGQGTLVTVSS

SEQ ID NO: 152 - protein, 60 aa, synthetic construct
  source 1..60: note = Hz FMC63 VH4-59 contact
QVQLQESGPGLVKPSETLSLTCTVSGGSIPDYGVSWIRQPPGKGLEWLGVIWGSETTYYN

SEQ ID NO: 153 - protein, 60 aa, synthetic construct; identical to SEQ ID NO: 147
  source 1..60: note = FMC63

SEQ ID NO: 154 - protein, 60 aa, synthetic construct
  source 1..60: note = Hz FMC63 VH4-59 contact
PSLKSRLTISVDTSKNQFSLKLSSVTAADTAVYYCAKHYYYGGSYAMDYWGQGTLVTVSS

SEQ ID NO: 155 - protein, 60 aa, synthetic construct; identical to SEQ ID NO: 150
  source 1..60: note = FMC63 VH

SEQ ID NO: 156 - protein, 107 aa, synthetic construct; identical to SEQ ID NO: 37
  source 1..107: note = Murine anti CD19 FMC63VK

SEQ ID NO: 157 - protein, 60 aa, synthetic construct
  source 1..60: note = O2-VK
DIQMTQSPSSLSASVGDRVTITCRASQSISSYLNWYQQKPGKAPKLLIYAASSLQSGVPS

SEQ ID NO: 158 - protein, 60 aa, synthetic construct
  source 1..60: note = FMC63 VK
DIQMTQTTSSLSASLGDRVTISCRASQDISKYLNWYQQKPDGTVKLLIYHTSRLHSGVPS

SEQ ID NO: 159 - protein, 60 aa, synthetic construct
  source 1..60: note = Hz FMC63 VK-O2
DIQMTQSPSSLSASVGDRVTITCRASQDISKYLNWYQQKPGKAPKLLIYHTSRLHSGVPS

SEQ ID NO: 160 - protein, 35 aa, synthetic construct
  source 1..35: note = O2-VK (b)
RFSGSGSGTDFTLTISSLQPEDFATYYCQQSYSTP

SEQ ID NO: 161 - protein, 47 aa, synthetic construct
  source 1..47: note = FMC63 VK (b)
RFSGSGSGTDYSLTISNLEQEDIATYFCQQGNTLPYTFGGGTKLEIT

SEQ ID NO: 162 - protein, 47 aa, synthetic construct
  source 1..47: note = Hz FMC63 VK-O2 (b)
RFSGSGSGTDYTLTISSLQPEDFATYYCQQGNTLPYTFGGGVKLEIK

SEQ ID NO: 163 - protein, 459 aa, synthetic construct
  source 1..459: note = FMC63-9 HzVH4-59_HzVKO2
METDTLLLWVLLLWVPGSTGQVQLQESGPGLVKPSETLSLTCTVSGGSISDYGVSWIRQPPGKGLEWIGVIWGSETTYYNSALKSRVTISVDTSKNQFSLKLSSVTAADTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGVKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - protein, 459 aa, synthetic construct
  source 1..459: note = FMC63-10 HzVH4-59(L29P30)_HzVKO2
METDTLLLWVLLLWVPGSTGQVQLQESGPGLVKPSETLSLTCTVSGGSLPDYGVSWIRQPPGKGLEWIGVIWGSETTYYNSALKSRVTISVDTSKNQFSLKLSSVTAADTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGVKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 165 - protein, 459 aa, synthetic construct
  source 1..459: note = FMC63-11 HzVH4-59(Con)_HzVKO2
METDTLLLWVLLLWVPGSTGQVQLQESGPGLVKPSETLSLTCTVSGGSIPDYGVSWIRQPPGKGLEWLGVIWGSETTYYNPSLKSRLTISVDTSKNQVSLKLSSVTAADTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGVKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - protein, 459 aa, synthetic construct
  source 1..459: note = FMC63-12 HzVH3-23_HzVKO2
METDTLLLWVLLLWVPGSTGEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGVKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 459 aa, synthetic construct
  source 1..459: note = FMC63-13 HzVH3-23(KV)_HzVKO2
METDTLLLWVLLLWVPGSTGEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGVKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 459 aa, synthetic construct
  source 1..459: note = Hz. FMC63-16: VH3-23; VKO2
METDTLLLWVLLLWVPGSTGEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - protein, 459 aa, synthetic construct
  source 1..459: note = Hz. FMC63-17: VH3-23; VKO2(L87F)
METDTLLLWVLLLWVPGSTGEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYFCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - protein, 459 aa, synthetic construct
  source 1..459: note = Hz. FMC63-18: VH3-23; VKO2(L22S)
METDTLLLWVLLLWVPGSTGEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTISCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - protein, 459 aa, synthetic construct
  source 1..459: note = Hz. FMC63-19: VH3-23; VKO2(L82I)
METDTLLLWVLLLWVPGSTGEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDIATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 172 - protein, 459 aa, synthetic construct
  source 1..459: note = Hz. FMC63-20: VH3-23(H71,87); VKO2
METDTLLLWVLLLWVPGSTGEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 173 - protein, 459 aa, synthetic construct
  source 1..459: note = Hz. FMC63-21: VH3-23(H71,87); VKO2(L87F)
METDTLLLWVLLLWVPGSTGEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYFCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 174 - protein, 459 aa, synthetic construct
  source 1..459: note = Hz. FMC63-22: VH3-23(H71,87); VKO2(L22S)
METDTLLLWVLLLWVPGSTGEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTISCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 175 - protein, 459 aa, synthetic construct
  source 1..459: note = Hz. FMC63-23: VH3-23(H71,87); VKO2(L82I)
METDTLLLWVLLLWVPGSTGEVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDIATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 176 - protein, 20 aa, synthetic construct
  source 1..20: note = mIgK leader Sequence
METDTLLLWVLLLWVPGSTG

SEQ ID NO: 177 - protein, 18 aa, synthetic construct
  source 1..18: note = Whitlow Linker
GSTSGSGKPGSGEGSTKG

SEQ ID NO: 178 - protein, 12 aa, synthetic construct
  source 1..12: note = IgG4 Hinge
ESKYGPPCPPCP

SEQ ID NO: 179 - protein, 27 aa, synthetic construct
  source 1..27: note = CD28 TM
FWVLVVVGGVLACYSLLVTVAFIIFWV

SEQ ID NO: 180 - protein, 42 aa, synthetic construct; identical to SEQ ID NO: 32
  source 1..42: note = 41BB

SEQ ID NO: 181 - protein, 112 aa, synthetic construct; identical to SEQ ID NO: 33
  source 1..112: note = CD3z

SEQ ID NO: 182 - protein, 725 aa, synthetic construct
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGVKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 183 - protein, 725 aa, synthetic construct
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGVKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 184 - protein, 725 aa, synthetic construct
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGVKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 185 - protein, 725 aa, synthetic construct
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDKSTSTVYMELSSLRSEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGVKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 186 - protein, 725 aa, synthetic construct
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGVKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 187 - protein, 725 aa, synthetic construct
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISADNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGVKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 188 - protein, 725 aa, synthetic construct
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 189 - protein, 725 aa, synthetic construct
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 190 - protein, 725 aa, synthetic construct
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 191 - protein, 725 aa, synthetic construct
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDKSTSTVYMELSSLRSEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 192 - protein, 725 aa, synthetic construct
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 193 - protein, 725 aa, synthetic construct
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISADNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 194 - protein, 725 aa, synthetic construct
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTISCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 195 - protein, 725 aa, synthetic construct
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTISCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 196 - protein, 725 aa, synthetic construct
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTISCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 197 - protein, 725 aa, synthetic construct
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDKSTSTVYMELSSLRSEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTISCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 198 - protein, 725 aa, synthetic construct
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTISCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 199 - protein, 725 aa, synthetic construct
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISADNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTISCRASQDISKYLNWYQQKPGKAPKLLIYHTSRLHSGVPSRFSGSGSGTDYTLTISSLQPEDFATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 200 - protein, 725 aa, synthetic construct
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDIATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 201 - protein, 725 aa, synthetic construct
METDTLLLWVLLLWVPGSTGDIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDIATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 202 - protein, 725 aa, synthetic construct; identical to SEQ ID NO: 52

SEQ ID NO: 203 - protein, 725 aa, synthetic construct; identical to SEQ ID NO: 53

SEQ ID NO: 204 - protein, 725 aa, synthetic construct
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDIATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 205 - protein, 725 aa, synthetic construct
METDTLLLWVLLLWVPGSTGEIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISADNSKNTLYLQMNSLRAEDTAVYYCARSNYYGSSYWFFDVWGQGTTVTVSSGGGGSGGGGSGGGGSGGGGSEVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDIATYYCQQGNTLPYTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 206 - protein, 725 aa, synthetic construct; identical to SEQ ID NO: 60

SEQ ID NO: 207 - protein, 725 aa, synthetic construct; identical to SEQ ID NO: 61

SEQ ID NO: 208 - protein, 725 aa, synthetic construct; identical to SEQ ID NO: 62

SEQ ID NO: 209 - protein, 725 aa, synthetic construct; identical to SEQ ID NO: 63

SEQ ID NO: 210 - protein, 725 aa, synthetic construct; identical to SEQ ID NO: 64

SEQ ID NO: 211 - protein, 725 aa, synthetic construct; identical to SEQ ID NO: 65

SEQ ID NO: 212 - protein, 725 aa, synthetic construct; identical to SEQ ID NO: 66

SEQ ID NO: 213 - protein, 725 aa, synthetic construct; identical to SEQ ID NO: 67

SEQ ID NO: 214 - protein, 725 aa, synthetic construct; identical to SEQ ID NO: 68

SEQ ID NO: 215 - protein, 725 aa, synthetic construct; identical to SEQ ID NO: 70

SEQ ID NO: 216 - protein, 725 aa, synthetic construct; identical to SEQ ID NO: 71

SEQ ID NO: 217 - protein, 725 aa, synthetic construct; identical to SEQ ID NO: 72

SEQ ID NO: 218 - protein, 245 aa, synthetic construct
  source 1..245: note = FMC63-12
EVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGVKLEIK

SEQ ID NO: 219 - protein, 245 aa, synthetic construct
  source 1..245: note = Hz. FMC63-16: VH3-23; VKO2
EVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIK

SEQ ID NO: 220 - protein, 245 aa, synthetic construct
  source 1..245: note = Hz. FMC63-18: VH3-23; VKO2(L22S)
EVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTISCRASQDISKYLNWYQQKPGKAPKLLIYHTSRLHSGVPSRFSGSGSGTDYTLTISSLQPEDFATYYCQQGNTLPYTFGGGTKVEIK

SEQ ID NO: 221 - protein, 245 aa, synthetic construct
  source 1..245: note = Hz. FMC63-19: VH3-23; VKO2(L82I)
EVQLLESGGGLVQPGGSLRLSCAASGFTFSDYGVSWVRQAPGKGLEWVSVIWGSETTYYNSALKSRFTISRDNSKNTLYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDIATYYCQQGNTLPYTFGGGTKVEIK

SEQ ID NO: 222 - protein, 245 aa, synthetic construct
  source 1..245: note = Hz. FMC63-20: VH3-23(H71,87); VKO2
EVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIK

SEQ ID NO: 223 - protein, 245 aa, synthetic construct
  source 1..245: note = Hz. FMC63-21: VH3-23(H71,87); VKO2(L87F)
EVQLLESGGGLVQPGGSLRLSCAASGFTFSDYGVSWVRQAPGKGLEWVSVIWGSETTYYNSALKSRFTISKDNSKNTVYLQMNSLRAEDTAVYYCAKHYYYGGSYAMDYWGQGTLVTVSSGSTSGSGKPGSGEGSTKGDIQMTQSPSSLSASVGDRVTITCRASQDISKYLNWYQQKPGKAPKLLIYHTSRLHSGVPSRFSGSGSGTDYTLTISSLQPEDFATYFCQQGNTLPYTFGGGTKVEIK

SEQ ID NO: 224 - protein, 246 aa, synthetic construct
  source 1..246: note = Hz.Leu16: 374
DIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSNYYGSSYWFFDVWGQGTTVTVSS

SEQ ID NO: 225 - protein, 246 aa, synthetic construct
  source 1..246: note = Hz.Leu16: 376
DIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCARSNYYGSSYWFFDVWGQGTTVTVSS

SEQ ID NO: 226 - protein, 246 aa, synthetic construct
  source 1..246: note = Hz.Leu16 :378
DIQMTQSPSSLSASVGDRVTITCRASSSVNYMDWYQQKPGKAPKLLIYATSNLASGVPSRFSGSGSGTDFTLTISSLQPEDFATYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSS

SEQ ID NO: 227 - protein, 246 aa, synthetic construct
  source 1..246: note = Hz.Leu16: 381
EIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSQVQLVQSGAEVKKPGASVKVSCKASGYTFTSYNMHWVRQAPGQGLEWMGAIYPGNGDTSYNQKFKGRVTMTRDKSTSTVYMELSSLRSEDTAVYYCARSNYYGSSYWFFDVWGQGTTVTVSS

SEQ ID NO: 228 - protein, 246 aa, synthetic construct
  source 1..246: note = CC384- HZ.Leu16_ A11/VH3-23(30T.49G) VL_VH CAR T
EIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISRDNSKNTLYLQMNSLRAEDTAVYYCARSNYYGSSYWFFDVWGQGTTVTVSS

SEQ ID NO: 229 - protein, 246 aa, synthetic construct
  source 1..246: note = CC385-Hz.Lue16_A11/VH3-23(30T.49G.71A) VL_VH CAR T
EIVLTQSPATLSLSPGERATLSCRASSSVNYMDWYQQKPGLAPRLLIYATSNLASGIPDRFSGSGSGTDFTLTISRLEPEDFAVYYCQQWSFNPPTFGGGTKVEIKGSTSGGGSGGGSGGGGSSEVQLLESGGGLVQPGGSLRLSCAASGFTFTSYNMHWVRQAPGKGLEWVGAIYPGNGDTSYNQKFKGRFTISADNSKNTLYLQMNSLRAEDTAVYYCARSNYYGSSYWFFDVWGQGTTVTVSS

SEQ ID NO: 230 - protein, 245 aa, synthetic construct
  source 1..245: note = CD20 scFV
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKKGKATLTADKSSSTAYMQLSSLTSEDSADYYCARSNYYGSSYWFFDVWGAGTTVTVSS

SEQ ID NO: 231 - protein, 245 aa, synthetic construct
  source 1..245: note = CD19 scFV
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 232 - protein, 12 aa, synthetic construct
  source 1..12: note = IgG4 Hinge
ESKYGPPCPPCP

SEQ ID NO: 233 - protein, 26 aa, synthetic construct
  source 1..26: note = CD28 transmembrane domain
FWVLVWGGVLACYSLLVTVAFIIFWV

SEQ ID NO: 234 - protein, 41 aa, synthetic construct
  source 1..41: note = CD28 cytoplasmic domain
RSKRSRGGHSDYMNMTPRRPGPTRKHYQPYAPPRDFAAYRS

SEQ ID NO: 235 - protein, 42 aa, synthetic construct; identical to SEQ ID NO: 32
  source 1..42: note = 4-1BB cytoplasmic domain

SEQ ID NO: 236 - protein, 112 aa, synthetic construct; identical to SEQ ID NO: 33

SEQ ID NO: 237 - protein, 24 aa, synthetic construct
  source 1..24: note = T2A self-cleaving peptide
LEGGGEGRGSLLTCGDVEENPGPR

SEQ ID NO: 238 - protein, 108 aa, synthetic construct
  source 1..108: note = IgG4 CH2
APEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAK

SEQ ID NO: 239 - protein, 107 aa, synthetic construct
  source 1..107: note = IgG4 CH3
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 240 - protein, 355 aa, synthetic construct
  source 1..355: note = Truncated epidermal growth factor receptor (EGFRt)
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAWSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLWALGIGLFM

(19) United States
(12) Patent Application Publication
(10) Pub. No.: US 2026/0152553 A1
(43) Pub. Date: Jun. 4, 2026

(54) BISPECIFIC CARS FOR THE TREATMENT OF LUPUS AND AUTOIMMUNE DISEASES

(71) Applicant: LYELL IMMUNOPHARMA, INC., South San Francisco, CA (US)

(72) Inventors: Orit Foord; James Johnston, West Hills, CA (US); Jenny Jiajia Cui, West Hills, CA (US)

(21) Appl. No.: 18/707,423

(22) PCT Filed: Mar. 13, 2024
(86) PCT No.: PCT/US2024/019750

§ 371 Date: May 3, 2024

Related U.S. Application Data
(60) Provisional application No. 63/451,912, filed on Mar. 13, 2023.
(60) Provisional application No. 63/451,914, filed on Mar. 13, 2023.
(60) Provisional application No. 63/451,917, filed on Mar. 13, 2023.

(51) Int. Cl. C07K 16/28; A61K 40/11; A61K 40/31; A61K 40/41; C07K 14/705; C07K 14/725; C12N 5/0783

(52) U.S. Cl. CPC C07K 16/2803; A61K 40/11; A61K 40/31; A61K 40/416; C07K 14/7051; C07K 14/70578; C07K 16/2887; C12N 5/0636; C07K 2317/24; C07K 2317/31; C07K 2317/53; C07K 2317/622; C07K 2319/02; C07K 2319/03; C12N 2510/00

(57) ABSTRACT

The present invention relates to novel Chimeric Antigen Receptor (CAR) constructs, including CAR-T constructs, compositions for use with said constructs, and methods of treating patients with cancer and autoimmune diseases.

### CROSS REFERENCE TO RELATED APPLICATIONS

This application claims priority to U.S. Provisional Application Nos. 63/451,912, 63/451,914, and 63/451,917, all of which were filed on Mar. 13, 2023, the contents of which are incorporated herein by reference in their entirety.

### REFERENCE TO AN ELECTRONIC SEQUENCE LISTING

The instant application contains a Sequence Listing, which has been submitted electronically in .xml format. The contents of the electronic sequence listing (67941WO_SL.xml; Size: 322,384 bytes; and Date of Creation: Mar. 11, 2024) is herein incorporated by reference in its entirety.

### INCORPORATION BY REFERENCE

All publications, patents, and patent applications cited herein are incorporated by reference to the same extent as if each individual publication, patent, or patent application was specifically and individually indicates to be incorporated by reference. In the event of a conflict between a term herein and a term in an incorporated reference, the term herein controls.

### FIELD

The present disclosure relates to novel Chimeric Antigen Receptor (CAR) constructs for the treatment of autoimmune diseases such as Lupus, Lupus Nephritis, Idiopathic inflammatory myopathies, Anca Associated Vasculitis, Systemic Sclerosis, and MS and methods of treatment comprising administration of said CARs.

### BACKGROUND

The present invention relates to novel bispecific CD19/CD20 CAR constructs and their methods of use. The invention further relates to plasmids, vectors and cell populations comprising these bispecific CD 19/CD20 CAR constructs and their methods of use in the treatment of a variety of diseases, including but not limited to, cancer, as well as autoimmune diseases such as lupus.

Systemic lupus erythematosus (SLE) is a chronic inflammatory disease due to loss of tolerance to self-antigens, characterized by the presence of autoantibodies to DNA and nuclear antigens, resulting in immune dysregulation associated with over-activation of B and T cells. SLE is the most common type of lupus, and is an autoimmune disease in which the immune system attacks its own tissues, causing widespread inflammation and tissue damage in the affected organs. It can affect the joints, skin, brain, lungs, kidneys, and blood vessels.

SLE is a heterogenous disease with complex pathogenesis involving both the innate and adaptive immune systems and resulting in inflammation of multiple organs including the skin, kidneys, joints, blood vessels, heart, lung, and nervous system. Patients with SLE experience a variety of symptoms that include fatigue, fever, weight changes, skin rashes, oral ulcers, arthritis, and cognitive dysfunction (Barber, 2021). The pathologic findings include immune complex deposition and infiltration by lymphocytes and neutrophils (Tsokos, 2011).

Lupus nephritis (LN) is one of the severe clinical manifestations of SLE. There is a high incidence of LN with >80,000 cases in the United States. Renal disease is diagnosed with detection of proteinuria and classified by histologic findings in kidney biopsies. LN classifications include minimal mesangial (I), mesangial proliferative (II), focal (Ill), diffuse (IV), membranous (V) and advanced sclerosing lupus nephritis (VI) (Weening 2004). Life expectancy for LN patients depends on symptom severity and response to medication. Standard-of-care treatment is administered in an induction phase followed by a maintenance phase. Immunosuppressive agents are included the cornerstone of both induction and maintenance phases and may include combinations of corticosteroids, mycophenolic acid, cyclophosphamide, azathioprine, and calcineurin inhibitors. Approximately 10-20% of patients with LN progress to end-stage kidney disease and may require hemodialysis or kidney transplantation.

CD19-targeting chimeric antigen receptor (CAR) T cell therapy has revolutionized the management of B-cell malignancies in recent years (Schroeder, 2022), with around 40% durable complete remissions even in heavily pretreated patients with relapse/refractory (r/r) B-cell malignancies including recent approvals as second-line therapies. Despite this, a recent study concluded that among real-world CD19 CAR-T treated DLBCL patients the risk of not achieving durable remission is considerable and effective salvage treatments are needed (Jalbert, 2022). Since CD19 antigen loss is frequently associated with relapse following CD19-targeted CAR T therapy (Spiegel, 2021), dual-antigen or multi-antigen targeting by CAR T cells may result in synergistic responses, thereby optimizing response rates and providing potential salvage options (Simon, 2020; Leonie, 2021).

The invention further relates to novel manufacturing methods for such CARs. Previously, the manufacturing of, e.g., CD19/CD20 bispecific CAR-Ts involved steps which could benefit from certain improvements. For example, a CD14+CD25+ depletion step where the percentage of CD14+ and/or CD25+ cells within a CD62L+ population was greater than 5%. The present invention has multiple advantages over this, including e.g., obviating the need for such a step.

Additionally, prior manufacturing processes relied upon open manufacturing steps (open system), including centrifugation, which made them vulnerable to contamination, reduced cellular function, and reduced manufacturing yield. Further, in prior methods, the duration of cell stimulation was reported as needed for 32-48 hours.

Previously, Cryostor CS5™ was a preferred cryopreservation medium, and epHIV7 CD 19/20 tEGFR was a preferred vector.

In prior methods, the MOI has been reported as 1.5 (and perhaps higher for second lot measurements). Improvement of this MOI is warranted.

Moreover, previous techniques incorporated the use of protamine sulfate as a transduction enhancer. This has proved problematic in that we have discovered that the cells expanded at a slower rate and the yield of CAR T positive cells would therefore be compromised in other methods that utilized transduction enhancers such as protamine sulphate. Moreover the use of such enhancers would necessitate the employment of methods to prove that the enhancer would be diluted to safe levels in the final product. This the removal of enhancers such as protamine sulfate both simplifies and improves the process. Addition of protamine sulfate during transduction was found to not improve transduction efficiency and in fact impedes expansion of T cells (see FIG. 10). T cell expansion was found to decrease from 30-fold to 25-fold in the presence of protamine sulfate.

There thus exists a need for improved treatments for SLE, Lupus Nephritis, Idiopathic inflammatory myopathies, Anca Associated Vasculitis, Systemic Sclerosis and MS and other autoimmune diseases.

### SUMMARY OF THE INVENTION

The invention relates to, among other things, bispecific CD19/CD20 CARs, including CAR-Ts, for the treatment of autoimmune diseases such as Lupus or SLE. Preferably, the CAR-Ts treat SLE, Idiopathic inflammatory myopathies, Anca Associated Vasculitis, Systemic Sclerosis and MS.

Specifically, the invention relates to methods of treating Lupus in a patient comprising administering to said patient an effective amount of an anti-CD19/CD20 engineered immune cell.

In some embodiments of the above aspects, the disclosure provides a polypeptide comprising a CD19/CD20 chimeric antigen receptor (CAR), the CAR comprising: an anti-CD20 scFv comprising (i) a light chain variable region having the amino acid sequence of SEQ ID NO: 34, and (ii) a heavy chain variable region having the amino acid sequences of SEQ ID NO: 35; a (G₄S)ₙ linker, wherein n is 1, 2, 3, or 4; an anti-CD19 scFv comprising (i) a heavy chain variable region having the amino acid sequence of SEQ ID NO: 36, and (ii) a light chain variable region having the amino acid sequences of SEQ ID NO: 37; a spacer domain comprising the amino acid sequence of any one selected from SEQ ID NOs: 21-29; a transmembrane domain comprising the amino acid sequence of SEQ ID NO: 30 or 31; a 4-1BB cytoplasmic signaling domain comprising the amino acid sequence of SEQ ID NO: 32; and a CD3 zeta signaling domain comprising the amino acid sequence of SEQ ID NO: 33.

In some embodiments of the above aspects, the disclosure provides at least one of the anti-CD20 scFv or anti-CD19 scFv comprises at least one CDR sequence isolated or derived from a nonhuman subject.

In some embodiments of the above aspects, the disclosure provides that the CAR comprises the formula from N terminus to C terminus: [anti-CD20 scFv]-[(G₄S)ₙ linker]-[anti-CD19 scFv]-[spacer domain]-[transmembrane domain]-[4-1BB cytoplasmic signaling domain]-[CD3 zeta signaling domain].

In some embodiments of the above aspects, the disclosure provides that the (G₄S)ₙ linker is (G₄S)₁. In some aspects, the (G₄S)₁ comprises the amino acid sequence of SEQ ID NO: 48.

In some embodiments of the above aspects, the disclosure provides that the (G₄S)ₙ linker is (G₄S)₂. In some aspects, the (G₄S)₂ comprises the amino acid sequence of SEQ ID NO: 49.

In some embodiments of the above aspects, the disclosure provides that the (G₄S)ₙ linker is (G₄S)₃. In some aspects, the (G₄S)₃ comprises the amino acid sequence of SEQ ID NO: 50.

In some embodiments of the above aspects, the disclosure provides that the (G₄S)ₙ linker is (G₄S)₄. In some aspects, the (G₄S)₄ comprises the amino acid sequence of SEQ ID NO: 51.

In another aspect, the disclosure provides a polypeptide comprising a CD19/CD20 chimeric antigen receptor (CAR), wherein the CAR comprises the amino acid sequence selected from the group consisting of SEQ ID NOs: 2, 4, 6, 8, 10, 12, 14, 16, 18, and 20.

In another aspect, the disclosure provides a polypeptide comprising a CD19 chimeric antigen receptor (CAR), comprising: an anti-CD19 scFv comprising (i) a heavy chain variable region having the amino acid sequence of SEQ ID NO: 36, and (ii) a light chain variable region having the amino acid sequences of SEQ ID NO: 37; a spacer domain comprising the amino acid sequence of any one selected from SEQ ID NOs: 21-29; a transmembrane domain comprising the amino acid sequence of SEQ ID NO: 30 or 31; a 4-1BB cytoplasmic signaling domain comprising the amino acid sequence of SEQ ID NO: 32; and a CD3 zeta signaling domain comprising the amino acid sequence of SEQ ID NO: 33.

In another aspect, the CAR comprises the formula from N terminus to C terminus: [anti-CD19 scFv]-[spacer domain]-[transmembrane domain]-[4-1BB cytoplasmic signaling domain]-[CD3 zeta signaling domain].

One aspect of the disclosure is a nucleic acid encoding any one of the disclosed polypeptides. One aspect of the disclosure is a plasmid comprising said nucleic acid. One aspect of the disclosure is a vector comprising said nucleic acid. One aspect of the disclosure is a cell comprising said nucleic acid.

One aspect of the disclosure is a cell comprising any one of the disclosed polypeptides. In an aspect, the cell is an immune cell. In an aspect, the immune cell is selected from the group consisting of T cell, NK cell, and Treg cell.

One aspect of the disclosure is a method of treating an autoimmune disease in a patient in need thereof, comprising administering to the patient an effective amount of a population of any of the disclosed cells. In an aspect, the population of cells are immune cells.

One aspect of the disclosure is a method of treating an autoimmune disease in a patient in need thereof, the method comprising: obtaining immune cells from a patient; engineering the immune cells, wherein the engineered immune cells comprise any one of the disclosed polypeptides; expanding the engineered immune cells; and administering an effective amount of the expanded engineered immune cells to the patient.

One aspect of the disclosure is a method of treating an autoimmune disease in a patient comprising administering to said patient an effective amount of an anti-CD19/CD20 engineered immune cells, wherein said engineered immune cells comprises the chimeric antigen receptor set forth in SEQ ID NO. 2. In an aspect, the immune cells are selected from the group consisting of T cell, NK cell, and Treg cell.

In some further embodiments of the above aspect, a method of treating an autoimmune disease in a patient in need thereof, comprising administering to the patient an effective amount of an engineered T cell, the engineered T cell comprising a polypeptide comprising a CD19/CD20 chimeric antigen receptor (CAR), the CAR comprising: an anti-CD20 scFv comprising (i) a light chain variable region having the amino acid sequence of SEQ ID NO: 34, and (ii) a heavy chain variable region having the amino acid sequences of SEQ ID NO: 35; a (G₄S)ₙ linker, wherein n is 1, 2, 3, or 4; an anti-CD19 scFv comprising (i) a heavy chain variable region having the amino acid sequence of SEQ ID NO: 36, and (ii) a light chain variable region having the amino acid sequences of SEQ ID NO: 37; a spacer domain comprising the amino acid sequence of any one selected from SEQ ID NOs: 21-29; a transmembrane domain comprising the amino acid sequence of SEQ ID NO: 30 or 31; a 4-1BB cytoplasmic signaling domain comprising the amino acid sequence of SEQ ID NO: 32; and a CD3 zeta signaling domain comprising the amino acid sequence of SEQ ID NO: 33.

In another aspect, the effective amount is between about 1×10⁶ and about 2×10⁸ cells. In another aspect, the effective amount is about 2×10⁶ cells, alternatively about 3×10⁶ cells, alternatively about 4×10⁶ cells, alternatively about 5×10⁶ cells, alternatively about 6×10⁶ cells, alternatively about 7×10⁶ cells, alternatively about 8×10⁶ cells, alternatively about 9×10⁶ cells, alternatively about 1×10⁷ cells, alternatively about 2×10⁷ cells, alternatively about 3×10⁷ cells, alternatively about 4×10⁷ cells, alternatively about 5×10⁷ cells, alternatively about 6×10⁷ cells, alternatively about 7×10⁷ cells, alternatively about 8×10⁷ cells, alternatively about 9×10⁷ cells, or alternatively about 1×10⁸ cells.

In another aspect, the disclosure provides a method of treating an autoimmune disease in a patient comprising administering to the patient an effective amount of an engineered T cell, the engineered T cell comprising a polypeptide comprising a CD19-OR-CD20 chimeric antigen receptor (CAR), wherein the CAR comprises from N-terminus to C-terminus: an anti-CD20 scFv with a variable heavy domain and a variable light domain obtained and/or derived from ofatumumab; a (G₄S)ₙ linker, wherein n is 1, 3, or 4; an anti-CD19 scFv comprising a variable heavy domain and a variable light domain; a spacer; a transmembrane domain; a co-stimulatory domain; and a CD3-zeta cytoplasmic signaling domain.

In some further embodiments of the above aspects, the autoimmune disease is selected from the group consisting of lupus, rheumatoid arthritis (RA), systemic lupus erythematosus (SLE), inflammatory bowel disease (IBD/Crohn's), Type 1 diabetes mellitus, Guillain-Barre syndrome, chronic inflammatory demyelinating polyneuropathy, psoriasis, Graves' Disease, Hashimoto's Thyroiditis, Myasthenia Gravis, scleroderma, systemic sclerosis, Multiple Sclerosis (MS), autoimmune neuropathy, transverse myelitis, optic neuritis, neuromyelitis optica, acute disseminated encephalomyelitis, autoimmune or paraneoplastic encephalitis, and spasticity, Idiopathic inflammatory myopathies, Anca Associated Vasculitis, and Systemic Sclerosis.

In some embodiments of the above aspects, the autoimmune disease is lupus, SLE, Idiopathic inflammatory myopathies, Anca Associated Vasculitis, Systemic Sclerosis and MS.

In some embodiments of the above aspects, the autoimmune disease is RA, IBD, or psoriasis.

Other aspects and embodiments of the disclosure will be apparent to one of skill in the art in light of the disclosure and illustrative examples that follow.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows a graphical representation of an exemplary CAR-T suitable for use in the present invention.

FIG. 2A shows % positive CD62L and CD45RA cells. T-cells were harvested on day 12, at the end of the process and then stained for CD45RA and CD62L. Data is representative of 6 donors. Greater than SO % of the cells were either central memory (predominant population) or nai:ve/stem cell memory cells, consistent with selection of CD62L+ cells on day 1.

FIG. 2B shows % positive CD4+ and CD8+ cells. T cells were harvested on day 12 and stained with anti-CD4 and anti-CD8 antibodies. Data is a summary of 6 donors, mean is represented by the horizontal line.

FIG. 3 shows cell growth and cell viability of cryopreserved CD19/20 CAR-T cells. Cells were thawed and either incubated in CTS optimizer media with or without cytokines (IL-2 and IL-15).

FIG. 4 shows secretion of IFN-γ by CD19/20 CAR T cells after co-incubation with target Raji lymphoma cells.

FIGS. 5A and 5B shows % cytotoxicity of CAR-T cells from healthy donors generated by lentiviral transduction with the CC314B vector at an MOI of 5.

FIG. 6A shows the CD19/CD20 CAR expression achieved by transducing TN/MEM cells on day 2 after TransAct™ activation with CD19/20 CAR lentiviral vector at MOIs from 0.5 to 20. Surface CAR expression was quantified on day 12 using anti-mouse F(ab)2 antibody. % CAR expression was found to correlate with titration of MOL At MOI of 5, −60% CAR positive cells were obtained. Data shown is an average of 3 donors.

FIG. 6B shows the CD19/20 CAR expression quantified on days 7, 10, and 12 post TransAct activation. For all MOIs, CAR expression remained stable over 12 days. Data shown is an average of 3 donors.

FIG. 7 shows that vector copy number (VCN; obtained by ddPCR) and % CAR expression (using flow cytometry) correlated linearly as MOI increased from 0.5 to 20.

FIG. 8 shows the relative cytotoxicity of CD19/20 CAR-TN/MEM cells which were co-cultured with Raji cells (with or without CD19 and CD20 protein) at a range of effector to target ratios for 48 hours. Cytotoxicity was measured by quantification of viable GFP+ target cells using flow cytometry.

FIG. 9 shows the in-vitro quantification of cytokine secretion from CD19/CD20-CAR T titrates by ELISA (IL-2).

FIG. 10 shows % CAR expression and cell expansion for cell populations prepared using: (a) no enhancer, (b) Lentiboost™, or (c) protamine sulfate.

FIG. 11 is a schematic representation of pALD lenti virus based self-inactivating vectors.

FIG. 12 shows a map of a pALD_CD19/CD20 CAR plasmid.

FIG. 13 is a representative manufacturing scheme of the present invention.

FIG. 14 shows that, according to the present invention, cells stained with anti-mouse F(ab)2 (smaller squares) or anti-human EGFR antibody (larger squares) behaved similarly at a range of MOIs, suggesting that anti-F(ab)2 antibody can be used to accurately and directly measure CAR expression level on the cell surface.

FIG. 15 is a side-by-side comparison of enrichment processes performed with and without IgG.

FIG. 16 shows the results of immunophenotyping at day 12 of cells enriched in the presence or absence of IgG.

FIG. 17 shows the level expansion of cells demonstrated by the present invention.

FIG. 18 illustrates that CAR transduction is higher in groups lacking protamine sulfate than in groups containing protamine sulfate.

FIG. 19 shows the level of expansion (FIG. 19A) and total number of T cells at day 12 (FIG. 19B) of an embodiment of the present invention.

FIG. 20 illustrates CAR T cell secretion of both proinflammatory cytokines after co-culture with CD19 and/or CD20 positive Raji cells.

FIG. 21A-21F shows that CD62L+ and TN/MEM of the present invention secrete less IFN-γ and IL-2 as compared to PBMCs.

FIG. 22A-22F shows the inflammatory effect achieved (TN/MEM vs PBMC).

FIG. 23 demonstrates the effects of increased secretion of IFN-γ by CART cells after co-culture with CD19 and/or CD20 positive Raji cells at increasing E:T ratios, demonstrating specific activity of both the anti-CD 19 and anti-CD20 elements of the present invention.

FIG. 24 shows a summary of anti-CD19/CD20 CAR Constructs.

FIG. 25A-D shows the Flow Cytometry analysis of CAR Expression from Donor 292 T-cells transduced with various anti-CD19/CD20 constructs.

FIG. 26A-D shows the Flow Cytometry analysis of CAR Expression from Donor 4091 T—cells transduced with various anti-CD19/CD20 constructs.

FIG. 27A-B illustrates the percentage of CAR positive T-cells obtained from Donor 292 and Donor 4091.

FIG. 29A-D shows percentage of cytotoxicity measured by luciferase signal from the remaining viable CD19+, CD20+, CD19+CD20+ or CD19−CD20− target cells remaining in co-culture.

FIG. 30A-D shows percentage of cytotoxicity measured by luciferase signal from the remaining viable CD19+, CD20+, CD19+CD20+ or CD19−CD20− target cells remaining in co-culture.

FIG. 31 shows plotted statistical comparison (EC50) of cytotoxicity between UTD and transduced cells were performed at each E to T ratio.

FIG. 32 shows plotted statistical comparison (EC50) of cytotoxicity between UTD and transduced cells were performed at each E to T ratio.

FIG. 33 shows IFN-γ concentration of the genetically modified TN/MEM in co-culture with antigen positive and negative tumor cells from Donor 292.

FIG. 34 shows IFN-γ concentration of the genetically modified TN/MEM in co-culture with antigen positive and negative tumor cells from Donor 4091.

FIG. 35A-C shows comparison of CAR-positivity and Cell Expansion in IMPT-514 Research-scale product.

FIG. 36A-B shows immunophenotype of IMPT-514 Research-scale product.

FIG. 37 demonstrates cytokine Secretion by IMPT-514 product following 24 hr Co-incubation with Autologous B Cells.

FIG. 38 demonstrates cytotoxicity of IMPT-514 Product from Healthy and IIM Donors Co-cultured with Autologous B Cells.

FIG. 39 shows IMPT-514-specific Elimination of Both B Cells and IgG⁺ B Cells.

FIG. 40 show the structures of the second generation (one co-stimulation domain of 4-1BB) humanized anti CD20 Leu16 CAR construct.

FIG. 41A-FIG. 41C and FIG. 42 show that humanized Leu16-CAR constructs were detected by FACS analysis with fluorescently labeled anti FLAG tag antibody. Humanized Leu16-CAR-positive cells were detected after transduction of lentiviral humanized Leu16-CAR into T cells at day 12 (FIG. 41A-FIG. 41C) and (FIG. 42) respectively.

FIG. 43 show in vitro CAR T cell expansion of humanized Leu16-CAR-T cells transduced with Humanized Leu16 constructs.

FIG. 44 show that humanized Leu16-CAR-T cells transduced with Humanized Leu16 constructs have Higher CM Cells Compared to the parental Murine Leu16 CAR.

FIG. 45 show that humanized Leu16-CAR-T cells transduced with Humanized Leu16 constructs, killed Raji tumor cell line. Cytotoxicity assay was used for detection of antiCD20-CAR-T cell cytotoxicity, compared to the parental murine Leu16 CAR and un-transduced T cells.

FIG. 46 shows that humanized Leu16-CAR-T cells secreted high level of IFN-γ with Raji positive cells, versus un-transduced T cells and Mock CAR-T cells.

FIG. 47 is a schematic figure of the structural elements of the humanized the second generation (one co-stimulation domain of 4-1BB) humanized anti CD19 FMC63 CAR construct.

FIGS. 48 and 49 show that humanized FMC63-CAR constructs were detected by FACS analysis with fluorescently labeled anti FLAG tag antibody. Humanized FMC63-CAR-positive cells were detected after transduction of lentiviral humanized FMC63-CAR into T cells at day 12 (FIG. 48—donor 292) and (FIG. 49—donor 217).

FIG. 50 shows in vitro CAR T cell expansion of humanized FMC63-CAR-T cells transduced with Humanized FMC63 constructs.

FIG. 51 shows that humanized FMC63-CAR-T cells transduced with Humanized FMC63 constructs, killed Raji tumor cell line. Cytotoxicity assay was used for detection of antiCD19-CAR-T cell cytotoxicity, compared to the parental murine FMC63 CAR and un-transduced T cells.

FIG. 52 shows that humanized FMC63-CAR-T cells secreted high level of IFN-gamma with Raji positive cells, versus un-transduced T cells and Mock CAR-T cells.

### DETAILED DESCRIPTION OF THE INVENTION

Disclosed here are bi-specific CAR immune cells. The present disclosure provides an exemplary bispecific CD19/CD20 chimeric antigen receptor (CAR) T cells. The disclosed CAR immune cells may be used for therapeutic purposes. In some embodiments, CAR T cells are used in the treatment of autoimmune diseases and disorders, including Lupus, SLE, Idiopathic inflammatory myopathies, Anca Associated Vasculitis, Systemic Sclerosis and MS. In some embodiments, CAR T cells are used in the treatment of certain cancers.

To facilitate an understanding of the present disclosure, a number of terms and phrases are defined below.

### Definitions

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., +/−10%). Thus, “about” can be understood to be within 10%, 9%, S %, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

It will be understood that disclosure of any range of numerical values includes the endpoints of said range and all intervening values between said endpoints, as well as all sub-ranges contained within said range. By way of illustration, reference to a range of between 1 and 5 or a range of from 1 to 5 is a disclosure of a range of values which is at least 1 and at most 5, as well as a range of values of, for example, at least 1.01 and at most 5, at least 1.02 and at most 5, at least I and at most 4.99, and so on.

The term “administering” as used herein refers to the physical introduction of an agent to a subject, such as a modified T cell disclosed herein, using any of the various methods and delivery systems known to those skilled in the art. Exemplary routes of administration for the formulations disclosed herein include intravenous, intramuscular, subcutaneous, intraperitoneal, spinal, or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms, “activated” and “activation” refer to the state of a T cell that has been sufficiently stimulated to induce detectable cellular proliferation. In one embodiment, activation may also be associated with induced cytokine production, and detectable effector functions. The term “activated T cells” refers to, among other things, T cells that are proliferating. Signals generated through the TCR alone may be insufficient for full activation of the T cell and one or more secondary or costimulatory signals may also be required. Thus, T cell activation comprises a primary stimulation signal through the TCR/CD3 complex and one or more secondary costimulatory signals. Costimulation may be evidenced by proliferation and/or cytokine production by T cells that have received a primary activation signal, such as stimulation through the TCR/CD3 complex.

The term “allogeneic” refers to any material derived from one individual which is then introduced to another individual of the same species, e.g., allogeneic T cell transplantation.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as V H) and a heavy chain constant region. The heavy chain constant region comprises three constant domains, CH1, CH2 and CH3. Each light chain comprises a light chain variable region (abbreviated herein as V L) and a light chain constant region. The light chain constant region comprises one constant domain, CL. The V H and V L regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

The term “human antibody” is intended to comprise antibodies having variable and constant domain sequences generated, assembled, or derived from human immunoglobulin sequences, or sequences indistinguishable therefrom. In some embodiments, antibodies (or antibody components) may be considered to be “human” even though their amino acid sequences comprise residues or elements not encoded by human germline immunoglobulin sequences (e.g., variations introduced by in vitro random or site-specific mutagenesis or introduced by in vivo somatic mutation). The term “humanized” is intended to comprise antibodies having a variable domain with a sequence derived from a variable domain of a non-human species (e.g., a mouse), modified to be more similar to a human germline encoded sequence. In some embodiments, a “humanized” antibody comprises one or more framework domains having substantially the amino acid sequence of a human framework domain, and one or more complementary determining regions having substantially the amino acid sequence as that of a non-human antibody. In some embodiments, a humanized antibody comprises at least a portion of an immunoglobulin constant region (Fe), generally that of a human immunoglobulin constant domain. In some embodiments, a humanized antibodies may comprise a CH1, hinge, CH2, CH3, and, optionally, a CH4 region of a human heavy chain constant domain.

Antibodies can include, e.g., monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen binding fragments of any of the above. In certain embodiments, antibodies described herein refer to polyclonal antibody populations. Antibodies may also comprise, for example, Fab′ fragments, Fd′ fragments, Fd fragments, isolated CDRs, single chain Fvs, polypeptide-Fe fusions, single domain antibodies (e.g., shark single domain antibodies such as IgNAR or fragments thereof), camelid antibodies, single chain or Tandem diabodies, Anticalins™, and the like.

An immunoglobulin may derive from any of the commonly known isotypes, including but not limited to IgA, secretory IgA, IgG, IgE and IgM. IgG subclasses are also well known to those in the art and include but are not limited to human IgG1, IgG2, IgG3 and IgG4. “Isotype” refers to the Ab class or subclass (e.g., IgM or IgG 1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. Where not expressly stated, and unless the context indicates otherwise, the term “antibody” also includes an antigen binding fragment or an antigen-binding portion of any of the aforementioned immunoglobulins, and includes a monovalent and a divalent fragment or portion, and a single chain Ab.

The terms “antigen binding molecule,” “antigen binding portion,” “antigen binding domain,” “antigen binding fragment,” or “antibody fragment” refer to any molecule that comprises the antigen binding parts of the CAR-Ts of the invention. An antigen binding molecule can include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules.

Peptibodies (i.e., Fe fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments an antigen binding molecule is a chimeric antigen receptor (CAR) or an engineered T cell receptor (TCR).

Amino acid sequences that specifically bind to desired antigens are known in the art or may be prepared using methods known in the art. Examples include immunoglobulins, variable regions of immunoglobulins (e.g. variable fragment (“Fv”) or bivalent variable fragment (“Fab”)), single chain antibodies, etc. In certain embodiments, the antigen binding molecule is an antibody fragment that specifically binds to the antigen, including one or more of the complementarity determining regions (CDRs) thereof. In further embodiments, the antigen binding molecule is a single chain variable fragment (scFv).

In some instances, a CDR can be substantially identical to one found in a reference antibody (e.g., an antibody of the present disclosure) and/or the sequence of a CDR provided in the present disclosure. In some embodiments, a CDR is substantially identical to a reference CDR (e.g., a CDR provided in the present disclosure) in that it is either identical in sequence or contains between 1, 2, 3, 4, or 5 (e.g., 1-5) amino acid substitutions as compared with the reference CDR. In some embodiments a CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR (e.g., 85-90%, 85-95%, 85-100%, 90-95%, 90-100%, or 95-100%). In some embodiments a CDR is substantially identical to a reference CDR in that it shows at least 96%, 96%, 97%, 98%, 99%, or 100% sequence identity with the reference CDR. In some embodiments a CDR is substantially identical to a reference CDR in that one amino acid within the CDR is deleted, added, or substituted as compared with the reference CDR while the CDR has an amino acid sequence that is otherwise identical with that of the reference CDR. In some embodiments a CDR is substantially identical to a reference CDR in that 2, 3, 4, or 5 (e.g., 2-5) amino acids within the CDR are deleted, added, or substituted as compared with the reference CDR while the CDR has an amino acid sequence that is otherwise identical to the reference CDR. In various embodiments, an antigen binding fragment binds a same antigen as a reference antibody. In various embodiments, an antigen binding fragment cross-competes with the reference antibody, for example, binding to substantially the same or identical epitope as the reference antibody.

The terms “variable region” or “variable domain” are used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “V L” and “V L domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

The terms “constant region” and “constant domain” are interchangeable and have a meaning common in the art. The constant region is an antibody portion, e.g., a carboxyl terminal portion of a light and/or heavy chain which is not directly involved in binding of an antibody to antigen but which can exhibit various effector functions, such as interaction with the Fe receptor.

The constant region of an immunoglobulin molecule generally has a more conserved amino acid sequence relative to an immunoglobulin variable domain.

The term “heavy chain” when used in reference to an antibody can refer to any distinct type, e.g., alpha, delta, epsilon, gamma, and mu, based on the amino acid sequence of the constant domain, which give rise to IgA, IgD, IgE, IgG and IgM classes of antibodies, respectively, including subclasses of IgG, e.g., IgG1, IgG2, IgG3 and IgG4.

The term “light chain” when used in reference to an antibody can refer to any distinct type, e.g., kappa or lambda based on the amino acid sequence of the constant domains. Light chain amino acid sequences are well known in the art. In specific embodiments, the light chain is a human light chain.

The term “antigen” refers to a compound, composition, or substance that may stimulate the production of antibodies or a T cell response in a human or animal, including compositions (such as one that includes a tumor-specific protein) that are injected or absorbed into a human or animal. An antigen reacts with the products of specific humoral or cellular immunity, including those induced by heterologous antigens, such as the disclosed antigens. A “target antigen” or “target antigen of interest” is an antigen that is not substantially found on the surface of other normal (desired) cells and to which a binding domain of a TCR or CAR contemplated herein, is designed to bind. A person of skill in the art would readily understand that any macromolecule, including virtually all proteins or peptides, can serve as an antigen. An antigen can be endogenously expressed, i.e. expressed by genomic DNA, or can be recombinantly expressed. An antigen can be specific to a certain tissue, such as a cancer cell, or it can be broadly expressed. In addition, fragments of larger molecules can act as antigens. In one embodiment, antigens are tumor antigens.

The term “autologous” refers to any material derived from the same individual to which it is later to be re-introduced. For example, engineered autologous cell therapy herein involves collection of lymphocytes from a patient, which are then engineered to express, e.g., a CAR construct, and then administered back to the same patient.

The term “binding affinity” generally refers to the strength of the sum total of non-covalent interactions between a single binding site of a molecule (e.g., an antibody) and its binding partner (e.g., an antigen). Unless indicated otherwise “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured and/or expressed in a number of ways known in the art, including, but not limited to, equilibrium dissociation constant (Kd), and equilibrium association constant (Ka). The Kd is calculated from the quotient of koff/kon, whereas Ka is calculated from the quotient of koff/kon−kon refers to the association rate constant of, e.g., an antibody to an antigen, and koff refers to the dissociation of, e.g., an antibody to an antigen. The kon and koff can be determined by techniques known to one of ordinary skill in the art, such as BIACORE™ or KinExA™.

The term “Kd” (M) refers to the dissociation equilibrium constant of a particular antibody-antigen interaction, or the dissociation equilibrium constant of an antibody or antibody-binding fragment binding to an antigen. There is an inverse relationship between Kd and binding affinity, therefore the smaller the Kd value, the higher, i.e. stronger, the affinity. Thus, the terms “higher affinity” or “stronger affinity” relate to a higher ability to form an interaction and therefore a smaller Kd value, and conversely the terms “lower affinity” or “weaker affinity” relate to a lower ability to form an interaction and therefore a larger Kd value. In some circumstances, a higher binding affinity (Kd) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio determined by dividing the larger Kd value (lower, or weaker, affinity) by the smaller Kd (higher, or stronger, affinity), for example expressed as 5-fold or 10-fold greater binding affinity, as the case may be.

The term “Ka” refers to the association rate constant of a particular antibody-antigen interaction, or the association rate constant of an antibody or antibody-binding fragment.

The term “binding” generally refers to a non-covalent association between or among two or more entities. Direct binding involves physical contact between entities or moieties. “Indirect” binding involves physical interaction by way of physical contact with one or more intermediate entities. Binding between two or more entities may be assessed in any of a variety of contexts, e.g., where interacting entities or moieties are studied in isolation or in the context of more complex systems (e.g., while covalently or otherwise associated with a carrier entity and/or in a biological system such as a cell).

The term “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods of the present disclosure include, but are not limited to, cancers of the immune system including lymphoma, leukemia, myeloma, and other leukocyte malignancies. In some embodiments, the methods of the present disclosure can be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, glioblastoma, pituitary adenoma, Kaposi's sarcoma, epidermoid cancer, squamous cell cancer, T cell lymphoma, environmentally induced cancers including those induced by asbestos, other B cell malignancies, and combinations of said cancers. In one particular embodiment, the cancer is multiple myeloma. The particular cancer can be responsive to chemo- or radiation therapy or the cancer can be refractory. A refractory cancer refers to a cancer that is not amendable to surgical intervention and the cancer is either initially unresponsive to chemo- or radiation therapy or the cancer becomes unresponsive over time. Cancer further includes relapsed or refractory large B-cell lymphoma after two or more lines of systemic therapy, including diffuse large B-cell lymphoma (DLBCL) not otherwise specified, primary mediastinal large B-cell lymphoma after two or more lines of systemic therapy, high grade B-cell lymphoma, and DLBCL arising from follicular lymphoma.

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1.alpha. (MIP-1.alpha., MIP-1a), MIP-1.beta. (MIP-1b), gamma-induced protein 10 (IP-10), and thymus and activation regulated chemokine (TARC or CCL17).

The terms “Chimeric Antigen Receptor” or “CAR” refer to a molecule engineered to comprise a binding motif and a means of activating immune cells (for example T cells such as naive T cells, central memory T cells, effector memory T cells or combination thereof), NK cells, Tregs, and other cell types upon antigen binding. CARs are also known as artificial T cell receptors, chimeric T cell receptors or chimeric immunoreceptors. In some embodiments, a CAR comprises a binding motif, an extracellular domain, a transmembrane domain, one or more costimulatory domains, and an intracellular signaling domain. A T cell that has been genetically engineered to express a chimeric antigen receptor may be referred to as a CAR-T cell.

“Extracellular domain” (or “ECD”) refers to a portion of a polypeptide that, when the polypeptide is present in a cell membrane, is understood to reside outside of the cell membrane, in the extracellular space.

The binding domain of the CAR may be followed by a “spacer,” which refers to the region that moves the antigen binding domain away from the effector cell surface to enable proper cell/cell contact, antigen binding and activation. The spacer may further comprise a hinge region or domain. The hinge region is typically membrane proximal, and is between the transmembrane (TM) and the binding domain. In certain embodiments, a hinge region is an immunoglobulin hinge region and may be a wild-type immunoglobulin hinge region or an altered wild-type immunoglobulin hinge region. Other exemplary hinge regions used in the CARs described herein include the hinge region derived from the extracellular regions of type 1 membrane proteins such as IgG (e.g., IgG1, IgG2, IgG3, and IgG4), CD8a, CD4, CD28, 4-1BB, and CD7, which may be wild-type hinge regions from these molecules or may be altered.

The “transmembrane” region or domain is the portion of the CAR that anchors the extracellular binding portion to the plasma membrane of the immune effector cell. The transmembrane domain may be for example those obtained from CD8a, CD4, CD28, CD45, CD9, CD16, CD22, CD33, CD64, CD80, CD86, CD134, CD137, CD3zeta, and CD154. In one embodiment, the transmembrane domain is the transmembrane domain of CD8a. In certain embodiments, the transmembrane domain is synthetic in which case it would comprise predominantly hydrophobic residues such as leucine and valine.

The “intracellular domain” comprises one or more costimulatory domain, and one or more intracellular signaling domains. The “intracellular signaling domain” is also referred to herein as the “activating domain” and is typically derived from CD3z. The “intracellular costimulatory domain” can be from, e.g., 4-1BB and/or CD28.

The “intracellular signaling domain” or “signaling domain” refers to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of the cell. Effector function of the T cell, for example, may be cytolytic activity or help or activity including the secretion of a cytokine. Thus, the terms “intracellular signaling domain” or “signaling domain,” are used interchangeably herein and refer to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal. The intracellular signaling domain is also known as the, “signal transduction domain,” or “activating domain” and is typically derived from portions of the human CD3 or FcRy chains. The intracellular signaling domain/activating domain can be, e.g., derived from CD3z.

It is known that signals generated through the T cell receptor alone are generally insufficient for full activation of the T cell and that a secondary, or costimulatory signal, is also required. Thus, T cell activation can be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Cytoplasmic signaling sequences that act in a costimulatory manner may contain signaling motifs which are known as immunoreceptor tyrosine-based activation motif or ITAMs.

Examples of IT AM containing primary cytoplasmic signaling sequences that are of particular use in the disclosure include those derived from CD3 zeta, FcR gamma, FcR beta, CD3 gamma, CD3 delta, CD3 epsilon, CD8, CD22, CD79a, CD79b, and CD66d.

As used herein, the term, “costimulatory signaling domain,” or “costimulatory domain”, refers to the portion of the CAR comprising the intracellular domain of a costimulatory molecule. “Costimulatory molecules” are cell surf ace molecules other than antigen receptors or Fe receptors that provide a second signal required for efficient activation and function of T lymphocytes upon binding to antigen. The inclusion of one or more costimulatory signaling domains may enhance the efficacy and expansion of T cells expressing CAR receptors. The intracellular signaling and costimulatory signaling domains may be linked in any order in tandem to the carboxyl terminus of the transmembrane domain. Suitable costimulatory molecules include, but are not limited to, 4-1BB/CD137, B7-H3, BAFFR, BLAME (SLAMF8), BTLA, CD 33, CD 45, CDIO0 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD8, CD64, CD69, CD7, CD80, CD83 ligand, CD84, CD86, CD8alpha, CD8beta, CD9, CD96 (Tactile), CDl-la, CDl-lb, CDl-lc, CDl-ld, CD8, CEACAMl, CRT AM, DAP-10, DNAMl (CD226), Fe gamma receptor, GADS, GITR, HVEM (LIGHTR), IA4, ICAM-1, ICAM-1, ICOS, lg alpha (CD79a), IL2R beta, IL2R gamma, IL 7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CD11a/CD18), MEW class I molecule, NKG2C, NKG2D, NKp30, NKp44, NKp46, NKp80 (KLRFl), OX40, PAG/Cbp, PD-1, PSGL1, SELPLG (CD162), signaling lymphocytic activation molecule, SLAM (SLAMFl; CD150; IPO-3), SLAMF4 (CD244; 2B4), SLAMF6 (NTB-A; Lyl08), SLAMF7, SLP-76, TNF, TNFr, TNFR2, Toll ligand receptor, TRANCE/RANKL, VLA1, or VLA-6, or fragments, truncations, or combinations thereof.

As will be appreciated, although scFv-based CARs engineered to contain a signaling domain from CD3 or FcR gamma have been shown to deliver a potent signal for T cell activation and effector function, they are not sufficient to elicit signals that promote T cell survival and expansion in the absence of a concomitant costimulatory signal. Thus, CARs containing a binding domain, a hinge, a transmembrane and the signaling domain derived from CD3zeta or FcR gamma together with one or more costimulatory signaling domains (e.g., intracellular costimulatory domains derived from CD28, CD 137, CD 134 and CD278) may more effectively direct antitumor activity as well as increased cytokine secretion, lytic activity, survival and proliferation in CAR expressing T cells in vitro, and in animal models and cancer patients. See Milone et al., Molecular Therapy, 2009; 17: 1453-1464; Zhong et al., Molecular Therapy, 2010; 18: 413-420 and Carpenito et al., PNAS, 2009; 106:3360-3365).

A “conservative amino acid substitution” is one in which the amino acid residue is replaced with an amino acid residue having a similar side chain. Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, praline, phenylalanine, methionine), beta-branched side chains (e.g., threonine, valine, isoleucine) and aromatic side chains (e.g., tyrosine, phenylalanine, tryptophan, histidine). In certain embodiments, one or more amino acid residues within a CDR(s) or within a framework region(s) of an antibody or antigen-binding molecule thereof can be replaced with an amino acid residue with a similar side chain. In general, two sequences are generally considered to be “substantially similar” if they contain a conservative amino acid substitution in corresponding positions. For example, certain amino acids are generally classified as “hydrophobic” or “hydrophilic” amino acids, and/or as having “polar” or “non-polar” side chains. Substitution of one amino acid for another of the same type may be considered a conservative substitution.

“Combination therapy” refers to those situations m which a subject is simultaneously exposed to two or more therapeutic regimens (e.g., two or more therapeutic moieties). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

The “control elements” or “regulatory sequences” present in an expression vector are those non-translated regions of the vector-origin of replication, selection cassettes, promoters, enhancers, translation initiation signals (Shine Dalgarno sequence or Kozak sequence), intrans, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters maybe used.

The term “dosing regimen” may be used to refer to a set of one or more unit doses that are administered individually to a subject. In some embodiments, a given therapeutic agent has a recommended dosing regimen, which may involve one or more doses. In some embodiments, a dosing regimen comprises a plurality of doses each of which is separated in time from other doses. In some embodiments, a dosing regimen comprises a plurality of doses and consecutive doses are separated from one another by time periods of equal length; in some embodiments, a dosing regimen comprises a plurality of doses and consecutive doses are separated from one another by time periods of at least two different lengths. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen is periodically adjusted to achieve a desired or beneficial outcome.

It will be appreciated that target doses for the immune cells of the invention can range from 1×10⁶ cells to 2×10¹⁰ cells, alternatively 2×10⁶ cells, alternatively about 3×10⁶ cells, alternatively about 4×10⁶ cells, alternatively about 5×10⁶ cells, alternatively about 6×10⁶ cells, alternatively about 7×10⁶ cells, alternatively about 8×10⁶ cells, alternatively about 9×10⁶ cells, alternatively about 1×10⁷ cells, alternatively about 2×10⁷ cells, alternatively about 3×10⁷ cells, alternatively about 4×10⁷ cells, alternatively about 5×10⁷ cells, alternatively about 6×10⁷ cells, alternatively about 7×10⁷ cells, alternatively about 8×10⁷ cells, alternatively about 9×10⁷ cells, alternatively about 1×10⁸ cells, alternatively about 2×10⁸ cells, alternatively about 3×10⁸ cells, alternatively about 4×10⁸ cells, alternatively about 5×10⁸ cells, alternatively about 6×10⁸ cells, alternatively about 7×10⁸ cells, alternatively about 8×10⁸ cells, alternatively about 9×10⁸ cells, alternatively about 1×10⁹ cells, alternatively about 2×10⁹ cells, alternatively about 3×10⁹ cells, alternatively about 4×10⁹ cells, alternatively about 5×10⁹ cells, alternatively about 6×10⁹ cells, alternatively about 7×10⁹ cells, alternatively about 8×10⁹ cells, or alternatively about 9×10⁹ cells.

In certain embodiments 1×10⁷ cells, 2×10⁷ cells, 5×10⁷ cells, 1×10⁸ cells, or 2×10⁸ cells may be the preferred dose. It will be appreciated that doses above and below this range may be appropriate for certain subjects, and appropriate dose levels can be determined by the healthcare provider as needed. Additionally, multiple doses of cells can be provided in accordance with the invention.

An “epitope” refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody:antigen crystals may be studied using well known X-ray diffraction techniques and may be refined using computer software such as X-PLOR (Yale University, 1992, distributed by Molecular Simulations, Inc.; see e.g. Meth Enzymol (1985) volumes 114 & 115, eds Wyckoff H Wet al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies may be accomplished using any method known to one of skill in the art. See, e.g., Champe Met al., (1995) J Biol Chem 270: 1388-1394 and Cunningham B C & Wells J A (1989) Science 244: 1081-1085 for a description of mutagenesis techniques, including alanine scanning mutagenesis techniques.

“Endogenous” with reference to a gene, protein, and/or nucleic acid refers to the natural presence of that gene, protein, and/or nucleic acid in a cell, such as an immune cell.

“Exogenous” refers to an introduced agent, such as a nucleic acid, gene, or protein, into a cell, for example from an outside source. A nucleic acid introduced into a cell is exogenous even if it encodes a protein which is naturally found in the cell. Such exogenous introduction of a nucleic acid encoding a protein can be used to increase the expression of the protein over the level that would naturally be found in the cell under similar conditions, e.g. without introduction of the exogenous nucleic acid.

A “fragment” or “portion” of a material or entity as described herein has a structure that comprises a discrete portion of the whole, e.g., of a physical entity or abstract entity. In some embodiments, a fragment lacks one or more moieties found in the whole. In some embodiments, a fragment consists of or comprises a characteristic structural element, domain or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer (e.g., 85-90%, 85-95%, 85-100%, 90-95%, 90-100%, or 95-100%). The whole material or entity may in some embodiments be referred to as the “parent” of the fragment.

The term “fusion polypeptide” or “fusion protein” generally refers to a polypeptide comprising at least two segments. Generally, a polypeptide containing at least two such segments is considered to be a fusion polypeptide if the two segments are moieties that (1) are not comprised in nature in the same peptide, and/or (2) have not previously been linked or connected to one another in a single polypeptide, and/or (3) have been linked or connected to one another through action of the hand of man. In embodiments, a CAR is a fusion protein.

A “T cell receptor” or “TCR” refers to antigen-recognition molecules present on the surface of T cells. During normal T cell development, each of the four TCR genes, alpha, beta, gamma, and delta, may rearrange leading to highly diverse TCR proteins.

The term “heterologous” means from any source other than naturally occurring sequences. For example, a heterologous sequence included as a part of a costimulatory protein is amino acids that do not naturally occur as, i.e., do not align with, the wild type human costimulatory protein. For example, a heterologous nucleotide sequence refers to a nucleotide sequence other than that of the wild type human costimulatory protein-encoding sequence.

The term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

The terms “improve,” “increase,” “inhibit,” and “reduce” indicate values that are relative to a baseline or other reference measurement. In some embodiments, an appropriate reference measurement may comprise a measurement in certain system (e.g., in a single individual) under otherwise comparable conditions absent presence of (e.g., prior to and/or after) an agent or treatment, or in presence of an appropriate comparable reference agent. In some embodiments, an appropriate reference measurement may comprise a measurement in comparable system known or expected to respond in a comparable way, in presence of the relevant agent or treatment.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies or NK cell therapies. T cell therapy can include adoptive T cell therapy, tumor-infiltrating lymphocyte (TIL) immunotherapy, autologous cell therapy, and allogeneic T cell transplantation.

As used herein, the term “autoimmune disease” can include Lupus, including SLE, as well as Idiopathic inflammatory myopathies, ANCA Associated Vasculitis, Systemic Sclerosis Rheumatoid arthritis, Systemic lupus erythematosus (lupus), Inflammatory Bowel Disease (IBD/Crohn's), Type 1 diabetes mellitus, Guillain-Barre syndrome, Chronic inflammatory demyelinating polyneuropathy, Psoriasis, Graves' disease, Hashimoto's thyroiditis, Myasthenia gravis, Scleroderma or systemic sclerosis. Additional autoimmune diseases include neurological autoimmune diseases, including but not limited to Multiple Sclerosis (MS), autoimmune neuropathy, transverse myelitis, optic neuritis, nemomyelitis optica, acute disseminated encephalomyelitis, autoimmune or pa.raneoplastic encephalitis, and spasticity. Additional autoimmune diseases include, e.g., ANCA vasculitis, Pemphigus vulgaris, Pemphigus foliaceus, membranous nephropathy, and polymyositis, As used herein, the term “SLE” refers to Systemic Lupus Erythematosus.

One of skill in the art will recognize techniques to enhance the effectiveness of cell therapy using, e.g., preconditioning techniques such as those found in U.S. Pat. Nos. 9,855,298 and 10,322,146, the contents of which are hereby incorporated by reference in their entirety.

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

The term “in vitro” refers to events occurring in an artificial environment, e.g., in a test tube, reaction vessel, cell culture, etc., rather than within a multi-cellular organism. The term “in vitro cell” refers to any cell which is cultured ex vivo. In particular, an in vitro cell can include a T cell. The term “in vivo” refers to events that occur within a multi-cellular organism, such as a human or a non-human animal.

The term “isolated” refers to a substance that (1) has been separated from at least some components with which it was associated at an earlier time or with which the substance would otherwise be associated, and/or (2) is present in a composition that comprises a limited or defined amount or concentration of one or more known or unknown contaminants. An isolated substance, in some embodiments, may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% (e.g., 85-90%, 85-95%, 85-100%, 90-95%, 90-100%, or 95-100%) of other non-substance components with which the substance was associated at an earlier time, e.g., other components or contaminants with which the substance was previously or otherwise would be associated. In certain instances, a substance is isolated if it is present in a composition that comprises a limited or reduced amount or concentration of molecules of a same or similar type. For instance, in certain instances, a nucleic acid, DNA, or RNA substance is isolated if it is present in a composition that comprises a limited or reduced amount or concentration of non-substance nucleic acid, DNA, or RNA molecules. For instance, in certain instances, a polypeptide substance is isolated if it is present in a composition that comprises a limited or reduced amount or concentration of non-substance polypeptide molecules. In certain embodiments, an amount may be, e.g., an amount measured relative to the amount of a desired substance present in a composition. In certain embodiments, a limited amount may be an amount that is no more than 100% of the amount of substance in a composition, e.g., no more than 1%, 5%, 10%, 20%, 30%, 40%, 50%, 60%, 70%, 80%, 90%, or 95% of the amount of substance in a composition (e.g., 85-90%, 85-95%, 85-100%, 90-95%, 90-100%, or 95-100%). In certain instances, a composition is pure or substantially pure with respect to a selected substance. In some embodiments, an isolated substance is about 80%, about 85%, about 90%, about 91%, about 92%, about 93%, about 94%, about 95%, about 96%, about 97%, about 98%, about 99%, or more than about 99% pure (e.g., 85-90%, 85-95%, 85-100%, 90-95%, 90-100%, or 95-100%). A substance is “pure” if it is substantially free of other components or of contaminants. In some embodiments, a substance may still be considered “isolated” or even “pure,” after having been combined with certain other components such as, for example, one or more carriers or excipients (e.g., buffer, solvent, water, etc.); in such embodiments, percent isolation or purity of the substance is calculated without comprising such carriers or excipients.

The term “lymphocyte” includes natural killer (NK) cells, T cells, or B cells. NK cells are a type of cytotoxic (cell toxic) lymphocyte that represent a component of the inherent immune system. NK cells reject tumors and cells infected by viruses. It works through the process of apoptosis or programmed cell death. They were termed “natural killers” because they do not require activation in order to kill cells. T cells play a role in cell-mediated-immunity (no antibody involvement). Its T cell receptors (TCR) differentiate themselves from other lymphocyte types. The thymus, a specialized organ of the immune system, is primarily responsible for the T cell's maturation. There are six types of T cells, namely: Helper T cells (e.g., CD4+ cells), Cytotoxic T cells (also known as TC, cytotoxic T lymphocyte, CTL, T-killer cell, cytolytic T cell, CD8+ T cells or killer T cell), Memory T cells ((i) stem memory TSCM cells, like naive cells, are CD45Ro−′CCR7+, CD45RA+, CD62L+(L-selectin), CD27+, CD28+ and IL-7R alpha+, but they also express large amounts of CD95, IL-2RB, CXCR3, and LFA-1, and show numerous functional attributes distinctive of memory cells); (ii) central memory T.sub.CM cells express L-selectin and the CCR7, they secrete IL-2, but not IFN-gamma or IL-4, and (iii) effector memory TEM cells, however, do not express L-selectin or CCR7 but produce effector cytokines like IFN-gamma and IL-4), Regulatory T cells (Tregs, suppressor T cells, or CD4+CD25+ regulatory T cells), Natural Killer T cells (NKT) and Gamma Delta T cells. B-cells, on the other hand, play a role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

The term “neutralizing” refers to an antigen binding molecule, scFv, antibody, or a fragment thereof, that binds to a ligand and prevents or reduces the biological effect of that ligand. In some embodiments, the antigen binding molecule, scFv, antibody, or a fragment thereof, directly blocking a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

The term “nucleic acid” refers to any polymeric chain of nucleotides. A nucleic acid may be DNA, RNA, or a combination thereof. In some embodiments, a nucleic acid comprises one or more natural nucleic acid residues. In some embodiments, a nucleic acid comprises of one or more nucleic acid analogs. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long (e.g., 20 to 100, 20 to 500, 20 to 1000, 20 to 2000, or 20 to 5000 or more residues). In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide.

The term “operably linked” refers to a juxtaposition where the components described are in a relationship permitting them to function in their intended manner. For example, a control element “operably linked” to a functional element is associated in such a way that expression and/or activity of the functional element is achieved under conditions compatible with the control element.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that can comprise a protein's or peptide's sequence. Polypeptides include any peptide or protein comprising two or more amino acids joined to each other by peptide bonds.

As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

In other embodiments, a vector for use in practicing the embodiments described herein including, but not limited to expression vectors and viral vectors, will include exogenous, endogenous, or heterologous sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” sequence is an exogenous sequence that may be from a different protein of the same species or a different species than the protein or cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In some embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances may function independent of their orientation relative to another control sequence. An enhancer may function cooperatively or additively with promoters and/or other enhancer elements. The term “promoter/enhancer” refers to a segment of DNA which contains sequences capable of providing both promoter and enhancer functions.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosa surfaces.

“Regulatory T cells” (“Treg”, “Treg cells”, or “Tregs”) refer to a lineage of CD4+T lymphocytes that participate in controlling certain immune activities, e.g., autoimmunity, allergy, and response to infection. Regulatory T cells may regulate the activities of T cell populations, and may also influence certain innate immune system cell types. Tregs may be identified by the expression of the biomarkers CD4, CD25 and Foxp3, and low expression of CD127. Naturally occurring Treg cells normally constitute about 5-10% of the peripheral CD4+T lymphocytes. However, Treg cells within a tumor microenvironment (i.e. tumor-infiltrating Treg cells), Treg cells may make up as much as 20-30% of the total CD4+T lymphocyte population.

“Single chain variable fragment”, “single-chain antibody variable fragments” or “scFv” antibodies refer to forms of antibodies comprising the variable regions of only the heavy and light chains, connected by a linker peptide.

The phrase “therapeutic agent” may refer to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms or human subjects. In some embodiments, an appropriate population may be defined by various criteria, such as a certain age group, gender, genetic background, preexisting clinical conditions, in accordance with presence or absence of a biomarker, etc. In some embodiments, a therapeutic agent is a substance that may be used to alleviate, ameliorate, relieve, inhibit, prevent, delay onset of, reduce severity of, and/or reduce incidence of one or more symptoms or features of a disease, disorder, and/or condition. In some embodiments, a therapeutic agent is an agent that has been or is required to be approved by a government agency before it may be marketed for administration to humans. In some embodiments, a therapeutic agent is an agent for which a medical prescription is required for administration to humans.

A “therapeutically effective amount,” “effective dose,” “effective amount,” or “therapeutically effective dosage” of a therapeutic agent, e.g., engineered CAR-T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “vector” or “lentiviral vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in some embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaL V), feline leukemia virus (FL V), spumavirus, Friend murine leukemia virus, Murine Stem Cell Virus (MSCV) and Rous Sarcoma Virus (RSV) and lentivirus.

As used herein, the term “lentivirus” refers to a group (or genus) of complex retroviruses. Illustrative lentiviruses include, but are not limited to: HIV (human immunodeficiency virus; including HIV type-1, and HIV type-2); visna-maedi virus (VMV) virus; the caprine arthritis encephalitis virus (CAEV); equine infectious anemia virus (EIA V); feline immunodeficiency virus (FIV); bovine immune deficiency virus (BIV); and simian immunodeficiency virus (SIV).

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

### Chimeric Antigen Receptors (CAR)

In some embodiments, the present disclosure provides polypeptide comprising a CD19/CD20 chimeric antigen receptor (CAR). In some aspects, the CAR comprises an anti-CD20 scFv comprising (i) a light chain variable region and (ii) a heavy chain variable region; a (G₄S)ₙ linker, wherein n is 1, 2, 3, or 4; an anti-CD19 scFv comprising (i) a heavy chain variable region and (ii) a light chain variable region; a spacer domain; a transmembrane domain; a 4-1BB cytoplasmic signaling domain; and a CD3 zeta signaling domain.

In some aspects, at least one of the anti-CD20 scFv or anti-CD19 scFv comprises at least one CDR sequence isolated or derived from a nonhuman subject. In other aspects, at least one of the anti-CD20 scFv or anti-CD19 scFv comprises at least one CDR sequence isolated or derived from a human subject.

In some aspects, the CAR comprises an anti-CD20 scFv comprising (i) a light chain variable region having the amino acid sequence of SEQ ID NO: 34, and (ii) a heavy chain variable region having the amino acid sequence of SEQ ID NO: 35; a (G₄S)₄ linker having the amino acid sequence of SEQ ID NO: 51; an anti-CD19 scFv comprising (i) a heavy chain variable region having the amino acid sequence of SEQ ID NO: 36, and (ii) a light chain variable region having the amino acid sequences of SEQ ID NO: 37; a spacer domain comprising the amino acid sequence of any one selected from SEQ ID NOs: 21-29; a transmembrane domain comprising the amino acid sequence of SEQ ID NO: 30 or 31; a 4-1BB cytoplasmic signaling domain comprising the amino acid sequence of SEQ ID NO: 32; and a CD3 zeta signaling domain comprising the amino acid sequence of SEQ ID NO: 33.

In some aspects, the CAR comprises an anti-CD20 scFv comprising (i) a light chain variable region having the amino acid sequence of SEQ ID NO: 34, and (ii) a heavy chain variable region having the amino acid sequence of SEQ ID NO: 35; a (G₄S)₃ linker having the amino acid sequence of SEQ ID NO: 50; an anti-CD19 scFv comprising (i) a heavy chain variable region having the amino acid sequence of SEQ ID NO: 36, and (ii) a light chain variable region having the amino acid sequences of SEQ ID NO: 37; a spacer domain comprising the amino acid sequence of any one selected from SEQ ID NOs: 21-29; a transmembrane domain comprising the amino acid sequence of SEQ ID NO: 30 or 31; a 4-1BB cytoplasmic signaling domain comprising the amino acid sequence of SEQ ID NO: 32; and a CD3 zeta signaling domain comprising the amino acid sequence of SEQ ID NO: 33.

In some aspects, the CAR comprises an anti-CD20 scFv comprising (i) a light chain variable region having the amino acid sequence of SEQ ID NO: 34, and (ii) a heavy chain variable region having the amino acid sequence of SEQ ID NO: 35; a (G₄S)₂ linker having the amino acid sequence of SEQ ID NO: 49; an anti-CD19 scFv comprising (i) a heavy chain variable region having the amino acid sequence of SEQ ID NO: 36, and (ii) a light chain variable region having the amino acid sequences of SEQ ID NO: 37; a spacer domain comprising the amino acid sequence of any one selected from SEQ ID NOs: 21-29; a transmembrane domain comprising the amino acid sequence of SEQ ID NO: 30 or 31; a 4-1BB cytoplasmic signaling domain comprising the amino acid sequence of SEQ ID NO: 32; and a CD3 zeta signaling domain comprising the amino acid sequence of SEQ ID NO: 33.

In some aspects, the CAR comprises an anti-CD20 scFv comprising (i) a light chain variable region having the amino acid sequence of SEQ ID NO: 34, and (ii) a heavy chain variable region having the amino acid sequence of SEQ ID NO: 35; a (G₄S)₁ linker having the amino acid sequence of SEQ ID NO: 48; an anti-CD19 scFv comprising (i) a heavy chain variable region having the amino acid sequence of SEQ ID NO: 36, and (ii) a light chain variable region having the amino acid sequences of SEQ ID NO: 37; a spacer domain comprising the amino acid sequence of any one selected from SEQ ID NOs: 21-29; a transmembrane domain comprising the amino acid sequence of SEQ ID NO: 30 or 31; a 4-1BB cytoplasmic signaling domain comprising the amino acid sequence of SEQ ID NO: 32; and a CD3 zeta signaling domain comprising the amino acid sequence of SEQ ID NO: 33.

The CAR may be configured from N terminus to C terminus and having the following formula:

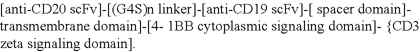

In some embodiments, the present disclosure provides polypeptide comprising a CD19 chimeric antigen receptor (CAR), comprising: an anti-CD19 scFv comprising (i) a heavy chain variable region, and (ii) a light chain variable region; a spacer domain; a transmembrane domain; a 4-1BB cytoplasmic signaling domain; and a CD3 zeta signaling domain.

In some aspects, the CAR comprises an anti-CD19 scFv comprising (i) a heavy chain variable region having the amino acid sequence of SEQ ID NO: 36, and (ii) a light chain variable region having the amino acid sequences of SEQ ID NO: 37; a spacer domain comprising the amino acid sequence of any one selected from SEQ ID NOs: 21-29; a transmembrane domain comprising the amino acid sequence of SEQ ID NO: 30 or 31; a 4-1BB cytoplasmic signaling domain comprising the amino acid sequence of SEQ ID NO: 32; and a CD3 zeta signaling domain comprising the amino acid sequence of SEQ ID NO: 33.

In some aspects, the anti-CD19 scFv comprises at least one CDR sequence isolated or derived from a nonhuman subject. In other aspects, the anti-CD19 scFv comprises at least one CDR sequence isolated or derived from a human subject.

The CAR may be configured from N terminus to C terminus and having the following formula:

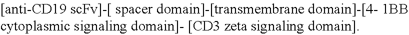

In an aspect, the variable heavy and variable light scFv sequences of human CD19, in either the VH-VL or VL-VH orientation) are connected to each other via a flexible GS linker with the amino acid sequence of: GSTSGGGSGGGSGGGGSS.

In an aspect, the variable heavy and variable light scFv sequences of human CD20, in either the VH-VL or VL-VH orientation) are connected to each other via a flexible GS linker with the amino acid sequence of: GSTSGGGSGGGSGGGGSS.

The sequences of SEQ ID NOs: 1-37, 48-78, and 230-240 are set forth in Table 1 below:

TABLE 1

| SEQ ID NO | Amino Acid Sequence | Annotation |
|---|---|---|
| 1 | ATGGAGACAGACACACTCCTGCTATGGGTGCTGCTGCT | CAR Construct 1 |
| | CTGGGTTCCAGGTTCCACAGGTGACATTGTGCTGACCC | |
| | AATCTCCAGCTATCCTGTCTGCATCTCCAGGGGAGAAG | |
| | GTCACAATGACTTGCAGGGCCAGCTCAAGTGTAAATTA | |
| | CATGGACTGGTACCAGAAGAAGCCAGGATCCTCCCCCA | |
| | AACCCTGGATTTATGCCACATCCAACCTGGCTTCTGGA | |
| | GTCCCTGCTCGCTTCAGTGGCAGTGGGTCTGGGACCTCT | |
| | TACTCTCTCACAATCAGCAGAGTGGAGGCTGAAGATGC | |
| | TGCCACTTATTACTGCCAGCAGTGGAGTTTTAATCCACC | |
| | CACGTTCGGAGGGGGGACCAAGCTGGAAATAAAAGGC | |
| | AGTACTAGCGGTGGTGGCTCCGGGGGCGGTTCCGGTGG | |
| | GGGCGGCAGCAGCGAGGTGCAGCTGCAGCAGTCTGGG | |
| | GCTGAGCTGGTGAAGCCTGGGGCCTCAGTGAAGATGTC | |
| | CTGCAAGGCTTCTGGCTACACATTTACCAGTTACAATAT | |
| | GCACTGGGTAAAGCAGACACCTGGACAGGGCCTGGAA | |
| | TGGATTGGAGCTATTTATCCAGGAAATGGTGATACTTC | |
| | CTACAATCAGAAGTTCAAAGGCAAGGCCACATTGACTG | |
| | CAGACAAATCCTCCAGCACAGCCTACATGCAGCTCAGC | |
| | AGCCTGACATCTGAGGACTCTGCGGACTATTACTGTGC | |
| | AAGATCTAATTATTACGGTAGTAGCTACTGGTTCTTCGA | |
| | TGTCTGGGGCGCAGGGACCACGGTCACAGTAAGTAGCG | |
| | GTGGAGGCGGCAGTGGCGGAGGTGGGAGCGGAGGGGG | |
| | CGGTTCCGGTGGCGGGGGATCTGAGGTGAAGCTGCAGG | |
| | AAAGCGGCCCTGGCCTGGTGGCCCCCAGCCAGAGCCTG | |
| | AGCGTGACCTGCACCGTGAGCGGCGTGAGCCTGCCCGA | |
| | CTACGGCGTGAGCTGGATCCGGCAGCCCCCCAGGAAGG | |
| | GCCTGGAATGGCTGGGCGTGATCTGGGGCAGCGAGACC | |
| | ACCTACTACAACAGCGCCCTGAAGAGCCGGCTGACCAT | |
| | CATCAAGGACAACAGCAAGAGCCAGGTGTTCCTGAAG | |
| | ATGAACAGCCTGCAGACCGACGACACCGCCATCTACTA | |
| | CTGCGCCAAGCACTACTACTACGGCGGCAGCTACGCCA | |
| | TGGACTACTGGGGCCAGGGCACCAGCGTGACCGTGAGC | |
| | AGCGGCAGCACCTCCGGCAGCGGCAAGCCTGGCAGCG | |
| | GCGAGGGCAGCACCAAGGGCGACATCCAGATGACCCA | |
| | GACCACCTCCAGCCTGAGCGCCAGCCTGGGCGACCGGG | |
| | TGACCATCAGCTGCCGGGCCAGCCAGGACATCAGCAAG | |
| | TACCTGAACTGGTATCAGCAGAAGCCCGACGGCACCGT | |
| | CAAGCTGCTGATCTACCACACCAGCCGGCTGCACAGCG | |
| | GCGTGCCCAGCCGGTTTAGCGGCAGCGGCTCCGGCACC | |
| | GACTACAGCCTGACCATCTCCAACCTGGAACAGGAAGA | |
| | TATCGCCACCTACTTTTGCCAGCAGGGCAACACACTGC | |
| | CCTACACCTTTGGCGGCGGAACAAAGCTGGAAATCACC | |
| | GAGAGCAAGTACGGACCGCCCTGCCCCCCTTGCCCTAT | |
| | GTTCTGGGTGCTGGTGGTGGTCGGAGGCGTGCTGGCCT | |
| | GCTACAGCCTGCTGGTCACCGTGGCCTTCATCATCTTTT | |
| | GGGTGAAACGGGGCAGAAAGAAACTCCTGTATATATTC | |
| | AAACAACCATTTATGAGACCAGTACAAACTACTCAAGA | |
| | GGAAGATGGCTGTAGCTGCCGATTTCCAGAAGAAGAAG | |
| | AAGGAGGATGTGAACTGCGGGTGAAGTTCAGCAGAAG | |
| | CGCCGACGCCCCTGCCTACCAGCAGGGCCAGAATCAGC | |
| | TGTACAACGAGCTGAACCTGGGCAGAAGGGAAGAGTA | |
| | CGACGTCCTGGATAAGCGGAGAGGCCGGGACCCTGAG | |
| | ATGGGCGGCAAGCCTCGGCGGAAGAACCCCCAGGAAG | |
| | GCCTGTATAACGAACTGCAGAAAGACAAGATGGCCGA | |
| | GGCCTACAGCGAGATCGGCATGAAGGGCGAGCGGAGG | |
| | CGGGGCAAGGGCCACGACGGCCTGTATCAGGGCCTGTC | |
| | CACCGCCACCAAGGATACCTACGACGCCCTGCACATGC | |
| | AGGCCCTGCCCCCAAGGTGA | |
| 2 | METDTLLLWVLLLWVPGSTGDIVLTQSPAILSASPGEKVT | CAR Construct 2 |
| | MTCRASSSVNYMDWYQKKPGSSPKPWIYATSNLASGVPA | |
| | RFSGSGSGTSYSLTISRVEAEDAATYYCQQWSFNPPTFGG | |
| | GTKLEIKGSTSGGGSGGGSGGGGSSEVQLQQSGAELVKPG | |
| | ASVKMSCKASGYTFTSYNMHWVKQTPGQGLEWIGAIYP | |
| | GNGDTSYNQKFKGKATLTADKSSSTAYMQLSSLTSEDSA | |
| | DYYCARSNYYGSSYWFFDVWGAGTTVTVSSGGGGSGGG | |
| | GSGGGGSGGGGSEVKLQESGPGLVAPSQSLSVTCTVSGVS | |
| | LPDYGVSWIRQPPRKGLEWLGVIWGSETTYYNSALKSRLT | |
| | IIKDNSKSQVFLKMNSLQTDDTAIYYCAKHYYYGGSYAM | |
| | DYWGQGTSVTVSSGSTSGSGKPGSGEGSTKGDIQMTQTTS | |
| | SLSASLGDRVTISCRASQDISKYLNWYQQKPDGTVKLLIY | |
| | HTSRLHSGVPSRFSGSGSGTDYSLTISNLEQEDIATYFCQQ | |
| | GNTLPYTFGGGTKLEITESKYGPPCPPCPMFWVLVVVGGV | |
| | LACYSLLVTVAFIIFWVKRGRKKLLYIFKQPFMRPVQTTQ | |
| | EEDGCSCRFPEEEEGGCELRVKFSRSADAPAYQQGQNQL | |
| | YNELNLGRREEYDVLDKRRGRDPEMGGKPRRKNPQEGL | |
| | YNELQKDKMAEAYSEIGMKGERRRGKGHDGLYQGLSTA | |
| | TKDTYDALHMQALPPR | |
| 3 | ATGGAGACAGACACACTCCTGCTATGGGTGCTGCTGCT | CAR Construct 3 |
| | CTGGGTTCCAGGTTCCACAGGTGACATTGTGCTGACCC | |
| | AATCTCCAGCTATCCTGTCTGCATCTCCAGGGGAGAAG | |
| | GTCACAATGACTTGCAGGGCCAGCTCAAGTGTAAATTA | |
| | CATGGACTGGTACCAGAAGAAGCCAGGATCCTCCCCCA | |
| | AACCCTGGATTTATGCCACATCCAACCTGGCTTCTGGA | |
| | GTCCCTGCTCGCTTCAGTGGCAGTGGGTCTGGGACCTCT | |
| | TACTCTCTCACAATCAGCAGAGTGGAGGCTGAAGATGC | |
| | TGCCACTTATTACTGCCAGCAGTGGAGTTTTAATCCACC | |
| | CACGTTCGGAGGGGGGACCAAGCTGGAAATAAAAGGC | |
| | AGTACTAGCGGTGGTGGCTCCGGGGGCGGTTCCGGTGG | |
| | GGGCGGCAGCAGCGAGGTGCAGCTGCAGCAGTCTGGG | |
| | GCTGAGCTGGTGAAGCCTGGGGCCTCAGTGAAGATGTC | |
| | CTGCAAGGCTTCTGGCTACACATTTACCAGTTACAATAT | |
| | GCACTGGGTAAAGCAGACACCTGGACAGGGCCTGGAA | |
| | TGGATTGGAGCTATTTATCCAGGAAATGGTGATACTTC | |
| | CTACAATCAGAAGTTCAAAGGCAAGGCCACATTGACTG | |
| | CAGACAAATCCTCCAGCACAGCCTACATGCAGCTCAGC | |
| | AGCCTGACATCTGAGGACTCTGCGGACTATTACTGTGC | |
| | AAGATCTAATTATTACGGTAGTAGCTACTGGTTCTTCGA | |
| | TGTCTGGGGCGCAGGGACCACGGTCACAGTAAGTAGCG | |
| | GTGGAGGCGGCAGTGGCGGAGGTGGGAGCGGAGGGGG | |
| | CGGTTCCGGTGGCGGGGGATCTGAGGTGAAGCTGCAGG | |
| | AAAGCGGCCCTGGCCTGGTGGCCCCCAGCCAGAGCCTG | |
| | AGCGTGACCTGCACCGTGAGCGGCGTGAGCCTGCCCGA | |
| | CTACGGCGTGAGCTGGATCCGGCAGCCCCCCAGGAAGG | |
| | GCCTGGAATGGCTGGGCGTGATCTGGGGCAGCGAGACC | |
| | ACCTACTACAACAGCGCCCTGAAGAGCCGGCTGACCAT | |
| | CATCAAGGACAACAGCAAGAGCCAGGTGTTCCTGAAG | |
| | ATGAACAGCCTGCAGACCGACGACACCGCCATCTACTA | |
| | CTGCGCCAAGCACTACTACTACGGCGGCAGCTACGCCA | |
| | TGGACTACTGGGGCCAGGGCACCAGCGTGACCGTGAGC | |
| | AGCGGCAGCACCTCCGGCAGCGGCAAGCCTGGCAGCG | |
| | GCGAGGGCAGCACCAAGGGCGACATCCAGATGACCCA | |
| | GACCACCTCCAGCCTGAGCGCCAGCCTGGGCGACCGGG | |
| | TGACCATCAGCTGCCGGGCCAGCCAGGACATCAGCAAG | |
| | TACCTGAACTGGTATCAGCAGAAGCCCGACGGCACCGT | |
| | CAAGCTGCTGATCTACCACACCAGCCGGCTGCACAGCG | |
| | GCGTGCCCAGCCGGTTTAGCGGCAGCGGCTCCGGCACC | |
| | GACTACAGCCTGACCATCTCCAACCTGGAACAGGAAGA | |
| | TATCGCCACCTACTTTTGCCAGCAGGGCAACACACTGC | |
| | CCTACACCTTTGGCGGCGGAACAAAGCTGGAAATCACC | |
| | ACTACAACTCCTGCCCCACGGCCACCTACACCTGCGCC | |
| | TACAATTGCCAGCCAGCCACTGTCCTTACGACCAGAGG | |
| | CTTGCCGCCCCGCTGCGGGAGGCGCCGTCCATACCCGG | |
| | GGCCTTGACTTCGCATGCGATATGTTCTGGGTGCTGGTG | |
| | GTGGTCGGAGGCGTGCTGGCCTGCTACAGCCTGCTGGT | |
| | CACCGTGGCCTTCATCATCTTTTGGGTGAAACGGGGCA | |
| | GAAAGAAACTCCTGTATATATTCAAACAACCATTTATG | |
| | AGACCAGTACAAACTACTCAAGAGGAAGATGGCTGTA | |
| | GCTGCCGATTTCCAGAAGAAGAAGAAGGAGGATGTGA | |
| | ACTGCGGGTGAAGTTCAGCAGAAGCGCCGACGCCCCTG | |
| | CCTACCAGCAGGGCCAGAATCAGCTGTACAACGAGCTG | |
| | AACCTGGGCAGAAGGGAAGAGTACGACGTCCTGGATA | |
| | AGCGGAGAGGCCGGGACCCTGAGATGGGCGGCAAGCC | |
| | TCGGCGGAAGAACCCCCAGGAAGGCCTGTATAACGAA | |
| | CTGCAGAAAGACAAGATGGCCGAGGCCTACAGCGAGA | |
| | TCGGCATGAAGGGCGAGCGGAGGCGGGGCAAGGGCCA | |
| | CGACGGCCTGTATCAGGGCCTGTCCACCGCCACCAAGG | |
| | ATACCTACGACGCCCTGCACATGCAGGCCCTGCCCCCA | |
| | AGGTGA | |
| 4 | METDTLLLWVLLLWVPGSTGDIVLTQSPAILSASPGEKVT | CAR Construct 4 |
| | MTCRASSSVNYMDWYQKKPGSSPKPWIYATSNLASGVPA | |
| | RFSGSGSGTSYSLTISRVEAEDAATYYCQQWSFNPPTFGG | |
| | GTKLEIKGSTSGGGSGGGSGGGGSSEVQLQQSGAELVKPG | |
| | ASVKMSCKASGYTFTSYNMHWVKQTPGQGLEWIGAIYP | |
| | GNGDTSYNQKFKGKATLTADKSSSTAYMQLSSLTSEDSA | |
| | DYYCARSNYYGSSYWFFDVWGAGTTVTVSSGGGGSGGG | |
| | GSGGGGSGGGGSEVKLQESGPGLVAPSQSLSVTCTVSGVS | |
| | LPDYGVSWIRQPPRKGLEWLGVIWGSETTYYNSALKSRLT | |
| | IIKDNSKSQVFLKMNSLQTDDTAIYYCAKHYYYGGSYAM | |
| | DYWGQGTSVTVSSGSTSGSGKPGSGEGSTKGDIQMTQTTS | |
| | SLSASLGDRVTISCRASQDISKYLNWYQQKPDGTVKLLIY | |
| | HTSRLHSGVPSRFSGSGSGTDYSLTISNLEQEDIATYFCQQ | |
| | GNTLPYTFGGGTKLEITTTTPAPRPPTPAPTIASQPLSLRPE | |
| | ACRPAAGGAVHTRGLDFACD | |
| | MFWVLVVVGGVLACYSLLVTVAFIIFWVKRGRKKLLYIF | |
| | KQPFMRPVQTTQEEDGCSCRFPEEEEGGCELRVKFSRSAD | |
| | APAYQQGQNQLYNELNLGRREEYDVLDKRRGRDPEMGG | |
| | KPRRKNPQEGLYNELQKDKMAEAYSEIGMKGERRRGKG | |
| | HDGLYQGLSTATKDTYDALHMQALPPR | |
| 5 | ATGGAGACAGACACACTCCTGCTATGGGTGCTGCTGCT | CAR Construct 5 |
| | CTGGGTTCCAGGTTCCACAGGTGACATTGTGCTGACCC | |
| | AATCTCCAGCTATCCTGTCTGCATCTCCAGGGGAGAAG | |
| | GTCACAATGACTTGCAGGGCCAGCTCAAGTGTAAATTA | |
| | CATGGACTGGTACCAGAAGAAGCCAGGATCCTCCCCCA | |
| | AACCCTGGATTTATGCCACATCCAACCTGGCTTCTGGA | |
| | GTCCCTGCTCGCTTCAGTGGCAGTGGGTCTGGGACCTCT | |
| | TACTCTCTCACAATCAGCAGAGTGGAGGCTGAAGATGC | |
| | TGCCACTTATTACTGCCAGCAGTGGAGTTTTAATCCACC | |
| | CACGTTCGGAGGGGGGACCAAGCTGGAAATAAAAGGC | |
| | AGTACTAGCGGTGGTGGCTCCGGGGGCGGTTCCGGTGG | |
| | GGGCGGCAGCAGCGAGGTGCAGCTGCAGCAGTCTGGG | |
| | GCTGAGCTGGTGAAGCCTGGGGCCTCAGTGAAGATGTC | |
| | CTGCAAGGCTTCTGGCTACACATTTACCAGTTACAATAT | |
| | GCACTGGGTAAAGCAGACACCTGGACAGGGCCTGGAA | |
| | TGGATTGGAGCTATTTATCCAGGAAATGGTGATACTTC | |
| | CTACAATCAGAAGTTCAAAGGCAAGGCCACATTGACTG | |
| | CAGACAAATCCTCCAGCACAGCCTACATGCAGCTCAGC | |
| | AGCCTGACATCTGAGGACTCTGCGGACTATTACTGTGC | |
| | AAGATCTAATTATTACGGTAGTAGCTACTGGTTCTTCGA | |
| | TGTCTGGGGCGCAGGGACCACGGTCACAGTAAGTAGCG | |
| | GTGGAGGCGGCAGTGGCGGAGGTGGGAGCGGAGGGGG | |
| | CGGTTCCGGTGGCGGGGGATCTGAGGTGAAGCTGCAGG | |
| | AAAGCGGCCCTGGCCTGGTGGCCCCCAGCCAGAGCCTG | |
| | AGCGTGACCTGCACCGTGAGCGGCGTGAGCCTGCCCGA | |
| | CTACGGCGTGAGCTGGATCCGGCAGCCCCCCAGGAAGG | |
| | GCCTGGAATGGCTGGGCGTGATCTGGGGCAGCGAGACC | |
| | ACCTACTACAACAGCGCCCTGAAGAGCCGGCTGACCAT | |
| | CATCAAGGACAACAGCAAGAGCCAGGTGTTCCTGAAG | |
| | ATGAACAGCCTGCAGACCGACGACACCGCCATCTACTA | |
| | CTGCGCCAAGCACTACTACTACGGCGGCAGCTACGCCA | |
| | TGGACTACTGGGGCCAGGGCACCAGCGTGACCGTGAGC | |
| | AGCGGCAGCACCTCCGGCAGCGGCAAGCCTGGCAGCG | |
| | GCGAGGGCAGCACCAAGGGCGACATCCAGATGACCCA | |
| | GACCACCTCCAGCCTGAGCGCCAGCCTGGGCGACCGGG | |
| | TGACCATCAGCTGCCGGGCCAGCCAGGACATCAGCAAG | |
| | TACCTGAACTGGTATCAGCAGAAGCCCGACGGCACCGT | |
| | CAAGCTGCTGATCTACCACACCAGCCGGCTGCACAGCG | |
| | GCGTGCCCAGCCGGTTTAGCGGCAGCGGCTCCGGCACC | |
| | GACTACAGCCTGACCATCTCCAACCTGGAACAGGAAGA | |
| | TATCGCCACCTACTTTTGCCAGCAGGGCAACACACTGC | |
| | CCTACACCTTTGGCGGCGGAACAAAGCTGGAAATCACC | |
| | ATTGAAGTTATGTATCCTCCTCCTTACCTAGACAATGAG | |
| | AAGAGCAATGGAACCATTATCCATGTGAAAGGGAAAC | |
| | ACCTTTGTCCAAGTCCCCTATTTCCCGGACCTTCTAAGC | |
| | CCATGTTCTGGGTGCTGGTGGTGGTCGGAGGCGTGCTG | |
| | GCCTGCTACAGCCTGCTGGTCACCGTGGCCTTCATCATC | |
| | TTTTGGGTGAAACGGGGCAGAAAGAAACTCCTGTATAT | |
| | ATTCAAACAACCATTTATGAGACCAGTACAAACTACTC | |
| | AAGAGGAAGATGGCTGTAGCTGCCGATTTCCAGAAGAA | |
| | GAAGAAGGAGGATGTGAACTGCGGGTGAAGTTCAGCA | |
| | GAAGCGCCGACGCCCCTGCCTACCAGCAGGGCCAGAAT | |
| | CAGCTGTACAACGAGCTGAACCTGGGCAGAAGGGAAG | |
| | AGTACGACGTCCTGGATAAGCGGAGAGGCCGGGACCCT | |
| | GAGATGGGCGGCAAGCCTCGGCGGAAGAACCCCCAGG | |
| | AAGGCCTGTATAACGAACTGCAGAAAGACAAGATGGC | |
| | CGAGGCCTACAGCGAGATCGGCATGAAGGGCGAGCGG | |
| | AGGCGGGGCAAGGGCCACGACGGCCTGTATCAGGGCC | |
| | TGTCCACCGCCACCAAGGATACCTACGACGCCCTGCAC | |
| | ATGCAGGCCCTGCCCCCAAGGTGA | |
| 6 | METDTLLLWVLLLWVPGSTGDIVLTQSPAILSASPGEKVT | CAR Construct 6 |
| | MTCRASSSVNYMDWYQKKPGSSPKPWIYATSNLASGVPA | |
| | RFSGSGSGTSYSLTISRVEAEDAATYYCQQWSFNPPTFGG | |
| | GTKLEIKGSTSGGGSGGGSGGGGSSEVQLQQSGAELVKPG | |
| | ASVKMSCKASGYTFTSYNMHWVKQTPGQGLEWIGAIYP | |
| | GNGDTSYNQKFKGKATLTADKSSSTAYMQLSSLTSEDSA | |
| | DYYCARSNYYGSSYWFFDVWGAGTTVTVSSGGGGSGGG | |
| | GSGGGGSGGGGSEVKLQESGPGLVAPSQSLSVTCTVSGVS | |
| | LPDYGVSWIRQPPRKGLEWLGVIWGSETTYYNSALKSRLT | |
| | IIKDNSKSQVFLKMNSLQTDDTAIYYCAKHYYYGGSYAM | |
| | DYWGQGTSVTVSSGSTSGSGKPGSGEGSTKGDIQMTQTTS | |
| | SLSASLGDRVTISCRASQDISKYLNWYQQKPDGTVKLLIY | |
| | HTSRLHSGVPSRFSGSGSGTDYSLTISNLEQEDIATYFCQQ | |
| | GNTLPYTFGGGTKLEITIEVMYPPPYLDNEKSNGTIIHVKG | |
| | KHLCPSPLFPGPSKP | |
| | MFWVLVVVGGVLACYSLLVTVAFIIFWVKRGRKKLLYIF | |
| | KQPFMRPVQTTQEEDGCSCRFPEEEEGGCELRVKFSRSAD | |
| | APAYQQGQNQLYNELNLGRREEYDVLDKRRGRDPEMGG | |
| | KPRRKNPQEGLYNELQKDKMAEAYSEIGMKGERRRGKG | |
| | HDGLYQGLSTATKDTYDALHMQALPPR | |
| 7 | GCTAGCGCCGCCACCATGGAGACAGACACACTCCTGCT | CAR Construct 7 |
| | ATGGGTGCTGCTGCTCTGGGTTCCAGGTTCCACAGGTG | |
| | ACATTGTGCTGACCCAATCTCCAGCTATCCTGTCTGCAT | |
| | CTCCAGGGGAGAAGGTCACAATGACTTGCAGGGCCAGC | |
| | TCAAGTGTAAATTACATGGACTGGTACCAGAAGAAGCC | |
| | AGGATCCTCCCCCAAACCCTGGATTTATGCCACATCCA | |
| | ACCTGGCTTCTGGAGTCCCTGCTCGCTTCAGTGGCAGTG | |
| | GGTCTGGGACCTCTTACTCTCTCACAATCAGCAGAGTG | |
| | GAGGCTGAAGATGCTGCCACTTATTACTGCCAGCAGTG | |
| | GAGTTTTAATCCACCCACGTTCGGAGGGGGGACCAAGC | |
| | TGGAAATAAAAGGCAGTACTAGCGGTGGTGGCTCCGGG | |
| | GGCGGTTCCGGTGGGGGCGGCAGCAGCGAGGTGCAGC | |
| | TGCAGCAGTCTGGGGCTGAGCTGGTGAAGCCTGGGGCC | |
| | TCAGTGAAGATGTCCTGCAAGGCTTCTGGCTACACATTT | |
| | ACCAGTTACAATATGCACTGGGTAAAGCAGACACCTGG | |
| | ACAGGGCCTGGAATGGATTGGAGCTATTTATCCAGGAA | |
| | ATGGTGATACTTCCTACAATCAGAAGTTCAAAGGCAAG | |
| | GCCACATTGACTGCAGACAAATCCTCCAGCACAGCCTA | |
| | CATGCAGCTCAGCAGCCTGACATCTGAGGACTCTGCGG | |
| | ACTATTACTGTGCAAGATCTAATTATTACGGTAGTAGCT | |
| | ACTGGTTCTTCGATGTCTGGGGCGCAGGGACCACGGTC | |
| | ACAGTAAGTAGCGGTGGAGGCGGCAGTGGCGGAGGTG | |
| | GGAGCGGAGGGGGCGGTTCCGGTGGCGGGGGATCTGA | |
| | GGTGAAGCTGCAGGAAAGCGGCCCTGGCCTGGTGGCCC | |
| | CCAGCCAGAGCCTGAGCGTGACCTGCACCGTGAGCGGC | |
| | GTGAGCCTGCCCGACTACGGCGTGAGCTGGATCCGGCA | |
| | GCCCCCCAGGAAGGGCCTGGAATGGCTGGGCGTGATCT | |
| | GGGGCAGCGAGACCACCTACTACAACAGCGCCCTGAA | |
| | GAGCCGGCTGACCATCATCAAGGACAACAGCAAGAGC | |
| | CAGGTGTTCCTGAAGATGAACAGCCTGCAGACCGACGA | |
| | CACCGCCATCTACTACTGCGCCAAGCACTACTACTACG | |
| | GCGGCAGCTACGCCATGGACTACTGGGGCCAGGGCACC | |
| | AGCGTGACCGTGAGCAGCGGCAGCACCTCCGGCAGCG | |
| | GCAAGCCTGGCAGCGGCGAGGGCAGCACCAAGGGCGA | |
| | CATCCAGATGACCCAGACCACCTCCAGCCTGAGCGCCA | |
| | GCCTGGGCGACCGGGTGACCATCAGCTGCCGGGCCAGC | |
| | CAGGACATCAGCAAGTACCTGAACTGGTATCAGCAGAA | |
| | GCCCGACGGCACCGTCAAGCTGCTGATCTACCACACCA | |
| | GCCGGCTGCACAGCGGCGTGCCCAGCCGGTTTAGCGGC | |
| | AGCGGCTCCGGCACCGACTACAGCCTGACCATCTCCAA | |
| | CCTGGAACAGGAAGATATCGCCACCTACTTTTGCCAGC | |
| | AGGGCAACACACTGCCCTACACCTTTGGCGGCGGAACA | |
| | AAGCTGGAAATCACCACTACAACTCCTGCCCCACGGCC | |
| | ACCTACACCTGCGCCTACAATTGCCAGCCAGCCACTGT | |
| | CCTTACGACCAGAGGCTTGCCGCCCCGCTGCGGGAGGC | |
| | GCCGTCCATACCCGGGGCCTTGACTTCGCATGCGATATT | |
| | TACATTTGGGCACCCTTGGCCGGCACCTGCGGAGTTTTA | |
| | CTTCTGAGCTTGGTGATAACGCTGTACTGTAAACGGGG | |
| | CAGAAAGAAACTCCTGTATATATTCAAACAACCATTTA | |
| | TGAGACCAGTACAAACTACTCAAGAGGAAGATGGCTGT | |
| | AGCTGCCGATTTCCAGAAGAAGAAGAAGGAGGATGTG | |
| | AACTGCGGGTGAAGTTCAGCAGAAGCGCCGACGCCCCT | |
| | GCCTACCAGCAGGGCCAGAATCAGCTGTACAACGAGCT | |
| | GAACCTGGGCAGAAGGGAAGAGTACGACGTCCTGGAT | |
| | AAGCGGAGAGGCCGGGACCCTGAGATGGGCGGCAAGC | |
| | CTCGGCGGAAGAACCCCCAGGAAGGCCTGTATAACGA | |
| | ACTGCAGAAAGACAAGATGGCCGAGGCCTACAGCGAG | |
| | ATCGGCATGAAGGGCGAGCGGAGGCGGGGCAAGGGCC | |
| | ACGACGGCCTGTATCAGGGCCTGTCCACCGCCACCAAG | |
| | GATACCTACGACGCCCTGCACATGCAGGCCCTGCCCCC | |
| | AAGGTGAGTCGAC | |
| 8 | METDTLLLWVLLLWVPGSTGDIVLTQSPAILSASPGEKVT | CAR Construct 8 |
| | MTCRASSSVNYMDWYQKKPGSSPKPWIYATSNLASGVPA | |
| | RFSGSGSGTSYSLTISRVEAEDAATYYCQQWSFNPPTFGG | |
| | GTKLEIKGSTSGGGSGGGSGGGGSSEVQLQQSGAELVKPG | |
| | ASVKMSCKASGYTFTSYNMHWVKQTPGQGLEWIGAIYP | |
| | GNGDTSYNQKFKGKATLTADKSSSTAYMQLSSLTSEDSA | |
| | DYYCARSNYYGSSYWFFDVWGAGTTVTVSSGGGGSGGG | |
| | GSGGGGSGGGGSEVKLQESGPGLVAPSQSLSVTCTVSGVS | |
| | LPDYGVSWIRQPPRKGLEWLGVIWGSETTYYNSALKSRLT | |
| | IIKDNSKSQVFLKMNSLQTDDTAIYYCAKHYYYGGSYAM | |
| | DYWGQGTSVTVSSGSTSGSGKPGSGEGSTKGDIQMTQTTS | |
| | SLSASLGDRVTISCRASQDISKYLNWYQQKPDGTVKLLIY | |
| | HTSRLHSGVPSRFSGSGSGTDYSLTISNLEQEDIATYFCQQ | |
| | GNTLPYTFGGGTKLEITTTTPAPRPPTPAPTIASQPLSLRPE | |
| | ACRPAAGGAVHTRGLDFACDIYIWAPLAGTCGVLLLSLVI | |
| | TLYC | |
| | KRGRKKLLYIFKQPFMRPVQTTQEEDGCSCRFPEEEEGGC | |
| | ELRVKFSRSADAPAYQQGQNQLYNELNLGRREEYDVLDK | |
| | RRGRDPEMGGKPRRKNPQEGLYNELQKDKMAEAYSEIG | |
| | MKGERRRGKGHDGLYQGLSTATKDTYDALHMQALPPR | |
| 9 | ATGGAGACAGACACACTCCTGCTATGGGTGCTGCTGCT | CAR Construct 9 |
| | CTGGGTTCCAGGTTCCACAGGTGACATTGTGCTGACCC | |
| | AATCTCCAGCTATCCTGTCTGCATCTCCAGGGGAGAAG | |
| | GTCACAATGACTTGCAGGGCCAGCTCAAGTGTAAATTA | |
| | CATGGACTGGTACCAGAAGAAGCCAGGATCCTCCCCCA | |
| | AACCCTGGATTTATGCCACATCCAACCTGGCTTCTGGA | |
| | GTCCCTGCTCGCTTCAGTGGCAGTGGGTCTGGGACCTCT | |
| | TACTCTCTCACAATCAGCAGAGTGGAGGCTGAAGATGC | |
| | TGCCACTTATTACTGCCAGCAGTGGAGTTTTAATCCACC | |
| | CACGTTCGGAGGGGGGACCAAGCTGGAAATAAAAGGC | |
| | AGTACTAGCGGTGGTGGCTCCGGGGGCGGTTCCGGTGG | |
| | GGGCGGCAGCAGCGAGGTGCAGCTGCAGCAGTCTGGG | |
| | GCTGAGCTGGTGAAGCCTGGGGCCTCAGTGAAGATGTC | |
| | CTGCAAGGCTTCTGGCTACACATTTACCAGTTACAATAT | |
| | GCACTGGGTAAAGCAGACACCTGGACAGGGCCTGGAA | |
| | TGGATTGGAGCTATTTATCCAGGAAATGGTGATACTTC | |
| | CTACAATCAGAAGTTCAAAGGCAAGGCCACATTGACTG | |
| | CAGACAAATCCTCCAGCACAGCCTACATGCAGCTCAGC | |
| | AGCCTGACATCTGAGGACTCTGCGGACTATTACTGTGC | |
| | AAGATCTAATTATTACGGTAGTAGCTACTGGTTCTTCGA | |
| | TGTCTGGGGCGCAGGGACCACGGTCACAGTAAGTAGCG | |
| | GTGGAGGCGGCAGTGGCGGAGGTGGGAGCGGAGGGGG | |
| | CGGTTCCGGTGGCGGGGGATCTGAGGTGAAGCTGCAGG | |
| | AAAGCGGCCCTGGCCTGGTGGCCCCCAGCCAGAGCCTG | |
| | AGCGTGACCTGCACCGTGAGCGGCGTGAGCCTGCCCGA | |
| | CTACGGCGTGAGCTGGATCCGGCAGCCCCCCAGGAAGG | |
| | GCCTGGAATGGCTGGGCGTGATCTGGGGCAGCGAGACC | |
| | ACCTACTACAACAGCGCCCTGAAGAGCCGGCTGACCAT | |
| | CATCAAGGACAACAGCAAGAGCCAGGTGTTCCTGAAG | |
| | ATGAACAGCCTGCAGACCGACGACACCGCCATCTACTA | |
| | CTGCGCCAAGCACTACTACTACGGCGGCAGCTACGCCA | |
| | TGGACTACTGGGGCCAGGGCACCAGCGTGACCGTGAGC | |
| | AGCGGCAGCACCTCCGGCAGCGGCAAGCCTGGCAGCG | |
| | GCGAGGGCAGCACCAAGGGCGACATCCAGATGACCCA | |
| | GACCACCTCCAGCCTGAGCGCCAGCCTGGGCGACCGGG | |
| | TGACCATCAGCTGCCGGGCCAGCCAGGACATCAGCAAG | |
| | TACCTGAACTGGTATCAGCAGAAGCCCGACGGCACCGT | |
| | CAAGCTGCTGATCTACCACACCAGCCGGCTGCACAGCG | |
| | GCGTGCCCAGCCGGTTTAGCGGCAGCGGCTCCGGCACC | |
| | GACTACAGCCTGACCATCTCCAACCTGGAACAGGAAGA | |
| | TATCGCCACCTACTTTTGCCAGCAGGGCAACACACTGC | |
| | CCTACACCTTTGGCGGCGGAACAAAGCTGGAAATCACC | |
| | TCTGGGCAAGTTCTTCTTGAGTCTAATATCAAGGTGCTG | |
| | CCCACCTGGTCTACTCCTGTCCAACCGATGTTCTGGGTG | |
| | CTGGTGGTGGTCGGAGGCGTGCTGGCCTGCTACAGCCT | |
| | GCTGGTCACCGTGGCCTTCATCATCTTTTGGGTGAAACG | |
| | GGGCAGAAAGAAACTCCTGTATATATTCAAACAACCAT | |
| | TTATGAGACCAGTACAAACTACTCAAGAGGAAGATGGC | |
| | TGTAGCTGCCGATTTCCAGAAGAAGAAGAAGGAGGAT | |
| | GTGAACTGCGGGTGAAGTTCAGCAGAAGCGCCGACGCC | |
| | CCTGCCTACCAGCAGGGCCAGAATCAGCTGTACAACGA | |
| | GCTGAACCTGGGCAGAAGGGAAGAGTACGACGTCCTG | |
| | GATAAGCGGAGAGGCCGGGACCCTGAGATGGGCGGCA | |
| | AGCCTCGGCGGAAGAACCCCCAGGAAGGCCTGTATAAC | |
| | GAACTGCAGAAAGACAAGATGGCCGAGGCCTACAGCG | |
| | AGATCGGCATGAAGGGCGAGCGGAGGCGGGGCAAGGG | |
| | CCACGACGGCCTGTATCAGGGCCTGTCCACCGCCACCA | |
| | AGGATACCTACGACGCCCTGCACATGCAGGCCCTGCCC | |
| | CCAAGGTGAGTCGAC | |
| 10 | METDTLLLWVLLLWVPGSTGDIVLTQSPAILSASPGEKVT | CAR Construct 10 |
| | MTCRASSSVNYMDWYQKKPGSSPKPWIYATSNLASGVPA | |
| | RFSGSGSGTSYSLTISRVEAEDAATYYCQQWSFNPPTFGG | |
| | GTKLEIKGSTSGGGSGGGSGGGGSSEVQLQQSGAELVKPG | |
| | ASVKMSCKASGYTFTSYNMHWVKQTPGQGLEWIGAIYP | |
| | GNGDTSYNQKFKGKATLTADKSSSTAYMQLSSLTSEDSA | |
| | DYYCARSNYYGSSYWFFDVWGAGTTVTVSSGGGGSGGG | |
| | GSGGGGSGGGGSEVKLQESGPGLVAPSQSLSVTCTVSGVS | |
| | LPDYGVSWIRQPPRKGLEWLGVIWGSETTYYNSALKSRLT | |
| | IIKDNSKSQVFLKMNSLQTDDTAIYYCAKHYYYGGSYAM | |
| | DYWGQGTSVTVSSGSTSGSGKPGSGEGSTKGDIQMTQTTS | |
| | SLSASLGDRVTISCRASQDISKYLNWYQQKPDGTVKLLIY | |
| | HTSRLHSGVPSRFSGSGSGTDYSLTISNLEQEDIATYFCQQ | |
| | GNTLPYTFGGGTKLEITSGQVLLESNIKVLPTWSTPVQP | |
| | MFWVLVVVGGVLACYSLLVTVAFIIFWVKRGRKKLLYIF | |
| | KQPFMRPVQTTQEEDGCSCRFPEEEEGGCELRVKFSRSAD | |
| | APAYQQGQNQLYNELNLGRREEYDVLDKRRGRDPEMGG | |
| | KPRRKNPQEGLYNELQKDKMAEAYSEIGMKGERRRGKG | |
| | HDGLYQGLSTATKDTYDALHMQALPPR | |
| 11 | ATGGAGACAGACACACTCCTGCTATGGGTGCTGCTGCT | CAR Construct 11 |
| | CTGGGTTCCAGGTTCCACAGGTGACATTGTGCTGACCC | |
| | AATCTCCAGCTATCCTGTCTGCATCTCCAGGGGAGAAG | |
| | GTCACAATGACTTGCAGGGCCAGCTCAAGTGTAAATTA | |
| | CATGGACTGGTACCAGAAGAAGCCAGGATCCTCCCCCA | |
| | AACCCTGGATTTATGCCACATCCAACCTGGCTTCTGGA | |
| | GTCCCTGCTCGCTTCAGTGGCAGTGGGTCTGGGACCTCT | |
| | TACTCTCTCACAATCAGCAGAGTGGAGGCTGAAGATGC | |
| | TGCCACTTATTACTGCCAGCAGTGGAGTTTTAATCCACC | |
| | CACGTTCGGAGGGGGGACCAAGCTGGAAATAAAAGGC | |
| | AGTACTAGCGGTGGTGGCTCCGGGGGCGGTTCCGGTGG | |
| | GGGCGGCAGCAGCGAGGTGCAGCTGCAGCAGTCTGGG | |
| | GCTGAGCTGGTGAAGCCTGGGGCCTCAGTGAAGATGTC | |
| | CTGCAAGGCTTCTGGCTACACATTTACCAGTTACAATAT | |
| | GCACTGGGTAAAGCAGACACCTGGACAGGGCCTGGAA | |
| | TGGATTGGAGCTATTTATCCAGGAAATGGTGATACTTC | |
| | CTACAATCAGAAGTTCAAAGGCAAGGCCACATTGACTG | |
| | CAGACAAATCCTCCAGCACAGCCTACATGCAGCTCAGC | |
| | AGCCTGACATCTGAGGACTCTGCGGACTATTACTGTGC | |
| | AAGATCTAATTATTACGGTAGTAGCTACTGGTTCTTCGA | |
| | TGTCTGGGGCGCAGGGACCACGGTCACAGTAAGTAGCG | |
| | GTGGAGGCGGCAGTGGCGGAGGTGGGAGCGGAGGGGG | |
| | CGGTTCCGGTGGCGGGGGATCTGAGGTGAAGCTGCAGG | |
| | AAAGCGGCCCTGGCCTGGTGGCCCCCAGCCAGAGCCTG | |
| | AGCGTGACCTGCACCGTGAGCGGCGTGAGCCTGCCCGA | |
| | CTACGGCGTGAGCTGGATCCGGCAGCCCCCCAGGAAGG | |
| | GCCTGGAATGGCTGGGCGTGATCTGGGGCAGCGAGACC | |
| | ACCTACTACAACAGCGCCCTGAAGAGCCGGCTGACCAT | |
| | CATCAAGGACAACAGCAAGAGCCAGGTGTTCCTGAAG | |
| | ATGAACAGCCTGCAGACCGACGACACCGCCATCTACTA | |
| | CTGCGCCAAGCACTACTACTACGGCGGCAGCTACGCCA | |
| | TGGACTACTGGGGCCAGGGCACCAGCGTGACCGTGAGC | |
| | AGCGGCAGCACCTCCGGCAGCGGCAAGCCTGGCAGCG | |
| | GCGAGGGCAGCACCAAGGGCGACATCCAGATGACCCA | |
| | GACCACCTCCAGCCTGAGCGCCAGCCTGGGCGACCGGG | |
| | TGACCATCAGCTGCCGGGCCAGCCAGGACATCAGCAAG | |
| | TACCTGAACTGGTATCAGCAGAAGCCCGACGGCACCGT | |
| | CAAGCTGCTGATCTACCACACCAGCCGGCTGCACAGCG | |
| | GCGTGCCCAGCCGGTTTAGCGGCAGCGGCTCCGGCACC | |
| | GACTACAGCCTGACCATCTCCAACCTGGAACAGGAAGA | |
| | TATCGCCACCTACTTTTGCCAGCAGGGCAACACACTGC | |
| | CCTACACCTTTGGCGGCGGAACAAAGCTGGAAATCACC | |
| | AGCCAGCCACTGTCCTTACGACCAGAGGCTTGCCGCCC | |
| | CGCTGCGGGAGGCGCCGTCCATACCCGGGGCCTTGACT | |
| | TCGCATGCGATATGTTCTGGGTGCTGGTGGTGGTCGGA | |
| | GGCGTGCTGGCCTGCTACAGCCTGCTGGTCACCGTGGC | |
| | CTTCATCATCTTTTGGGTGAAACGGGGCAGAAAGAAAC | |
| | TCCTGTATATATTCAAACAACCATTTATGAGACCAGTAC | |
| | AAACTACTCAAGAGGAAGATGGCTGTAGCTGCCGATTT | |
| | CCAGAAGAAGAAGAAGGAGGATGTGAACTGCGGGTGA | |
| | AGTTCAGCAGAAGCGCCGACGCCCCTGCCTACCAGCAG | |
| | GGCCAGAATCAGCTGTACAACGAGCTGAACCTGGGCAG | |
| | AAGGGAAGAGTACGACGTCCTGGATAAGCGGAGAGGC | |
| | CGGGACCCTGAGATGGGCGGCAAGCCTCGGCGGAAGA | |
| | ACCCCCAGGAAGGCCTGTATAACGAACTGCAGAAAGA | |
| | CAAGATGGCCGAGGCCTACAGCGAGATCGGCATGAAG | |
| | GGCGAGCGGAGGCGGGGCAAGGGCCACGACGGCCTGT | |
| | ATCAGGGCCTGTCCACCGCCACCAAGGATACCTACGAC | |
| | GCCCTGCACATGCAGGCCCTGCCCCCAAGGTGAGTCGA | |
| | C | |
| 12 | METDTLLLWVLLLWVPGSTGDIVLTQSPAILSASPGEKVT | CAR Construct 12 |
| | MTCRASSSVNYMDWYQKKPGSSPKPWIYATSNLASGVPA | |
| | RFSGSGSGTSYSLTISRVEAEDAATYYCQQWSFNPPTFGG | |
| | GTKLEIKGSTSGGGSGGGSGGGGSSEVQLQQSGAELVKPG | |
| | ASVKMSCKASGYTFTSYNMHWVKQTPGQGLEWIGAIYP | |
| | GNGDTSYNQKFKGKATLTADKSSSTAYMQLSSLTSEDSA | |
| | DYYCARSNYYGSSYWFFDVWGAGTTVTVSSGGGGSGGG | |
| | GSGGGGSGGGGSEVKLQESGPGLVAPSQSLSVTCTVSGVS | |
| | LPDYGVSWIRQPPRKGLEWLGVIWGSETTYYNSALKSRLT | |
| | IIKDNSKSQVFLKMNSLQTDDTAIYYCAKHYYYGGSYAM | |
| | DYWGQGTSVTVSSGSTSGSGKPGSGEGSTKGDIQMTQTTS | |
| | SLSASLGDRVTISCRASQDISKYLNWYQQKPDGTVKLLIY | |
| | HTSRLHSGVPSRFSGSGSGTDYSLTISNLEQEDIATYFCQQ | |
| | GNTLPYTFGGGTKLEITSQPLSLRPEACRPAAGGAVHTRG | |
| | LDFACDMFWVLVVVGGVLACYSLLVTVAFIIFWVKRGRK | |
| | KLLYIFKQPFMRPVQTTQEEDGCSCRFPEEEEGGCELRVK | |
| | FSRSADAPAYQQGQNQLYNELNLGRREEYDVLDKRRGR | |
| | DPEMGGKPRRKNPQEGLYNELQKDKMAEAYSEIGMKGE | |
| | RRRGKGHDGLYQGLSTATKDTYDALHMQALPPR | |
| 13 | ATGGAGACAGACACACTCCTGCTATGGGTGCTGCTGCT | CAR Construct 13 |
| | CTGGGTTCCAGGTTCCACAGGTGACATTGTGCTGACCC | |
| | AATCTCCAGCTATCCTGTCTGCATCTCCAGGGGAGAAG | |
| | GTCACAATGACTTGCAGGGCCAGCTCAAGTGTAAATTA | |
| | CATGGACTGGTACCAGAAGAAGCCAGGATCCTCCCCCA | |
| | AACCCTGGATTTATGCCACATCCAACCTGGCTTCTGGA | |
| | GTCCCTGCTCGCTTCAGTGGCAGTGGGTCTGGGACCTCT | |
| | TACTCTCTCACAATCAGCAGAGTGGAGGCTGAAGATGC | |
| | TGCCACTTATTACTGCCAGCAGTGGAGTTTTAATCCACC | |
| | CACGTTCGGAGGGGGGACCAAGCTGGAAATAAAAGGC | |
| | AGTACTAGCGGTGGTGGCTCCGGGGGCGGTTCCGGTGG | |
| | GGGCGGCAGCAGCGAGGTGCAGCTGCAGCAGTCTGGG | |
| | GCTGAGCTGGTGAAGCCTGGGGCCTCAGTGAAGATGTC | |
| | CTGCAAGGCTTCTGGCTACACATTTACCAGTTACAATAT | |
| | GCACTGGGTAAAGCAGACACCTGGACAGGGCCTGGAA | |
| | TGGATTGGAGCTATTTATCCAGGAAATGGTGATACTTC | |
| | CTACAATCAGAAGTTCAAAGGCAAGGCCACATTGACTG | |
| | CAGACAAATCCTCCAGCACAGCCTACATGCAGCTCAGC | |
| | AGCCTGACATCTGAGGACTCTGCGGACTATTACTGTGC | |
| | AAGATCTAATTATTACGGTAGTAGCTACTGGTTCTTCGA | |
| | TGTCTGGGGCGCAGGGACCACGGTCACAGTAAGTAGCG | |
| | GTGGAGGCGGCAGTGGCGGAGGTGGGAGCGGAGGGGG | |
| | CGGTTCCGGTGGCGGGGGATCTGAGGTGAAGCTGCAGG | |
| | AAAGCGGCCCTGGCCTGGTGGCCCCCAGCCAGAGCCTG | |
| | AGCGTGACCTGCACCGTGAGCGGCGTGAGCCTGCCCGA | |
| | CTACGGCGTGAGCTGGATCCGGCAGCCCCCCAGGAAGG | |
| | GCCTGGAATGGCTGGGCGTGATCTGGGGCAGCGAGACC | |
| | ACCTACTACAACAGCGCCCTGAAGAGCCGGCTGACCAT | |
| | CATCAAGGACAACAGCAAGAGCCAGGTGTTCCTGAAG | |
| | ATGAACAGCCTGCAGACCGACGACACCGCCATCTACTA | |
| | CTGCGCCAAGCACTACTACTACGGCGGCAGCTACGCCA | |
| | TGGACTACTGGGGCCAGGGCACCAGCGTGACCGTGAGC | |
| | AGCGGCAGCACCTCCGGCAGCGGCAAGCCTGGCAGCG | |
| | GCGAGGGCAGCACCAAGGGCGACATCCAGATGACCCA | |
| | GACCACCTCCAGCCTGAGCGCCAGCCTGGGCGACCGGG | |
| | TGACCATCAGCTGCCGGGCCAGCCAGGACATCAGCAAG | |
| | TACCTGAACTGGTATCAGCAGAAGCCCGACGGCACCGT | |
| | CAAGCTGCTGATCTACCACACCAGCCGGCTGCACAGCG | |
| | GCGTGCCCAGCCGGTTTAGCGGCAGCGGCTCCGGCACC | |
| | GACTACAGCCTGACCATCTCCAACCTGGAACAGGAAGA | |
| | TATCGCCACCTACTTTTGCCAGCAGGGCAACACACTGC | |
| | CCTACACCTTTGGCGGCGGAACAAAGCTGGAAATCACC | |
| | CGCCCCGCTGCGGGAGGCGCCGTCCATACCCGGGGCCT | |
| | TGACTTCGCATGCGATATGTTCTGGGTGCTGGTGGTGGT | |
| | CGGAGGCGTGCTGGCCTGCTACAGCCTGCTGGTCACCG | |
| | TGGCCTTCATCATCTTTTGGGTGAAACGGGGCAGAAAG | |
| | AAACTCCTGTATATATTCAAACAACCATTTATGAGACC | |
| | AGTACAAACTACTCAAGAGGAAGATGGCTGTAGCTGCC | |
| | GATTTCCAGAAGAAGAAGAAGGAGGATGTGAACTGCG | |
| | GGTGAAGTTCAGCAGAAGCGCCGACGCCCCTGCCTACC | |
| | AGCAGGGCCAGAATCAGCTGTACAACGAGCTGAACCTG | |
| | GGCAGAAGGGAAGAGTACGACGTCCTGGATAAGCGGA | |
| | GAGGCCGGGACCCTGAGATGGGCGGCAAGCCTCGGCG | |
| | GAAGAACCCCCAGGAAGGCCTGTATAACGAACTGCAG | |
| | AAAGACAAGATGGCCGAGGCCTACAGCGAGATCGGCA | |
| | TGAAGGGCGAGCGGAGGCGGGGCAAGGGCCACGACGG | |
| | CCTGTATCAGGGCCTGTCCACCGCCACCAAGGATACCT | |
| | ACGACGCCCTGCACATGCAGGCCCTGCCCCCAAGGTGA | |
| | GTCGAC | |
| 14 | METDTLLLWVLLLWVPGSTGDIVLTQSPAILSASPGEKVT | CAR Construct 14 |
| | MTCRASSSVNYMDWYQKKPGSSPKPWIYATSNLASGVPA | |
| | RFSGSGSGTSYSLTISRVEAEDAATYYCQQWSFNPPTFGG | |
| | GTKLEIKGSTSGGGSGGGSGGGGSSEVQLQQSGAELVKPG | |
| | ASVKMSCKASGYTFTSYNMHWVKQTPGQGLEWIGAIYP | |
| | GNGDTSYNQKFKGKATLTADKSSSTAYMQLSSLTSEDSA | |
| | DYYCARSNYYGSSYWFFDVWGAGTTVTVSSGGGGSGGG | |
| | GSGGGGSGGGGSEVKLQESGPGLVAPSQSLSVTCTVSGVS | |
| | LPDYGVSWIRQPPRKGLEWLGVIWGSETTYYNSALKSRLT | |
| | IIKDNSKSQVFLKMNSLQTDDTAIYYCAKHYYYGGSYAM | |
| | DYWGQGTSVTVSSGSTSGSGKPGSGEGSTKGDIQMTQTTS | |
| | SLSASLGDRVTISCRASQDISKYLNWYQQKPDGTVKLLIY | |
| | HTSRLHSGVPSRFSGSGSGTDYSLTISNLEQEDIATYFCQQ | |
| | GNTLPYTFGGGTKLEITRPAAGGAVHTRGLDFACDMFWV | |
| | LVVVGGVLACYSLLVTVAFIIFWVKRGRKKLLYIFKQPFM | |
| | RPVQTTQEEDGCSCRFPEEEEGGCELRVKFSRSADAPAYQ | |
| | QGQNQLYNELNLGRREEYDVLDKRRGRDPEMGGKPRRK | |
| | NPQEGLYNELQKDKMAEAYSEIGMKGERRRGKGHDGLY | |
| | QGLSTATKDTYDALHMQALPPR | |
| 15 | GCTAGCGCCGCCACCATGGAGACAGACACACTCCTGCT | CAR Construct 15 |
| | ATGGGTGCTGCTGCTCTGGGTTCCAGGTTCCACAGGTG | |
| | ACATTGTGCTGACCCAATCTCCAGCTATCCTGTCTGCAT | |
| | CTCCAGGGGAGAAGGTCACAATGACTTGCAGGGCCAGC | |
| | TCAAGTGTAAATTACATGGACTGGTACCAGAAGAAGCC | |
| | AGGATCCTCCCCCAAACCCTGGATTTATGCCACATCCA | |
| | ACCTGGCTTCTGGAGTCCCTGCTCGCTTCAGTGGCAGTG | |
| | GGTCTGGGACCTCTTACTCTCTCACAATCAGCAGAGTG | |
| | GAGGCTGAAGATGCTGCCACTTATTACTGCCAGCAGTG | |
| | GAGTTTTAATCCACCCACGTTCGGAGGGGGGACCAAGC | |
| | TGGAAATAAAAGGCAGTACTAGCGGTGGTGGCTCCGGG | |
| | GGCGGTTCCGGTGGGGGCGGCAGCAGCGAGGTGCAGC | |
| | TGCAGCAGTCTGGGGCTGAGCTGGTGAAGCCTGGGGCC | |
| | TCAGTGAAGATGTCCTGCAAGGCTTCTGGCTACACATTT | |
| | ACCAGTTACAATATGCACTGGGTAAAGCAGACACCTGG | |
| | ACAGGGCCTGGAATGGATTGGAGCTATTTATCCAGGAA | |
| | ATGGTGATACTTCCTACAATCAGAAGTTCAAAGGCAAG | |
| | GCCACATTGACTGCAGACAAATCCTCCAGCACAGCCTA | |
| | CATGCAGCTCAGCAGCCTGACATCTGAGGACTCTGCGG | |
| | ACTATTACTGTGCAAGATCTAATTATTACGGTAGTAGCT | |
| | ACTGGTTCTTCGATGTCTGGGGCGCAGGGACCACGGTC | |
| | ACAGTAAGTAGCGGTGGAGGCGGCAGTGGCGGAGGTG | |
| | GGAGCGGAGGGGGCGGTTCCGGTGGCGGGGGATCTGA | |
| | GGTGAAGCTGCAGGAAAGCGGCCCTGGCCTGGTGGCCC | |
| | CCAGCCAGAGCCTGAGCGTGACCTGCACCGTGAGCGGC | |
| | GTGAGCCTGCCCGACTACGGCGTGAGCTGGATCCGGCA | |
| | GCCCCCCAGGAAGGGCCTGGAATGGCTGGGCGTGATCT | |
| | GGGGCAGCGAGACCACCTACTACAACAGCGCCCTGAA | |
| | GAGCCGGCTGACCATCATCAAGGACAACAGCAAGAGC | |
| | CAGGTGTTCCTGAAGATGAACAGCCTGCAGACCGACGA | |
| | CACCGCCATCTACTACTGCGCCAAGCACTACTACTACG | |
| | GCGGCAGCTACGCCATGGACTACTGGGGCCAGGGCACC | |
| | AGCGTGACCGTGAGCAGCGGCAGCACCTCCGGCAGCG | |
| | GCAAGCCTGGCAGCGGCGAGGGCAGCACCAAGGGCGA | |
| | CATCCAGATGACCCAGACCACCTCCAGCCTGAGCGCCA | |
| | GCCTGGGCGACCGGGTGACCATCAGCTGCCGGGCCAGC | |
| | CAGGACATCAGCAAGTACCTGAACTGGTATCAGCAGAA | |
| | GCCCGACGGCACCGTCAAGCTGCTGATCTACCACACCA | |
| | GCCGGCTGCACAGCGGCGTGCCCAGCCGGTTTAGCGGC | |
| | AGCGGCTCCGGCACCGACTACAGCCTGACCATCTCCAA | |
| | CCTGGAACAGGAAGATATCGCCACCTACTTTTGCCAGC | |
| | AGGGCAACACACTGCCCTACACCTTTGGCGGCGGAACA | |
| | AAGCTGGAAATCACCGAGCCTAAAAGTTGTGATAAAAC | |
| | GCATACCTGTCCAATGTTCTGGGTGCTGGTGGTGGTCG | |
| | GAGGCGTGCTGGCCTGCTACAGCCTGCTGGTCACCGTG | |
| | GCCTTCATCATCTTTTGGGTGAAACGGGGCAGAAAGAA | |
| | ACTCCTGTATATATTCAAACAACCATTTATGAGACCAGT | |
| | ACAAACTACTCAAGAGGAAGATGGCTGTAGCTGCCGAT | |
| | TTCCAGAAGAAGAAGAAGGAGGATGTGAACTGCGGGT | |
| | GAAGTTCAGCAGAAGCGCCGACGCCCCTGCCTACCAGC | |
| | AGGGCCAGAATCAGCTGTACAACGAGCTGAACCTGGGC | |
| | AGAAGGGAAGAGTACGACGTCCTGGATAAGCGGAGAG | |
| | GCCGGGACCCTGAGATGGGCGGCAAGCCTCGGCGGAA | |
| | GAACCCCCAGGAAGGCCTGTATAACGAACTGCAGAAA | |
| | GACAAGATGGCCGAGGCCTACAGCGAGATCGGCATGA | |
| | AGGGCGAGCGGAGGCGGGGCAAGGGCCACGACGGCCT | |
| | GTATCAGGGCCTGTCCACCGCCACCAAGGATACCTACG | |
| | ACGCCCTGCACATGCAGGCCCTGCCCCCAAGGTGAGTC | |
| | GAC | |
| 16 | METDTLLLWVLLLWVPGSTGDIVLTQSPAILSASPGEKVT | CAR Construct 16 |
| | MTCRASSSVNYMDWYQKKPGSSPKPWIYATSNLASGVPA | |
| | RFSGSGSGTSYSLTISRVEAEDAATYYCQQWSFNPPTFGG | |
| | GTKLEIKGSTSGGGSGGGSGGGGSSEVQLQQSGAELVKPG | |
| | ASVKMSCKASGYTFTSYNMHWVKQTPGQGLEWIGAIYP | |
| | GNGDTSYNQKFKGKATLTADKSSSTAYMQLSSLTSEDSA | |
| | DYYCARSNYYGSSYWFFDVWGAGTTVTVSSGGGGSGGG | |
| | GSGGGGSGGGGSEVKLQESGPGLVAPSQSLSVTCTVSGVS | |
| | LPDYGVSWIRQPPRKGLEWLGVIWGSETTYYNSALKSRLT | |
| | IIKDNSKSQVFLKMNSLQTDDTAIYYCAKHYYYGGSYAM | |
| | DYWGQGTSVTVSSGSTSGSGKPGSGEGSTKGDIQMTQTTS | |
| | SLSASLGDRVTISCRASQDISKYLNWYQQKPDGTVKLLIY | |
| | HTSRLHSGVPSRFSGSGSGTDYSLTISNLEQEDIATYFCQQ | |
| | GNTLPYTFGGGTKLEITEPKSCDKTHTCP | |
| | MFWVLVVVGGVLACYSLLVTVAFIIFWVKRGRKKLLYIF | |
| | KQPFMRPVQTTQEEDGCSCRFPEEEEGGCELRVKFSRSAD | |
| | APAYQQGQNQLYNELNLGRREEYDVLDKRRGRDPEMGG | |
| | KPRRKNPQEGLYNELQKDKMAEAYSEIGMKGERRRGKG | |
| | HDGLYQGLSTATKDTYDALHMQALPPR | |
| 17 | GCTAGCGCCGCCACCATGGAGACAGACACACTCCTGCT | CAR Construct 17 |
| | ATGGGTGCTGCTGCTCTGGGTTCCAGGTTCCACAGGTG | |
| | ACATTGTGCTGACCCAATCTCCAGCTATCCTGTCTGCAT | |
| | CTCCAGGGGAGAAGGTCACAATGACTTGCAGGGCCAGC | |
| | TCAAGTGTAAATTACATGGACTGGTACCAGAAGAAGCC | |
| | AGGATCCTCCCCCAAACCCTGGATTTATGCCACATCCA | |
| | ACCTGGCTTCTGGAGTCCCTGCTCGCTTCAGTGGCAGTG | |
| | GGTCTGGGACCTCTTACTCTCTCACAATCAGCAGAGTG | |
| | GAGGCTGAAGATGCTGCCACTTATTACTGCCAGCAGTG | |
| | GAGTTTTAATCCACCCACGTTCGGAGGGGGGACCAAGC | |
| | TGGAAATAAAAGGCAGTACTAGCGGTGGTGGCTCCGGG | |
| | GGCGGTTCCGGTGGGGGCGGCAGCAGCGAGGTGCAGC | |
| | TGCAGCAGTCTGGGGCTGAGCTGGTGAAGCCTGGGGCC | |
| | TCAGTGAAGATGTCCTGCAAGGCTTCTGGCTACACATTT | |
| | ACCAGTTACAATATGCACTGGGTAAAGCAGACACCTGG | |
| | ACAGGGCCTGGAATGGATTGGAGCTATTTATCCAGGAA | |
| | ATGGTGATACTTCCTACAATCAGAAGTTCAAAGGCAAG | |
| | GCCACATTGACTGCAGACAAATCCTCCAGCACAGCCTA | |
| | CATGCAGCTCAGCAGCCTGACATCTGAGGACTCTGCGG | |
| | ACTATTACTGTGCAAGATCTAATTATTACGGTAGTAGCT | |
| | ACTGGTTCTTCGATGTCTGGGGCGCAGGGACCACGGTC | |
| | ACAGTAAGTAGCGGTGGAGGCGGCAGTGGCGGAGGTG | |
| | GGAGCGGAGGGGGCGGTTCCGGTGGCGGGGGATCTGA | |
| | GGTGAAGCTGCAGGAAAGCGGCCCTGGCCTGGTGGCCC | |
| | CCAGCCAGAGCCTGAGCGTGACCTGCACCGTGAGCGGC | |
| | GTGAGCCTGCCCGACTACGGCGTGAGCTGGATCCGGCA | |
| | GCCCCCCAGGAAGGGCCTGGAATGGCTGGGCGTGATCT | |
| | GGGGCAGCGAGACCACCTACTACAACAGCGCCCTGAA | |
| | GAGCCGGCTGACCATCATCAAGGACAACAGCAAGAGC | |
| | CAGGTGTTCCTGAAGATGAACAGCCTGCAGACCGACGA | |
| | CACCGCCATCTACTACTGCGCCAAGCACTACTACTACG | |
| | GCGGCAGCTACGCCATGGACTACTGGGGCCAGGGCACC | |
| | AGCGTGACCGTGAGCAGCGGCAGCACCTCCGGCAGCG | |
| | GCAAGCCTGGCAGCGGCGAGGGCAGCACCAAGGGCGA | |
| | CATCCAGATGACCCAGACCACCTCCAGCCTGAGCGCCA | |
| | GCCTGGGCGACCGGGTGACCATCAGCTGCCGGGCCAGC | |
| | CAGGACATCAGCAAGTACCTGAACTGGTATCAGCAGAA | |
| | GCCCGACGGCACCGTCAAGCTGCTGATCTACCACACCA | |
| | GCCGGCTGCACAGCGGCGTGCCCAGCCGGTTTAGCGGC | |
| | AGCGGCTCCGGCACCGACTACAGCCTGACCATCTCCAA | |
| | CCTGGAACAGGAAGATATCGCCACCTACTTTTGCCAGC | |
| | AGGGCAACACACTGCCCTACACCTTTGGCGGCGGAACA | |
| | AAGCTGGAAATCACCGAGCCAAAGTCATGTGATACACC | |
| | TCCTCCCTGTCCCATGTTCTGGGTGCTGGTGGTGGTCGG | |
| | AGGCGTGCTGGCCTGCTACAGCCTGCTGGTCACCGTGG | |
| | CCTTCATCATCTTTTGGGTGAAACGGGGCAGAAAGAAA | |
| | CTCCTGTATATATTCAAACAACCATTTATGAGACCAGTA | |
| | CAAACTACTCAAGAGGAAGATGGCTGTAGCTGCCGATT | |
| | TCCAGAAGAAGAAGAAGGAGGATGTGAACTGCGGGTG | |
| | AAGTTCAGCAGAAGCGCCGACGCCCCTGCCTACCAGCA | |
| | GGGCCAGAATCAGCTGTACAACGAGCTGAACCTGGGCA | |
| | GAAGGGAAGAGTACGACGTCCTGGATAAGCGGAGAGG | |
| | CCGGGACCCTGAGATGGGCGGCAAGCCTCGGCGGAAG | |
| | AACCCCCAGGAAGGCCTGTATAACGAACTGCAGAAAG | |
| | ACAAGATGGCCGAGGCCTACAGCGAGATCGGCATGAA | |
| | GGGCGAGCGGAGGCGGGGCAAGGGCCACGACGGCCTG | |
| | TATCAGGGCCTGTCCACCGCCACCAAGGATACCTACGA | |
| | CGCCCTGCACATGCAGGCCCTGCCCCCAAGGTGAGTCG | |
| | AC | |
| 18 | METDTLLLWVLLLWVPGSTGDIVLTQSPAILSASPGEKVT | CAR Construct 18 |
| | MTCRASSSVNYMDWYQKKPGSSPKPWIYATSNLASGVPA | |
| | RFSGSGSGTSYSLTISRVEAEDAATYYCQQWSFNPPTFGG | |
| | GTKLEIKGSTSGGGSGGGSGGGGSSEVQLQQSGAELVKPG | |
| | ASVKMSCKASGYTFTSYNMHWVKQTPGQGLEWIGAIYP | |
| | GNGDTSYNQKFKGKATLTADKSSSTAYMQLSSLTSEDSA | |
| | DYYCARSNYYGSSYWFFDVWGAGTTVTVSSGGGGSGGG | |
| | GSGGGGSGGGGSEVKLQESGPGLVAPSQSLSVTCTVSGVS | |
| | LPDYGVSWIRQPPRKGLEWLGVIWGSETTYYNSALKSRLT | |
| | IIKDNSKSQVFLKMNSLQTDDTAIYYCAKHYYYGGSYAM | |
| | DYWGQGTSVTVSSGSTSGSGKPGSGEGSTKGDIQMTQTTS | |
| | SLSASLGDRVTISCRASQDISKYLNWYQQKPDGTVKLLIY | |
| | HTSRLHSGVPSRFSGSGSGTDYSLTISNLEQEDIATYFCQQ | |
| | GNTLPYTFGGGTKLEITEPKSCDTPPPCP | |
| | MFWVLVVVGGVLACYSLLVTVAFIIFWVKRGRKKLLYIF | |
| | KQPFMRPVQTTQEEDGCSCRFPEEEEGGCELRVKFSRSAD | |
| | APAYQQGQNQLYNELNLGRREEYDVLDKRRGRDPEMGG | |
| | KPRRKNPQEGLYNELQKDKMAEAYSEIGMKGERRRGKG | |
| | HDGLYQGLSTATKDTYDALHMQALPPR | |
| 19 | GCTAGCGCCGCCACCATGGAGACAGACACACTCCTGCT | CAR Construct 19 |
| | ATGGGTGCTGCTGCTCTGGGTTCCAGGTTCCACAGGTG | |
| | ACATTGTGCTGACCCAATCTCCAGCTATCCTGTCTGCAT | |
| | CTCCAGGGGAGAAGGTCACAATGACTTGCAGGGCCAGC | |
| | TCAAGTGTAAATTACATGGACTGGTACCAGAAGAAGCC | |
| | AGGATCCTCCCCCAAACCCTGGATTTATGCCACATCCA | |
| | ACCTGGCTTCTGGAGTCCCTGCTCGCTTCAGTGGCAGTG | |
| | GGTCTGGGACCTCTTACTCTCTCACAATCAGCAGAGTG | |
| | GAGGCTGAAGATGCTGCCACTTATTACTGCCAGCAGTG | |
| | GAGTTTTAATCCACCCACGTTCGGAGGGGGGACCAAGC | |
| | TGGAAATAAAAGGCAGTACTAGCGGTGGTGGCTCCGGG | |
| | GGCGGTTCCGGTGGGGGCGGCAGCAGCGAGGTGCAGC | |
| | TGCAGCAGTCTGGGGCTGAGCTGGTGAAGCCTGGGGCC | |
| | TCAGTGAAGATGTCCTGCAAGGCTTCTGGCTACACATTT | |
| | ACCAGTTACAATATGCACTGGGTAAAGCAGACACCTGG | |
| | ACAGGGCCTGGAATGGATTGGAGCTATTTATCCAGGAA | |
| | ATGGTGATACTTCCTACAATCAGAAGTTCAAAGGCAAG | |
| | GCCACATTGACTGCAGACAAATCCTCCAGCACAGCCTA | |
| | CATGCAGCTCAGCAGCCTGACATCTGAGGACTCTGCGG | |
| | ACTATTACTGTGCAAGATCTAATTATTACGGTAGTAGCT | |
| | ACTGGTTCTTCGATGTCTGGGGCGCAGGGACCACGGTC | |
| | ACAGTAAGTAGCGGTGGAGGCGGCAGTGGCGGAGGTG | |
| | GGAGCGGAGGGGGCGGTTCCGGTGGCGGGGGATCTGA | |
| | GGTGAAGCTGCAGGAAAGCGGCCCTGGCCTGGTGGCCC | |
| | CCAGCCAGAGCCTGAGCGTGACCTGCACCGTGAGCGGC | |
| | GTGAGCCTGCCCGACTACGGCGTGAGCTGGATCCGGCA | |
| | GCCCCCCAGGAAGGGCCTGGAATGGCTGGGCGTGATCT | |
| | GGGGCAGCGAGACCACCTACTACAACAGCGCCCTGAA | |
| | GAGCCGGCTGACCATCATCAAGGACAACAGCAAGAGC | |
| | CAGGTGTTCCTGAAGATGAACAGCCTGCAGACCGACGA | |
| | CACCGCCATCTACTACTGCGCCAAGCACTACTACTACG | |
| | GCGGCAGCTACGCCATGGACTACTGGGGCCAGGGCACC | |
| | AGCGTGACCGTGAGCAGCGGCAGCACCTCCGGCAGCG | |
| | GCAAGCCTGGCAGCGGCGAGGGCAGCACCAAGGGCGA | |
| | CATCCAGATGACCCAGACCACCTCCAGCCTGAGCGCCA | |
| | GCCTGGGCGACCGGGTGACCATCAGCTGCCGGGCCAGC | |
| | CAGGACATCAGCAAGTACCTGAACTGGTATCAGCAGAA | |
| | GCCCGACGGCACCGTCAAGCTGCTGATCTACCACACCA | |
| | GCCGGCTGCACAGCGGCGTGCCCAGCCGGTTTAGCGGC | |
| | AGCGGCTCCGGCACCGACTACAGCCTGACCATCTCCAA | |
| | CCTGGAACAGGAAGATATCGCCACCTACTTTTGCCAGC | |
| | AGGGCAACACACTGCCCTACACCTTTGGCGGCGGAACA | |
| | AAGCTGGAAATCACCGTACCCTGTCGAGTACCGCCTCC | |
| | GCCCCCATGCTGCCACCCGATGTTCTGGGTGCTGGTGGT | |
| | GGTCGGAGGCGTGCTGGCCTGCTACAGCCTGCTGGTCA | |
| | CCGTGGCCTTCATCATCTTTTGGGTGAAACGGGGCAGA | |
| | AAGAAACTCCTGTATATATTCAAACAACCATTTATGAG | |
| | ACCAGTACAAACTACTCAAGAGGAAGATGGCTGTAGCT | |
| | GCCGATTTCCAGAAGAAGAAGAAGGAGGATGTGAACT | |
| | GCGGGTGAAGTTCAGCAGAAGCGCCGACGCCCCTGCCT | |
| | ACCAGCAGGGCCAGAATCAGCTGTACAACGAGCTGAA | |
| | CCTGGGCAGAAGGGAAGAGTACGACGTCCTGGATAAG | |
| | CGGAGAGGCCGGGACCCTGAGATGGGCGGCAAGCCTC | |
| | GGCGGAAGAACCCCCAGGAAGGCCTGTATAACGAACT | |
| | GCAGAAAGACAAGATGGCCGAGGCCTACAGCGAGATC | |
| | GGCATGAAGGGCGAGCGGAGGCGGGGCAAGGGCCACG | |
| | ACGGCCTGTATCAGGGCCTGTCCACCGCCACCAAGGAT | |
| | ACCTACGACGCCCTGCACATGCAGGCCCTGCCCCCAAG | |
| | GTGAGTCGAC | |
| 20 | METDTLLLWVLLLWVPGSTGDIVLTQSPAILSASPGEKVT | CAR Construct 20 |
| | MTCRASSSVNYMDWYQKKPGSSPKPWIYATSNLASGVPA | |
| | RFSGSGSGTSYSLTISRVEAEDAATYYCQQWSFNPPTFGG | |
| | GTKLEIKGSTSGGGSGGGSGGGGSSEVQLQQSGAELVKPG | |
| | ASVKMSCKASGYTFTSYNMHWVKQTPGQGLEWIGAIYP | |
| | GNGDTSYNQKFKGKATLTADKSSSTAYMQLSSLTSEDSA | |
| | DYYCARSNYYGSSYWFFDVWGAGTTVTVSSGGGGSGGG | |
| | GSGGGGSGGGGSEVKLQESGPGLVAPSQSLSVTCTVSGVS | |
| | LPDYGVSWIRQPPRKGLEWLGVIWGSETTYYNSALKSRLT | |
| | IIKDNSKSQVFLKMNSLQTDDTAIYYCAKHYYYGGSYAM | |
| | DYWGQGTSVTVSSGSTSGSGKPGSGEGSTKGDIQMTQTTS | |
| | SLSASLGDRVTISCRASQDISKYLNWYQQKPDGTVKLLIY | |
| | HTSRLHSGVPSRFSGSGSGTDYSLTISNLEQEDIATYFCQQ | |
| | GNTLPYTFGGGTKLEITVPCRVPPPPPCCHP | |
| | MFWVLVVVGGVLACYSLLVTVAFIIFWVKRGRKKLLYIF | |
| | KQPFMRPVQTTQEEDGCSCRFPEEEEGGCELRVKFSRSAD | |
| | APAYQQGQNQLYNELNLGRREEYDVLDKRRGRDPEMGG | |
| | KPRRKNPQEGLYNELQKDKMAEAYSEIGMKGERRRGKG | |
| | HDGLYQGLSTATKDTYDALHMQALPPR | |
| 52 | METDTLLLWVLLLWVPGSTGDIQMTQSPSSLSASVGDRV | CAR Construct 21 |
| | TITCRASSSVNYMDWYQQKPGKAPKLLIYATSNLASGVPS | |
| | RFSGSGSGTDFTLTISSLQPEDFATYYCQQWSFNPPTFGGG | |
| | TKVEIKGSTSGGGSGGGSGGGGSSEVQLLESGGGLVQPGG | |
| | SLRLSCAASGFTFTSYNMHWVRQAPGKGLEWVGAIYPGN | |
| | GDTSYNQKFKGRFTISRDNSKNTLYLQMNSLRAEDTAVY | |
| | YCARSNYYGSSYWFFDVWGQGTTVTVSSGGGGSGGGGS | |
| | GGGGSGGGGSEVQLLESGGGLVQPGGSLRLSCAASGFTFS | |
| | DYGVSWVRQAPGKGLEWVSVIWGSETTYYNSALKSRFTI | |
| | SRDNSKNTLYLQMNSLRAEDTAVYYCAKHYYYGGSYAM | |
| | DYWGQGTLVTVSSGSTSGSGKPGSGEGSTKGDIQMTQSPS | |
| | SLSASVGDRVTITCRASQDISKYLNWYQQKPGKAPKLLIY | |
| | HTSRLHSGVPSRFSGSGSGTDYTLTISSLQPEDIATYYCQQ | |
| | GNTLPYTFGGGTKVEIKESKYGPPCPPCPMFWVLVVVGG | |
| | VLACYSLLVTVAFIIFWVKRGRKKLLYIFKQPFMRPVQTT | |
| | QEEDGCSCRFPEEEEGGCELRVKFSRSADAPAYQQGQNQ | |
| | LYNELNLGRREEYDVLDKRRGRDPEMGGKPRRKNPQEGL | |
| | YNELQKDKMAEAYSEIGMKGERRRGKGHDGLYQGLSTA | |
| | TKDTYDALHMQALPPR | |
| 53 | METDTLLLWVLLLWVPGSTGEIVLTQSPATLSLSPGERAT | CAR Construct 22 |
| | LSCRASSSVNYMDWYQQKPGLAPRLLIYATSNLASGIPDR | |
| | FSGSGSGTDFTLTISRLEPEDFAVYYCQQWSFNPPTFGGGT | |
| | KVEIKGSTSGGGSGGGSGGGGSSQVQLVQSGAEVKKPGA | |
| | SVKVSCKASGYTFTSYNMHWVRQAPGQGLEWMGAIYPG | |
| | NGDTSYNQKFKGRVTMTRDKSTSTVYMELSSLRSEDTAV | |
| | YYCARSNYYGSSYWFFDVWGQGTTVTVSSGGGGSGGGG | |
| | SGGGGSGGGGSEVQLLESGGGLVQPGGSLRLSCAASGFTF | |
| | SDYGVSWVRQAPGKGLEWVSVIWGSETTYYNSALKSRFT | |
| | ISRDNSKNTLYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDIATYYCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 54 | METDTLLLWVLLLWVPGSTGEIVLTQSPATLSLSPGERAT | CAR Construct 23 |
| | LSCRASSSVNYMDWYQQKPGLAPRLLIYATSNLASGIPDR | |
| | FSGSGSGTDFTLTISRLEPEDFAVYYCQQWSFNPPTFGGGT | |
| | KVEIKGSTSGGGSGGGSGGGGSSQVQLVQSGAEVKKPGA | |
| | SVKVSCKASGYTFTSYNMHWVRQAPGQGLEWMGAIYPG | |
| | NGDTSYNQKFKGRVTMTRDKSTSTVYMELSSLRSEDTAV | |
| | YYCARSNYYGSSYWFFDVWGQGTTVTVSSGGGGSGGGG | |
| | SGGGGSGGGGSEVQLLESGGGLVQPGGSLRLSCAASGFTF | |
| | SDYGVSWVRQAPGKGLEWVSVIWGSETTYYNSALKSRFT | |
| | ISRDNSKNTLYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDIATYYCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 55 | METDTLLLWVLLLWVPGSTGEIVLTQSPATLSLSPGERAT | CAR Construct 24 |
| | LSCRASSSVNYMDWYQQKPGLAPRLLIYATSNLASGIPDR | |
| | FSGSGSGTDFTLTISRLEPEDFAVYYCQQWSFNPPTFGGGT | |
| | KVEIKGSTSGGGSGGGSGGGGSSQVQLVQSGAEVKKPGA | |
| | SVKVSCKASGYTFTSYNMHWVRQAPGQGLEWMGAIYPG | |
| | NGDTSYNQKFKGRVTMTRDKSTSTVYMELSSLRSEDTAV | |
| | YYCARSNYYGSSYWFFDVWGQGTTVTVSSGGGGSGGGG | |
| | SGGGGSGGGGSEVQLLESGGGLVQPGGSLRLSCAASGFTF | |
| | SDYGVSWVRQAPGKGLEWVSVIWGSETTYYNSALKSRFT | |
| | ISRDNSKNTLYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDIATYYCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 60 | METDTLLLWVLLLWVPGSTGDIQMTQSPSSLSASVGDRV | CAR Construct 25 |
| | TITCRASSSVNYMDWYQQKPGKAPKLLIYATSNLASGVPS | |
| | RFSGSGSGTDFTLTISSLQPEDFATYYCQQWSFNPPTFGGG | |
| | TKVEIKGSTSGGGSGGGSGGGGSSQVQLVQSGAEVKKPG | |
| | ASVKVSCKASGYTFTSYNMHWVRQAPGQGLEWMGAIYP | |
| | GNGDTSYNQKFKGRVTMTRDTSTSTVYMELSSLRSEDTA | |
| | VYYCARSNYYGSSYWFFDVWGQGTTVTVSSGGGGSGGG | |
| | GSGGGGSGGGGSEVQLLESGGGLVQPGGSLRLSCAASGF | |
| | TFSDYGVSWVRQAPGKGLEWVSVIWGSETTYYNSALKSR | |
| | FTISKDNSKNTVYLQMNSLRAEDTAVYYCAKHYYYGGSY | |
| | AMDYWGQGTLVTVSSGSTSGSGKPGSGEGSTKGDIQMTQ | |
| | SPSSLSASVGDRVTITCRASQDISKYLNWYQQKPGKAPKL | |
| | LIYHTSRLHSGVPSRFSGSGSGTDYTLTISSLQPEDFATYYC | |
| | QQGNTLPYTFGGGTKVEIKESKYGPPCPPCPMFWVLVVV | |
| | GGVLACYSLLVTVAFIIFWVKRGRKKLLYIFKQPFMRPVQ | |
| | TTQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 61 | METDTLLLWVLLLWVPGSTGDIQMTQSPSSLSASVGDRV | CAR Construct 26 |
| | TITCRASSSVNYMDWYQQKPGKAPKLLIYATSNLASGVPS | |
| | RFSGSGSGTDFTLTISSLQPEDFATYYCQQWSFNPPTFGGG | |
| | TKVEIKGSTSGGGSGGGSGGGGSSEVQLLESGGGLVQPGG | |
| | SLRLSCAASGFTFSSYNMHWVRQAPGKGLEWVSAIYPGN | |
| | GDTSYNQKFKGRFTISRDNSKNTLYLQMNSLRAEDTAVY | |
| | YCARSNYYGSSYWFFDVWGQGTTVTVSSGGGGSGGGGS | |
| | GGGGSGGGGSEVQLLESGGGLVQPGGSLRLSCAASGFTFS | |
| | DYGVSWVRQAPGKGLEWVSVIWGSETTYYNSALKSRFTI | |
| | SKDNSKNTVYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDFATYYCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 62 | METDTLLLWVLLLWVPGSTGDIQMTQSPSSLSASVGDRV | CAR Construct 27 |
| | TITCRASSSVNYMDWYQQKPGKAPKLLIYATSNLASGVPS | |
| | RFSGSGSGTDFTLTISSLQPEDFATYYCQQWSFNPPTFGGG | |
| | TKVEIKGSTSGGGSGGGSGGGGSSEVQLLESGGGLVQPGG | |
| | SLRLSCAASGFTFTSYNMHWVRQAPGKGLEWVGAIYPGN | |
| | GDTSYNQKFKGRFTISRDNSKNTLYLQMNSLRAEDTAVY | |
| | YCARSNYYGSSYWFFDVWGQGTTVTVSSGGGGSGGGGS | |
| | GGGGSGGGGSEVQLLESGGGLVQPGGSLRLSCAASGFTFS | |
| | DYGVSWVRQAPGKGLEWVSVIWGSETTYYNSALKSRFTI | |
| | SKDNSKNTVYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDFATYYCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 63 | METDTLLLWVLLLWVPGSTGEIVLTQSPATLSLSPGERAT | CAR Construct 28 |
| | LSCRASSSVNYMDWYQQKPGLAPRLLIYATSNLASGIPDR | |
| | FSGSGSGTDFTLTISRLEPEDFAVYYCQQWSFNPPTFGGGT | |
| | KVEIKGSTSGGGSGGGSGGGGSSQVQLVQSGAEVKKPGA | |
| | SVKVSCKASGYTFTSYNMHWVRQAPGQGLEWMGAIYPG | |
| | NGDTSYNQKFKGRVTMTRDKSTSTVYMELSSLRSEDTAV | |
| | YYCARSNYYGSSYWFFDVWGQGTTVTVSSGGGGSGGGG | |
| | SGGGGSGGGGSEVQLLESGGGLVQPGGSLRLSCAASGFTF | |
| | SDYGVSWVRQAPGKGLEWVSVIWGSETTYYNSALKSRFT | |
| | ISKDNSKNTVYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDFATYYCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 64 | METDTLLLWVLLLWVPGSTGEIVLTQSPATLSLSPGERAT | CAR Construct 29 |
| | LSCRASSSVNYMDWYQQKPGLAPRLLIYATSNLASGIPDR | |
| | FSGSGSGTDFTLTISRLEPEDFAVYYCQQWSFNPPTFGGGT | |
| | KVEIKGSTSGGGSGGGSGGGGSSEVQLLESGGGLVQPGGS | |
| | LRLSCAASGFTFTSYNMHWVRQAPGKGLEWVGAIYPGN | |
| | GDTSYNQKFKGRFTISRDNSKNTLYLQMNSLRAEDTAVY | |
| | YCARSNYYGSSYWFFDVWGQGTTVTVSSGGGGSGGGGS | |
| | GGGGSGGGGSEVQLLESGGGLVQPGGSLRLSCAASGFTFS | |
| | DYGVSWVRQAPGKGLEWVSVIWGSETTYYNSALKSRFTI | |
| | SKDNSKNTVYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDFATYYCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 65 | METDTLLLWVLLLWVPGSTGEIVLTQSPATLSLSPGERAT | CAR Construct 30 |
| | LSCRASSSVNYMDWYQQKPGLAPRLLIYATSNLASGIPDR | |
| | FSGSGSGTDFTLTISRLEPEDFAVYYCQQWSFNPPTFGGGT | |
| | KVEIKGSTSGGGSGGGSGGGGSSEVQLLESGGGLVQPGGS | |
| | LRLSCAASGFTFTSYNMHWVRQAPGKGLEWVGAIYPGN | |
| | GDTSYNQKFKGRFTISADNSKNTLYLQMNSLRAEDTAVY | |
| | YCARSNYYGSSYWFFDVWGQGTTVTVSSGGGGSGGGGS | |
| | GGGGSGGGGSEVQLLESGGGLVQPGGSLRLSCAASGFTFS | |
| | DYGVSWVRQAPGKGLEWVSVIWGSETTYYNSALKSRFTI | |
| | SKDNSKNTVYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDFATYYCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 66 | METDTLLLWVLLLWVPGSTGDIQMTQSPSSLSASVGDRV | CAR Construct 31 |
| | TITCRASSSVNYMDWYQQKPGKAPKLLIYATSNLASGVPS | |
| | RFSGSGSGTDFTLTISSLQPEDFATYYCQQWSFNPPTFGGG | |
| | TKVEIKGSTSGGGSGGGSGGGGSSQVQLVQSGAEVKKP | |
| | GASVKVSCKASGYTFTSYNMHWVRQAPGQGLEWMGAIY | |
| | PGNGDTSYNQKFKGRVTMTRDTSTSTVYMELSSLRSEDT | |
| | AVYYCARSNYYGSSYWFFDVWGQGTTVTVSSGGGGSG | |
| | GGGSGGGGSGGGGSEVQLLESGGGLVQPGGSLRLSCAA | |
| | SGFTFSDYGVSWVRQAPGKGLEWVSVIWGSETTYYNSAL | |
| | KSRFTISKDNSKNTVYLQMNSLRAEDTAVYYCAKHYYYG | |
| | GSYAMDYWGQGTLVTVSSGSTSGSGKPGSGEGSTKGDIQ | |
| | MTQSPSSLSASVGDRVTITCRASQDISKYLNWYQQKPGKA | |
| | PKLLIYHTSRLHSGVPSRFSGSGSGTDYTLTISSLQPEDFAT | |
| | YFCQQGNTLPYTFGGGTKVEIKESKYGPPCPPCPMFWVLV | |
| | VVGGVLACYSLLVTVAFIIFWVKRGRKKLLYIFKQPFMRP | |
| | VQTTQEEDGCSCRFPEEEEGGCELRVKFSRSADAPAYQQG | |
| | QNQLYNELNLGRREEYDVLDKRRGRDPEMGGKPRRKNP | |
| | QEGLYNELQKDKMAEAYSEIGMKGERRRGKGHDGLYQG | |
| | LSTATKDTYDALHMQALPPR* | |
| 67 | METDTLLLWVLLLWVPGSTGDIQMTQSPSSLSASVGDRV | CAR Construct 32 |
| | TITCRASSSVNYMDWYQQKPGKAPKLLIYATSNLASGVPS | |
| | RFSGSGSGTDFTLTISSLQPEDFATYYCQQWSFNPPTFGGG | |
| | TKVEIKGSTSGGGSGGGSGGGGSSEVQLLESGGGLVQPGG | |
| | SLRLSCAASGFTFSSYNMHWVRQAPGKGLEWVSAIYPGN | |
| | GDTSYNQKFKGRFTISRDNSKNTLYLQMNSLRAEDTAVY | |
| | YCARSNYYGSSYWFFDVWGQGTTVTVSSGGGGSGGGGS | |
| | GGGGSGGGGSEVQLLESGGGLVQPGGSLRLSCAASGFTFS | |
| | DYGVSWVRQAPGKGLEWVSVIWGSETTYYNSALKSRFTI | |
| | SKDNSKNTVYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDFATYFCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 68 | METDTLLLWVLLLWVPGSTGDIQMTQSPSSLSASVGDRV | CAR Construct 33 |
| | TITCRASSSVNYMDWYQQKPGKAPKLLIYATSNLASGVPS | |
| | RFSGSGSGTDFTLTISSLQPEDFATYYCQQWSFNPPTFGGG | |
| | TKVEIKGSTSGGGSGGGSGGGGSSEVQLLESGGGLVQPGG | |
| | SLRLSCAASGFTFTSYNMHWVRQAPGKGLEWVGAIYPGN | |
| | GDTSYNQKFKGRFTISRDNSKNTLYLQMNSLRAEDTAVY | |
| | YCARSNYYGSSYWFFDVWGQGTTVTVSSGGGGSGGGGS | |
| | GGGGSGGGGSEVQLLESGGGLVQPGGSLRLSCAASGFTFS | |
| | DYGVSWVRQAPGKGLEWVSVIWGSETTYYNSALKSRFTI | |
| | SKDNSKNTVYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDFATYFCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 70 | METDTLLLWVLLLWVPGSTGEIVLTQSPATLSLSPGERAT | CAR Construct 34 |
| | LSCRASSSVNYMDWYQQKPGLAPRLLIYATSNLASGIPDR | |
| | FSGSGSGTDFTLTISRLEPEDFAVYYCQQWSFNPPTFGGGT | |
| | KVEIKGSTSGGGSGGGSGGGGSSQVQLVQSGAEVKKPGA | |
| | SVKVSCKASGYTFTSYNMHWVRQAPGQGLEWMGAIYPG | |
| | NGDTSYNQKFKGRVTMTRDKSTSTVYMELSSLRSEDTAV | |
| | YYCARSNYYGSSYWFFDVWGQGTTVTVSSGGGGSGGGG | |
| | SGGGGSGGGGSEVQLLESGGGLVQPGGSLRLSCAASGFTF | |
| | SDYGVSWVRQAPGKGLEWVSVIWGSETTYYNSALKSRFT | |
| | ISKDNSKNTVYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDFATYFCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 71 | METDTLLLWVLLLWVPGSTGEIVLTQSPATLSLSPGERAT | CAR Construct 35 |
| | LSCRASSSVNYMDWYQQKPGLAPRLLIYATSNLASGIPDR | |
| | FSGSGSGTDFTLTISRLEPEDFAVYYCQQWSFNPPTFGGGT | |
| | KVEIKGSTSGGGSGGGSGGGGSSEVQLLESGGGLVQPGGS | |
| | LRLSCAASGFTFTSYNMHWVRQAPGKGLEWVGAIYPGN | |
| | GDTSYNQKFKGRFTISRDNSKNTLYLQMNSLRAEDTAVY | |
| | YCARSNYYGSSYWFFDVWGQGTTVTVSSGGGGSGGGGS | |
| | GGGGSGGGGSEVQLLESGGGLVQPGGSLRLSCAASGFTFS | |
| | DYGVSWVRQAPGKGLEWVSVIWGSETTYYNSALKSRFTI | |
| | SKDNSKNTVYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDFATYFCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 72 | METDTLLLWVLLLWVPGSTGEIVLTQSPATLSLSPGERAT | CAR Construct 36 |
| | LSCRASSSVNYMDWYQQKPGLAPRLLIYATSNLASGIPDR | |
| | FSGSGSGTDFTLTISRLEPEDFAVYYCQQWSFNPPTFGGGT | |
| | KVEIKGSTSGGGSGGGSGGGGSSEVQLLESGGGLVQPGGS | |
| | LRLSCAASGFTFTSYNMHWVRQAPGKGLEWVGAIYPGN | |
| | GDTSYNQKFKGRFTISADNSKNTLYLQMNSLRAEDTAVY | |
| | YCARSNYYGSSYWFFDVWGQGTTVTVSSGGGGSGGGGS | |
| | GGGGSGGGGSEVQLLESGGGLVQPGGSLRLSCAASGFTFS | |
| | DYGVSWVRQAPGKGLEWVSVIWGSETTYYNSALKSRFTI | |
| | SKDNSKNTVYLQMNSLRAEDTAVYYCAKHYYYGGSYA | |
| | MDYWGQGTLVTVSSGSTSGSGKPGSGEGSTKGDIQMTQS | |
| | PSSLSASVGDRVTITCRASQDISKYLNWYQQKPGKAPKLLI | |
| | YHTSRLHSGVPSRFSGSGSGTDYTLTISSLQPEDFATYFCQ | |
| | QGNTLPYTFGGGTKVEIKESKYGPPCPPCPMFWVLVVVG | |
| | GVLACYSLLVTVAFIIFWVKRGRKKLLYIFKQPFMRPVQT | |
| | TQEEDGCSCRFPEEEEGGCELRVKFSRSADAPAYQQGQN | |
| | QLYNELNLGRREEYDVLDKRRGRDPEMGGKPRRKNPQE | |
| | GLYNELQKDKMAEAYSEIGMKGERRRGKGHDGLYQGLS | |
| | TATKDTYDALHMQALPPR* | |
| 21 | ESKYGPPCPPCP | IgG4 hinge |
| 22 | TTTPAPRPPTPAPTIASQPLSLRPEACRPAAGGAVHTRGLD | CD8a hinge (45 aa) |
| | FACD | |
| 23 | IEVMYPPPYLDNEKSNGTIIHVKGKHLCPSPLFPGPSKP | CD28 hinge |
| 24 | SGQVLLESNIKVLPTWSTPVQP | CD4 hinge |
| 25 | SQPLSLRPEACRPAAGGAVHTRGLDFACD | CD8α hinge (29 aa) |
| 26 | RPAAGGAVHTRGLDFACD | CD8α hinge (18 aa) |
| 27 | EPKSCDKTHTCP | IgG1 hinge |
| 28 | EPKSCDTPPPCP | IgG3 hinge |
| 29 | VPCRVPPPPPCCHP | IgA2 hinge |
| 30 | FWVLVVVGGVLACYSLLVTVAFIIFWV | CD28 TM |
| 31 | IYIWAPLAGTCGVLLLSLVITLYC | CD8α TM |
| 32 | KRGRKKLLYIFKQPFMRPVQTTQEEDGCSCRFPEEEEGGC | 4-1BB signaling |
| | EL | domain |
| 33 | RVKFSRSADAPAYQQGQNQLYNELNLGRREEYDVLDKR | CD3 zeta signaling |
| | RGRDPEMGGKPRRKNPQEGLYNELQKDKMAEAYSEIGM | domain |
| | KGERRRGKGHDGLYQGLSTATKDTYDALHMQALPPR | |
| 34 | DIVLTQSPAILSASPGEKVTMTCRASSSVNYMDWYQKKP | Murine anti CD20 |
| | GSSPKPWIYATSNLASGVPARFSGSGSGTSYSLTISRVEAE | Leu16 hybridoma VK |
| | DAATYYCQQWSFNPPTFGGGTKLEIK | sequence |
| 73 | RASSSVNYMD | Murine anti CD20 |
| | | Leu16 hybridoma |
| | | CDRL1 |
| 74 | YATSNLAS | Murine anti CD20 |
| | | Leu16 hybridoma |
| | | CDRL2 |
| 75 | QQWSFNPPT | Murine anti CD20 |
| | | Leu16 hybridoma |
| | | CDRL3 |
| 35 | EVQLQQSGAELVKPGASVKMSCKASGYTFTSYNMHWVK | Murine anti CD20 |
| | QTPGQGLEWIGAIYPGNGDTSYNQKFKGKATLTADKSSST | hybridoma VH |
| | AYMQLSSLTSEDSADYYCARSNYYGSSYWFFDVWGAGT | sequence |
| | TVTVSS | |
| 76 | SYNMH | Murine anti CD20 |
| | | hybridoma CDRH1 |
| 77 | AIYPGNGDTSYNQKFKG | Murine anti CD20 |
| | | hybridoma CDRH2 |
| 78 | SNYYGSSYWFFDV | Murine anti CD20 |
| | | hybridoma CDRH3 |
| 36 | EVKLQESGPGLVAPSQSLSVTCTVSGVSLPDYGVSWIRQP | Murine anti CD19 VH |
| | PRKGLEWLGVIWGSETTYYNSALKSRLTIIKDNSKSQVFL | sequence |
| | KMNSLQTDDTAIYYCAKHYYYGGSYAMDYWGQGTSVT | |
| | VSS | |
| 37 | DIQMTQTTSSLSASLGDRVTISCRASQDISKYLNWYQQKP | Murine anti CD19 VK |
| | DGTVKLLIYHTSRLHSGVPSRFSGSGSGTDYSLTISNLEQE | sequence |
| | DIATYFCQQGNTLPYTFGGGTKLEIT | |
| 48 | GGGGS | (G₄S)₁ |
| 49 | GGGGSGGGGS | (G₄S)₂ |
| 50 | GGGGSGGGGSGGGGS | (G₄S)₃ |
| 51 | GGGGSGGGGSGGGGSGGGGS | (G₄S)₄ |
| 230 | DIVLTQSPAILSASPGEKVTMTCRASSSVNYMDWYQKKP | CD20 scFV |
| | GSSPKPWIYATSNLASGVPARFSGSGSGTSYSLTISRVEAE | |
| | DAATYYCQQWSFNPPTFGGGTKLEIKGSTSGGGSGGGSG | |
| | GGGSSEVQLQQSGAELVKPGASVKMSCKASGYTFTSYNM | |
| | HWVKQTPGQGLEWIGAIYPGNGDTSYNQKKGKATLTAD | |
| | KSSSTAYMQLSSLTSEDSADYYCARSNYYGSSYWFFDVW | |
| | GAGTTVTVSS | |
| 231 | DIQMTQTTSSLSASLGDRVTISCRASQDISKYLNWYQQKP | CD19 scFV |
| | DGTVKLLIYHTSRLHSGVPSRFSGSGSGTDYSLTISNLEQE | |
| | DIATYFCQQGNTLPYTFGGGTKLEITGSTSGSGKPGSGEGS | |
| | TKGEVKLQESGPGLVAPSQSLSVTCTVSGVSLPDYGVSWI | |
| | RQPPRKGLEWLGVIWGSETTYYNSALKSRLTIIKDNSKSQ | |
| | VFLKMNSLQTDDTAIYYCAKHYYYGGSYAMDYWGQGTS | |
| | VTVSS | |
| 232 | ESKYGPPCPPCP | IgG4 Hinge |
| 233 | FWVLVWGGVLACYSLLVTVAFIIFWV | CD28 transmembrane |
| | | domain |
| 234 | RSKRSRGGHSDYMNMTPRRPGPTRKHYQPYAPPRDFAAY | CD28 cytoplasmic |
| | RS | domain |
| 235 | KRGRKKLLYIFKQPFMRPVQTTQEEDGCSCRFPEEEEGGC | 4-1BB cytoplasmic |
| | EL | domain |
| 236 | RVKFSRSADAPAYQQGQNQLYNELNLGRREEYDVLDKR | CD32 cytoplasmic |
| | RGRDPEMGGKPRRKNPQEGLYNELQKDKMAEAYSEIGM | domain |
| | KGERRRGKGHDGLYQGLSTATKDTYDALHMQALPPR | |
| 237 | LEGGGEGRGSLLTCGDVEENPGPR | T2A self-cleaving |
| | | peptide |
| 238 | APEFLGGPSVFLFPPKPKDTLMISRTPEVTCWVDVSQEDPE | IgG4 CH2 |
| | VQFNWYVDGVEVHNAKTKPREEQFNSTYRWSVLTVLHQ | |
| | DWLNGKEYKCKVSNKGLPSSIEKTISKAK | |
| 239 | GQPREPQVYTLPPSQEEMTKNQVSLTCLVKGFYPSDIAVE | IgG4 CH3 |
| | WESNGQPENNYKTTPPVLDSDGSFFLYSRLTVDKSRWQE | |
| | GNVFSCSVMHEALHNHYTQKSLSLSLGK | |
| 240 | MLLLVTSLLLCELPHPAFLLIPRKVCNGIGIGEFKDSLSINA | Truncated epidermal |
| | TNIKHFKNCTSISGDLHILPVAFRGDSFTHTPPLDPQELDIL | growth factor receptor |
| | KTVKEITGFLLIQAWPENRTDLHAFENLEIIRGRTKQHGQF | (EGFRt) |
| | SLAWSLNITSLGLRSLKEISDGDVIISGNKNLCYANTINWK | |
| | KLFGTSGQKTKIISNRGENSCKATGQVCHALCSPEGCWGP | |
| | EPRDCVSCRNVSRGRECVDKCNLLEGEPREFVENSECIQC | |
| | HPECLPQAMNITCTGRGPDNCIQCAHYIDGPHCVKTCPAG | |
| | VMGENNTLVWKYADAGHVCHLCHPNCTYGCTGPGLEGC | |
| | PTNGPKIPSIATGMVGALLLLLWALGIGLFM | |

Other exemplary CAR sequences are disclosed in WO2016100232A1, which is incorporated by reference in its entirety.

In some embodiments, the CAR has at least 80% sequence identity the amino acid sequence selected from the group consisting of SEQ ID NOs: 2, 4, 6, 8, 10, 12, 14, 16, 18, and 20. In some embodiments, the CAR has at least 85% sequence identity the amino acid sequence selected from the group consisting of SEQ ID NOs: 2, 4, 6, 8, 10, 12, 14, 16, 18, and 20. In some embodiments, the CAR has at least 90% sequence identity the amino acid sequence selected from the group consisting of SEQ ID NOs: 2, 4, 6, 8, 10, 12, 14, 16, 18, and 20. In some embodiments, the CAR has at least 95% sequence identity the amino acid sequence selected from the group consisting of SEQ ID NOs: 2, 4, 6, 8, 10, 12, 14, 16, 18, and 20. In some embodiments, the CAR has at least 98% sequence identity the amino acid sequence selected from the group consisting of SEQ ID NOs: 2, 4, 6, 8, 10, 12, 14, 16, 18, and 20. In some embodiments, the CAR has at least 99% sequence identity the amino acid sequence selected from the group consisting of SEQ ID NOs: 2, 4, 6, 8, 10, 12, 14, 16, 18, and 20. In some embodiments, the CAR has at least 100% sequence identity and/or is identical to the amino acid sequence selected from the group consisting of SEQ ID NOs: 2, 4, 6, 8, 10, 12, 14, 16, 18, and 20.

In some embodiments, the CAR has at least 80% sequence identity the amino acid sequence selected from the group consisting of SEQ ID NOs: 1, 3, 5, 7, 9, 11, 13, 15, 17, 19, and 52-72. In some embodiments, the CAR has at least 85% sequence identity the amino acid sequence selected from the group consisting of SEQ ID NOs: 1, 3, 5, 7, 9, 11, 13, 15, 17, 19, and 52-72. In some embodiments, the CAR has at least 90% sequence identity the amino acid sequence selected from the group consisting of SEQ ID NOs: 1, 3, 5, 7, 9, 11, 13, 15, 17, 19, and 52-72. In some embodiments, the CAR has at least 95% sequence identity the amino acid sequence selected from the group consisting of SEQ ID NOs: 1, 3, 5, 7, 9, 11, 13, 15, 17, 19, and 52-72. In some embodiments, the CAR has at least 98% sequence identity the amino acid sequence selected from the group consisting of SEQ ID NOs: 1, 3, 5, 7, 9, 11, 13, 15, 17, 19, and 52-72. In some embodiments, the CAR has at least 99% sequence identity the amino acid sequence selected from the group consisting of SEQ ID NOs: 1, 3, 5, 7, 9, 11, 13, 15, 17, 19, and 52-72. In some embodiments, the CAR has at least 100% sequence identity and/or is identical to the amino acid sequence selected from the group consisting of SEQ ID NOs: 1, 3, 5, 7, 9, 11, 13, 15, 17, 19, and 52-72.

In some embodiments, the CAR may comprise a humanized anti CD20 construct. Exemplary sequences of said construct are listed in Table 2.

TABLE 2
Exemplary humanized anti CD20 construct sequences.

| SEQ ID NO | Amino Acid Sequence | Annotation |
|---|---|---|
| 79 | QVQLVQSGAEVKKPGASVKVSCKASGYTFTSYN | Hz. Leu16(a) VH |
| | MHWVRQAPGQGLEWMGAIYPGNGDTSYNQKFK | |
| | GRVTMTRDTSTSTVYMELSSLRSEDTAVYYCARS | |
| | NYYGSSYWFFDVWGQGTTVTVSS | |
| 80 | SYNMH | Hz. Leu16 (a) VH CDRH1 |
| 81 | AIYPGNGDTSYNQKFK | Hz. Leu16 (a) VH CDRH2 |
| 82 | RSNYYGSSYWFFDV | Hz. Leu16 (a) VH CDRH3 |
| 83 | QVQLVQSGAEVKKPGASVKVSCKASGYTFTSYN | Hz. Leu16 (b) VH |
| | MHWVRQAPGQGLEWMGAIYPGNGDTSYNQKFK | |
| | GRVTMTRDKSTSTVYMELSSLRSEDTAVYYCARS | |
| | NYYGSSYWFFDVWGQGTTVTVSS | |
| 84 | SYNMH | Hz. Leu16 (b) VH CDRH1 |
| 85 | AIYPGNGDTSYNQKFKG | Hz. Leu16 (b) VH CDRH2 |
| 86 | RSNYYGSSYWFFDV | Hz. Leu16 (b) VH CDRH3 |
| 87 | EVQLLESGGGLVQPGGSLRLSCAASGFTFSSYNM | Hz. Leu16 (c) VH |
| | HWVRQAPGKGLEWVSAIYPGNGDTSYNQKFKGR | |
| | FTISRDNSKNTLYLQMNSLRAEDTAVYYCARSNY | |
| | YGSSYWFFDVWGQGTTVTVS | |
| 88 | SYNMH | Hz. Leu16 (c) VH CDRH1 |
| 89 | AIYPGNGDTSYNQKFKG | Hz. Leu16 (c) VH CDRH2 |
| 90 | RSNYYGSSYWFFDV | Hz. Leu16 (c) VH CDRH3 |
| 91 | EVQLLESGGGLVQPGGSLRLSCAASGFTFTSYNM | Hz. Leu16 (d) VH |
| | HWVRQAPGKGLEWVSAIYPGNGDTSYNQKFKGR | |
| | FTISRDNSKNTLYLQMNSLRAEDTAVYYCARSNY | |
| | YGSSYWFFDVWGQGTTVTVSS | |
| 92 | SYNMH | Hz. Leu16 (d) VH CDRH1 |
| 93 | AIYPGNGDTSYNQKFKG | Hz. Leu16 (d) VH CDRH2 |
| 94 | RSNYYGSSYWFFDV | Hz. Leu16 (d) VH CDRH3 |
| 95 | EVQLLESGGGLVQPGGSLRLSCAASGFTFTSYNM | Hz. Leu16 (e) VH |
| | HWVRQAPGKGLEWVGAIYPGNGDTSYNQKFKG | |
| | RFTISRDNSKNTLYLQMNSLRAEDTAVYYCARSN | |
| | YYGSSYWFFDVWGQGTTVTVSS | |
| 96 | SYNMH | Hz. Leu16 (e) VH CDRH1 |
| 97 | AIYPGNGDTSYNQKFKG | Hz. Leu16 (e) VH CDRH2 |
| 98 | RSNYYGSSYWFFDV | Hz. Leu16 (e) VH CDRH3 |
| 99 | EVQLLESGGGLVQPGGSLRLSCAASGFTFTSYNM | Hz. Leu16 (f) VH |
| | HWVRQAPGKGLEWVGAIYPGNGDTSYNQKFKG | |
| | RFTISADNSKNTLYLQMNSLRAEDTAVYYCARSN | |
| | YYGSSYWFFDVWGQGTTVTVSS | |
| 100 | SYNMH | Hz. Leu16 (f) VH CDRH1 |
| 101 | AIYPGNGDTSYNQKFKG | Hz. Leu16 (f) VH CDRH2 |
| 102 | RSNYYGSSYWFFDV | Hz. Leu16 (f) VH CDRH3 |
| 103 | DIVLTQSPAILSASPGEKVTMTCRASSSVNYMDW | Murine anti CD20 Leu16 |
| | YQKKPGSSPKPWIYATSNLASGVPARFSGSGSGTS | |
| | YSLTISRVEAEDAATYYCQQWSFNPPTFGGGTKL | |
| | EIK | |
| 104 | DIQMTQSPSSLSASVGDRVTITCRASSSVNYMDW | Hz. Leu16(a) VL |
| | YQQKPGKAPKLLIYATSNLASGVPSRFSGSGSGTD | |
| | FTLTISSLQPEDFATYYCQQWSFNPPTFGGGTKVEI | |
| | K | |
| 105 | RASSSVNYMD | Hz. Leu16 (a) VL CDRH1 |
| 106 | YATSNLAS | Hz. Leu16 (a) VL CDRH2 |
| 107 | QQWSFNPPT | Hz. Leu16 (a) VL CDRH3 |
| 108 | EIVLTQSPATLSLSPGERATLSCRASSSVNYMDWY | Hz. Leu16 (b) VL |
| | QQKPGLAPRLLIYATSNLASGIPDRFSGSGSGTDFT | |
| | LTISRLEPEDFAVYYCQQWSFNPPTFGGGTKVEIK | |
| 109 | RASSSVNYMD | Hz. Leu16 (b) VL CDRH1 |
| 110 | YATSNLAS | Hz. Leu16 (b) VL CDRH2 |
| 111 | QQWSFNPPT | Hz. Leu16 (b) VL CDRH3 |
| 112 | QVQLVQSGAEVKKPGASVKVSCKASGYTFTSYN | Leu16 VH1-46 |
| | MHWVRQAPGQGLEWMGAIYPGNGDTSY | |
| 113 | EVQLLESGGGLVQPGGSLRLSCAASGFTFSSYNM | Leu16 VH3-23 |
| | HWVRQAPGKGLEWVSAIYPGNGDTSY | |
| 114 | EVQLQQSGAELVKPGASVKMSCKASGYTFTSYN | Leu16 VH |
| | MHWVKQTPGQGLEWIGAIYPGNGDTSY | |
| 115 | NQKFKGRVTMTRDTSTSTVYMELSSLRSEDTAVY | Leu16 VH1-46 |
| | YCARSNYYGSSYWFFDVWGQGTTVTV | |
| 116 | NQKFKGRFTISRDNSKNTLYLQMNSLRAEDTAVY | Leu16 VH3-23 |
| | YCARSNYYGSSYWFFDVWGQGTTVTV | |
| 117 | NQKFKGKATLTADKSSSTAYMQLSSLTSEDSADY | Leu16 VH |
| | YCARSNYYGSSYWFFDVWGAGTTVTV | |
| 118 | EIVLTQSPATLSLSPGERATLSCRASSSVNYMDWY | Leu16 A11 |
| | QQKPGLAPRLLIYATSNLASGIPDR | |
| 119 | DIQMTQSPSSLSASVGDRVTITCRASSSVNYMDW | Leu16 O2 |
| | YQQKPGKAPKLLIYATSNLASGVPSR | |
| 120 | DIVLTQSPAILSASPGEKVTMTCRASSSVNYMDW | Leu16 VK |
| | YQKKPGSSPKPWIYATSNLASGVPAR | |
| 121 | FSGSGSGTDFTLTISRLEPEDFAVYYCQQWSFNPP | Leu16 A11 |
| | TFGGGTKVEIK | |
| 122 | FSGSGSGTDFTLTISSLQPEDFATYYCQQWSFNPPT | Leu16 O2 |
| | FGGGTKVEIK | |
| 123 | FSGSGSGTSYSLTISRVEAEDAATYYCQQWSFNPP | Leu16 VK |
| | TFGGGTKLEIK | |
| 124 | METDTLLLWVLLLWVPGSTGDIVLTQSPAILSASP | CC373 murine Leu16 VL_VH |
| | GEKVTMTCRASSSVNYMDWYQKKPGSSPKPWIY | CAR T |
| | ATSNLASGVPARFSGSGSGTSYSLTISRVEAEDAA | |
| | TYYCQQWSFNPPTFGGGTKLEIKGSTSGGGSGGG | |
| | SGGGGSSEVQLQQSGAELVKPGASVKMSCKASG | |
| | YTFTSYNMHWVKQTPGQGLEWIGAIYPGNGDTS | |
| | YNQKFKGKATLTADKSSSTAYMQLSSLTSEDSAD | |
| | YYCARSNYYGSSYWFFDVWGAGTTVTVSSESKY | |
| | GPPCPPCPAPEFEGGPSVFLFPPKPKDTLMISRTPE | |
| | VTCVVVDVSQEDPEVQFNWYVDGVEVHNAKTK | |
| | PREEQFQSTYRVVSVLTVLHQDWLNGKEYKCKV | |
| | SNKGLPSSIEKTISKAKGQPREPQVYTLPPSQEEMT | |
| | KNQVSLTCLVKGFYPSDIAVEWESNGQPENNYKT | |
| | TPPVLDSDGSFFLYSRLTVDKSRWQEGNVFSCSV | |
| | MHEALHNHYTQKSLSLSLGKMFWVLVVVGGVL | |
| | ACYSLLVTVAFIIFWVKRGRKKLLYIFKQPFMRPV | |
| | QTTQEEDGCSCRFPEEEEGGCELRVKFSRSADAPA | |
| | YQQGQNQLYNELNLGRREEYDVLDKRRGRDPEM | |
| | GGKPRRKNPQEGLYNELQKDKMAEAYSEIGMKG | |
| | ERRRGKGHDGLYQGLSTATKDTYDALHMQALPP | |
| | R** | |
| 125 | METDTLLLWVLLLWVPGSTGDIQMTQSPSSLSAS | CC374_Hz.Leu16_O2/VH1-46 |
| | VGDRVTITCRASSSVNYMDWYQQKPGKAPKLLIY | VL_VH CAR T |
| | ATSNLASGVPSRFSGSGSGTDFTLTISSLQPEDFAT | |
| | YYCQQWSFNPPTFGGGTKVEIKGSTSGGGSGGGS | |
| | GGGGSSQVQLVQSGAEVKKPGASVKVSCKASGY | |
| | TFTSYNMHWVRQAPGQGLEWMGAIYPGNGDTS | |
| | YNQKFKGRVTMTRDTSTSTVYMELSSLRSEDTAV | |
| | YYCARSNYYGSSYWFFDVWGQGTTVTVSSESKY | |
| | GPPCPPCPAPEFEGGPSVFLFPPKPKDTLMISRTPE | |
| | VTCVVVDVSQEDPEVQFNWYVDGVEVHNAKTK | |
| | PREEQFQSTYRVVSVLTVLHQDWLNGKEYKCKV | |
| | SNKGLPSSIEKTISKAKGQPREPQVYTLPPSQEEMT | |
| | KNQVSLTCLVKGFYPSDIAVEWESNGQPENNYKT | |
| | TPPVLDSDGSFFLYSRLTVDKSRWQEGNVFSCSV | |
| | MHEALHNHYTQKSLSLSLGKMFWVLVVVGGVL | |
| | ACYSLLVTVAFIIFWVKRGRKKLLYIFKQPFMRPV | |
| | QTTQEEDGCSCRFPEEEEGGCELRVKFSRSADAPA | |
| | YQQGQNQLYNELNLGRREEYDVLDKRRGRDPEM | |
| | GGKPRRKNPQEGLYNELQKDKMAEAYSEIGMKG | |
| | ERRRGKGHDGLYQGLSTATKDTYDALHMQALPP | |
| | R** | |
| 126 | METDTLLLWVLLLWVPGSTGDIQMTQSPSSLSAS | CC375_Hz.Leu16_O2_VH1- |
| | VGDRVTITCRASSSVNYMDWYQQKPGKAPKLLIY | 46(73K) VL_VH CAR T |
| | ATSNLASGVPSRFSGSGSGTDFTLTISSLQPEDFAT | |
| | YYCQQWSFNPPTFGGGTKVEIKGSTSGGGSGGGS | |
| | GGGGSSQVQLVQSGAEVKKPGASVKVSCKASGY | |
| | TFTSYNMHWVRQAPGQGLEWMGAIYPGNGDTS | |
| | YNQKFKGRVTMTRDKSTSTVYMELSSLRSEDTAV | |
| | YYCARSNYYGSSYWFFDVWGQGTTVTVSSESKY | |
| | GPPCPPCPAPEFEGGPSVFLFPPKPKDTLMISRTPE | |
| | VTCVVVDVSQEDPEVQFNWYVDGVEVHNAKTK | |
| | PREEQFQSTYRVVSVLTVLHQDWLNGKEYKCKV | |
| | SNKGLPSSIEKTISKAKGQPREPQVYTLPPSQEEMT | |
| | KNQVSLTCLVKGFYPSDIAVEWESNGQPENNYKT | |
| | TPPVLDSDGSFFLYSRLTVDKSRWQEGNVFSCSV | |
| | MHEALHNHYTQKSLSLSLGKMFWVLVVVGGVL | |
| | ACYSLLVTVAFIIFWVKRGRKKLLYIFKQPFMRPV | |
| | QTTQEEDGCSCRFPEEEEGGCELRVKFSRSADAPA | |
| | YQQGQNQLYNELNLGRREEYDVLDKRRGRDPEM | |
| | GGKPRRKNPQEGLYNELQKDKMAEAYSEIGMKG | |
| | ERRRGKGHDGLYQGLSTATKDTYDALHMQALPP | |
| | R** | |
| 127 | METDTLLLWVLLLWVPGSTGDIQMTQSPSSLSAS | CC376_Hz.Leu16_O2_VH3-23 |
| | VGDRVTITCRASSSVNYMDWYQQKPGKAPKLLIY | VL_VH CAR T |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FSSYNMHWVRQAPGKGLEWVSAIYPGNGDTSYN | |
| | QKFKGRFTISRDNSKNTLYLQMNSLRAEDTAVYY | |
| | CARSNYYGSSYWFFDVWGQGTTVTVSSESKYGP | |
| | PCPPCPAPEFEGGPSVFLFPPKPKDTLMISRTPEVT | |
| | CVVVDVSQEDPEVQFNWYVDGVEVHNAKTKPRE | |
| | EQFQSTYRVVSVLTVLHQDWLNGKEYKCKVSNK | |
| | GLPSSIEKTISKAKGQPREPQVYTLPPSQEEMTKN | |
| | QVSLTCLVKGFYPSDIAVEWESNGQPENNYKTTP | |
| | PVLDSDGSFFLYSRLTVDKSRWQEGNVFSCSVMH | |
| | EALHNHYTQKSLSLSLGKMFWVLVVVGGVLACY | |
| | SLLVTVAFIIFWVKRGRKKLLYIFKQPFMRPVQTT | |
| | QEEDGCSCRFPEEEEGGCELRVKFSRSADAPAYQ | |
| | QGQNQLYNELNLGRREEYDVLDKRRGRDPEMGG | |
| | KPRRKNPQEGLYNELQKDKMAEAYSEIGMKGER | |
| | RRGKGHDGLYQGLSTATKDTYDALHMQALPPR* | |
| | * | |
| 128 | METDTLLLWVLLLWVPGSTGDIQMTQSPSSLSAS | CC377_Hz.Leu16_O2_VH3- |
| | VGDRVTITCRASSSVNYMDWYQQKPGKAPKLLIY | 23(30T) VL_VH CAR T |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FTSYNMHWVRQAPGKGLEWVSAIYPGNGDTSYN | |
| | QKFKGRFTISRDNSKNTLYLQMNSLRAEDTAVYY | |
| | CARSNYYGSSYWFFDVWGQGTTVTVSSESKYGP | |
| | PCPPCPAPEFEGGPSVFLFPPKPKDTLMISRTPEVT | |
| | CVVVDVSQEDPEVQFNWYVDGVEVHNAKTKPRE | |
| | EQFQSTYRVVSVLTVLHQDWLNGKEYKCKVSNK | |
| | GLPSSIEKTISKAKGQPREPQVYTLPPSQEEMTKN | |
| | QVSLTCLVKGFYPSDIAVEWESNGQPENNYKTTP | |
| | PVLDSDGSFFLYSRLTVDKSRWQEGNVFSCSVMH | |
| | EALHNHYTQKSLSLSLGKMFWVLVVVGGVLACY | |
| | SLLVTVAFIIFWVKRGRKKLLYIFKQPFMRPVQTT | |
| | QEEDGCSCRFPEEEEGGCELRVKFSRSADAPAYQ | |
| | QGQNQLYNELNLGRREEYDVLDKRRGRDPEMGG | |
| | KPRRKNPQEGLYNELQKDKMAEAYSEIGMKGER | |
| | RRGKGHDGLYQGLSTATKDTYDALHMQALPPR* | |
| | * | |
| 129 | METDTLLLWVLLLWVPGSTGDIQMTQSPSSLSAS | CC378-Hz.Leu16_O2_VH3- |
| | VGDRVTITCRASSSVNYMDWYQQKPGKAPKLLIY | 3(30T.49G) VL_VH CAR T |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FTSYNMHWVRQAPGKGLEWVGAIYPGNGDTSYN | |
| | QKFKGRFTISRDNSKNTLYLQMNSLRAEDTAVYY | |
| | CARSNYYGSSYWFFDVWGQGTTVTVSSESKYGP | |
| | PCPPCPAPEFEGGPSVFLFPPKPKDTLMISRTPEVT | |
| | CVVVDVSQEDPEVQFNWYVDGVEVHNAKTKPRE | |
| | EQFQSTYRVVSVLTVLHQDWLNGKEYKCKVSNK | |
| | GLPSSIEKTISKAKGQPREPQVYTLPPSQEEMTKN | |
| | QVSLTCLVKGFYPSDIAVEWESNGQPENNYKTTP | |
| | PVLDSDGSFFLYSRLTVDKSRWQEGNVFSCSVMH | |
| | EALHNHYTQKSLSLSLGKMFWVLVVVGGVLACY | |
| | SLLVTVAFIIFWVKRGRKKLLYIFKQPFMRPVQTT | |
| | QEEDGCSCRFPEEEEGGCELRVKFSRSADAPAYQ | |
| | QGQNQLYNELNLGRREEYDVLDKRRGRDPEMGG | |
| | KPRRKNPQEGLYNELQKDKMAEAYSEIGMKGER | |
| | RRGKGHDGLYQGLSTATKDTYDALHMQALPPR* | |
| | * | |
| 130 | METDTLLLWVLLLWVPGSTGDIQMTQSPSSLSAS | CC379_Hz.Leu16_O2_VH3- |
| | VGDRVTITCRASSSVNYMDWYQQKPGKAPKLLIY | 23(30T.49G.71A) VL_VH CAR |
| | ATSNLASGVPSRFSGSGSGTDFTLTISSLQPEDFAT | T |
| | YYCQQWSFNPPTFGGGTKVEIKGSTSGGGSGGGS | |
| | GGGGSSEVQLLESGGGLVQPGGSLRLSCAASGFT | |
| | FTSYNMHWVRQAPGKGLEWVGAIYPGNGDTSYN | |
| | QKFKGRFTISADNSKNTLYLQMNSLRAEDTAVYY | |
| | CARSNYYGSSYWFFDVWGQGTTVTVSSESKYGP | |
| | PCPPCPAPEFEGGPSVFLFPPKPKDTLMISRTPEVT | |
| | CVVVDVSQEDPEVQFNWYVDGVEVHNAKTKPRE | |
| | EQFQSTYRVVSVLTVLHQDWLNGKEYKCKVSNK | |
| | GLPSSIEKTISKAKGQPREPQVYTLPPSQEEMTKN | |
| | QVSLTCLVKGFYPSDIAVEWESNGQPENNYKTTP | |
| | PVLDSDGSFFLYSRLTVDKSRWQEGNVFSCSVMH | |
| | EALHNHYTQKSLSLSLGKMFWVLVVVGGVLACY | |
| | SLLVTVAFIIFWVKRGRKKLLYIFKQPFMRPVQTT | |
| | QEEDGCSCRFPEEEEGGCELRVKFSRSADAPAYQ | |
| | QGQNQLYNELNLGRREEYDVLDKRRGRDPEMGG | |
| | KPRRKNPQEGLYNELQKDKMAEAYSEIGMKGER | |
| | RRGKGHDGLYQGLSTATKDTYDALHMQALPPR* | |
| | * | |
| 131 | METDTLLLWVLLLWVPGSTGEIVLTQSPATLSLSP | CC380_Hz.Leu16_A11_VH1- |
| | GERATLSCRASSSVNYMDWYQQKPGLAPRLLIYA | 46 VL_VH CAR T |
| | TSNLASGIPDRFSGSGSGTDFTLTISRLEPEDFAVY | |
| | YCQQWSFNPPTFGGGTKVEIKGSTSGGGSGGGSG | |
| | GGGSSQVQLVQSGAEVKKPGASVKVSCKASGYT | |
| | FTSYNMHWVRQAPGQGLEWMGAIYPGNGDTSY | |
| | NQKFKGRVTMTRDTSTSTVYMELSSLRSEDTAVY | |
| | YCARSNYYGSSYWFFDVWGQGTTVTVSSESKYG | |
| | PPCPPCPAPEFEGGPSVFLFPPKPKDTLMISRTPEV | |
| | TCVVVDVSQEDPEVQFNWYVDGVEVHNAKTKPR | |
| | EEQFQSTYRVVSVLTVLHQDWLNGKEYKCKVSN | |
| | KGLPSSIEKTISKAKGQPREPQVYTLPPSQEEMTK | |
| | NQVSLTCLVKGFYPSDIAVEWESNGQPENNYKTT | |
| | PPVLDSDGSFFLYSRLTVDKSRWQEGNVFSCSVM | |
| | HEALHNHYTQKSLSLSLGKMFWVLVVVGGVLAC | |
| | YSLLVTVAFIIFWVKRGRKKLLYIFKQPFMRPVQT | |
| | TQEEDGCSCRFPEEEEGGCELRVKFSRSADAPAY | |
| | QQGQNQLYNELNLGRREEYDVLDKRRGRDPEMG | |
| | GKPRRKNPQEGLYNELQKDKMAEAYSEIGMKGE | |
| | RRRGKGHDGLYQGLSTATKDTYDALHMQALPPR | |
| | ** | |
| 132 | METDTLLLWVLLLWVPGSTGEIVLTQSPATLSLSP | CC381-Hz.Lue16_A11/VH1- |
| | GERATLSCRASSSVNYMDWYQQKPGLAPRLLIYA | 46(73K) VL_VH CAR T |
| | TSNLASGIPDRFSGSGSGTDFTLTISRLEPEDFAVY | |
| | YCQQWSFNPPTFGGGTKVEIKGSTSGGGSGGGSG | |
| | GGGSSQVQLVQSGAEVKKPGASVKVSCKASGYT | |
| | FTSYNMHWVRQAPGQGLEWMGAIYPGNGDTSY | |
| | NQKFKGRVTMTRDKSTSTVYMELSSLRSEDTAVY | |
| | YCARSNYYGSSYWFFDVWGQGTTVTVSSESKYG | |
| | PPCPPCPAPEFEGGPSVFLFPPKPKDTLMISRTPEV | |
| | TCVVVDVSQEDPEVQFNWYVDGVEVHNAKTKPR | |
| | EEQFQSTYRVVSVLTVLHQDWLNGKEYKCKVSN | |
| | KGLPSSIEKTISKAKGQPREPQVYTLPPSQEEMTK | |
| | NQVSLTCLVKGFYPSDIAVEWESNGQPENNYKTT | |
| | PPVLDSDGSFFLYSRLTVDKSRWQEGNVFSCSVM | |
| | HEALHNHYTQKSLSLSLGKMFWVLVVVGGVLAC | |
| | YSLLVTVAFIIFWVKRGRKKLLYIFKQPFMRPVQT | |
| | TQEEDGCSCRFPEEEEGGCELRVKFSRSADAPAY | |
| | QQGQNQLYNELNLGRREEYDVLDKRRGRDPEMG | |
| | GKPRRKNPQEGLYNELQKDKMAEAYSEIGMKGE | |
| | RRRGKGHDGLYQGLSTATKDTYDALHMQALPPR | |
| | ** | |
| 133 | METDTLLLWVLLLWVPGSTGEIVLTQSPATLSLSP | CC382_Hz.Leu16_A11/VH3- |
| | GERATLSCRASSSVNYMDWYQQKPGLAPRLLIYA | 23 VL_VH CAR T |
| | TSNLASGIPDRESGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFS | |
| | SYNMHWVRQAPGKGLEWVSAIYPGNGDTSYNQ | |
| | KFKGRFTISRDNSKNTLYLQMNSLRAEDTAVYYC | |
| | ARSNYYGSSYWFFDVWGQGTTVTVSSESKYGPP | |
| | CPPCPAPEFEGGPSVFLFPPKPKDTLMISRTPEVTC | |
| | VVVDVSQEDPEVQFNWYVDGVEVHNAKTKPREE | |
| | QFQSTYRVVSVLTVLHQDWLNGKEYKCKVSNKG | |
| | LPSSIEKTISKAKGQPREPQVYTLPPSQEEMTKNQ | |
| | VSLTCLVKGFYPSDIAVEWESNGQPENNYKTTPP | |
| | VLDSDGSFFLYSRLTVDKSRWQEGNVFSCSVMHE | |
| | ALHNHYTQKSLSLSLGKMFWVLVVVGGVLACYS | |
| | LLVTVAFIIFWVKRGRKKLLYIFKQPFMRPVQTTQ | |
| | EEDGCSCRFPEEEEGGCELRVKFSRSADAPAYQQ | |
| | GQNQLYNELNLGRREEYDVLDKRRGRDPEMGGK | |
| | PRRKNPQEGLYNELQKDKMAEAYSEIGMKGERR | |
| | RGKGHDGLYQGLSTATKDTYDALHMQALPPR** | |
| 134 | METDTLLLWVLLLWVPGSTGEIVLTQSPATLSLSP | CC383_HZ.Lue16_A11_VH3- |
| | GERATLSCRASSSVNYMDWYQQKPGLAPRLLIYA | 23(30T) VL_VH CAR T |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVSAIYPGNGDTSYNQ | |
| | KFKGRFTISRDNSKNTLYLQMNSLRAEDTAVYYC | |
| | ARSNYYGSSYWFFDVWGQGTTVTVSSESKYGPP | |
| | CPPCPAPEFEGGPSVFLFPPKPKDTLMISRTPEVTC | |
| | VVVDVSQEDPEVQFNWYVDGVEVHNAKTKPREE | |
| | QFQSTYRVVSVLTVLHQDWLNGKEYKCKVSNKG | |
| | LPSSIEKTISKAKGQPREPQVYTLPPSQEEMTKNQ | |
| | VSLTCLVKGFYPSDIAVEWESNGQPENNYKTTPP | |
| | VLDSDGSFFLYSRLTVDKSRWQEGNVFSCSVMHE | |
| | ALHNHYTQKSLSLSLGKMFWVLVVVGGVLACYS | |
| | LLVTVAFIIFWVKRGRKKLLYIFKQPFMRPVQTTQ | |
| | EEDGCSCRFPEEEEGGCELRVKFSRSADAPAYQQ | |
| | GQNQLYNELNLGRREEYDVLDKRRGRDPEMGGK | |
| | PRRKNPQEGLYNELQKDKMAEAYSEIGMKGERR | |
| | RGKGHDGLYQGLSTATKDTYDALHMQALPPR** | |
| 135 | METDTLLLWVLLLWVPGSTGEIVLTQSPATLSLSP | CC384-HZ.Leu16_A11/VH3- |
| | GERATLSCRASSSVNYMDWYQQKPGLAPRLLIYA | 23(30T.49G) VL_VH CAR T |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISRDNSKNTLYLQMNSLRAEDTAVYYC | |
| | ARSNYYGSSYWFFDVWGQGTTVTVSSESKYGPP | |
| | CPPCPAPEFEGGPSVFLFPPKPKDTLMISRTPEVTC | |
| | VVVDVSQEDPEVQFNWYVDGVEVHNAKTKPREE | |
| | QFQSTYRVVSVLTVLHQDWLNGKEYKCKVSNKG | |
| | LPSSIEKTISKAKGQPREPQVYTLPPSQEEMTKNQ | |
| | VSLTCLVKGFYPSDIAVEWESNGQPENNYKTTPP | |
| | VLDSDGSFFLYSRLTVDKSRWQEGNVFSCSVMHE | |
| | ALHNHYTQKSLSLSLGKMFWVLVVVGGVLACYS | |
| | LLVTVAFIIFWVKRGRKKLLYIFKQPFMRPVQTTQ | |
| | EEDGCSCRFPEEEEGGCELRVKFSRSADAPAYQQ | |
| | GQNQLYNELNLGRREEYDVLDKRRGRDPEMGGK | |
| | PRRKNPQEGLYNELQKDKMAEAYSEIGMKGERR | |
| | RGKGHDGLYQGLSTATKDTYDALHMQALPPR** | |
| 136 | METDTLLLWVLLLWVPGSTGEIVLTQSPATLSLSP | CC385-Hz.Lue16_A11/VH3- |
| | GERATLSCRASSSVNYMDWYQQKPGLAPRLLIYA | 23(30T.49G.71A) VL_VH CAR |
| | TSNLASGIPDRFSGSGSGTDFTLTISRLEPEDFAVY | T |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISADNSKNTLYLQMNSLRAEDTAVYYC | |
| | ARSNYYGSSYWFFDVWGQGTTVTVSSESKYGPP | |
| | CPPCPAPEFEGGPSVFLFPPKPKDTLMISRTPEVTC | |
| | VVVDVSQEDPEVQFNWYVDGVEVHNAKTKPREE | |
| | QFQSTYRVVSVLTVLHQDWLNGKEYKCKVSNKG | |
| | LPSSIEKTISKAKGQPREPQVYTLPPSQEEMTKNQ | |
| | VSLTCLVKGFYPSDIAVEWESNGQPENNYKTTPP | |
| | VLDSDGSFFLYSRLTVDKSRWQEGNVFSCSVMHE | |
| | ALHNHYTQKSLSLSLGKMFWVLVVVGGVLACYS | |
| | LLVTVAFIIFWVKRGRKKLLYIFKQPFMRPVQTTQ | |
| | EEDGCSCRFPEEEEGGCELRVKFSRSADAPAYQQ | |
| | GQNQLYNELNLGRREEYDVLDKRRGRDPEMGGK | |
| | PRRKNPQEGLYNELQKDKMAEAYSEIGMKGERR | |
| | RGKGHDGLYQGLSTATKDTYDALHMQALPPR** | |
| 137 | METDTLLLWVLLLWVPGSTG | mIgK leader Sequence |
| 138 | GSTSGGGSGGGSGGGGSS | GS(18) Linker |
| 139 | FWVLVVVGGVLACYSLLVTVAFIIFW | CD28 TM |
| 140 | VKRGRKKLLYIFKQPFMRPVQTTQEEDGCSCRFP | 41BB |
| | EEEEGGCEL | |
| 141 | RVKFSRSADAPAYQQGQNQLYNELNLGRREEYD | CD3z |
| | VLDKRRGRDPEMGGKPRRKNPQEGLYNELQKDK | |
| | MAEAYSEIGMKGERRRGKGHDGLYQGLSTATKD | |
| | TYDALHMQALPPR | |

In some embodiments, the CAR may comprises a humanized anti CD19 construct. Exemplary sequences of said construct are listed in Table 3.

TABLE 3
Exemplary humanized anti CD19 construct sequences.

| SEQ ID NO | Amino Acid Sequence | Annotation |
|---|---|---|
| 142 | EVKLQESGPGLVAPSQSLSVTCTVSGVSLPDYGVS | Murine anti CD19 FMC63VH |
| | WIRQPPRKGLEWLGVIWGSETTYYNSALKSRLTII | sequence |
| | KDNSKSQVFLKMNSLQTDDTAIYYCAKHYYYGG | |
| | SYAMDYWGQGTSVTVSS | |
| 143 | DYGVS | CDRH1 |
| 144 | VIWGSETTYYNSALKS | CDRH2 |
| 145 | HYYYGGSYAMDY | CDRH3 |
| 146 | QVQLQESGPGLVKPSETLSLTCTVSGGSISSYYWS | VH4-59 (a) |
| | WIRQPPGKGLEWIGYIYYSGSTNYN | |
| 147 | EVKLQESGPGLVAPSQSLSVTCTVSGVSLPDYGVS | FMC63 VH (a) |
| | WIRQPPRKGLEWLGVIWGSETTYYN | |
| 148 | QVQLQESGPGLVKPSETLSLTCTVSGGSISDYGVS | Hz FMC63 VH4-59 (a) |
| | WIRQPPGKGLEWIGVIWGSETTYYN | |
| 149 | PSLKSRVTISVDTSKNQFSLKLSSVTAADTAVYYC | VH4-59 (b) |
| | AR | |
| 150 | SALKSRLTIIKDNSKSQVFLKMNSLQTDDTAIYYC | FMC63 VH (b) |
| | AKHYYYGGSYAMDYWGQGTSVTVSS | |
| 151 | SALKSRVTISVDTSKNQFSLKLSSVTAADTAVYYC | Hz FMC63 VH4-59 (b) |
| | AKHYYYGGSYAMDYWGQGTLVTVSS | |
| 152 | QVQLQESGPGLVKPSETLSLTCTVSGGSIPDYGVS | Hz FMC63 VH4-59 contact |
| | WIRQPPGKGLEWLGVIWGSETTYYN | |
| 153 | EVKLQESGPGLVAPSQSLSVTCTVSGVSLPDYGVS | FMC63 VH (c) |
| | WIRQPPRKGLEWLGVIWGSETTYYN | |
| 154 | PSLKSRLTISVDTSKNQFSLKLSSVTAADTAVYYC | Hz FMC63 VH4-59 contact |
| | AKHYYYGGSYAMDYWGQGTLVTVSS | |
| 155 | SALKSRLTIIKDNSKSQVFLKMNSLQTDDTAIYYC | FMC63 VH |
| | AKHYYYGGSYAMDYWGQGTSVTVSS | |
| 156 | DIQMTQTTSSLSASLGDRVTISCRASQDISKYLNW | Murine anti CD19 FMC63VK |
| | YQQKPDGTVKLLIYHTSRLHSGVPSRFSGSGSGTD | sequence |
| | YSLTISNLEQEDIATYFCQQGNTLPYTFGGGTKLEI | |
| | T | |
| 157 | DIQMTQSPSSLSASVGDRVTITCRASQSISSYLNW | O2-VK |
| | YQQKPGKAPKLLIYAASSLQSGVPS | |
| 158 | DIQMTQTTSSLSASLGDRVTISCRASQDISKYLNW | FMC63 VK |
| | YQQKPDGTVKLLIYHTSRLHSGVPS | |
| 159 | DIQMTQSPSSLSASVGDRVTITCRASQDISKYLNW | Hz FMC63 VK-O2 |
| | YQQKPGKAPKLLIYHTSRLHSGVPS | |
| 160 | RFSGSGSGTDFTLTISSLQPEDFATYYCQQSYSTP | O2-VK (b) |
| 161 | RFSGSGSGTDYSLTISNLEQEDIATYFCQQGNTLP | FMC63 VK (b) |
| | YTFGGGTKLEIT | |
| 162 | RFSGSGSGTDYTLTISSLQPEDFATYYCQQGNTLP | Hz FMC63 VK-O2 (b) |
| | YTFGGGVKLEIK | |
| 163 | METDTLLLWVLLLWVPGSTGQVQLQESGPGLVK | FMC63-9 HzVH4-59_HzVKO2 |
| | PSETLSLTCTVSGGSISDYGVSWIRQPPGKGLEWI | |
| | GVIWGSETTYYNSALKSRVTISVDTSKNQFSLKLS | |
| | SVTAADTAVYYCAKHYYYGGSYAMDYWGQGTL | |
| | VTVSSGSTSGSGKPGSGEGSTKGDIQMTQSPSSLS | |
| | ASVGDRVTITCRASQDISKYLNWYQQKPGKAPKL | |
| | LIYHTSRLHSGVPSRFSGSGSGTDYTLTISSLQPED | |
| | FATYYCQQGNTLPYTFGGGVKLEIKESKYGPPCPP | |
| | CPMFWVLVVVGGVLACYSLLVTVAFIIFWVKRG | |
| | RKKLLYIFKQPFMRPVQTTQEEDGCSCRFPEEEEG | |
| | GCELRVKFSRSADAPAYQQGQNQLYNELNLGRR | |
| | EEYDVLDKRRGRDPEMGGKPRRKNPQEGLYNEL | |
| | QKDKMAEAYSEIGMKGERRRGKGHDGLYQGLST | |
| | ATKDTYDALHMQALPPR* | |
| 164 | METDTLLLWVLLLWVPGSTGQVQLQESGPGLVK | FMC63-10 HzVH4- |
| | PSETLSLTCTVSGGSLPDYGVSWIRQPPGKGLEWI | 59(L29P30)_HzVKO2 |
| | GVIWGSETTYYNSALKSRVTISVDTSKNQFSLKLS | |
| | SVTAADTAVYYCAKHYYYGGSYAMDYWGQGTL | |
| | VTVSSGSTSGSGKPGSGEGSTKGDIQMTQSPSSLS | |
| | ASVGDRVTITCRASQDISKYLNWYQQKPGKAPKL | |
| | LIYHTSRLHSGVPSRFSGSGSGTDYTLTISSLQPED | |
| | FATYYCQQGNTLPYTFGGGVKLEIKESKYGPPCPP | |
| | CPMFWVLVVVGGVLACYSLLVTVAFIIFWVKRG | |
| | RKKLLYIFKQPFMRPVQTTQEEDGCSCRFPEEEEG | |
| | GCELRVKFSRSADAPAYQQGQNQLYNELNLGRR | |
| | EEYDVLDKRRGRDPEMGGKPRRKNPQEGLYNEL | |
| | QKDKMAEAYSEIGMKGERRRGKGHDGLYQGLST | |
| | ATKDTYDALHMQALPPR* | |
| 165 | METDTLLLWVLLLWVPGSTGQVQLQESGPGLVK | FMC63-11 HzVH4- |
| | PSETLSLTCTVSGGSIPDYGVSWIRQPPGKGLEWL | 59(Con)_HzVKO2 |
| | GVIWGSETTYYNPSLKSRLTISVDTSKNQVSLKLS | |
| | SVTAADTAVYYCAKHYYYGGSYAMDYWGQGTL | |
| | VTVSSGSTSGSGKPGSGEGSTKGDIQMTQSPSSLS | |
| | ASVGDRVTITCRASQDISKYLNWYQQKPGKAPKL | |
| | LIYHTSRLHSGVPSRFSGSGSGTDYTLTISSLQPED | |
| | FATYYCQQGNTLPYTFGGGVKLEIKESKYGPPCPP | |
| | CPMFWVLVVVGGVLACYSLLVTVAFIIFWVKRG | |
| | RKKLLYIFKQPFMRPVQTTQEEDGCSCRFPEEEEG | |
| | GCELRVKFSRSADAPAYQQGQNQLYNELNLGRR | |
| | EEYDVLDKRRGRDPEMGGKPRRKNPQEGLYNEL | |
| | QKDKMAEAYSEIGMKGERRRGKGHDGLYQGLST | |
| | ATKDTYDALHMQALPPR* | |
| 166 | METDTLLLWVLLLWVPGSTGEVQLLESGGGLVQ | FMC63-12 HzVH3- |
| | PGGSLRLSCAASGFTFSDYGVSWVRQAPGKGLE | 23_HzVKO2 |
| | WVSVIWGSETTYYNSALKSRFTISRDNSKNTLYL | |
| | QMNSLRAEDTAVYYCAKHYYYGGSYAMDYWG | |
| | QGTLVTVSSGSTSGSGKPGSGEGSTKGDIQMTQSP | |
| | SSLSASVGDRVTITCRASQDISKYLNWYQQKPGK | |
| | APKLLIYHTSRLHSGVPSRFSGSGSGTDYTLTISSL | |
| | QPEDFATYYCQQGNTLPYTFGGGVKLEIKESKYG | |
| | PPCPPCPMFWVLVVVGGVLACYSLLVTVAFIIFW | |
| | VKRGRKKLLYIFKQPFMRPVQTTQEEDGCSCRFP | |
| | EEEEGGCELRVKFSRSADAPAYQQGQNQLYNELN | |
| | LGRREEYDVLDKRRGRDPEMGGKPRRKNPQEGL | |
| | YNELQKDKMAEAYSEIGMKGERRRGKGHDGLY | |
| | QGLSTATKDTYDALHMQALPPR* | |
| 167 | METDTLLLWVLLLWVPGSTGEVQLLESGGGLVQ | FMC63-13 HzVH3- |
| | PGGSLRLSCAASGFTFSDYGVSWVRQAPGKGLE | 23(KV)_HzVKO2 |
| | WVSVIWGSETTYYNSALKSRFTISKDNSKNTVYL | |
| | QMNSLRAEDTAVYYCAKHYYYGGSYAMDYWG | |
| | QGTLVTVSSGSTSGSGKPGSGEGSTKGDIQMTQSP | |
| | SSLSASVGDRVTITCRASQDISKYLNWYQQKPGK | |
| | APKLLIYHTSRLHSGVPSRFSGSGSGTDYTLTISSL | |
| | QPEDFATYYCQQGNTLPYTFGGGVKLEIKESKYG | |
| | PPCPPCPMFWVLVVVGGVLACYSLLVTVAFIIFW | |
| | VKRGRKKLLYIFKQPFMRPVQTTQEEDGCSCRFP | |
| | EEEEGGCELRVKFSRSADAPAYQQGQNQLYNELN | |
| | LGRREEYDVLDKRRGRDPEMGGKPRRKNPQEGL | |
| | YNELQKDKMAEAYSEIGMKGERRRGKGHDGLY | |
| | QGLSTATKDTYDALHMQALPPR* | |
| 168 | METDTLLLWVLLLWVPGSTGEVQLLESGGGLVQ | Hz.FMC63-16: |
| | PGGSLRLSCAASGFTFSDYGVSWVRQAPGKGLE | VH3-23; VKO2 |
| | WVSVIWGSETTYYNSALKSRFTISRDNSKNTLYL | |
| | QMNSLRAEDTAVYYCAKHYYYGGSYAMDYWG | |
| | QGTLVTVSSGSTSGSGKPGSGEGSTKGDIQMTQSP | |
| | SSLSASVGDRVTITCRASQDISKYLNWYQQKPGK | |
| | APKLLIYHTSRLHSGVPSRFSGSGSGTDYTLTISSL | |
| | QPEDFATYYCQQGNTLPYTFGGGTKVEIKESKYG | |
| | PPCPPCPMFWVLVVVGGVLACYSLLVTVAFIIFW | |
| | VKRGRKKLLYIFKQPFMRPVQTTQEEDGCSCRFP | |
| | EEEEGGCELRVKFSRSADAPAYQQGQNQLYNELN | |
| | LGRREEYDVLDKRRGRDPEMGGKPRRKNPQEGL | |
| | YNELQKDKMAEAYSEIGMKGERRRGKGHDGLY | |
| | QGLSTATKDTYDALHMQALPPR** | |
| 169 | METDTLLLWVLLLWVPGSTGEVQLLESGGGLVQ | Hz.FMC63-17: VH3-23; |
| | PGGSLRLSCAASGFTFSDYGVSWVRQAPGKGLE | VKO2(L87F) |
| | WVSVIWGSETTYYNSALKSRFTISRDNSKNTLYL | |
| | QMNSLRAEDTAVYYCAKHYYYGGSYAMDYWG | |
| | QGTLVTVSSGSTSGSGKPGSGEGSTKGDIQMTQSP | |
| | SSLSASVGDRVTITCRASQDISKYLNWYQQKPGK | |
| | APKLLIYHTSRLHSGVPSRFSGSGSGTDYTLTISSL | |
| | QPEDFATYFCQQGNTLPYTFGGGTKVEIKESKYG | |
| | PPCPPCPMFWVLVVVGGVLACYSLLVTVAFIIFW | |
| | VKRGRKKLLYIFKQPFMRPVQTTQEEDGCSCRFP | |
| | EEEEGGCELRVKFSRSADAPAYQQGQNQLYNELN | |
| | LGRREEYDVLDKRRGRDPEMGGKPRRKNPQEGL | |
| | YNELQKDKMAEAYSEIGMKGERRRGKGHDGLY | |
| | QGLSTATKDTYDALHMQALPPR** | |
| 170 | METDTLLLWVLLLWVPGSTGEVQLLESGGGLVQ | Hz.FMC63-18: VH3-23; |
| | PGGSLRLSCAASGFTFSDYGVSWVRQAPGKGLE | VKO2(L22S) |
| | WVSVIWGSETTYYNSALKSRFTISRDNSKNTLYL | |
| | QMNSLRAEDTAVYYCAKHYYYGGSYAMDYWG | |
| | QGTLVTVSSGSTSGSGKPGSGEGSTKGDIQMTQSP | |
| | SSLSASVGDRVTISCRASQDISKYLNWYQQKPGK | |
| | APKLLIYHTSRLHSGVPSRFSGSGSGTDYTLTISSL | |
| | QPEDFATYYCQQGNTLPYTFGGGTKVEIKESKYG | |
| | PPCPPCPMFWVLVVVGGVLACYSLLVTVAFIIFW | |
| | VKRGRKKLLYIFKQPFMRPVQTTQEEDGCSCRFP | |
| | EEEEGGCELRVKFSRSADAPAYQQGQNQLYNELN | |
| | LGRREEYDVLDKRRGRDPEMGGKPRRKNPQEGL | |
| | YNELQKDKMAEAYSEIGMKGERRRGKGHDGLY | |
| | QGLSTATKDTYDALHMQALPPR** | |
| 171 | METDTLLLWVLLLWVPGSTGEVQLLESGGGLVQ | Hz.FMC63-19: VH3-23; |
| | PGGSLRLSCAASGFTFSDYGVSWVRQAPGKGLE | VKO2(L82I) |
| | WVSVIWGSETTYYNSALKSRFTISRDNSKNTLYL | |
| | QMNSLRAEDTAVYYCAKHYYYGGSYAMDYWG | |
| | QGTLVTVSSGSTSGSGKPGSGEGSTKGDIQMTQSP | |
| | SSLSASVGDRVTITCRASQDISKYLNWYQQKPGK | |
| | APKLLIYHTSRLHSGVPSRFSGSGSGTDYTLTISSL | |
| | QPEDIATYYCQQGNTLPYTFGGGTKVEIKESKYGP | |
| | PCPPCPMFWVLVVVGGVLACYSLLVTVAFIIFWV | |
| | KRGRKKLLYIFKQPFMRPVQTTQEEDGCSCRFPEE | |
| | EEGGCELRVKFSRSADAPAYQQGQNQLYNELNL | |
| | GRREEYDVLDKRRGRDPEMGGKPRRKNPQEGLY | |
| | NELQKDKMAEAYSEIGMKGERRRGKGHDGLYQ | |
| | GLSTATKDTYDALHMQALPPR** | |
| 172 | METDTLLLWVLLLWVPGSTGEVQLLESGGGLVQ | Hz.FMC63-20: VH3- |
| | PGGSLRLSCAASGFTFSDYGVSWVRQAPGKGLE | 23(H71,87); VKO2 |
| | WVSVIWGSETTYYNSALKSRFTISKDNSKNTVYL | |
| | QMNSLRAEDTAVYYCAKHYYYGGSYAMDYWG | |
| | QGTLVTVSSGSTSGSGKPGSGEGSTKGDIQMTQSP | |
| | SSLSASVGDRVTITCRASQDISKYLNWYQQKPGK | |
| | APKLLIYHTSRLHSGVPSRFSGSGSGTDYTLTISSL | |
| | QPEDFATYYCQQGNTLPYTFGGGTKVEIKESKYG | |
| | PPCPPCPMFWVLVVVGGVLACYSLLVTVAFIIFW | |
| | VKRGRKKLLYIFKQPFMRPVQTTQEEDGCSCRFP | |
| | EEEEGGCELRVKFSRSADAPAYQQGQNQLYNELN | |
| | LGRREEYDVLDKRRGRDPEMGGKPRRKNPQEGL | |
| | YNELQKDKMAEAYSEIGMKGERRRGKGHDGLY | |
| | QGLSTATKDTYDALHMQALPPR** | |
| 173 | METDTLLLWVLLLWVPGSTGEVQLLESGGGLVQ | Hz.FMC63-21: VH3- |
| | PGGSLRLSCAASGFTFSDYGVSWVRQAPGKGLE | 23(H71,87); VKO2(L87F) |
| | WVSVIWGSETTYYNSALKSRFTISKDNSKNTVYL | |
| | QMNSLRAEDTAVYYCAKHYYYGGSYAMDYWG | |
| | QGTLVTVSSGSTSGSGKPGSGEGSTKGDIQMTQSP | |
| | SSLSASVGDRVTITCRASQDISKYLNWYQQKPGK | |
| | APKLLIYHTSRLHSGVPSRFSGSGSGTDYTLTISSL | |
| | QPEDFATYFCQQGNTLPYTFGGGTKVEIKESKYG | |
| | PPCPPCPMFWVLVVVGGVLACYSLLVTVAFIIFW | |
| | VKRGRKKLLYIFKQPFMRPVQTTQEEDGCSCRFP | |
| | EEEEGGCELRVKFSRSADAPAYQQGQNQLYNELN | |
| | LGRREEYDVLDKRRGRDPEMGGKPRRKNPQEGL | |
| | YNELQKDKMAEAYSEIGMKGERRRGKGHDGLY | |
| | QGLSTATKDTYDALHMQALPPR** | |
| 174 | METDTLLLWVLLLWVPGSTGEVQLLESGGGLVQ | Hz.FMC63-22: VH3- |
| | PGGSLRLSCAASGFTFSDYGVSWVRQAPGKGLE | 23(H71,87); VKO2(L22S) |
| | WVSVIWGSETTYYNSALKSRFTISKDNSKNTVYL | |
| | QMNSLRAEDTAVYYCAKHYYYGGSYAMDYWG | |
| | QGTLVTVSSGSTSGSGKPGSGEGSTKGDIQMTQSP | |
| | SSLSASVGDRVTISCRASQDISKYLNWYQQKPGK | |
| | APKLLIYHTSRLHSGVPSRFSGSGSGTDYTLTISSL | |
| | QPEDFATYYCQQGNTLPYTFGGGTKVEIKESKYG | |
| | PPCPPCPMFWVLVVVGGVLACYSLLVTVAFIIFW | |
| | VKRGRKKLLYIFKQPFMRPVQTTQEEDGCSCRFP | |
| | EEEEGGCELRVKFSRSADAPAYQQGQNQLYNELN | |
| | LGRREEYDVLDKRRGRDPEMGGKPRRKNPQEGL | |
| | YNELQKDKMAEAYSEIGMKGERRRGKGHDGLY | |
| | QGLSTATKDTYDALHMQALPPR** | |
| 175 | METDTLLLWVLLLWVPGSTGEVQLLESGGGLVQ | Hz.FMC63-23: VH3- |
| | PGGSLRLSCAASGFTFSDYGVSWVRQAPGKGLE | 23(H71,87); VKO2(L82I) |
| | WVSVIWGSETTYYNSALKSRFTISKDNSKNTVYL | |
| | QMNSLRAEDTAVYYCAKHYYYGGSYAMDYWG | |
| | QGTLVTVSSGSTSGSGKPGSGEGSTKGDIQMTQSP | |
| | SSLSASVGDRVTITCRASQDISKYLNWYQQKPGK | |
| | APKLLIYHTSRLHSGVPSRFSGSGSGTDYTLTISSL | |
| | QPEDIATYYCQQGNTLPYTFGGGTKVEIKESKYGP | |
| | PCPPCPMFWVLVVVGGVLACYSLLVTVAFIIFWV | |
| | KRGRKKLLYIFKQPFMRPVQTTQEEDGCSCRFPEE | |
| | EEGGCELRVKFSRSADAPAYQQGQNQLYNELNL | |
| | GRREEYDVLDKRRGRDPEMGGKPRRKNPQEGLY | |
| | NELQKDKMAEAYSEIGMKGERRRGKGHDGLYQ | |
| | GLSTATKDTYDALHMQALPPR** | |
| 176 | METDTLLLWVLLLWVPGSTG | mIgK leader Sequence |
| 177 | GSTSGSGKPGSGEGSTKG | Whitlow Linker |
| 178 | ESKYGPPCPPCP | IgG4 Hinge |
| 179 | FWVLVVVGGVLACYSLLVTVAFIIFWV | CD28 TM |
| 180 | KRGRKKLLYIFKQPFMRPVQTTQEEDGCSCRFPEE | 41BB |
| | EEGGCEL | |
| 181 | RVKFSRSADAPAYQQGQNQLYNELNLGRREEYD | CD3z |
| | VLDKRRGRDPEMGGKPRRKNPQEGLYNELQKDK | |
| | MAEAYSEIGMKGERRRGKGHDGLYQGLSTATKD | |
| | TYDALHMQALPPR | |

In some embodiments, the CAR may be a humanized CD19/CD20. Exemplary CAR sequences are listed in tables 4 and 5.

TABLE 4
Humanized CD19/20 (Leu16/FMC63) bi-specific CAR Summary of Sequences

| SEQ | Hz. Lu16 | | | Hz. FMC63 | | |
|---|---|---|---|---|---|---|
| ID | CD20 | VH | VK | CD19 | VH | VK |
| 182 | 374 | VH1-46 | O2 | Hz.FMC63-12 | VH3-23 | VKO2 |
| 183 | 376 | VH3-23 | O2 | Hz.FMC63-12 | VH3-23 | VKO2 |
| 184 | 378 | VH3-3(30T.49G) | O2 | Hz.FMC63-12 | VH3-23 | VKO2 |
| 185 | 381 | VH1-46(73K) | A11 | Hz.FMC63-12 | VH3-23 | VKO2 |
| 186 | 384 | VH3-23(30T.49G) | A11 | Hz.FMC63-12 | VH3-23 | VKO2 |
| 187 | 385 | VH3-23(30T.49G.71A | A11 | Hz.FMC63-12 | VH3-23 | VKO2 |
| 188 | 374 | VH1-46 | O2 | Hz.FMC63-16 | VH3-23 | VKO2 |
| 189 | 376 | VH3-23 | O2 | Hz.FMC63-16 | VH3-23 | VKO2 |
| 190 | 378 | VH3-3(30T.49G) | O2 | Hz.FMC63-16 | VH3-23 | VKO2 |
| 191 | 381 | VH1-46(73K) | A11 | Hz.FMC63-16 | VH3-23 | VKO2 |
| 192 | 384 | VH3-23(30T.49G) | A11 | Hz.FMC63-16 | VH3-23 | VKO2 |
| 193 | 385 | VH3-23(30T.49G.71A | A11 | Hz.FMC63-16 | VH3-23 | VKO2 |
| 194 | 374 | VH1-46 | O2 | Hz.FMC63-18 | VH3-23 | VKO2(L22S) |
| 195 | 376 | VH3-23 | O2 | Hz.FMC63-18 | VH3-23 | VKO2(L22S) |
| 196 | 378 | VH3-3(30T.49G) | O2 | Hz.FMC63-18 | VH3-23 | VKO2(L22S) |
| 197 | 381 | VH1-46(73K) | A11 | Hz.FMC63-18 | VH3-23 | VKO2(L22S) |
| 198 | 384 | VH3-23(30T.49G) | A11 | Hz.FMC63-18 | VH3-23 | VKO2(L22S) |
| 199 | 385 | VH3-23(30T.49G.71A | A11 | Hz.FMC63-18 | VH3-23 | VKO2(L22S) |
| 200 | 374 | VH1-46 | O2 | Hz.FMC63-19 | VH3-23 | VKO2(L82I) |
| 201 | 376 | VH3-23 | O2 | Hz.FMC63-19 | VH3-23 | VKO2(L82I) |
| 202 | 378 | VH3-3(30T.49G) | O2 | Hz.FMC63-19 | VH3-23 | VKO2(L82I) |
| 203 | 381 | VH1-46(73K) | A11 | Hz.FMC63-19 | VH3-23 | VKO2(L82I) |
| 204 | 384 | VH3-23(30T.49G) | A11 | Hz.FMC63-19 | VH3-23 | VKO2(L82I) |
| 205 | 385 | VH3-23(30T.49G.71A | A11 | Hz.FMC63-19 | VH3-23 | VKO2(L82I) |
| 206 | 374 | VH1-46 | O2 | Hz.FMC63-20 | VH3-23(H71,87) | VKO2 |
| 207 | 376 | VH3-23 | O2 | Hz.FMC63-20 | VH3-23(H71,87) | VKO2 |
| 208 | 378 | VH3-3(30T.49G) | O2 | Hz.FMC63-20 | VH3-23(H71,87) | VKO2 |
| 209 | 381 | VH1-46(73K) | A11 | Hz.FMC63-20 | VH3-23(H71,87) | VKO2 |
| 210 | 384 | VH3-23(30T.49G) | A11 | Hz.FMC63-20 | VH3-23(H71,87) | VKO2 |
| 211 | 385 | VH3-23(30T.49G.71A | A11 | Hz.FMC63-20 | VH3-23(H71,87) | VKO2 |
| 212 | 374 | VH1-46 | O2 | Hz.FMC63-21 | VH3-23(H71,87) | VKO2(L87F) |
| 213 | 376 | VH3-23 | O2 | Hz.FMC63-21 | VH3-23(H71,87) | VKO2(L87F) |
| 214 | 378 | VH3-3(30T.49G) | O2 | Hz.FMC63-21 | VH3-23(H71,87) | VKO2(L87F) |
| 215 | 381 | VH1-46(73K) | A11 | Hz.FMC63-21 | VH3-23(H71,87) | VKO2(L87F) |
| 216 | 384 | VH3-23(30T.49G) | A11 | Hz.FMC63-21 | VH3-23(H71,87) | VKO2(L87F) |
| 217 | 385 | VH3-23(30T.49G.71A | A11 | Hz.FMC63-21 | VH3-23(H71,87) | VKO2(L87F) |

TABLE 5
Humanized CD19/20 (Leu16/FMC63) bi-specific CAR Sequences

| SEQ ID NO | Amino Acid Sequence | Annotation |
|---|---|---|
| 182 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSQVQLVQSGAEVKKPGASVKVSCKASGY | |
| | TFTSYNMHWVRQAPGQGLEWMGAIYPGNGDTS | |
| | YNQKFKGRVTMTRDTSTSTVYMELSSLRSEDTAV | |
| | YYCARSNYYGSSYWFFDVWGQGTTVTVSSGGGG | |
| | SGGGGSGGGGSGGGGSEVQLLESGGGLVQPGGSL | |
| | RLSCAASGFTFSDYGVSWVRQAPGKGLEWVSVI | |
| | WGSETTYYNSALKSRFTISRDNSKNTLYLQMNSL | |
| | RAEDTAVYYCAKHYYYGGSYAMDYWGQGTLVT | |
| | VSSGSTSGSGKPGSGEGSTKGDIQMTQSPSSLSAS | |
| | VGDRVTITCRASQDISKYLNWYQQKPGKAPKLLI | |
| | YHTSRLHSGVPSRFSGSGSGTDYTLTISSLQPEDFA | |
| | TYYCQQGNTLPYTFGGGVKLEIKESKYGPPCPPCP | |
| | MFWVLVVVGGVLACYSLLVTVAFIIFWVKRGRK | |
| | KLLYIFKQPFMRPVQTTQEEDGCSCRFPEEEEGGC | |
| | ELRVKFSRSADAPAYQQGQNQLYNELNLGRREE | |
| | YDVLDKRRGRDPEMGGKPRRKNPQEGLYNELQK | |
| | DKMAEAYSEIGMKGERRRGKGHDGLYQGLSTAT | |
| | KDTYDALHMQALPPR* | |
| 183 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FSSYNMHWVRQAPGKGLEWVSAIYPGNGDTSYN | |
| | QKFKGRFTISRDNSKNTLYLQMNSLRAEDTAVYY | |
| | CARSNYYGSSYWFFDVWGQGTTVTVSSGGGGSG | |
| | GGGSGGGGSGGGGSEVQLLESGGGLVQPGGSLRL | |
| | SCAASGFTFSDYGVSWVRQAPGKGLEWVSVIWG | |
| | SETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDFATYYC | |
| | QQGNTLPYTFGGGVKLEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 184 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FTSYNMHWVRQAPGKGLEWVGAIYPGNGDTSYN | |
| | QKFKGRFTISRDNSKNTLYLQMNSLRAEDTAVYY | |
| | CARSNYYGSSYWFFDVWGQGTTVTVSSGGGGSG | |
| | GGGSGGGGSGGGGSEVQLLESGGGLVQPGGSLRL | |
| | SCAASGFTFSDYGVSWVRQAPGKGLEWVSVIWG | |
| | SETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDFATYYC | |
| | QQGNTLPYTFGGGVKLEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 185 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSQVQLVQSGAEVKKPGASVKVSCKASGYT | |
| | FTSYNMHWVRQAPGQGLEWMGAIYPGNGDTSY | |
| | NQKFKGRVTMTRDKSTSTVYMELSSLRSEDTAVY | |
| | YCARSNYYGSSYWFFDVWGQGTTVTVSSGGGGS | |
| | GGGGSGGGGSGGGGSEVQLLESGGGLVQPGGSL | |
| | RLSCAASGFTFSDYGVSWVRQAPGKGLEWVSVI | |
| | WGSETTYYNSALKSRFTISRDNSKNTLYLQMNSL | |
| | RAEDTAVYYCAKHYYYGGSYAMDYWGQGTLVT | |
| | VSSGSTSGSGKPGSGEGSTKGDIQMTQSPSSLSAS | |
| | VGDRVTITCRASQDISKYLNWYQQKPGKAPKLLI | |
| | YHTSRLHSGVPSRFSGSGSGTDYTLTISSLQPEDFA | |
| | TYYCQQGNTLPYTFGGGVKLEIKESKYGPPCPPCP | |
| | MFWVLVVVGGVLACYSLLVTVAFIIFWVKRGRK | |
| | KLLYIFKQPFMRPVQTTQEEDGCSCRFPEEEEGGC | |
| | ELRVKFSRSADAPAYQQGQNQLYNELNLGRREE | |
| | YDVLDKRRGRDPEMGGKPRRKNPQEGLYNELQK | |
| | DKMAEAYSEIGMKGERRRGKGHDGLYQGLSTAT | |
| | KDTYDALHMQALPPR* | |
| 186 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISRDNSKNTLYLQMNSLRAEDTAVYYC | |
| | ARSNYYGSSYWFFDVWGQGTTVTVSSGGGGSGG | |
| | GGSGGGGSGGGGSEVQLLESGGGLVQPGGSLRLS | |
| | CAASGFTFSDYGVSWVRQAPGKGLEWVSVIWGS | |
| | ETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDFATYYC | |
| | QQGNTLPYTFGGGVKLEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 187 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISADNSKNTLYLQMNSLRAEDTAVYYC | |
| | ARSNYYGSSYWFFDVWGQGTTVTVSSGGGGSGG | |
| | GGSGGGGSGGGGSEVQLLESGGGLVQPGGSLRLS | |
| | CAASGFTFSDYGVSWVRQAPGKGLEWVSVIWGS | |
| | ETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDFATYYC | |
| | QQGNTLPYTFGGGVKLEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 188 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSQVQLVQSGAEVKKPGASVKVSCKASGY | |
| | TFTSYNMHWVRQAPGQGLEWMGAIYPGNGDTS | |
| | YNQKFKGRVTMTRDTSTSTVYMELSSLRSEDTAV | |
| | YYCARSNYYGSSYWFFDVWGQGTTVTVSSGGGG | |
| | SGGGGSGGGGSGGGGSEVQLLESGGGLVQPGGSL | |
| | RLSCAASGFTFSDYGVSWVRQAPGKGLEWVSVI | |
| | WGSETTYYNSALKSRFTISRDNSKNTLYLQMNSL | |
| | RAEDTAVYYCAKHYYYGGSYAMDYWGQGTLVT | |
| | VSSGSTSGSGKPGSGEGSTKGDIQMTQSPSSLSAS | |
| | VGDRVTITCRASQDISKYLNWYQQKPGKAPKLLI | |
| | YHTSRLHSGVPSRFSGSGSGTDYTLTISSLQPEDFA | |
| | TYYCQQGNTLPYTFGGGTKVEIKESKYGPPCPPCP | |
| | MFWVLVVVGGVLACYSLLVTVAFIIFWVKRGRK | |
| | KLLYIFKQPFMRPVQTTQEEDGCSCRFPEEEEGGC | |
| | ELRVKFSRSADAPAYQQGQNQLYNELNLGRREE | |
| | YDVLDKRRGRDPEMGGKPRRKNPQEGLYNELQK | |
| | DKMAEAYSEIGMKGERRRGKGHDGLYQGLSTAT | |
| | KDTYDALHMQALPPR* | |
| 189 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FSSYNMHWVRQAPGKGLEWVSAIYPGNGDTSYN | |
| | QKFKGRFTISRDNSKNTLYLQMNSLRAEDTAVYY | |
| | CARSNYYGSSYWFFDVWGQGTTVTVSSGGGGSG | |
| | GGGSGGGGSGGGGSEVQLLESGGGLVQPGGSLRL | |
| | SCAASGFTFSDYGVSWVRQAPGKGLEWVSVIWG | |
| | SETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDFATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 190 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FTSYNMHWVRQAPGKGLEWVGAIYPGNGDTSYN | |
| | QKFKGRFTISRDNSKNTLYLQMNSLRAEDTAVYY | |
| | CARSNYYGSSYWFFDVWGQGTTVTVSSGGGGSG | |
| | GGGSGGGGSGGGGSEVQLLESGGGLVQPGGSLRL | |
| | SCAASGFTFSDYGVSWVRQAPGKGLEWVSVIWG | |
| | SETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDFATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 191 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSQVQLVQSGAEVKKPGASVKVSCKASGYT | |
| | FTSYNMHWVRQAPGQGLEWMGAIYPGNGDTSY | |
| | NQKFKGRVTMTRDKSTSTVYMELSSLRSEDTAVY | |
| | YCARSNYYGSSYWFFDVWGQGTTVTVSSGGGGS | |
| | GGGGSGGGGSGGGGSEVQLLESGGGLVQPGGSL | |
| | RLSCAASGFTFSDYGVSWVRQAPGKGLEWVSVI | |
| | WGSETTYYNSALKSRFTISRDNSKNTLYLQMNSL | |
| | RAEDTAVYYCAKHYYYGGSYAMDYWGQGTLVT | |
| | VSSGSTSGSGKPGSGEGSTKGDIQMTQSPSSLSAS | |
| | VGDRVTITCRASQDISKYLNWYQQKPGKAPKLLI | |
| | YHTSRLHSGVPSRFSGSGSGTDYTLTISSLQPEDFA | |
| | TYYCQQGNTLPYTFGGGTKVEIKESKYGPPCPPCP | |
| | MFWVLVVVGGVLACYSLLVTVAFIIFWVKRGRK | |
| | KLLYIFKQPFMRPVQTTQEEDGCSCRFPEEEEGGC | |
| | ELRVKFSRSADAPAYQQGQNQLYNELNLGRREE | |
| | YDVLDKRRGRDPEMGGKPRRKNPQEGLYNELQK | |
| | DKMAEAYSEIGMKGERRRGKGHDGLYQGLSTAT | |
| | KDTYDALHMQALPPR* | |
| 192 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISRDNSKNTLYLQMNSLRAEDTAVYYC | |
| | ARSNYYGSSYWFFDVWGQGTTVTVSSGGGGSGG | |
| | GGSGGGGSGGGGSEVQLLESGGGLVQPGGSLRLS | |
| | CAASGFTFSDYGVSWVRQAPGKGLEWVSVIWGS | |
| | ETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDFATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 193 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISADNSKNTLYLQMNSLRAEDTAVYYC | |
| | ARSNYYGSSYWFFDVWGQGTTVTVSSGGGGSGG | |
| | GGSGGGGSGGGGSEVQLLESGGGLVQPGGSLRLS | |
| | CAASGFTFSDYGVSWVRQAPGKGLEWVSVIWGS | |
| | ETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDFATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 194 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSQVQLVQSGAEVKKPGASVKVSCKASGY | |
| | TFTSYNMHWVRQAPGQGLEWMGAIYPGNGDTS | |
| | YNQKFKGRVTMTRDTSTSTVYMELSSLRSEDTAV | |
| | YYCARSNYYGSSYWFFDVWGQGTTVTVSSGGGG | |
| | SGGGGSGGGGSGGGGSEVQLLESGGGLVQPGGSL | |
| | RLSCAASGFTFSDYGVSWVRQAPGKGLEWVSVI | |
| | WGSETTYYNSALKSRFTISRDNSKNTLYLQMNSL | |
| | RAEDTAVYYCAKHYYYGGSYAMDYWGQGTLVT | |
| | VSSGSTSGSGKPGSGEGSTKGDIQMTQSPSSLSAS | |
| | VGDRVTISCRASQDISKYLNWYQQKPGKAPKLLI | |
| | YHTSRLHSGVPSRFSGSGSGTDYTLTISSLQPEDFA | |
| | TYYCQQGNTLPYTFGGGTKVEIKESKYGPPCPPCP | |
| | MFWVLVVVGGVLACYSLLVTVAFIIFWVKRGRK | |
| | KLLYIFKQPFMRPVQTTQEEDGCSCRFPEEEEGGC | |
| | ELRVKFSRSADAPAYQQGQNQLYNELNLGRREE | |
| | YDVLDKRRGRDPEMGGKPRRKNPQEGLYNELQK | |
| | DKMAEAYSEIGMKGERRRGKGHDGLYQGLSTAT | |
| | KDTYDALHMQALPPR* | |
| 195 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FSSYNMHWVRQAPGKGLEWVSAIYPGNGDTSYN | |
| | QKFKGRFTISRDNSKNTLYLQMNSLRAEDTAVYY | |
| | CARSNYYGSSYWFFDVWGQGTTVTVSSGGGGSG | |
| | GGGSGGGGSGGGGSEVQLLESGGGLVQPGGSLRL | |
| | SCAASGFTFSDYGVSWVRQAPGKGLEWVSVIWG | |
| | SETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTISCRASQDISKYLNWYQQKPGKAPKLLIYHTS | |
| | RLHSGVPSRFSGSGSGTDYTLTISSLQPEDFATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 196 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FTSYNMHWVRQAPGKGLEWVGAIYPGNGDTSYN | |
| | QKFKGRFTISRDNSKNTLYLQMNSLRAEDTAVYY | |
| | CARSNYYGSSYWFFDVWGQGTTVTVSSGGGGSG | |
| | GGGSGGGGSGGGGSEVQLLESGGGLVQPGGSLRL | |
| | SCAASGFTFSDYGVSWVRQAPGKGLEWVSVIWG | |
| | SETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTISCRASQDISKYLNWYQQKPGKAPKLLIYHTS | |
| | RLHSGVPSRFSGSGSGTDYTLTISSLQPEDFATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 197 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSQVQLVQSGAEVKKPGASVKVSCKASGYT | |
| | FTSYNMHWVRQAPGQGLEWMGAIYPGNGDTSY | |
| | NQKFKGRVTMTRDKSTSTVYMELSSLRSEDTAVY | |
| | YCARSNYYGSSYWFFDVWGQGTTVTVSSGGGGS | |
| | GGGGSGGGGSGGGGSEVQLLESGGGLVQPGGSL | |
| | RLSCAASGFTFSDYGVSWVRQAPGKGLEWVSVI | |
| | WGSETTYYNSALKSRFTISRDNSKNTLYLQMNSL | |
| | RAEDTAVYYCAKHYYYGGSYAMDYWGQGTLVT | |
| | VSSGSTSGSGKPGSGEGSTKGDIQMTQSPSSLSAS | |
| | VGDRVTISCRASQDISKYLNWYQQKPGKAPKLLI | |
| | YHTSRLHSGVPSRFSGSGSGTDYTLTISSLQPEDFA | |
| | TYYCQQGNTLPYTFGGGTKVEIKESKYGPPCPPCP | |
| | MFWVLVVVGGVLACYSLLVTVAFIIFWVKRGRK | |
| | KLLYIFKQPFMRPVQTTQEEDGCSCRFPEEEEGGC | |
| | ELRVKFSRSADAPAYQQGQNQLYNELNLGRREE | |
| | YDVLDKRRGRDPEMGGKPRRKNPQEGLYNELQK | |
| | DKMAEAYSEIGMKGERRRGKGHDGLYQGLSTAT | |
| | KDTYDALHMQALPPR* | |
| 198 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISRDNSKNTLYLQMNSLRAEDTAVYYC | |
| | ARSNYYGSSYWFFDVWGQGTTVTVSSGGGGSGG | |
| | GGSGGGGSGGGGSEVQLLESGGGLVQPGGSLRLS | |
| | CAASGFTFSDYGVSWVRQAPGKGLEWVSVIWGS | |
| | ETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTISCRASQDISKYLNWYQQKPGKAPKLLIYHTS | |
| | RLHSGVPSRFSGSGSGTDYTLTISSLQPEDFATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 199 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISADNSKNTLYLQMNSLRAEDTAVYYC | |
| | ARSNYYGSSYWFFDVWGQGTTVTVSSGGGGSGG | |
| | GGSGGGGSGGGGSEVQLLESGGGLVQPGGSLRLS | |
| | CAASGFTFSDYGVSWVRQAPGKGLEWVSVIWGS | |
| | ETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTISCRASQDISKYLNWYQQKPGKAPKLLIYHTS | |
| | RLHSGVPSRFSGSGSGTDYTLTISSLQPEDFATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 200 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSQVQLVQSGAEVKKPGASVKVSCKASGY | |
| | TFTSYNMHWVRQAPGQGLEWMGAIYPGNGDTS | |
| | YNQKFKGRVTMTRDTSTSTVYMELSSLRSEDTAV | |
| | YYCARSNYYGSSYWFFDVWGQGTTVTVSSGGGG | |
| | SGGGGSGGGGSGGGGSEVQLLESGGGLVQPGGSL | |
| | RLSCAASGFTFSDYGVSWVRQAPGKGLEWVSVI | |
| | WGSETTYYNSALKSRFTISRDNSKNTLYLQMNSL | |
| | RAEDTAVYYCAKHYYYGGSYAMDYWGQGTLVT | |
| | VSSGSTSGSGKPGSGEGSTKGDIQMTQSPSSLSAS | |
| | VGDRVTITCRASQDISKYLNWYQQKPGKAPKLLI | |
| | YHTSRLHSGVPSRFSGSGSGTDYTLTISSLQPEDIA | |
| | TYYCQQGNTLPYTFGGGTKVEIKESKYGPPCPPCP | |
| | MFWVLVVVGGVLACYSLLVTVAFIIFWVKRGRK | |
| | KLLYIFKQPFMRPVQTTQEEDGCSCRFPEEEEGGC | |
| | ELRVKFSRSADAPAYQQGQNQLYNELNLGRREE | |
| | YDVLDKRRGRDPEMGGKPRRKNPQEGLYNELQK | |
| | DKMAEAYSEIGMKGERRRGKGHDGLYQGLSTAT | |
| | KDTYDALHMQALPPR* | |
| 201 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FSSYNMHWVRQAPGKGLEWVSAIYPGNGDTSYN | |
| | QKFKGRFTISRDNSKNTLYLQMNSLRAEDTAVYY | |
| | CARSNYYGSSYWFFDVWGQGTTVTVSSGGGGSG | |
| | GGGSGGGGSGGGGSEVQLLESGGGLVQPGGSLRL | |
| | SCAASGFTFSDYGVSWVRQAPGKGLEWVSVIWG | |
| | SETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDIATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 202 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FTSYNMHWVRQAPGKGLEWVGAIYPGNGDTSYN | |
| | QKFKGRFTISRDNSKNTLYLQMNSLRAEDTAVYY | |
| | CARSNYYGSSYWFFDVWGQGTTVTVSSGGGGSG | |
| | GGGSGGGGSGGGGSEVQLLESGGGLVQPGGSLRL | |
| | SCAASGFTFSDYGVSWVRQAPGKGLEWVSVIWG | |
| | SETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDIATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 203 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSQVQLVQSGAEVKKPGASVKVSCKASGYT | |
| | FTSYNMHWVRQAPGQGLEWMGAIYPGNGDTSY | |
| | NQKFKGRVTMTRDKSTSTVYMELSSLRSEDTAVY | |
| | YCARSNYYGSSYWFFDVWGQGTTVTVSSGGGGS | |
| | GGGGSGGGGSGGGGSEVQLLESGGGLVQPGGSL | |
| | RLSCAASGFTFSDYGVSWVRQAPGKGLEWVSVI | |
| | WGSETTYYNSALKSRFTISRDNSKNTLYLQMNSL | |
| | RAEDTAVYYCAKHYYYGGSYAMDYWGQGTLVT | |
| | VSSGSTSGSGKPGSGEGSTKGDIQMTQSPSSLSAS | |
| | VGDRVTITCRASQDISKYLNWYQQKPGKAPKLLI | |
| | YHTSRLHSGVPSRFSGSGSGTDYTLTISSLQPEDIA | |
| | TYYCQQGNTLPYTFGGGTKVEIKESKYGPPCPPCP | |
| | MFWVLVVVGGVLACYSLLVTVAFIIFWVKRGRK | |
| | KLLYIFKQPFMRPVQTTQEEDGCSCRFPEEEEGGC | |
| | ELRVKFSRSADAPAYQQGQNQLYNELNLGRREE | |
| | YDVLDKRRGRDPEMGGKPRRKNPQEGLYNELQK | |
| | DKMAEAYSEIGMKGERRRGKGHDGLYQGLSTAT | |
| | KDTYDALHMQALPPR* | |
| 204 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISRDNSKNTLYLQMNSLRAEDTAVYYC | |
| | ARSNYYGSSYWFFDVWGQGTTVTVSSGGGGSGG | |
| | GGSGGGGSGGGGSEVQLLESGGGLVQPGGSLRLS | |
| | CAASGFTFSDYGVSWVRQAPGKGLEWVSVIWGS | |
| | ETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDIATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 205 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISADNSKNTLYLQMNSLRAEDTAVYYC | |
| | ARSNYYGSSYWFFDVWGQGTTVTVSSGGGGSGG | |
| | GGSGGGGSGGGGSEVQLLESGGGLVQPGGSLRLS | |
| | CAASGFTFSDYGVSWVRQAPGKGLEWVSVIWGS | |
| | ETTYYNSALKSRFTISRDNSKNTLYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDIATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 206 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSQVQLVQSGAEVKKPGASVKVSCKASGY | |
| | TFTSYNMHWVRQAPGQGLEWMGAIYPGNGDTS | |
| | YNQKFKGRVTMTRDTSTSTVYMELSSLRSEDTAV | |
| | YYCARSNYYGSSYWFFDVWGQGTTVTVSSGGGG | |
| | SGGGGSGGGGSGGGGSEVQLLESGGGLVQPGGSL | |
| | RLSCAASGFTFSDYGVSWVRQAPGKGLEWVSVI | |
| | WGSETTYYNSALKSRFTISKDNSKNTVYLQMNSL | |
| | RAEDTAVYYCAKHYYYGGSYAMDYWGQGTLVT | |
| | VSSGSTSGSGKPGSGEGSTKGDIQMTQSPSSLSAS | |
| | VGDRVTITCRASQDISKYLNWYQQKPGKAPKLLI | |
| | YHTSRLHSGVPSRFSGSGSGTDYTLTISSLQPEDFA | |
| | TYYCQQGNTLPYTFGGGTKVEIKESKYGPPCPPCP | |
| | MFWVLVVVGGVLACYSLLVTVAFIIFWVKRGRK | |
| | KLLYIFKQPFMRPVQTTQEEDGCSCRFPEEEEGGC | |
| | ELRVKFSRSADAPAYQQGQNQLYNELNLGRREE | |
| | YDVLDKRRGRDPEMGGKPRRKNPQEGLYNELQK | |
| | DKMAEAYSEIGMKGERRRGKGHDGLYQGLSTAT | |
| | KDTYDALHMQALPPR* | |
| 207 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FSSYNMHWVRQAPGKGLEWVSAIYPGNGDTSYN | |
| | QKFKGRFTISRDNSKNTLYLQMNSLRAEDTAVYY | |
| | CARSNYYGSSYWFFDVWGQGTTVTVSSGGGGSG | |
| | GGGSGGGGSGGGGSEVQLLESGGGLVQPGGSLRL | |
| | SCAASGFTFSDYGVSWVRQAPGKGLEWVSVIWG | |
| | SETTYYNSALKSRFTISKDNSKNTVYLQMNSLRA | |
| | EDTAVYYCAKHYYYGGSYAMDYWGQGTLVTVS | |
| | SGSTSGSGKPGSGEGSTKGDIQMTQSPSSLSASVG | |
| | DRVTITCRASQDISKYLNWYQQKPGKAPKLLIYH | |
| | TSRLHSGVPSRFSGSGSGTDYTLTISSLQPEDFATY | |
| | YCQQGNTLPYTFGGGTKVEIKESKYGPPCPPCPMF | |
| | WVLVVVGGVLACYSLLVTVAFIIFWVKRGRKKL | |
| | LYIFKQPFMRPVQTTQEEDGCSCRFPEEEEGGCEL | |
| | RVKFSRSADAPAYQQGQNQLYNELNLGRREEYD | |
| | VLDKRRGRDPEMGGKPRRKNPQEGLYNELQKDK | |
| | MAEAYSEIGMKGERRRGKGHDGLYQGLSTATKD | |
| | TYDALHMQALPPR* | |
| 208 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FTSYNMHWVRQAPGKGLEWVGAIYPGNGDTSYN | |
| | QKFKGRFTISRDNSKNTLYLQMNSLRAEDTAVYY | |
| | CARSNYYGSSYWFFDVWGQGTTVTVSSGGGGSG | |
| | GGGSGGGGSGGGGSEVQLLESGGGLVQPGGSLRL | |
| | SCAASGFTFSDYGVSWVRQAPGKGLEWVSVIWG | |
| | SETTYYNSALKSRFTISKDNSKNTVYLQMNSLRA | |
| | EDTAVYYCAKHYYYGGSYAMDYWGQGTLVTVS | |
| | SGSTSGSGKPGSGEGSTKGDIQMTQSPSSLSASVG | |
| | DRVTITCRASQDISKYLNWYQQKPGKAPKLLIYH | |
| | TSRLHSGVPSRFSGSGSGTDYTLTISSLQPEDFATY | |
| | YCQQGNTLPYTFGGGTKVEIKESKYGPPCPPCPMF | |
| | WVLVVVGGVLACYSLLVTVAFIIFWVKRGRKKL | |
| | LYIFKQPFMRPVQTTQEEDGCSCRFPEEEEGGCEL | |
| | RVKFSRSADAPAYQQGQNQLYNELNLGRREEYD | |
| | VLDKRRGRDPEMGGKPRRKNPQEGLYNELQKDK | |
| | MAEAYSEIGMKGERRRGKGHDGLYQGLSTATKD | |
| | TYDALHMQALPPR* | |
| 209 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSQVQLVQSGAEVKKPGASVKVSCKASGYT | |
| | FTSYNMHWVRQAPGQGLEWMGAIYPGNGDTSY | |
| | NQKFKGRVTMTRDKSTSTVYMELSSLRSEDTAVY | |
| | YCARSNYYGSSYWFFDVWGQGTTVTVSSGGGGS | |
| | GGGGGGGGSGGGGSEVQLLESGGGLVQPGGSL | |
| | RLSCAASGFTFSDYGVSWVRQAPGKGLEWVSVI | |
| | WGSETTYYNSALKSRFTISKDNSKNTVYLQMNSL | |
| | RAEDTAVYYCAKHYYYGGSYAMDYWGQGTLVT | |
| | VSSGSTSGSGKPGSGEGSTKGDIQMTQSPSSLSAS | |
| | VGDRVTITCRASQDISKYLNWYQQKPGKAPKLLI | |
| | YHTSRLHSGVPSRFSGSGSGTDYTLTISSLQPEDFA | |
| | TYYCQQGNTLPYTFGGGTKVEIKESKYGPPCPPCP | |
| | MFWVLVVVGGVLACYSLLVTVAFIIFWVKRGRK | |
| | KLLYIFKQPFMRPVQTTQEEDGCSCRFPEEEEGGC | |
| | ELRVKFSRSADAPAYQQGQNQLYNELNLGRREE | |
| | YDVLDKRRGRDPEMGGKPRRKNPQEGLYNELQK | |
| | DKMAEAYSEIGMKGERRRGKGHDGLYQGLSTAT | |
| | KDTYDALHMQALPPR* | |
| 210 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISRDNSKNTLYLQMNSLRAEDTAVYYC | |
| | ARSNYYGSSYWFFDVWGQGTTVTVSSGGGGSGG | |
| | GGSGGGGSGGGGSEVQLLESGGGLVQPGGSLRLS | |
| | CAASGFTFSDYGVSWVRQAPGKGLEWVSVIWGS | |
| | ETTYYNSALKSRFTISKDNSKNTVYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDFATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 211 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISADNSKNTLYLQMNSLRAEDTAVYYC | |
| | ARSNYYGSSYWFFDVWGQGTTVTVSSGGGGSGG | |
| | GGSGGGGSGGGGSEVQLLESGGGLVQPGGSLRLS | |
| | CAASGFTFSDYGVSWVRQAPGKGLEWVSVIWGS | |
| | ETTYYNSALKSRFTISKDNSKNTVYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDFATYYC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 212 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSQVQLVQSGAEVKKPGASVKVSCKASGY | |
| | TFTSYNMHWVRQAPGQGLEWMGAIYPGNGDTS | |
| | YNQKFKGRVTMTRDTSTSTVYMELSSLRSEDTAV | |
| | YYCARSNYYGSSYWFFDVWGQGTTVTVSSGGGG | |
| | SGGGGSGGGGSGGGGSEVQLLESGGGLVQPGGSL | |
| | RLSCAASGFTFSDYGVSWVRQAPGKGLEWVSVI | |
| | WGSETTYYNSALKSRFTISKDNSKNTVYLQMNSL | |
| | RAEDTAVYYCAKHYYYGGSYAMDYWGQGTLVT | |
| | VSSGSTSGSGKPGSGEGSTKGDIQMTQSPSSLSAS | |
| | VGDRVTITCRASQDISKYLNWYQQKPGKAPKLLI | |
| | YHTSRLHSGVPSRFSGSGSGTDYTLTISSLQPEDFA | |
| | TYFCQQGNTLPYTFGGGTKVEIKESKYGPPCPPCP | |
| | MFWVLVVVGGVLACYSLLVTVAFIIFWVKRGRK | |
| | KLLYIFKQPFMRPVQTTQEEDGCSCRFPEEEEGGC | |
| | ELRVKFSRSADAPAYQQGQNQLYNELNLGRREE | |
| | YDVLDKRRGRDPEMGGKPRRKNPQEGLYNELQK | |
| | DKMAEAYSEIGMKGERRRGKGHDGLYQGLSTAT | |
| | KDTYDALHMQALPPR* | |
| 213 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FSSYNMHWVRQAPGKGLEWVSAIYPGNGDTSYN | |
| | QKFKGRFTISRDNSKNTLYLQMNSLRAEDTAVYY | |
| | CARSNYYGSSYWFFDVWGQGTTVTVSSGGGGSG | |
| | GGGSGGGGSGGGGSEVQLLESGGGLVQPGGSLRL | |
| | SCAASGFTFSDYGVSWVRQAPGKGLEWVSVIWG | |
| | SETTYYNSALKSRFTISKDNSKNTVYLQMNSLRA | |
| | EDTAVYYCAKHYYYGGSYAMDYWGQGTLVTVS | |
| | SGSTSGSGKPGSGEGSTKGDIQMTQSPSSLSASVG | |
| | DRVTITCRASQDISKYLNWYQQKPGKAPKLLIYH | |
| | TSRLHSGVPSRFSGSGSGTDYTLTISSLQPEDFATY | |
| | FCQQGNTLPYTFGGGTKVEIKESKYGPPCPPCPMF | |
| | WVLVVVGGVLACYSLLVTVAFIIFWVKRGRKKL | |
| | LYIFKQPFMRPVQTTQEEDGCSCRFPEEEEGGCEL | |
| | RVKFSRSADAPAYQQGQNQLYNELNLGRREEYD | |
| | VLDKRRGRDPEMGGKPRRKNPQEGLYNELQKDK | |
| | MAEAYSEIGMKGERRRGKGHDGLYQGLSTATKD | |
| | TYDALHMQALPPR* | |
| 214 | METDTLLLWVLLLWVPGSTGDIQMTQSPSSLSAS | |
| | VGDRVTITCRASSSVNYMDWYQQKPGKAPKLLIY | |
| | ATSNLASGVPSRFSGSGSGTDFTLTISSLQPEDFAT | |
| | YYCQQWSFNPPTFGGGTKVEIKGSTSGGGSGGGS | |
| | GGGGSSEVQLLESGGGLVQPGGSLRLSCAASGFT | |
| | FTSYNMHWVRQAPGKGLEWVGAIYPGNGDTSYN | |
| | QKFKGRFTISRDNSKNTLYLQMNSLRAEDTAVYY | |
| | CARSNYYGSSYWFFDVWGQGTTVTVSSGGGGSG | |
| | GGGSGGGGSGGGGSEVQLLESGGGLVQPGGSLRL | |
| | SCAASGFTFSDYGVSWVRQAPGKGLEWVSVIWG | |
| | SETTYYNSALKSRFTISKDNSKNTVYLQMNSLRA | |
| | EDTAVYYCAKHYYYGGSYAMDYWGQGTLVTVS | |
| | SGSTSGSGKPGSGEGSTKGDIQMTQSPSSLSASVG | |
| | DRVTITCRASQDISKYLNWYQQKPGKAPKLLIYH | |
| | TSRLHSGVPSRFSGSGSGTDYTLTISSLQPEDFATY | |
| | FCQQGNTLPYTFGGGTKVEIKESKYGPPCPPCPMF | |
| | WVLVVVGGVLACYSLLVTVAFIIFWVKRGRKKL | |
| | LYIFKQPFMRPVQTTQEEDGCSCRFPEEEEGGCEL | |
| | RVKFSRSADAPAYQQGQNQLYNELNLGRREEYD | |
| | VLDKRRGRDPEMGGKPRRKNPQEGLYNELQKDK | |
| | MAEAYSEIGMKGERRRGKGHDGLYQGLSTATKD | |
| | TYDALHMQALPPR* | |
| 215 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSQVQLVQSGAEVKKPGASVKVSCKASGYT | |
| | FTSYNMHWVRQAPGQGLEWMGAIYPGNGDTSY | |
| | NQKFKGRVTMTRDKSTSTVYMELSSLRSEDTAVY | |
| | YCARSNYYGSSYWFFDVWGQGTTVTVSSGGGGS | |
| | GGGGSGGGGSGGGGSEVQLLESGGGLVQPGGSL | |
| | RLSCAASGFTFSDYGVSWVRQAPGKGLEWVSVI | |
| | WGSETTYYNSALKSRFTISKDNSKNTVYLQMNSL | |
| | RAEDTAVYYCAKHYYYGGSYAMDYWGQGTLVT | |
| | VSSGSTSGSGKPGSGEGSTKGDIQMTQSPSSLSAS | |
| | VGDRVTITCRASQDISKYLNWYQQKPGKAPKLLI | |
| | YHTSRLHSGVPSRFSGSGSGTDYTLTISSLQPEDFA | |
| | TYFCQQGNTLPYTFGGGTKVEIKESKYGPPCPPCP | |
| | MFWVLVVVGGVLACYSLLVTVAFIIFWVKRGRK | |
| | KLLYIFKQPFMRPVQTTQEEDGCSCRFPEEEEGGC | |
| | ELRVKFSRSADAPAYQQGQNQLYNELNLGRREE | |
| | YDVLDKRRGRDPEMGGKPRRKNPQEGLYNELQK | |
| | DKMAEAYSEIGMKGERRRGKGHDGLYQGLSTAT | |
| | KDTYDALHMQALPPR* | |
| 216 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISRDNSKNTLYLQMNSLRAEDTAVYYC | |
| | ARSNYYGSSYWFFDVWGQGTTVTVSSGGGGSGG | |
| | GGSGGGGSGGGGSEVQLLESGGGLVQPGGSLRLS | |
| | CAASGFTFSDYGVSWVRQAPGKGLEWVSVIWGS | |
| | ETTYYNSALKSRFTISKDNSKNTVYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDFATYFC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 217 | METDTLLLWVLLLWVPGSTGEIVLTQSPATLSLSP | |
| | GERATLSCRASSSVNYMDWYQQKPGLAPRLLIYA | |
| | TSNLASGIPDRFSGSGSGTDFTLTISRLEPEDFAVY | |
| | YCQQWSFNPPTFGGGTKVEIKGSTSGGGSGGGSG | |
| | GGGSSEVQLLESGGGLVQPGGSLRLSCAASGFTFT | |
| | SYNMHWVRQAPGKGLEWVGAIYPGNGDTSYNQ | |
| | KFKGRFTISADNSKNTLYLQMNSLRAEDTAVYYC | |
| | ARSNYYGSSYWFFDVWGQGTTVTVSSGGGGSGG | |
| | GGSGGGGSGGGGSEVQLLESGGGLVQPGGSLRLS | |
| | CAASGFTFSDYGVSWVRQAPGKGLEWVSVIWGS | |
| | ETTYYNSALKSRFTISKDNSKNTVYLQMNSLRAE | |
| | DTAVYYCAKHYYYGGSYAMDYWGQGTLVTVSS | |
| | GSTSGSGKPGSGEGSTKGDIQMTQSPSSLSASVGD | |
| | RVTITCRASQDISKYLNWYQQKPGKAPKLLIYHTS | |
| | RLHSGVPSRFSGSGSGTDYTLTISSLQPEDFATYFC | |
| | QQGNTLPYTFGGGTKVEIKESKYGPPCPPCPMFW | |
| | VLVVVGGVLACYSLLVTVAFIIFWVKRGRKKLLY | |
| | IFKQPFMRPVQTTQEEDGCSCRFPEEEEGGCELRV | |
| | KFSRSADAPAYQQGQNQLYNELNLGRREEYDVL | |
| | DKRRGRDPEMGGKPRRKNPQEGLYNELQKDKM | |
| | AEAYSEIGMKGERRRGKGHDGLYQGLSTATKDT | |
| | YDALHMQALPPR* | |
| 218 | EVQLLESGGGLVQPGGSLRLSCAASGFTFSDYGV | FMC63-12 |
| | SWVRQAPGKGLEWVSVIWGSETTYYNSALKSRF | |
| | TISRDNSKNTLYLQMNSLRAEDTAVYYCAKHYY | |
| | YGGSYAMDYWGQGTLVTVSSGSTSGSGKPGSGE | |
| | GSTKGDIQMTQSPSSLSASVGDRVTITCRASQDIS | |
| | KYLNWYQQKPGKAPKLLIYHTSRLHSGVPSRFSG | |
| | SGSGTDYTLTISSLQPEDFATYYCQQGNTLPYTFG | |
| | GGVKLEIK | |
| 219 | EVQLLESGGGLVQPGGSLRLSCAASGFTFSDYGV | Hz.FMC63-16: VH3-23; VKO2 |
| | SWVRQAPGKGLEWVSVIWGSETTYYNSALKSRF | |
| | TISRDNSKNTLYLQMNSLRAEDTAVYYCAKHYY | |
| | YGGSYAMDYWGQGTLVTVSSGSTSGSGKPGSGE | |
| | GSTKGDIQMTQSPSSLSASVGDRVTITCRASQDIS | |
| | KYLNWYQQKPGKAPKLLIYHTSRLHSGVPSRFSG | |
| | SGSGTDYTLTISSLQPEDFATYYCQQGNTLPYTFG | |
| | GGTKVEIK | |
| 220 | EVQLLESGGGLVQPGGSLRLSCAASGFTFSDYGV | Hz.FMC63-18: VH3-23; |
| | SWVRQAPGKGLEWVSVIWGSETTYYNSALKSRF | VKO2(L22S) |
| | TISRDNSKNTLYLQMNSLRAEDTAVYYCAKHYY | |
| | YGGSYAMDYWGQGTLVTVSSGSTSGSGKPGSGE | |
| | GSTKGDIQMTQSPSSLSASVGDRVTISCRASQDISK | |
| | YLNWYQQKPGKAPKLLIYHTSRLHSGVPSRFSGS | |
| | GSGTDYTLTISSLQPEDFATYYCQQGNTLPYTFGG | |
| | GTKVEIK | |
| 221 | EVQLLESGGGLVQPGGSLRLSCAASGFTFSDYGV | Hz.FMC63-19: VH3-23; |
| | SWVRQAPGKGLEWVSVIWGSETTYYNSALKSRF | VKO2(L82I) |
| | TISRDNSKNTLYLQMNSLRAEDTAVYYCAKHYY | |
| | YGGSYAMDYWGQGTLVTVSSGSTSGSGKPGSGE | |
| | GSTKGDIQMTQSPSSLSASVGDRVTITCRASQDIS | |
| | KYLNWYQQKPGKAPKLLIYHTSRLHSGVPSRFSG | |
| | SGSGTDYTLTISSLQPEDIATYYCQQGNTLPYTFG | |
| | GGTKVEIK | |
| 222 | EVQLLESGGGLVQPGGSLRLSCAASGFTFSDYGV | Hz.FMC63-20: |
| | SWVRQAPGKGLEWVSVIWGSETTYYNSALKSRF | VH3-23(H71,87); VKO2 |
| | TISKDNSKNTVYLQMNSLRAEDTAVYYCAKHYY | |
| | YGGSYAMDYWGQGTLVTVSSGSTSGSGKPGSGE | |
| | GSTKGDIQMTQSPSSLSASVGDRVTITCRASQDIS | |
| | KYLNWYQQKPGKAPKLLIYHTSRLHSGVPSRFSG | |
| | SGSGTDYTLTISSLQPEDFATYYCQQGNTLPYTFG | |
| | GGTKVEIK | |
| 223 | EVQLLESGGGLVQPGGSLRLSCAASGFTFSDYGV | Hz.FMC63-21: |
| | SWVRQAPGKGLEWVSVIWGSETTYYNSALKSRF | VH3-23(H71,87); VKO2(L87F) |
| | TISKDNSKNTVYLQMNSLRAEDTAVYYCAKHYY | |
| | YGGSYAMDYWGQGTLVTVSSGSTSGSGKPGSGE | |
| | GSTKGDIQMTQSPSSLSASVGDRVTITCRASQDIS | |
| | KYLNWYQQKPGKAPKLLIYHTSRLHSGVPSRFSG | |
| | SGSGTDYTLTISSLQPEDFATYFCQQGNTLPYTFG | |
| | GGTKVEIK | |
| 224 | DIQMTQSPSSLSASVGDRVTITCRASSSVNYMDW | Hz.Leu16: 374 |
| | YQQKPGKAPKLLIYATSNLASGVPSRFSGSGSGTD | |
| | FTLTISSLQPEDFATYYCQQWSFNPPTFGGGTKVEI | |
| | KGSTSGGGSGGGSGGGGSSQVQLVQSGAEVKKP | |
| | GASVKVSCKASGYTFTSYNMHWVRQAPGQGLE | |
| | WMGAIYPGNGDTSYNQKFKGRVTMTRDTSTSTV | |
| | YMELSSLRSEDTAVYYCARSNYYGSSYWFFDVW | |
| | GQGTTVTVSS | |
| 225 | DIQMTQSPSSLSASVGDRVTITCRASSSVNYMDW | Hz.Leu16: 376 |
| | YQQKPGKAPKLLIYATSNLASGVPSRFSGSGSGTD | |
| | FTLTISSLQPEDFATYYCQQWSFNPPTFGGGTKVEI | |
| | KGSTSGGGSGGGSGGGGSSEVQLLESGGGLVQPG | |
| | GSLRLSCAASGFTFSSYNMHWVRQAPGKGLEWV | |
| | SAIYPGNGDTSYNQKFKGRFTISRDNSKNTLYLQ | |
| | MNSLRAEDTAVYYCARSNYYGSSYWFFDVWGQ | |
| | GTTVTVSS | |
| 226 | DIQMTQSPSSLSASVGDRVTITCRASSSVNYMDW | Hz. eu16: 378 |
| | YQQKPGKAPKLLIYATSNLASGVPSRFSGSGSGTD | |
| | FTLTISSLQPEDFATYYCQQWSFNPPTFGGGTKVEI | |
| | KGSTSGGGSGGGSGGGGSSEVQLLESGGGLVQPG | |
| | GSLRLSCAASGFTFTSYNMHWVRQAPGKGLEWV | |
| | GAIYPGNGDTSYNQKFKGRFTISRDNSKNTLYLQ | |
| | MNSLRAEDTAVYYCARSNYYGSSYWFFDVWGQ | |
| | GTTVTVSS | |
| 227 | EIVLTQSPATLSLSPGERATLSCRASSSVNYMDWY | Hz.Leu16: 381 |
| | QQKPGLAPRLLIYATSNLASGIPDRFSGSGSGTDFT | |
| | LTISRLEPEDFAVYYCQQWSFNPPTFGGGTKVEIK | |
| | GSTSGGGSGGGSGGGGSSQVQLVQSGAEVKKPG | |
| | ASVKVSCKASGYTFTSYNMHWVRQAPGQGLEW | |
| | MGAIYPGNGDTSYNQKFKGRVTMTRDKSTSTVY | |
| | MELSSLRSEDTAVYYCARSNYYGSSYWFFDVWG | |
| | QGTTVTVSS | |
| 228 | EIVLTQSPATLSLSPGERATLSCRASSSVNYMDWY | CC384-HZ.Leu16_A11/ |
| | QQKPGLAPRLLIYATSNLASGIPDRFSGSGSGTDFT | VH3-23(30T.49G) VL_VH CAR T |
| | LTISRLEPEDFAVYYCQQWSFNPPTFGGGTKVEIK | |
| | GSTSGGGSGGGSGGGGSSEVQLLESGGGLVQPGG | |
| | SLRLSCAASGFTFTSYNMHWVRQAPGKGLEWVG | |
| | AIYPGNGDTSYNQKFKGRFTISRDNSKNTLYLQM | |
| | NSLRAEDTAVYYCARSNYYGSSYWFFDVWGQGT | |
| | TVTVSS | |
| 229 | EIVLTQSPATLSLSPGERATLSCRASSSVNYMDWY | CC385-Hz.Lue16_A11/ |
| | QQKPGLAPRLLIYATSNLASGIPDRFSGSGSGTDFT | VH3-23(30T.49G.71A) |
| | LTISRLEPEDFAVYYCQQWSFNPPTFGGGTKVEIK | VL_VH CAR T |
| | GSTSGGGSGGGSGGGGSSEVQLLESGGGLVQPGG | |
| | SLRLSCAASGFTFTSYNMHWVRQAPGKGLEWVG | |
| | AIYPGNGDTSYNQKFKGRFTISADNSKNTLYLQM | |
| | NSLRAEDTAVYYCARSNYYGSSYWFFDVWGQGT | |
| | TVTVSS | |

The present disclosure provides for a closed system method of manufacturing a population of T cells. In certain embodiments of the disclosed method, a population of TM/MEM cells is enriched by, for example, contacting the TN/MEM cells with 1-100 μL CliniMACs or 5-80 μL CliniMACs or 10-60 μL CliniMACs or 15-40 μL CliniMACs. In certain embodiments, the cells are contacted with about 15-25 μL CliniMACs or about 20 μL CliniMACs. The step of contacting the cells may also preferably involve contacting the TN/MEM cells with microbeads.

In certain embodiments, the cells are activated by being contacted with TransAct™ for about 1 hour to about 84 hours, or about 6 hours to about 72 hours, or about 12 hours to about 60 hours, or about 18 hours to about 48 hours, or about 24 hours to about 48 hours, or from about 30 hours to about 48 hours.

Without limitation, the activation step may be performed for about 6 hours, or about 12 hours, or about 18 hours, or about 24 hours, or about 30 hours, or about 36 hours, or about 42 hours, or about 48 hours, or about 54 hours, or about 60 hours. In certain embodiments, the activation step is performed for about 42 hours.

In certain embodiments, the activation step may comprise contacting the cells with OpTmizer™ cell culture media, preferably containing plus rhIL-2 and rhIL-15 and TransAct®.

In certain embodiments, following activation, the cells are transduced with a lentiviral vector, e.g. in the absence of protamine sulfate.

In certain, non-limiting embodiments, the lentiviral vector may comprise an EFla promoter and CD 19/20 CAR in the pALD backbone.

Following transduction, the cells are preferably maintained for at least about 1 day, or at least about 2 days, or from about 1 day to about 14 days, or from about 1 day to about 10 days, or from about 1 day to about 7 days, or from about 2 days to about 14 days, or from about 2 days to about 10 days, or from about 2 days to about 7 days. In certain embodiments, the cells may be maintained for about 1 day, or about 2 days, or about 3 days, or about 4 days, or about 5 days, or about 6 days, or about 7 days, or about 8 days, or about 9 days, or about 10 days.

In certain embodiments, the cells are optionally cryopreserved.

Cryopreserving the TM/MEM cells may be carried out by, for example, contacting the cells with Sepax C-Pro in a ratio of about 1:1 saline/HAS:CryoStorlO.

In some embodiments, the maintaining step comprises adding a cell culture media 1-10 days after enriching is performed, or 2-8 days after enriching is performed, or 3-7 days after enriching is performed, or about 1 day after enriching, or about 2 days after enriching, or about 3 days after enriching, or about 5 days after enriching, or about 6 days after enriching, or about 7 days after enriching, or about 8 days after enriching, or about 9 days after enriching, or about 10 days after enriching.

The maintaining step may also comprise washing with Sepax C-Pro, optionally followed by transferring the cells to a new cell culture container, such as a cell culture bag or other cell culture vessel.

Said washing may be performed 1-10 days after enriching is performed, or 2-8 days after enriching is performed, or 3-7 days after enriching is performed, or about 1 day after enriching, or about 2 days after enriching, or about 3 days after enriching, or about 5 days after enriching, or about 6 days after enriching, or about 7 days after enriching, or about 8 days after enriching, or about 9 days after enriching, or about 10 days after enriching.

In embodiments where the cells are transferred to a new cell culture container, the cells are transferred to the new cell culture container at a rate of about 0.1*10⁶ cells/mL to about 5.0*10⁶ cells/mL, or about 0.25*10⁶ cells/mL to about 2.0*10⁶ cells/mL, or about 0.5*10⁶ cells/mL to about 1.5*10⁶ cells/mL. Alternatively, in embodiments where the cells are transferred to a new cell culture container, the cells are transferred to the new cell culture container at a rate of about such as about 0.1*10⁶ cells/mL, or about 0.2*10⁶ cells/mL, or about 0.3*10⁶ cells/mL, or about 0.4*10⁶ cells/mL, or about 0.5*106 cells/mL, or about 0.6*10⁶ cells/mL, or about 0.7*10⁶ cells/mL, or about 0.8*10⁶ cells/mL, or about 0.9*10⁶ cells/mL, or about 1.0*10⁶ cells/mL.

In an aspect, CAR molecules disclosed herein comprise four main domains: an scFv, an extracellular spacer, a transmembrane domain, and a cytoplasmic tail including costimulatory signals and the CD3z activation chain.

In an aspect, a method described herein does not comprise a CD14+/CD25+ depletion step. In a further aspect, the presently described method achieves similar or superior results as methods which comprise a CD14+/CD25+ depletion step.

The extracellular domain of a CD19/CD20 CAR described herein can be derived from, for example and without limitation, the antigen binding domain of Leu 16 murine antibody that recognizes human CD20, and the antigen binding domain of FMC63, recognizing human CD19 to create bi specific CAR configuration. Bispecific configuration enables bivalent binding to CD19 and/or CD20 on B cells. The scFvs fragments are connected via a GS flexible linker.

In an aspect, the extracellular spacer (e.g., an IgG4 extracellular spacer) serves as the hinge connecting the Leu16 scFv to the CD28 TM domain, and intracellular domains. A person of ordinary skill will appreciate that the hinge domain plays an important role in the function of CAR-T cells, the properties of the hinge region can influence CAR-T cell cytokine production and AICD (Alabanza et al., Function of Novel Anti-CD19 Chimeric Antigen Receptors with Human Variable Regions Is Affected by Hinge and transmembrane Domains. Mol Ther J Am Soc Gene Ther (2017) 25(11):2452-65.

### Therapeutic Applications, Compositions and Administration

In some aspects, the disclosed CARs, polypeptides, and/or engineered immune cells may be formulated as a composition for administration to a patient. The term “patient” refers to a subject or individual who is in need of prevention or treatment of a disease or disorder. In some aspects, the composition is a pharmaceutical composition or a pharmaceutically effective composition. In some aspects, the composition is a therapeutic composition or a therapeutically effective composition

The present disclosure provides methods for the treatment or amelioration of an autoimmune disease in a patient in need thereof. In some aspects, the present disclosure provides methods for the treatment or amelioration of a cancer in a patient in need thereof.

In some aspects, the autoimmune disease is selected from the group consisting of lupus, rheumatoid arthritis (RA), systemic lupus erythematosus (SLE), inflammatory bowel disease (IBD/Crohn's), Type 1 diabetes mellitus, Guillain-Barre syndrome, chronic inflammatory demyelinating polyneuropathy, psoriasis, Graves' Disease, Hashimoto's Thyroiditis, Myasthenia Gravis, scleroderma, systemic sclerosis, Multiple Sclerosis (MS), autoimmune neuropathy, transverse myelitis, optic neuritis, neuromyelitis optica, acute disseminated encephalomyelitis, autoimmune or paraneoplastic encephalitis, and spasticity.

In some aspect, the disclosed CARs, polypeptides, and/or engineered immune cells (and related compositions) may be used in the treatment of RA, IBD, or psoriasis.

In some aspect, the disclosed CARs, polypeptides, and/or engineered immune cells (and related compositions) may be used in the treatment of lupus or SLE.

In some aspects, the autoimmune disease is lupus or SLE. SLE is the most common form of lupus in which the immune system attacks its own healthy tissues resulting in widespread inflammation and tissue damage of joints, skin, brain, lungs, kidneys, and blood vessels. Common symptoms of this disorder include but are not limited to Skin rashes, fatigue, low fevers, pain or swelling of the joints, lung problems, kidney problems, and heart problems and it often co-exists with conditions such as Lupus nephritis, rheumatoid arthritis, cardiovascular disease, osteoporosis, Sjögren's syndrome, vascular disease, Graves disease, and Hashimoto's thyroiditis.

Described herein, in some embodiments, are methods of treating a patient in need thereof having an autoimmune disease, comprising administering to the patient a population of engineered immune cells described herein.

The disclosed therapeutic compositions are administered to alleviate, reduce, modulate, ameliorate or eliminate one or more of these symptoms or conditions and results in the improvement of the condition, disease, disorder, and the like, or ameliorating a symptom thereof. For instance, a treatment can result in a reduction in one or more symptoms of an autoimmune disease, e.g., depression and fatigue, bladder dysfunction, spasticity, pain, ataxia, and intention tremor. A therapeutically effective amount can be an amount sufficient to prevent the onset of an acute episode or to shorten the duration of an acute episode, or to decrease the severity of one or more symptoms, e.g., heat sensitivity, internuclear ophthalmoplegia, optic neuritis, and Lhermitte symptom. In some embodiments, a therapeutically effective amount is an amount sufficient to prevent the appearance of, delay or prevent the growth (i.e., increase in size) of, or promote the healing of a demyelinated lesion in one or more of the brain, optic nerves, and spinal cord of the subject, e.g., as demonstrated on MRI. In some aspects, administration of an effective amount of the disclosed CARs, polypeptide and/or immune cells may be used in the treatment of cancer

One aspect of the disclosure includes a method of treating an autoimmune disease in a patient in need thereof, comprising administering to the patient an effective amount of a population of cells, wherein the population of cells comprise a polypeptide comprising a monospecific CAR described herein. One aspect of the disclosure includes a method of treating an autoimmune disease in a patient in need thereof, comprising administering to the patient an effective amount of a population of cells, wherein the population of cells comprise a polypeptide comprising a bispecific CAR described herein.

In one aspect of the disclosure, the method of treating an autoimmune disease includes obtaining immune cells from a patient; engineering the immune cells, wherein the engineered immune cells comprise a polypeptide and/or CAR disclosed herein; expanding the engineered immune cells; and administering an effective amount of the expanded engineered immune cells to the patient. The immune cells can be any suitable immune cell type including, but not limited to, T cells, NK cells, and Treg cells.

In some embodiments, the compositions contemplated herein are used to treat a patient with SLE. In clinical trials, SLE disease activity is assessed by several instruments like component or composite scores. Commonly used SLE instruments and component scores include:

Systemic Lupus Erythematosus Disease Activity Index (SLEDAI), which measures presence/absence of 24 disease features, from which a weighted total score can be derived; British Isles Lupus Assessment Group (BILAG) index, which evaluates eight individual organ systems; the organ systems included in this analysis included: musculoskeletal, mucocutaneous, or renal; Physician's Global Assessment (PGA) on a 0 (none) to 3 (severe) visual analogue scale; and Average prednisone-equivalent daily corticosteroid dose (aPEDD).

In certain instances, the SLE is active SLE. In some cases, the human subject has active, autoantibody-positive SLE. In certain cases, wherein the human subject has active, autoantibody-positive SLE and the human subject is receiving the non-biologic standard of care therapy for SLE. In some cases, the SLE is moderate SLE. In certain cases, the SLE is severe SLE. In some cases, the SLE is active SLE with active joint and/or skin manifestations. In some cases, the human subject has a SLEDAI-2K≥6 excluding alopecia, lupus-related headache and organic brain disease at initiation of treatment. In certain cases, the human subject has a clinical SLEDAI-2K≥4 excluding alopecia, lupus-related headache and organic brain disease, anti-ds DNA, low complement C3 and/or C4, or fever, at initiation of treatment. In certain cases, the human subject has BILAG-2004 grade A in ≥1 organ system or BILAG-2004 grade B in ≥2 organ systems at initiation of treatment.

In some aspects, the effective amount of a composition administered to a patient is between from 1×10⁶ cells to 2×10¹⁰ cells, alternatively 2×10⁶ cells, alternatively about 3×10⁶ cells, alternatively about 4×10⁶ cells, alternatively about 5×10⁶ cells, alternatively about 6×10⁶ cells, alternatively about 7×10⁶ cells, alternatively about 8×10⁶ cells, alternatively about 9×10⁶ cells, alternatively about 1×10⁷ cells, alternatively about 2×10⁷ cells, alternatively about 3×10⁷ cells, alternatively about 4×10⁷ cells, alternatively about 5×10⁷ cells, alternatively about 6×10⁷ cells, alternatively about 7×10⁷ cells, alternatively about 8×10⁷ cells, alternatively about 9×10⁷ cells, alternatively about 1×10⁸ cells, alternatively about 2×10⁸ cells, alternatively about 3×10⁸ cells, alternatively about 4×10⁸ cells, alternatively about 5×10⁸ cells, alternatively about 6×10⁸ cells, alternatively about 7×10⁸ cells, alternatively about 8×10⁸ cells, alternatively about 9×10⁸ cells, alternatively about 1×10⁹ cells, alternatively about 2×10⁹ cells, alternatively about 3×10⁹ cells, alternatively about 4×10⁹ cells, alternatively about 5×10⁹ cells, alternatively about 6×10⁹ cells, alternatively about 7×10⁹ cells, alternatively about 8×10⁹ cells, or alternatively about 9×10⁹ cells.

In certain embodiments 1×10⁷ cells, 2×10⁷ cells, 5×10⁷ cells, 1×10⁸ cells, or 2×10⁸ cells may be the preferred dose.

In some aspects, the composition may be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations may also be emulsified. The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases, the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, histidine, procaine and the like. A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients. Upon formulation, solutions will be administered in a manner compatible with the dosage formulation and in such amount as is therapeutically or prophylactically effective. The formulations are easily administered in a variety of dosage forms, such as the type of injectable solutions described herein.

In some aspects, the composition is administered once. In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

In an aspect, the disclosure provides for a method of treating lupus in a patient in need thereof, comprising administering to the patient an effective amount of an engineered T cell, the engineered T cell comprising a polypeptide comprising a CD19/CD20 chimeric antigen receptor (CAR), the CAR comprising: an anti-CD20 scFv comprising (i) a light chain variable region having the amino acid sequence of SEQ ID NO: 34, and (ii) a heavy chain variable region having the amino acid sequences of SEQ ID NO: 35; a (G₄S)ₙ linker, wherein n is 1, 2, 3, or 4; an anti-CD19 scFv comprising (i) a heavy chain variable region having the amino acid sequence of SEQ ID NO: 36, and (ii) a light chain variable region having the amino acid sequences of SEQ ID NO: 37; a spacer domain comprising the amino acid sequence of any one selected from SEQ ID NOs: 21-29; a transmembrane domain comprising the amino acid sequence of SEQ ID NO: 30 or 31; a 4-1BB cytoplasmic signaling domain comprising the amino acid sequence of SEQ ID NO: 32; and a CD3 zeta signaling domain comprising the amino acid sequence of SEQ ID NO: 33.

In an aspect, the disclosure provides for a method of treating SLE in a patient in need thereof, comprising administering to the patient an effective amount of an engineered T cell, the engineered T cell comprising a polypeptide comprising a CD19/CD20 chimeric antigen receptor (CAR), the CAR comprising: an anti-CD20 scFv comprising (i) a light chain variable region having the amino acid sequence of SEQ ID NO: 34, and (ii) a heavy chain variable region having the amino acid sequences of SEQ ID NO: 35; a (G₄S)ₙ linker, wherein n is 1, 2, 3, or 4; an anti-CD19 scFv comprising (i) a heavy chain variable region having the amino acid sequence of SEQ ID NO: 36, and (ii) a light chain variable region having the amino acid sequences of SEQ ID NO: 37; a spacer domain comprising the amino acid sequence of any one selected from SEQ ID NOs: 21-29; a transmembrane domain comprising the amino acid sequence of SEQ ID NO: 30 or 31; a 4-1BB cytoplasmic signaling domain comprising the amino acid sequence of SEQ ID NO: 32; and a CD3 zeta signaling domain comprising the amino acid sequence of SEQ ID NO: 33.

In an aspect, the disclosure provides for a method of treating an autoimmune disease in a patient in need thereof, comprising administering to the patient an effective amount of an engineered T cell, the engineered T cell comprising a polypeptide comprising a CD19 chimeric antigen receptor (CAR), comprising: an anti-CD19 scFv comprising (i) a heavy chain variable region having the amino acid sequence of SEQ ID NO: 36, and (ii) a light chain variable region having the amino acid sequences of SEQ ID NO: 37; a spacer domain comprising the amino acid sequence of any one selected from SEQ ID NOs: 21-29; a transmembrane domain comprising the amino acid sequence of SEQ ID NO: 30 or 31; a 4-1BB cytoplasmic signaling domain comprising the amino acid sequence of SEQ ID NO: 32; and a CD3 zeta signaling domain comprising the amino acid sequence of SEQ ID NO: 33.

In an aspect, the disclosure provides a method of treating an autoimmune disease in a patient comprising administering to the patient an effective amount of an engineered T cell, the engineered T cell comprising a polypeptide comprising a CD19-OR-CD20 chimeric antigen receptor (CAR), wherein the CAR comprises from N-terminus to C-terminus: an anti-CD20 scFv with a variable heavy domain and a variable light domain obtained and/or derived from ofatumumab; a (G₄S)ₙ linker, wherein n is 1, 3, or 4; an anti-CD19 scFv comprising a variable heavy domain from SEQ ID NO: 256 and a variable light domain from SEQ ID NO: 256; a spacer of SEQ ID NO: 257; a transmembrane domain of SEQ ID NO:258; a co-stimulatory domain of SEQ ID NO: 260; and a CD3-zeta cytoplasmic signaling domain of SEQ ID NO:261.

### Combination Therapies

It will be appreciated that the CARs, polypeptides and/or immune cells disclosed herein may be administered in combination with other agents or treatment regimens. These combination therapies can include administration of the immune cells as described herein in combination with agents and/or treatments know to those skilled in the art to treat autoimmune disease, including Lupus. These can include, but are not limited to:

Immunosuppressives such as Methotrexate (Rheumatrex®), Mycophenolate mofetil (Cellcept®), Azathioprine (Imuran®), Cyclophosphamide (Cytoxan®), and Voclosporin (Lupkynis™);

Steroids such as corticosteroids, glucocorticoids or cortisone, including, e.g., prednisone;

Therapies including belimumab (Benlysta®), anifrolurnab-fnia (Saphnelo®), adahrnurnab (Humira®), etanercept (Enbrel®); tofacitinib (Xeljanz®), infliximab (Remicade®), golimumab (Simponi Aria®), certolizumab (Cimzia®), tocilizumab (Actemra®), sarilumab (Kevzara®), Anakinra (Kineret®), canakinumab (Haris), rilonacept (Arcalyst®), eculizumab, cyclosporine, abatacept (Orencia®), secukinumab (Cosentyx®), ixekizumab (Taltz®), brodalumab (Siliq®), guselkumab (Tremfya®), ustekinumab (Stelara®), dupilumab (Dupixent®), vedolizumab (Entyvio®); hydroxychloroquine (Plaquenil®); NSAIDs such as ibuprofen (e.g., Advil® or Motrin®), naproxen (e.g., Aleve® or Naprosyn®), indomethacin (Indocin®), Nabumetone (Relafen®), celecoxib (Celebrex®), and aspirin; and blood thinners such as heparin injections (Calciparine® or Liquaemin®), or warfarin pills (Coumadin®).

In certain embodiments, the compositions described herein are administered in conjunction with a cytokine. The term “cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSP (M-CSF); granulocyte-macrophage-CSF (GM-CSF); and granulocyte-CSF (G-CSF); interleukins (ILs) such as IL-1, IL-I alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In some embodiments, compositions contemplated herein comprise an effective amount of an expanded modified T cell composition, alone or in combination with one or more therapeutic agents. In some embodiments, the T cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics and anti-viral agents. Such therapeutic agents may be accepted in the art as a treatment for a disease state as described herein, such as a cancer. In one embodiment the compositions contemplated herein may also be administered with inhibitors of TGF-.beta., for example the small molecule inhibitor LY55299. Exemplary therapeutic agents contemplated include cytokines, growth factors, steroids, NSAIDs, DMARDs, anti-inflammatories, chemotherapeutics, radiotherapeutics, therapeutic antibodies, or other active and ancillary agents. In some embodiments, the T cell compositions may be administered alone or in combination with other known autoimmune disease or disorder treatments.

In certain embodiments, a therapeutic method described herein does not produce or otherwise elicit an inflammatory response in a subject.

In an aspect, the inflammatory effect or response is reduced as indicated by reduced levels of interferon gamma, while maintaining substantially the same cytotoxicity.

In an aspect, the inflammatory effect or response is reduced as indicated by reduced levels of interferon gamma and IL-2, while maintaining substantially the same cytotoxicity.

In certain embodiments, compositions comprising T cells contemplated herein may be administered in conjunction with any number of chemotherapeutic agents. Illustrative examples of chemotherapeutic agents include but are not limited to alkylating agents such as thiotepa and cyclophosphamide (Cytoxan™); alkyl sulfonates such as busulfan, improsulfan and piposulfan; aziridines such as benzodopa, carboquone, meturedopa, and uredopa; ethylenimines and me thy lamelamines including altretamine, triethylenemelamine, trietylenephosphoramide, triethylenethiophosphaoramide and trimethylolomelamine resume; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (Taxol®, Bristol-Myers Squibb, Princeton, N.J.) and doxetaxel (Taxotere™, Sanofi, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RPS 2000; difluoromethylomithine (DMFO); retinoic acid derivatives such as Targretin™ (bexarotene), Panretin™ (alitretinoin); ONTAK™ (denileukin diftitox); esperam1cms; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

A variety of therapeutic agents may be used in conjunction with the compositions described herein. In one embodiment, the composition comprising T cells is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

One skilled in the art will also appreciate that the current chimeric receptors can be administered in combination with one or more checkpoint inhibitors. Suitable checkpoint inhibitors include, but are not limited to, pembrolizumab (Keytruda™), ipilimumab (Yervoy™) nivolumab (Opdivo™) and atezolizumab (Tecentriq™). See for example, https://www.cancerresearchuk.org/about-cancer/cancer-in-general/treatment/immunotherapy/types/checkpoint-inhibitors.

In other embodiments, the therapeutic antibodies suitable for combination with the CAR modified T cells contemplated herein, include but are not limited to, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, namatumab, naptumomab, necitumumab, nimotuzumab, nofetumomab, ocaratuzumab, ofatumumab, obinutuzmnab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49 and 3F8. The radioactive monoclonal antibody ibritumomab (Zevalin) is also suitable for use in accordance with the invention.

In certain embodiments, editing a gene locus comprises using a CRISPR (Clustered Regularly Interspaced Short Palindromic Repeats)/Cas9 system, a zinc finger nuclease (ZFN), a T ALEN, a MegaT AL, a meganuclease, Cpfl, homologous recombination, or a single stranded oligodeoxynucleotide (ssODN), and the like can be used.

Retroviral-based gene therapy vectors (e.g., gammaretroviral, lentiviral) are the predominant choice for CAR transduction due to the stable integration of these vectors, which results in long-term expression of the CAR. Critical vector quality attributes (e.g., titer, potency, purity) are directly determine the number of copies stably integrated into the target cells, and therefore to a large extent determine the potency of the CAR T cell product.

The invention is further described in the context of the examples below.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Clinical Results

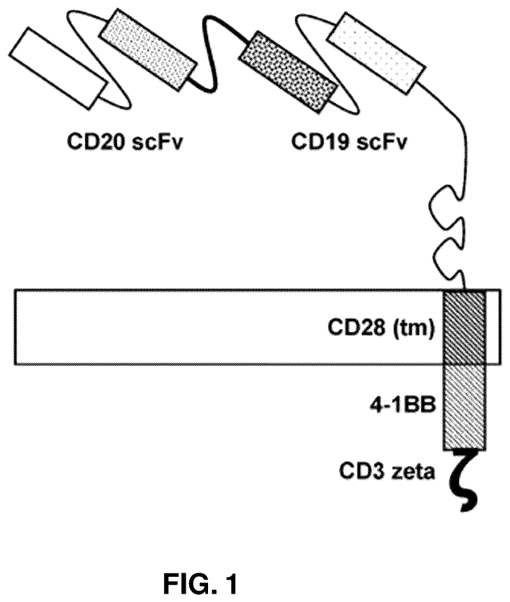

Using a CD19/CD20 tandem bispecific CAR-T as described in Zah et al. (2016), a systematic methodology was employed to optimize an improved construct design, including the target binding single-chain variable fragments (scFv), the length of the spacers, and the flexibility of the linkers. As noted in Zah, the construct that performed best positioned the Leu16-derived CD20-binding scFv membrane-distal, and connected via a (G₄S)₄ flexible linker to FMC63-derived CD19-binding scFv membrane-proximal. This targeting region of the tandem bispecific molecule was attached through a short IgG4 hinge to a CD28-derived transmembrane region, followed by the intracellular signaling region comprised of the cytoplasmic co-stimulatory domain of human 4-1BB, and the cytoplasmic domain of human CD3s at the C terminus. The transmembrane domain derived from human CD28 provides the physical link between the hinge and the intracellular domain. FIG. 1 shows a graphical representation of an exemplary CAR-T suitable for use in the present invention, e.g., bispecific anti CD19/CD20 CARs encoding the CD19 single-chain variable fragment (scFv) derived from the FMC63 mAb, the CD20 scFv derived from the Leu-16 mAb, an IgG4-based extracellular spacer, the CD28 transmembrane domain, and the cytoplasmic domains of 4-1BB and CD3s.

In an ongoing Phase I clinical study, enrolled patients had high tumor burden and had undergone 3 or more lines of therapy for DLBCL, mantle cell lymphoma, or follicular lymphoma. 10 patients were treated with autologous T cells transduced with the tandem CD19-20 CAR as described above. The therapy was well tolerated when compared to other CAR T therapies, with no dose-limiting toxicities identified at DLI (5×10⁷ CAR-T cells) and one prolonged cytopenia noted at DL2 (2×10⁸ CAR-T cell).

High efficacy of 90% ORR (9/10) and 70% CR (7/10) was observed accompanied with limited toxicity—only grade I CRS was reported, and no patients had observed neurotoxicity. At a median follow up of 17 months with 7 of 10 patients achieving CR and median DOR has not been reached. Two patients treated at DLI had a PR and the 1 patient who did not respond had early disease progression, with CD19 and CD20 negative mediastinal lymphoma evident on day 14 after CAR T cell infusion.

### Example 2: Phenotypic Characterization of Engineered Cells

While the initial cytotoxicity of a CAR-T therapy is critical for tumor clearance, T-cell phenotypes with increased persistence have been reported to prolong the durability of a patient's tumor response (see Kawalekar, 2016). The CD19/20 CAR-T clinical data above demonstrated a high rate of durable CRs accompanied by long-term detection of CAR+ T cells in treated patients.

Prior processes enriched CD62L+TN/MEM cells that contained naive, central memory and naive stem-like cells that were reported to possess superior self-renewal, multipotency, and long-term proliferative capacity compared to their CD62L− counterparts (Tsui, 2022). However, to improve this process, CD62L+TN/MEM cells were instead enriched at the beginning of the process.

It was thus important to assess the phenotype of the TN/MEM transduced cells. To do so, CAR-T cells were stained with antibodies against CD45RA and CD62L to examine distribution of naive/stem cell memory, central memory, effector memory, and effector/exhausted populations. In addition, CAR-T cells were stained with anti-CD4 and anti-CD8 antibodies to assess distribution of cytotoxic vs. helper T cell populations. In these studies the cell products after the 12-day process were consistently observed to be >80% positive for naive/stem cell memory (CD45RA+/CD62L+) and central memory (CD45RA−/CD62L+). Cells remained >80% positive for CD62L by day 12 with the two predominant populations being naive/stem cell memory and central memory. The presence of these populations correlated with a more naive T cell phenotype, and are thus associated with longer persistence and reduced toxicity in vivo (see e.g., Khalid, Blood 2018, and Golubovskaya, Cancer 2016).

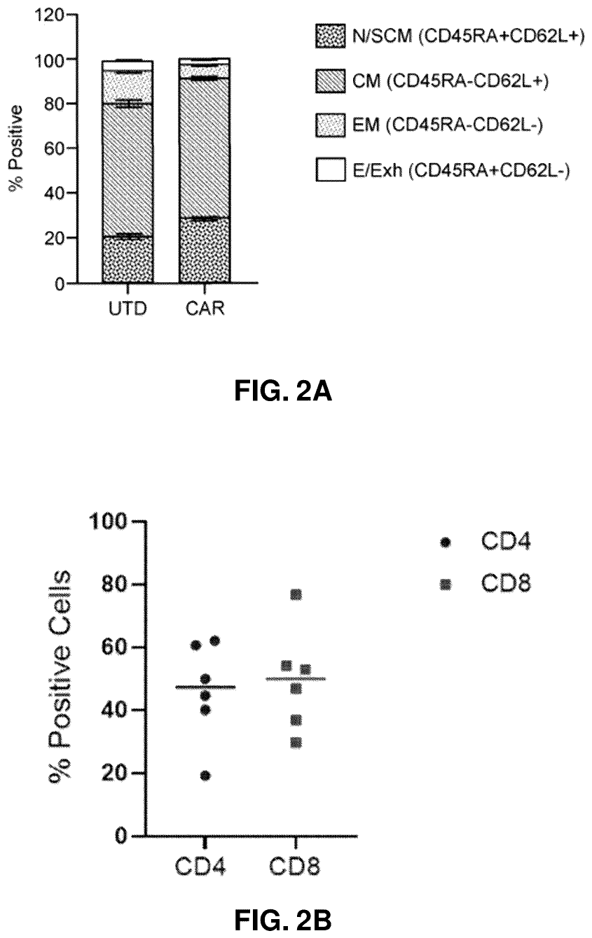

Phenotype data was similar for untransduced (UTD) cells or cells transduced with the CC314B vector (see FIG. 2A), suggesting that transduction did not change the final product phenotype. This finding is consistent with initial selection of CD62L+ positive cells and reproduced with many donors, and illustrates maintenance of the largely naive/memory population enriched in self-renewing lymphocytes. As can be seen in FIG. 2A, T-cells were harvested on day 12, at the end of the process and stained for CD45RA and CD62L. Data is from D8374 and representative of 6 donors. Greater than 80% of the cells were either central memory (predominant population) or naive/stem cell memory cells, consistent with selection of CD62L+ cells on day 1. Samples were stained and measured in triplicates and mean and standard deviations are graphed. Abbreviations used were as follows: N/SCS—naive/stem cell memory, CM—central memory, EM—effector memory, E/Exh—effector/exhausted.

Cells were also stained for CD4 and CD8 on day 12. Although the CD4+/CD8+ ratio varied between donors, an average of −50% of CD4 positive cells and −50% of CD8 positive cells was observed when the data from all donors were analyzed in one figure (see FIG. 2B). In FIG. 2B, T cells were harvested on day 12 and stained with anti-CD4 and anti-CD8 antibodies. Data is a summary of 6 donors, mean is represented by a horizontal line.

CAR-T cells were stained using a phenotyping panel against CD45RA, CD62L, CD4, and CD8 cells in this study. Cells were >80% positive for naive/memory (CD45RA+/CD62L+) and central memory (CD45RA−/CD62L+). This finding is consistent with initial selection of CD62L+ positive cells and dependably, across many donors, illustrates that the manufacturing process maintains the largely naive/memory population.

### Example 3: Assessment of Cytokine Independent Growth of CD19/20 CAR-T

Studies have shown that CAR-T cells can grow independently of activation and at times independently of cytokines. This is thought to result from tonic signaling that can mimic T cell activation and can quickly render the T cells more exhausted and, as a consequence, limit responses in patients. The CD 19/20 CAR-T of the invention was next examined for the ability to grow independently of cytokines.

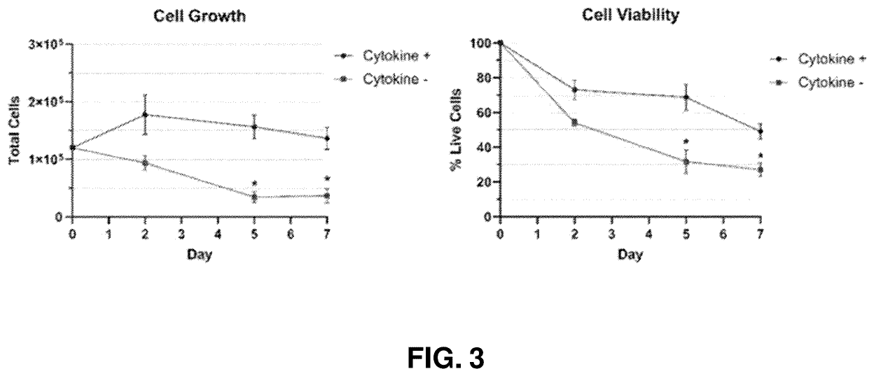

To do so, cryopreserved CD19/20 CAR-T cells were produced using the processes described herein and assessed for factor-independent growth. Proliferation and viability of the CD19/20 CART cells were determined with trypan blue staining and cell counting. After 7 days the viability and cell number of the population deprived of cytokine had reduced significantly compared to cells maintained in cytokine. The results are set forth in FIG. 3. As can be seen in FIG. 3, Cryopreserved CD 19/20 CART cells were thawed and either incubated in CTS optimizer media with or without cytokines (IL-2 and IL-15). They were then seeded at equal confluency and cell growth, and viability assessed every 2-3 days for 7 days using trypan blue and a Countess® cell counter.

These results demonstrate that both cell number and viability of CD 19/20 CAR-T cells decline drastically in the absence of cytokine supplementation, suggesting that these cells do not activate in the absence of antigen and/or cytokine stimulation.

These findings were reproduced using cells from three independent donors. In addition, the data suggests that the CD19/20 CAR-T cells did not grow independent of cytokines and support the conclusion that CD19/20 CART cells do not exhibit detectable tonic signaling.

### Example 4: Potency Assessment by Cytokine Release of CD19/20 CAR-T Cells

Cells were developed to enable functional potency assessment of both the CD19− and CD20− targeting portions of the CD19/20 CAR-T product. To do so, Raji cells (as a surrogate for B cells) which endogenously express CD19 and CD20 were engineered to create Raji lines which express Firefly Luciferase GFP (FFluc GFP) and have either CD19 deleted, CD19−/CD20+(CD19KO) or CD20 deleted, CD19+/CD20− (CD20KO).

Raji cells were transduced with lentivirus to create stable GFP FFluc expressing cell and CRISPR/Cas9 was used to edit the Raji GFP FFLuc line to create CD19KO and CD20KO lines for use in CTL assays.

To assess the potency of the CD19/20 CAR-T product made using this process we initially utilized cytokine release assays to evaluate interferon-y (IFN-γ) and interleukin-2 (IL-2) production. To achieve this, ELISA assays were performed to quantify IFN-γ and IL-2 production when the CD19/20 CAR-T product was co-cultured with wild-type Raji cells as a surrogate for B cells that express both antigens CD19 and CD20, single antigen positive cells (Raji CD19KO, Raji CD20KO) or antigen negative tumor cells (the CEM target cells).

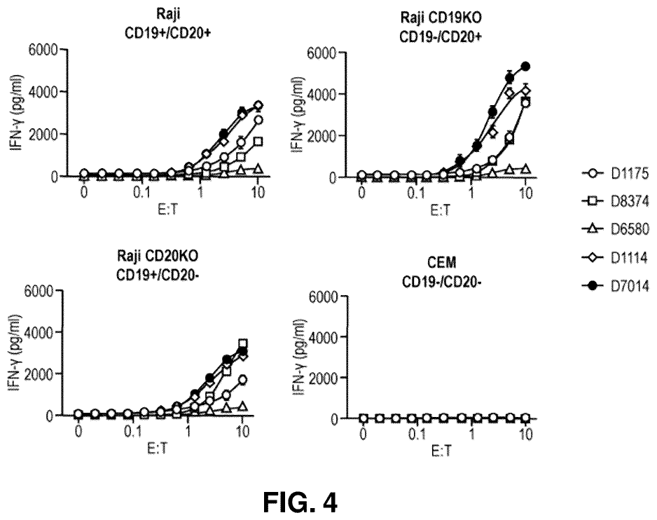

The results are set forth in FIG. 4. FIG. 4 shows secretion of IFN-γ (B) by CD19/20 CAR-T cells after co-incubation with target Raji lymphoma cells for 24 hrs as detected by ELISA. The values shown are the means of technical repeats, error bars indicate standard deviation.

The quantification of IL-2 and IFN-γ set forth in FIG. 4 demonstrate the ability of CD19/20 CAR-T cells to recognize and produce cytokines in response to antigen-expressing tumor cells (Raji, Raji CD19KO, and Raji CD20KO) and not to antigen-negative tumor cells (CEM). The ability of the CD19/20 CAR-T cells to respond to dual antigens thus can impart a greater resistance to antigen escape seen in a large percentage of relapsed patients following anti-CD-19 CAR-T-cell therapy.

These observations demonstrate that the CD19/20 CAR-T cells produce both IFN-γ and IL-2>1,000 μg/ml when incubated at a 1:1 effector:target cell ratio, or greater, for 24 hours. These responses were evident with target cells that lack either CD19 or CD20 expression, but not with target cells that lack expression of both CD 19 and CD20. The data further confirm that the production of IFN-γ and IL-2 observed with the single antigen positive tumors cells is as robust, as the response to the CD19+/CD20+ double-positive target cells. These results suggest that the CD19/20 CAR-T cells are likely to have increased resistance to CD19 antigen escape, without additional risk of cytokine release syndrome (CRS).

### Example 5: Assessment of Cytotoxic Potency of CD19/20 CART Cells

The cytotoxic potency of the CD19/20 CAR-T cells towards CD19 and/or CD20 antigen expressing tumor cells was measured by a luciferase signal generated following co-culture with wild-type, single antigen positive Raji B cells, and compared to untransduced T cells from the same donor that have undergone the same manufacturing process. Across 3 donors, T cells expressing the CD19/20 CAR-T products exhibited robust Raji-cell lysis compared to the untransduced after a 72 hr coculture.

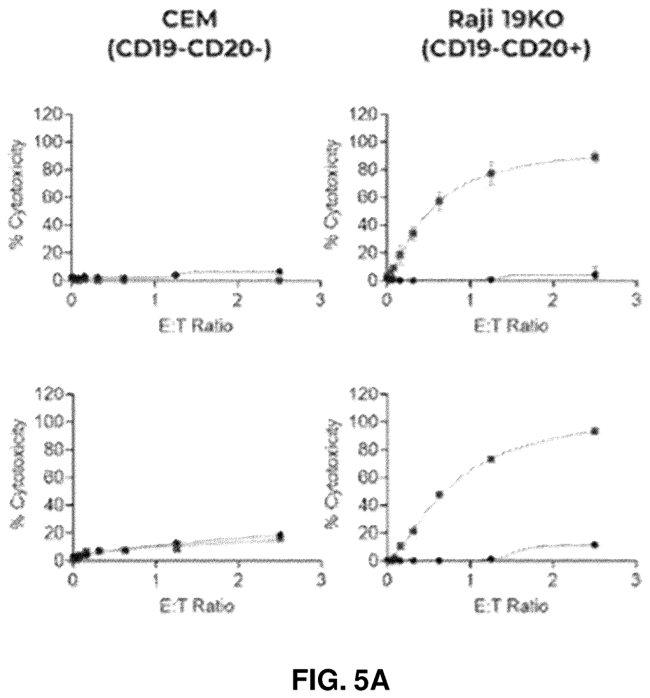

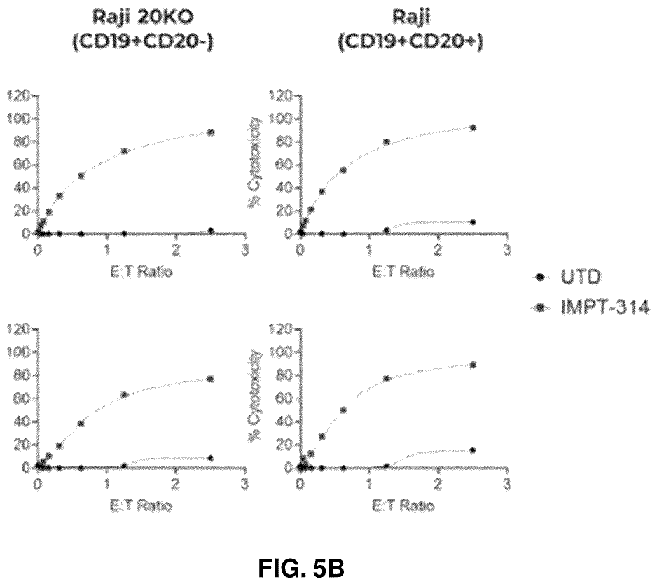

The cytotoxic potential of the CAR-T cells was then measured by luciferase signal from the remaining viable CD19+, CD20+, or CD19+/CD20+ target B cells remaining in co-culture. Across 3 donors, T cells expressing CD19/20 CAR exhibited robust Raji B cell lysis compared to the untransduced after a 72-hour coculture. Results are shown in FIG. 5.

As shown in FIG. 5, CAR-T cells from healthy donors were generated by lentiviral transduction with the CC314B vector at an MOI of 5. CAR-T cells were co-incubated with Raji cells (CEM, Raji WT, Raji 19KO or Raji 20KO) at a 2.5:1 E:T ratio, with a serial 2-fold dilution at a total of 7 dilutions in a 96-well plate for 72 hours. Cytotoxicity was then detected by measurement of the luciferase signal from the remaining target cells. The values shown are the means of technical replicates. Mean and standard deviation (error bar) are plotted.

Additionally, the CD19/20 CAR-T product showed equivalent cytotoxicity against wild-type, CD19 expressing (Raji 20KO) and CD20 expressing (Raji B cells CD19KO) target cells, indicating the potency of the bispecific CAR against each target antigen was comparable. These findings further support the conclusion that CD19/20 CAR-T exhibits equivalent cytotoxicity capacity against CD19+ and CD19− cells and therefore this dual antigen-targeting CAR product should eliminate pathogenic B cells relapse and enhance the probability that a patient can achieve durable complete remission from their autoimmune disease.

### Example 6: Construction of Chimeric Antigen Receptor (CAR) Expressing Vectors

Exemplary tandem targeting constructs for CD 19/CD20 as described herein were generated. The amino acid sequences of these exemplary constructs are set forth in SEQ ID NOs: 2, 4, 6, 8, 10, 12, 14, 16, 18 and 20. The constructs were generated by the following protocol.

In each case, a full length insert spanning an EFla promoter was chemically synthesized by linking a CD20 single-chain variable fragment (scFv) derived from a Leu16 monoclonal antibody and a CD19 single-chain variable fragment (scFv) derived from an FMC63 linked in sequence by a flexible interchain linker, in frame with the IgG4 hinge, CD28 transmembrane domain, and cytoplasmic domains of 4-1BB and CD3 zeta.

A leader sequence is derived from mIGg Kappa leader sequence included in all constructs. Full length CAR constructs sequences were cloned into the BstBI-Sall restriction sites of the third generation lentiviral plasmid backbone of pALD-Lenti EGFP-K (Aldevron Fargo, ND), replacing the sFFV promoter and EGFP gene.

Lentiviral vector (L V) containing supernatants were generated by transient transfection of HEK 293T cells.). Harvested pelleted lentiviral supernatants were stored at −80° C.

CD62L+ T cells were isolated from healthy donor whole blood obtained from Stem Cell Technology. Isolated cells were stimulated with CD3/CD28 TransAct™, a Human T cell expander CD3/CD28 (Miltenyi Biotec), and cultured in OpTmizer™ media (ThermoFisher) with Ix GlutaMAX™ (ThermoFisher), Interleukin-2 (IL-2) and IL-15.

T cells were lentivirally transduced 38-42 hours later at a multiplicity of infection of 5. After 6 or 7 days in TransAct-containing media, the cells were centrifuged, and the media was replaced by fresh media without TransAct. T cells transduced with anti-CD19/20 bispecific CAR were harvested and cryopreserved once a sufficient amount was obtained for administration.

The novel anti-CD19/CD20 CARs having the amino acid sequences of SEQ ID NOs: 2, 4, 6, 8, 10, 12, 14, 16, 18 and 20 contained the tandem scFv in their extracellular domain. T cells expressing these CARs specifically recognized both CD19+ and CD20+ target cells and carried out functions including degranulation, cytokine release, and proliferation. These CARs were compared with hinge and transmembrane domains derived from different human B cells (IgG) and human T cell molecules to evaluate the potency of transduced CAR T cells to eliminate established tumors in mice and human.

### Example 7: CAR Detection—CAR Expression is Stable Post-Transduction

CD62L+ naive/memory T cells (TN!M) were isolated from healthy donor Leukopaks without performing depletion of CD14+ and c25+ cells. Enrichment of CD62L+ cells was performed using magnetic microbeads from Miltenyi Biotec. TNIM cells (1.5×106/mL) were activated with GMP-grade TransAct™ Human T cell expander CD3/CD28 (Miltenyi Biotec) at a 1:35 dilution and T cells were then cultured in CTS OpTmizer™ media supplemented with GMP-grade recombinant human IL-2 (50 U/mL, Miltenyi Biotec) and GMP-grade recombinant IL-15 (0.5 ng/mL, Miltenyi Biotec). Two days after activation, 1×106/mL TNIM cells were transduced with vector at a titration of multiplicity of infection (MOI) from 0.5 to 20 and GMP-grade LentiBOOST™ (Sirion Biotech) at a dilution of 1:100. TNIM cells were incubated with lentivirus for 4 hours and then diluted to a final concentration of 0.5×106 cells/mL with CTS OpTmizer™ media supplemented with IL-2 (50 U/mL) and IL-15 (0.5 ng/mL) and cultured at 37° C. with fresh IL-2 and IL-15 were added to the media every Monday, Wednesday, and Friday. TransAct was removed from cell culture 7 days after activation.

Surface transgene express10n analysis was performed using flow cytometry 7 day (immediately after removing TransAct™), 10 days, and 12 days after activation. Cells were washed using Flow Cytometry Staining Buffer (BD) and stained with antibodies for 30 minutes at 4° C. Stained cells were washed using Staining Buffer (BD) and resuspended at 1×10 6 cells/mL for flow cytometry analysis. Single cells were identified based on forward and side scatter distributions and viable cells were identified based on live/dead cell stain. Trans gene positive cells were identified by surface staining with anti-mouse IgG F(ab)2-Biotin, which stains light chain of CD 19/20 CAR.

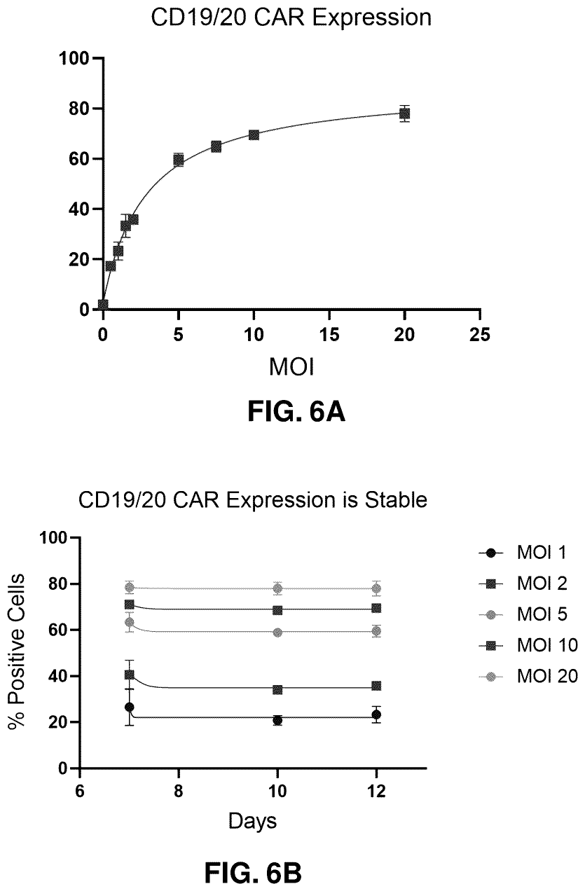

FIG. 6A and FIG. 6B. FIG. 6A shows that % CAR expression correlated with titration of MOI and, at MOI of 5, −60% CAR positive cells were obtained. FIG. 6B shows that CAR expression remained stable over 12 days. Data illustrated FIG. 6A and FIG. 6B are based on an average of 3 donors.

### Example 8: Vector Copy Number Linearly Correlates with Surface CAR Expression

T cells were harvested on Day 12. Vector copy number analysis was performed on transduced cells using droplet digital PCR (ddPCR). The total number of copies of the vector per transduced cell was determined by ddPCR. Genomic DNA was extracted from transduced T cells using the Qiagen DNeasy™ Blood and Tissue Kit (Qiagen) following manufacturer's protocol. The concentration of genomic DNA samples were measured using NanoDrop™ One/Onec Microvolume UV-Vis Spectrophotometer (Thermo Fisher Scientific). Probes and forward and reverse primers for ddPCR were synthesized by Integrated DNA Technologies (IDT). Primers and a FAM-labeled probe specific to the Leu16 scFv and FMC63 scFv element of the lentiviral vectors were used for trans gene detection (Table 6, Set A). As a reference gene, syndecan-4 (NCBI Gene ID: 6385; HGNC:10 661) with a HEX-labeled probe was used (Table 6, Set B).

TABLE 6
Primer and probe sequences.

| | | Sequences (5′-3′) |
|---|---|---|
| | Primers/probes | of primers and probe used for ddPCR |
| Set A: | Set A: Leu16 scFv | Forward: CTTATTACTGCCAGCAGTGGAG |
| CD19/20 | (Target) | (SEQ ID NO: 38) |
| CAR | | Reverse: CAGGACATCTTCACTGAGGC |
| | | (SEQ ID NO: 39) |
| | FAM/Iowa Black FQ | Probe: CGGAGCCACCACCGCTAGTACTG |
| | | (SEQ ID NO: 40) |
| Set B: | Set B: FMC63 scFv | Forward: CGACATCCAGATGACCCAG |
| CD19/20 | (Target) | (SEQ ID NO: 41) |
| CAR | | Reverse: GTTCCAGGTTGGAGATGGTC |
| | | (SEQ ID NO: 42) |
| | HEX/ZEN/Iowa Black | Probe: |
| | FQ | CACCGTCAAGCTGCTGATCTACCACACCA |
| | | (SEQ ID NO: 43) |
| Set D: | Set D: SDC4 (Reference) | Forward: CAGGGTCTGGGAGCCAAGT |
| Reference | | (SEQ ID NO: 44) |
| | | Reverse: GCACAGTGCTGGACATTGACA |
| | | (SEQ ID NO: 45) |
| | HEX/ZEN/Iowa Black | Probe: |
| | FQ | CCCACCGAACCCAAGAAACTAGAGGAGAAT |
| | | (SEQ ID NO: 46) |

PCR reaction mix was prepared using 10 μL of 2×ddPCR supermix for probes (No dUTP) (BioRad), primers at final concentrations of 500 nM, probes at final concentrations of 250 nM. Prior to ddPCR, 15-40 ng of genomic DNA was linearized using Bsp14071 (Thermo Fisher Scientific) for 30 mins at 37° C. Afterwards, 20 μL of PCR mixes and 70 μL of droplet generation oil (Bio-Rad) were loaded into the DG8 Cartridges with gaskets, yielding a final volume of 40 μL product from the QX200™ Droplet Generator (Bio-Rad). The droplet emulsion was transferred to a 96-well PCR plate and DNAs were amplified at following conditions in a C1000 Touch Thermal Cycler (Bio-Rad): denaturation at 95° C. for 10 min; 40 cycles at 94° C. for 30 s, 60° C. for 1 min and 98° C. for 10 min; hold at 12° C. Data acquisition was done within 24 hours in the QX200™ Droplet Reader (Bio-Rad) and analysis was performed with the QuantaSoft™ Software (Bio-Rad). No template controls (NTC) were used to monitor contaminations of reagents and the formation of primer dimers. Untransduced T cells (UTD) from the same donor was used as negative control to evaluate off-target amplification.

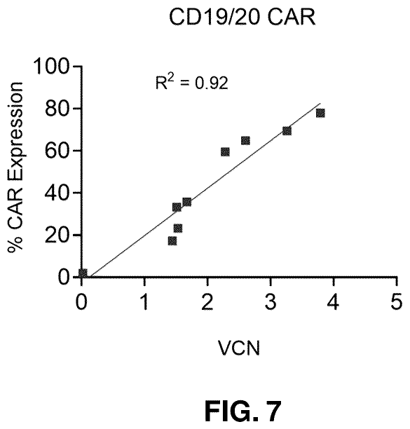

See FIG. 7 which shows that VCN (ddPCR) and % CAR expression (measured using flow cytometry) correlated linearly as MOI increased from 0.5 to 20.

### Example 9: Cytotoxic T Lymphocyte Assay

To evaluate the potency of CD 19/20 CAR transduced T NJM cells, transduced T cells were incubated with target cells with and without CD 19 and CD20 antigen for 24 hours to quantify antigen specific CAR-T cell cytotoxicity. CD19 and CD20 negative target cell lines were generated from parental wildtype Raji cells (CD19+ and CD20+) purchased from ATCC. CD19 and C20 antigens were knocked out using CRISPR/Cas9, expanded from single cell clones, and sequence verified using NGS.

CD19/20 CAR-TNJM cells were incubated with 10,000 luciferase expressing Raji cells at an effector to target (E:T) ratio of 10: 1 (10 CAR positive T cells to 1 Raji cell) and titrated serially down by a factor to 2 for 9 dilutions in a 96 well plate. The last well served as a target cell only control. Cells were incubated for 18 hours in a 37° C., 5% CO2 incubator, after which 120 μL of supernatant containing secreted cytokines was removed and stored at −20° C. for further analysis (14.2.4). 20 μL Bio-Glo Luciferase reagent (Promega) was added to the remainder of the cell suspension and incubated at room temperature, protected from light, for 15 minutes. Luminescence was read on the Varioskan™ (Thermo Fisher).

CTL data was collected across 3 donors. T cell effector function was quantified based on target cell viability after 18 hours of co-culture. CD 19/20 CAR-T cells demonstrated killing of Raji WT, Raji CD19 KO, and Raji CD20 KO cells with >60% CTL observed at E:T of 1:1.

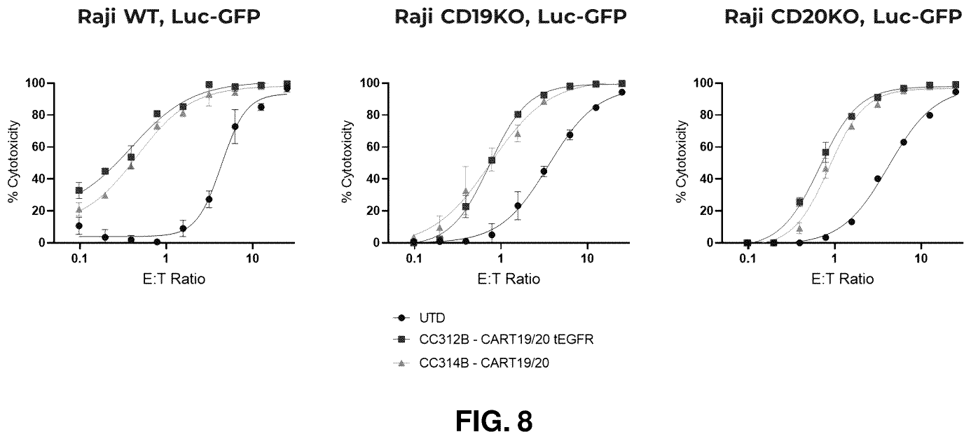

FIG. 8 illustrates the cytotoxicity of CD 19/20 CAR-T N/MEM cells co-cultured with Raji cells (with or without CD19 and CD20 protein) at a range of effector to target ratios for 48 hours. Cytotoxicity was measured by quantification of viable GFP+ target cells using flow cytometry.

### Example 10: Cytokine Secretion Assay

To measure IFN-γ and IL-2 secreted in cell-mediated cytotoxicity assays, T cells and target cells were co-cultured at varying ratios of effector to target (E:T) in the absence of exogenous cytokines and incubated for 18 hours at 37° C. and 5% CO2. After incubation, supernatants were harvested, diluted l0×, and analyzed for secretion of IL-2 and IFN-γ using R&D Systems ELISA kits (D2050 and DIF50C, respectively) following manufacturer's protocol. In both assays, 100 μL of Assay Diluent and 100 μL of either cytokine standard or sample were added into each well and incubated at room temperature for 2 hours. Samples were washed and incubated with the anti-IL-2 or IFN-γ antibody conjugates at room temperature for 2 hours. After an additional washing step, substrate solution was added and incubated at room temperature, protected from light, for 20 minutes. Finally, stop solution was added and optical density was read at 450 nm and 540 nm. Values read at 540 nm were subtracted from the values at 450 nm which corrects for optical imperfections of the plate. Quantification of cytokine secretion is based on the optical density from the reaction of enzyme-linked polyclonal antibody in contact with a substrate and detected on a spectrophotometer. All samples were performed in triplicates, unless otherwise noted.

Consistent with observations in the CTL assay, CD19/20 CAR-TN/M Cells secreted IL-2 and IFN-γ when co-cultured with either Raji WT, Raji CD19 KO, or Raji CD20 KO cell lines. Peak secreted IL-2 and IFN-γ was observed at E:T ratio of 10:1.

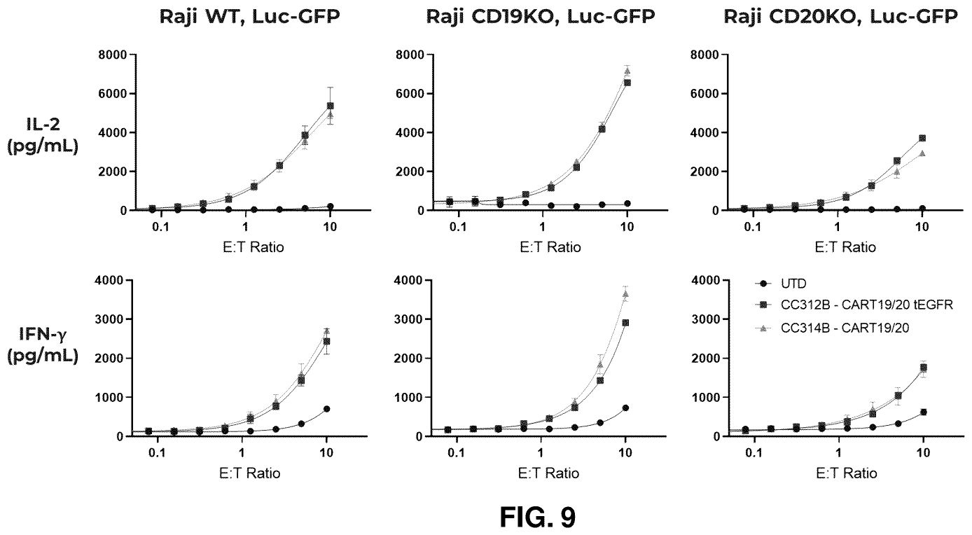

FIG. 9 illustrates the in vitro quantification of cytokine secretion by ELISA. IL-2 secretion by CAR-T transduced cells which have been cocultured with target cells (Raji) overnight and assayed by ELISA.

As can be seen from FIG. 9, IL-2 and IFN-γ secretion from the CD19/CD20-CAR T titrates with the E:T ratio and is significantly higher than CD19-CAR T cells.

### Example 11: Manufacture

Approximately 14-28 days prior to infusion of transduced cells, patients will undergo leukapheresis to collect white blood cells (Day 0). The following day (Day 1), the leukapheresis product will be enriched for CD62L+ cells. The CD62L+ cells are called naive memory T cells, (TN/MEM, also abbreviated as Tnm or TN/M) Within 2 hours of CD62L+ enrichment, TN/MEM cells will be stimulated with clinical-grade CD3/CD28 TransAct™ (Miltenyi Biotec) and fed with 50 U/mL interleukin (IL)-2 and 0.5 ng/mL IL-15. On M-Day 3, cells will be transduced with the lentivirus vector CC314B. Transduced cells (henceforth referred to as CART19/20 cells) will be expanded ex vivo, with the addition of IL-2 and IL-15 (50 U/mL and 0.5 ng/mL final concentration, respectively) every other day. Additional culture media will be added and total cell culture volume will be expanded as necessary to maintain a cell density of 0.3-0.6×106 cells/mL. TransAct™ will be removed on M-Day 7, and cell expansion will continue until sufficient cohort-specific CAR+ cell numbers have been generated, with manufacturing estimated to finish on M-Day 12.

For example, in one embodiment, TransAct™ will be removed on Day 7, and cell expansion will continue for 5 days or more after transduction until sufficient viable CAR+ cell numbers (e.g., 2×108 total cells) have been generated, with manufacturing estimated to finish on Day 12-14.

### Example 12: Comparison of Transduction Enhancers

Generation of CAR-T cells through lentiviral transduction can be improved by adding transduction enhancers such as polybrene (RetroNectin, Takara Bio), protamine sulfate (Fresenius Kabi), LentiBoost™ (Sirion Biotech, Germany), Vectofusin-1 (Miltenyi Biotech, Germany) etc. [0422] Transduction efficiency using no transduction enhancer, protamine sulfate and LentiBoost™ were compared in this study. CD62L+ naive/memory T cells (TNJM) were isolated from healthy donors and transduced in a composition comprising transduction enhancer, IL-2, IL-15, TransAct™ and a lentivirus comprising a nucleic acid encoding the CD19/CD20 bi-specific CAR in pALD backbone (Lentigen). The cells were transduced at a concentration of 1×106 cells/mL at multiplicity of infection (MOI) of 0.5 and 5 based on manufacture's titer. Cells were incubated with virus, IL-2 and/or IL-15 with or without transduction enhancers for 6 hours prior to equal volume of media addition.

At Day 12 after initial activation of T cells, CAR expression, vector copy number and potency assays were performed. At low MOI, T cells transduced using LentiBoost™ yields higher transduction efficiency than using protamine sulfate or without addition of transduction enhancer.

### Example 13: Production of IMPT-314 (pALD-Leu16-FMC63-CD28_BBz)

IMPT-314 (pALD-Leu16-FMC63-BBz) is produced by obtaining autologous T cells which are then transduced with lentiviral vector to express the anti-CD19/20 bispecific CAR.

Autologous T cells are collected by leukapheresis. A CD14+/CD25+ depletion step is not performed. The production of IMPT-314 from subject apheresis material is a continuous process without any hold steps between production of drug substance and drug product. The IMPT-314 drug product is CART19/20 cells formulated in a 1:1 mixture of CryoStor CS-10 and Plasmalyte/5% human serum albumin (HSA), for a final concentration of 5% DMSO and 2.5% plasmalyte. IMPT-314 drug product will include both transduced (CAR+) and untransduced (CAR) cells.

CD 14+/CD25+ depletion step: Prior protocols used in the art required and included a CD 14+/CD25+ cells negative selection (i.e., depletion) step. At the beginning of the protocol, the leukapheresis sample was analyzed for CBC and by flow cytometry for CD3+, CD14+, CD25+, and CD62L+ cells. This next step in the process was dependent on the flow cytometry results. If % of CD62L+ cells that were also either CD25+ or CD14+ was >5% then a depletion step was performed by removal of CD14+/CD25+ cells using the ClinMACS column. However, if the % CD14+ and/or CD25+ within CD62L+ population is <5% the protocol permits the CD14+/CD25+ depletion step to be bypassed and move directly to CD62L enrichment using the ClinMACS column. This CD14+/CD25+ depletion step was bypassed in manufacturing the products for 3 patients.

TABLE 7
Comparison of Previous Manufacturing Method and Method of the Present Invention

| | Previous | Method of the | |
|---|---|---|---|
| | Manufacturing | Present | |
| Process step | Methods | Disclosure | Observation |
| CD62L+ enrichment | Addition of Human | CliniMACS Plus | No difference has been observed in the |
| step | Immune globulin—IgG | enrichment | level of enrichment with and without |
| | (Gammagard, Takeda) at | | IgG addition. |
| | 0.28% | | |
| CD14+/CD25+ | Performed if % CD14+ | Not performed | No difference in transduction efficiency |
| depletion step | and/or CD25+ within | | has been observed between the |
| | CD62L+ population | | manufacturing process with and without |
| | is >5% | | the depletion step. |
| Cell washing (buffer | Manual Centrifugation | Sepax C-Pro | Use of an automated, closed system to |
| exchange) at Day 0, | | | reduce contamination risks and |
| Day 7 and Day 12 | | | potential human errors. |
| Cell activation prior | Duration of 32-48 hours | Duration of 38-46 | Reduction of the activation window to |
| to transduction | | hours (target 42 | improve process consistency. |
| | | hours) | |
| Transduction | Protamine sulfate, as a | No enhancer added | No significant or consistent |
| | transduction enhancer. | | improvement in transduction efficiency |
| | | | has been observed with or without |
| | | | protamine sulfate. |
| Drug product | Cryostor CS5 | Cryostor CS10 and | Use of automated closed system (Sepax |
| formulation | | Plasmalyte/5% | C-Pro) for the final wash step prior to |
| | | HSA (1:1 ratio) | formulation: cells are resuspended in |
| | | | Plasmalyte at 5% HSA. |
| | | | The formulation maintains a final 5% |
| | | | DMSO concentration, which is critical |
| | | | for cryopreservation. |

### Example 14: Vector Drug Substance

In accordance with the present invention, the CD19/CD20 CAR-T utilizes a lentiviral backbone, the pALD, Aldevron's (Fargo, North Dakota) Lenti expression plasmid, to replace the bispecific CD19/20 CARs viral cloning plasmid and moved to an alternative manufacturer.

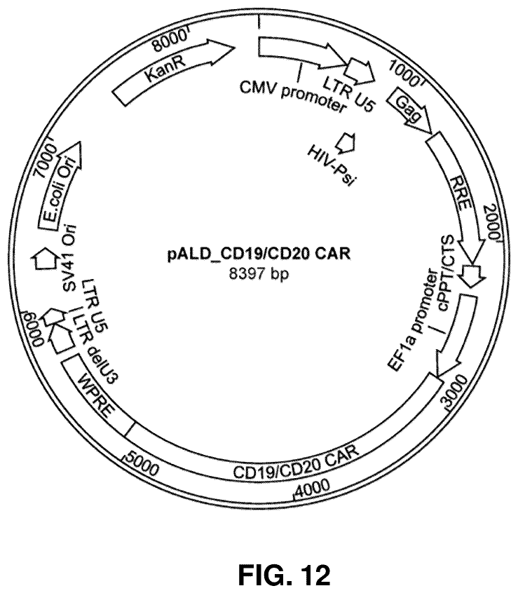

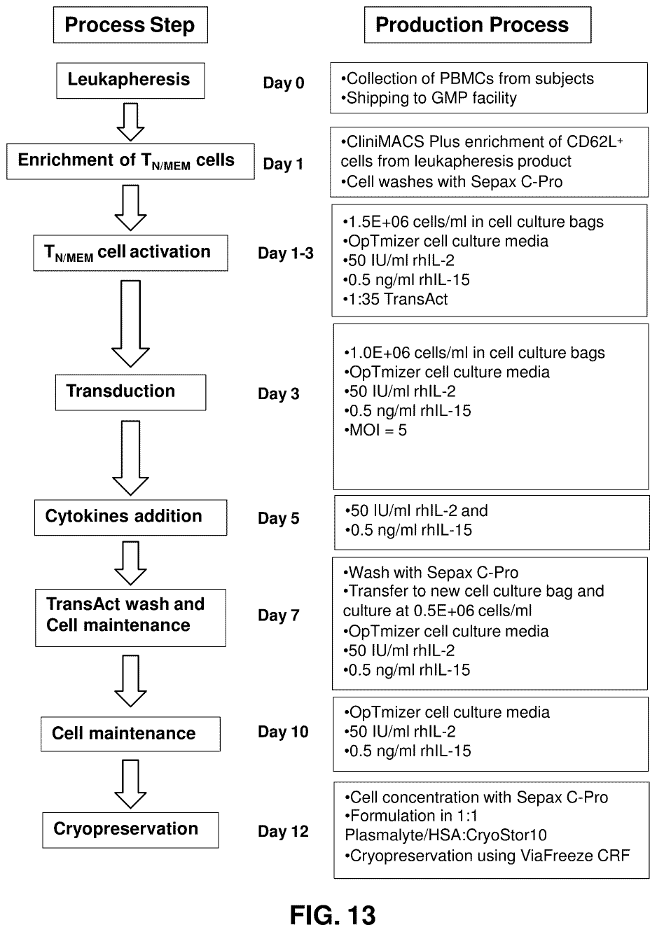

Both the bispecific CD19/20 CAR transgene and the EFla promoter are incorporated into the pALD plasmid. The plasmid containing the insert is 8,397 kb in size and contains lentiviral regulatory elements. A map of the pALD_CD19/CD20 CAR plasmid is shown in FIG. 12.

The antibiotic selectable marker in pALD is Kanamycin. Aminoglycosides such as kanamycin and neomycin are currently preferred since they are rarely used in the clinic.

The WPRE is a common feature of contemporary retroviral vectors that can improve production and potentially gene expression in target cells. This sequence is driven from the woodchuck hepatitis virus post-transcriptional regulatory element and prevents poly(A) site readthrough, to promote RNA processing and maturation, while increasing nuclear export of RNA. Safety concerns have been raised regarding the potential oncogenic activity of the truncated woodchuck hepatitis virus X protein encoded in this element. Therefore, in accordance with the invention, this capability was abrogated by mutating the WPRE ORF translation to prevent potential expression of the X protein start codon from ATG to TTG. It was found that the native form or mutated derivatives of WPRE function equivalently.

### Lentiviral Vector Components

The third generation pALD lentiviral vector is a replication-incompetent and self-inactivating vector, due to the number of essential genes that have been deleted. It includes an additional number of safety features; an altered 3′ long terminal repeat (LTR) renders the vector “self-inactivating” to prevent integrated genes from being repackaged and a heterologous coat protein (e.g., VSV-G) is used in place of the native HIV-1 envelope protein. Key components of the pALD vector are described in Section below.

CMV promoter: The cytomegalovirus (CMV) promoter replaces the U3 LTR in SIN vectors and drives transcription of viral ribonucleic acid (RNA) in packaging cells. This RNA is then packaged into live virus.

5′ LTR-AU3: This is a deleted version of the HIV-1 5′ long terminal repeat. The LTRs carry both promoter and polyadenylation function. In the pALD plasmid, 5′ LTR-L1 U3 is deleted for safety. This does not affect the production of viral RNA during packaging because the promoter function is supplemented by the CMV promoter engineered upstream of 5′LTR-L1 U3 LTR.

‘P: HIV-1 packaging signal required for the packaging of viral RNA into virus.

RRE: HIV-1 Rev Response Element (RRE). RRE permits the nuclear export of viral RNA by the viral Rev protein during viral packaging.

cPPT: HIV-1 Central Polypurine Tract (cPPT). cPPT creates a “DNA flap” that increases nuclear import of the viral genome during target cell infection. This improves vector integration into the host genome, resulting in higher transduction efficiency.

Incorporation of a cPPT and a posttranscriptional regulatory element (PRE) into lentivirus vectors provides increased transduction efficiency and transgene expression.

Hybrid EFla/HTLVl promoter: The hEFla-HTLV promoter is a composite promoter comprising the human Elongation Factor-la (EF-la) core promoter and the R segment and part of the U 5 sequence (R-U 5′) of the Human T-Cell Leukemia Virus (HTL V) Type 1 Long Terminal Repeat. The EF-1 a promoter exhibits a strong activity and yields long lasting expression of a transgene in vivo. The promoter drives the expression of the CD19/CD20 CAR proteins.

Kozak: Kozak consensus sequence The Kozak consensus sequence is placed in front of the start codon of the opening reading frame (ORF) of interest to facilitate translation initiation in eukaryotes.

CD19/CD20 ORF: The open reading frame of the tandem CAR T proteins.

WPRE: Woodchuck hepatitis virus posttranscriptional regulatory element (WPRE). The WPRE enhances viral RNA stability in packaging cells, leading to higher titer of packaged lentiviral particles. Here, a mutation was introduced into the translation initiation (ATG) site of the X protein to eliminate any potential translation through the WPRE promoter sequence.

3′ LTR-AU3: A truncated version of the HIV-1 3′ long terminal repeat that deletes the U3 region. This leads to the self-inactivation of the promoter activity of the 5′ LTR upon viral vector integration into the host genome (since the 3′ LTR is copied onto 5′ LTR during viral integration). The polyadenylation signal contained in 3′ LTR-L1 U3 serves to terminates all upstream transcripts produced both during viral packaging and after viral integration into the host genome.

pUC ori: pUC origin of replication. Plasmids carrying this origin exist in high copy numbers in E. coli.

Extracellular Signaling Domains: The extracellular domain of the pALD CD 19/20 CAR is derived from the antigen binding domain of Leu16 murine antibody that recognizes human CD20, and the antigen binding domain of FMC63 (recognizing CD19) to create a tandem-CAR configuration. The bispecific configuration enables bivalent binding to CD19 and/or CD20 on B cells. The scFv fragments are connected via GS flexible linkers. The IgG4 extracellular hinge connects the Leu16 scFv to the CD28 TM domain, and the intracellular domains derived from the human cytoplasmic domain of 4-1BB costimulatory domain and CD3 zeta activation domain.

scFv domains: The bispecific scFv domain of a CAR provides the targeting function by specifically recognizing the tumor antigens. The specificity of the scFv is a crucial determinant for the CART cell safety profile. Given that both CD19 and CD20 expression is restricted within the B-cell lineage, the tandem CARs scFv are considered reasonably safe with respect to off target cross reactivity.

Spacer and transmembrane domain. In the tandem CD19/CD20 CAR, the non-signaling Extracellular Spacer Domain is derived from the IgG4 Hinge region, while the transmembrane domain is derived from human CD28. The spacer provides a flexible link between the scFv and the transmembrane domains. It allows the antigen-binding domain to accommodate different orientations to facilitate antigen recognition. The transmembrane domain provides a physical link between the spacer and intracellular signaling domains. The length and topology of the spacer and transmembrane domains are critical in providing an appropriate steric orientation for specific antigen recognition and subsequent T cell activation.

Intracellular signaling domains. The CAR intracellular signaling domains play crucial roles in T cell activation, persistence, and effector functions. Although the CD3□ chain is adequate for T cell activation, one or more costimulatory domains are also needed to fully activate T cells and to promote CART cell persistence (Milone, 2009). These domains are typically derived from the intracellular domains of costimulatory proteins such as 4-1BB (CD 137). Differences in costimulatory domain function may impact product safety and activity by affecting CAR T cell cytokine production, expansion, cytotoxicity and persistence after administration. The intracellular signaling domains of the CD19/CD20 CAR are derived from human 4-1BB and CD3ζ.

Plasmid DNA was prepared from a vial of pALD_CD19/CD20 CAR MCB according to written procedures at Aldevron. Details of the plasmid production process will be provided in the IND with a letter of cross-reference to the Aldevron Drug Master File (DMF).

### Example 15: Lentiviral Vector Manufacturing

Production of the lentiviral vector is as follows. Using the current serum-free suspension manufacturing process, viral vector is manufactured by transient transfection using Lentigen's four plasmid system, which includes research grade transfer plasmid and Lentigen's three helper plasmids. The downstream process includes clarification, Benzonase® treatment, tangential flow filtration, and column chromatography. After purification, the Vector will be concentrated approximately 100-fold, formulated in formulation buffer, sterile filtered, and filled into 1 mL vials.

In preparation for GMP production of the lentiviral batch, one 2 ml research-grade batch and 4L non-GMP batch was prepared by Lentigen.

### Production and Release of IMPT-314 Drug Product

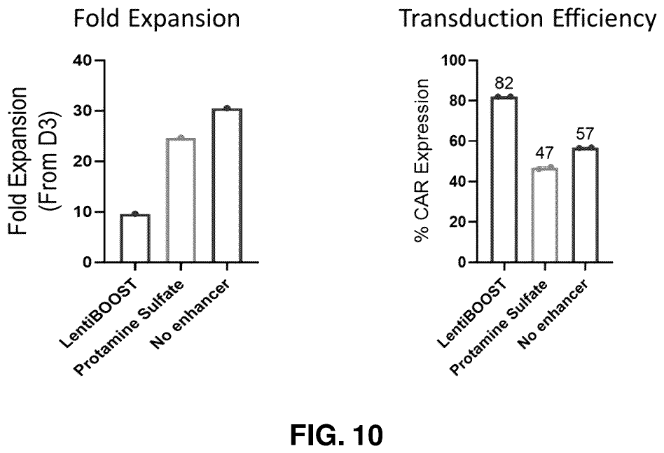

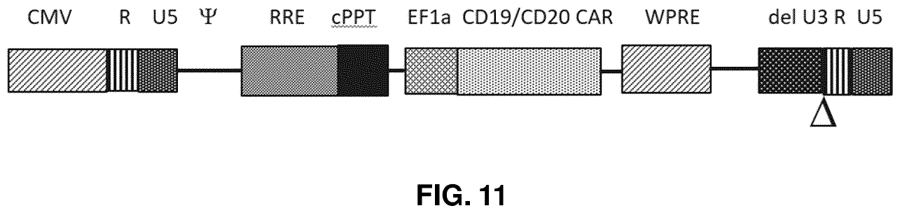

IMPT-314 Manufacturing Procedure. The proposed manufacturing process for production of IMPT-314 is provided in FIG. 10. The manufacturing process consists of the following steps and timing:

Day 0: Starting Material (Leukapheresis). Autologous peripheral blood mononuclear cells (PBMCs) is collected by leukapheresis according to written procedures. The leukapheresis product is collected into sterile bags at the collection site and shipped to ImmPACT Bio at 2-8° C. in a temperature-controlled container for processing and cell product manufacture.

Day 1: Enrichment and Activation of TN/MEM cells. The leukapheresis product is then enriched for CD62L+ cells using GMP-grade CD62L microbeads and a CliniMACS Plus (Miltenyi Biotec). Briefly, the leukapheresis product is washed with CliniMACS PBS/EDTA buffer supplemented with 0.5% HSA. The cells are then incubated with the anti-CD62L microbeads and washed with the PBS/EDTA/HSA buffer using a Sepax C-Pro (Cytiva). CD62L microbead labeled cells are then added and run through the CliniMACS plus. The resultant selected CD62L+ cells are called naive/memory T cells (TN/MEM). TN/MEM cells will be resuspended in OpTmizer™ media supplemented with 50 IU/mL human IL-2 and 0.5 ng/mL human IL-15 (Miltenyi Biotec) and transferred to static cell culture bag (VueLife). The cells are then stimulated with GMP-grade TransAct Human T cell expander CD3/CD28 (Miltenyi Biotec) added at a ratio of 1:35 and cultured for 42 hours at 37° C./5% CO2 in a humidified incubator.

Day 3: Lentiviral Transduction. Genetically modified T cells expressing an anti-CD19/20 bispecific CAR are manufactured by transduction of stimulated TN/MEM cells with lentiviral vector (Lentigen). To perform this unit operation, activated TN/MEM cells are harvested from the static cell culture bag and re-cultured in a new cell culture bag in fully supplemented OpTmizer™ media at a density of 1.0E+10⁰⁶ cells/mL. Lentiviral vector is then be added to the cells at a multiplicity of infection (MOI) of 5. The cells with the added lentiviral vector are then incubated for 6 hours at 37° C./5% CO2 in a humidified incubator. Following this incubation, an equal volume of media is added to the cell culture bag to have a final cell concentration of 0.5E+10⁰⁶ cells/mL and incubated at 37° C./5% CO2 for a further 2 days.

Day 5: Cytokines addition. On day 5 post-stimulation, 50 IU/mL human IL-2 and 0.5 ng/mL human IL-15 (Miltenyi Biotec) is added to the culture bag containing the T cells. Cells are incubated at 37° C./5% CO2 for a further 2 days.

Day 7: TransAct Wash and Cell Maintenance. After 7 days of culture, transduced TN/MEM cells are washed out of the media containing TransAct using a Sepax C-Pro (Cytiva) and re-cultured in a new cell culture bag with fully supplemented fresh media at a density of 0.5E+10⁰⁶ cells/mL. Cells will be incubated at 37° C./5% CO2 for a further 3 days.

Day 10: Cell Maintenance. On day 10 post-stimulation, fully supplemented media is added to dilute the cells at a cell density of 0.5E+10⁰⁶ cells/mL. Cells are incubated at 37° C./5% CO2 for a further 2 days.

Day 12: Cryopreservation. After 12 total days of culture, transduced TN/MEM cells are harvested and cryopreserved at a fixed dose of CAR+ cells. Cells are washed out of media and into Plasmalyte plus HSA using a Sepax C-Pro (Cytiva). The concentrated cells are then mixed at a 1: 1 ratio with a cryopreservation solution containing DMSO (CryoStor CS 10 Freezing Solution, BioLife). Cells are then frozen using a ViaFreeze™ controlled rate freezer (Cytiva), cryopreserved in a labeled cryopreservation-bag and stored in a centrally-monitored vapor phase liquid nitrogen freezer. The Drug Product is cryopreserved in CryoMACS Freezing Bag 50 or 250. A portion of the cells are then cryopreserved in small aliquots along with the bulk final product for final lot-release testing.

Table 8 describes the product contact materials that will be used for manufacture of IMPT-314 (pALD-Leu16-FMC63-BBz).

TABLE 8
Reagents/Materials Used in Manufacture.

| Reagent/Material | Vendor-Supplier | Use |
|---|---|---|
| Human serum albumin (HSA) | Nova Biologics | Cell enrichment |
| Plasmalyte | Baxter | Final product formulation |
| CliniMACS CD62L GMP | Miltenyi | Cell enrichment |
| MicroBeads | | |
| MACS GMP T Cell TransAct | Miltenyi | Cell activation |
| CliniMACS PBS/EDTA Buffer | Miltenyi | Cell Enrichment/Depletion |
| research | | |
| GMP Recombinant Human IL-2 | Miltenyi | Cell culture media |
| GMP Recombinant Human IL-15 | Miltenyi | Cell culture media |
| GlutaMAX-I Supplement | Gibco | Cell culture media |
| CTS ™ OpTmizer T-Cell | Gibco | Cell culture media |
| Expansion Supplement | | |
| CTS ™ OpTmizer T-Cell | Gibco | Cell culture media |
| Expansion Basal Medium | | |
| Cryostor CS10 Cryopreservation | BioLife | Final product formulation |
| media | | |
| Cell culture bag | Saint-Gobain 3 197-C | Culture bag |
| Cell culture bag | Saint-Gobain 3 290-C | Culture bag |
| CryoMACS Freezing Bag 50 | Miltenyi | Final product |
| CryoMACS Freezing Bag 250 | Miltenyi | Final product |

### In Process Testing Strategy

In-Process Testing Samples are collected before, during, and at the end of the cell-manufacturing procedure. The planned list of control points is provided in Table 9 as follows:

TABLE 9

| Unit Operation | Process Step | Testing Purpose |
|---|---|---|
| Day 1: Apheresis Collection | Apheresis before Sepax wash and | Total cell count and viability |
| | volume reduction | Leukocyte subsets composition and T-cell |
| | | phenotype |
| | | Sample taken for sterility if needed |
| Day 1: CD62L Enrichment | Enriched CD62L cells before Sepax | Total cell count and viability |
| | wash | |
| | Enriched CD62L cells after Sepax wash | CD62L purity check |
| Day 7: Cell Maintenance | Before media addition | Total cell count and viability |
| Day 10: Cell Maintenance | Before Sepax wash | Total cell count and viability |

Each Drug product will be tested for release criteria (See Table 10).

TABLE 10
IMPT-314 Release Criteria

| Attributes | Product metric | Method | Limits |
|---|---|---|---|
| Purity | Appearance | Visual inspection | Off-white, product |
| | | | free of particulates |
| Dose | Number of CAR T cells | % CAR+ by Flow Cytometry | DL1: 50E6 +/− 10E6 |
| | manufactured | CAR+ Cell count by | CAR+ cells |
| | | NucleoCounter | DL2: 150E6 +/− |
| | | | 30E6 CAR+ cells |
| Potency | Viability | NucleoCounter | ≥70% |
| | CAR+ expression | CAR+ by F(ab)₂ detection | ≥10% |
| | | Flow Cytometry | |
| | Tumor reactivity | IFN-γ secretion by ELISA | CD19+ target |
| | | | cells: ≥100 pg/ml |
| | | | CD20+ target |
| | | | cells: ≥100 pg/ml |
| | CTL | Flow Cytometry | TBD |
| Purity | CD3% | Flow Cytometry | ≥80% |
| | Endotoxin | EndoSafe-PTS | ≤5 EU/ml |
| Identity | Target vector inserted gene | ddPCR using Leu16 specific | Amplified above |
| | | primers/probe | LOD |
| Safety | VCN | ddPCR | ≤5 copies/cell |
| | RCL | qPCR | Not detected |
| Sterility | Bacterial and Fungal | USP <71> or Bac-T Alert | No Growth |
| | culture | | |
| | Gram stain | Staining | Negative |
| Mycoplasma | Mycoplasma | MycoAlert | Not detected |

### Analytical Methods

### Viability and Cell Count

The Number of cells and viability is assessed using an automated cell counter system against mouse F(ab)2. Live/Dead stain is included in the panel to discriminate the positive cell population. Isotype control is used to identify the non-specific binding. Flow Cytometry assays are performed on a representative aliquot of the Drug Product collected and cryopreserved at day 12.

Vector Copy Number: Vector copy number (VCN) estimates the number of vector copies in each transduced cell, which may correlate with the amount of CAR protein expressed on the cell surface. VCN is determined by ddPCR, using primers/probes sets specific to amplify the CD20 scFv derived from the Leu-16 mAb. The average number of integrations per CAR-positive cell is determined.

Cytokine secretion (IFN-γ secretion): To assess potency of the IMPT-314, a representative aliquot of the cryopreserved Final Product is thawed to perform cytokine assays. Drug Product and target cells are co-cultured at varying ratios of effector to target (E:T) in the absence of exogenous cytokines and incubated for 16-18 hours. Raji target cells are engineered to express 1) CD19 antigen only, and 2) CD20 antigen only to assess the activity of both elements in the CAR. CD19 and CD20 knockout (KO) target cell lines are generated from parental wildtype Raji cells (CD19+ and CD20+) using CRISPR/Cas9 and expanded from single cell clones. CEM cells, which do not express CD19 or CD20, were used as a negative control. After incubation, supernatants are harvested, diluted 1OX, and analyzed for secretion of IFN-γ using R&D Systems Quantikine ELISA kits.

Cytotoxic T Lymphocytes assay (CTL): To show specific killing against CD19 and CD20 expressing Raji cells, a co-culture of GFP-expressing Raji cells (wildtype, CD19KO and CD20KO) or CEM (CD19−/CD20−) with transduced T cells at E:T titrations is used. Raji cell viability is analyzed by Flow Cytometry after 18 hrs and gated on Sytox Blue negative and GFP positive target cell counts.

Sterility: A representative aliquot of the Final Drug Product is tested for bacterial and fungal growth by USP <71> or B ac-T Alert. A sample of the leukapheresis starting material is retained for post hoc testing in the event of a Final Drug Product sterility test failure.

Endotoxin: Endotoxin levels in the Final Drug Product is assessed using the Charles River EndoSafe™ Portable Test system.

Mycoplasma: Mycoplasma is tested using the MycoAlert Mycoplasma Real-Time PCR Kit. The MycoAlert™ system is a biochemical reaction test that detects the presence of mycoplasmal enzymes that are not found in eukaryotic cells.

RCL: The Drug Product is assayed to detect RCL using qPCR for VSV-G sequences. qPCR assay can be called negative if the final value is less than the LOQ. Optional RCL-cell culture assay with amplifying/indicator cell lines is submitted and performed only if the qPCR criteria is not met, for confirmation.

Direct Detection of CAR+ cells by Flow Cytometry. Direct measurement was used to detect expression of CD 19/20 CAR. Because quantification of a tEGFR protein may not provide an accurate measurement of surface CAR expression, tEGFR was removed from the Lentiviral vector as described herein, and instead utilized antibodies which bound to the scFv domains of the CD19/20 CAR protein to report surface CAR expression. To identify the best detection reagent, a panel of antibodies, proteins, and peptides was screened, which found that one antibody in particular—anti-mouse F(ab)2 antibody (Jackson Immuno Research) showed superior CAR detection in multiple donors.

TABLE 11
Comparison of antibodies, proteins,
peptides to detect CD19/20 CAR

| | | % Positive |
|---|---|---|
| Antibody/Protein | Vendor | Cells |
| CD19 Fc HisTag | Sino Biotech | 16.2 |
| CD19 Fc | R&D Systems | 39.2 |
| CD20 Protein-Biotin | Acros | 68.1 |
| CD20 Peptide-FAM | GenScript | 8.3 |
| Byotinilated anti-FMC63 | Acros | 59.6 |
| Anti-mouse F(ab)2 | Jackson | 84.9 |
| | Immuno Research | |
| Goat anti-Ms IgG (H + L) -AF488 | Abcam | 77.6 |
| Goat anti-Ms IgG (H + L) -AF 647 | Abcam | 77.8 |
| Goat anti-Ms IgG (H + L) -AF488 | Invitrogen | 78.0 |

T cells from donor 1917 transduced with CD 19/20 CAR lentiviral vector were washed and stained using various antibodies, proteins, or peptides. Antibodies were titrated from 1:25 to 1:200. Anti-F(ab)2 biotin antibody (Jackson Immuno Research) and anti-ms IgG (H&L) 647 (Abcam) stained cells with similar efficacy compared to EGFR stain.

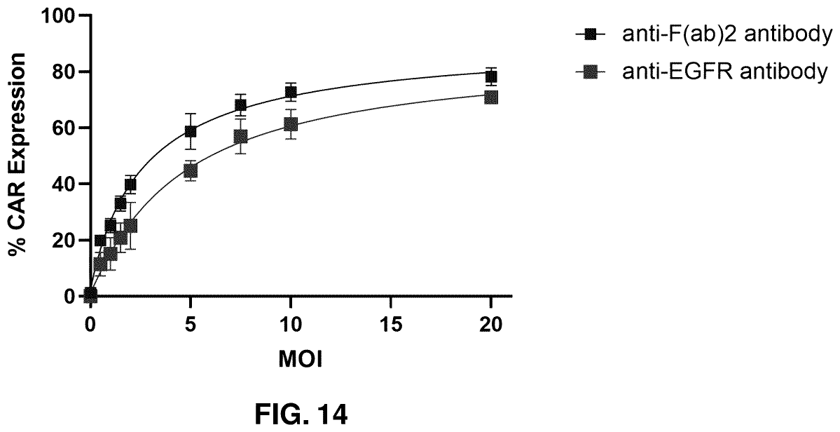

Next, to compare the direct CAR detection method to a tEGFR detection method, a lentiviral vector containing the CD19/CD20 CAR and tEGFR transgenes was prepared, and had this material prepared at Lentigen, (i.e., a comparator vector). Leukapheresis material from 3 different donors was processed following CD62L+ enrichment and activation steps described herein. T cells were transduced at day 3 with CC310B and expanded in culture until day 12, following manufacturing protocol herein. T cells were collected and stained with either anti-mouse F(ab)2 (Jackson ImmunoResearch) or anti-human EGFR antibodies against tEGFR. As shown in FIG. 14, cells stained with anti-mouse F(ab)2 or anti-human EGFR behaved similarly at a range of MOIs, suggesting that anti-F(ab)2 antibody can be used to accurately and directly measure CAR expression level on the cell surface. CAR detection with the anti-mouse F(ab)2 antibody is thus useful to measure CAR transduction—all process runs in this section were performed using anti-F(ab)2 antibody to measure CAR expression.

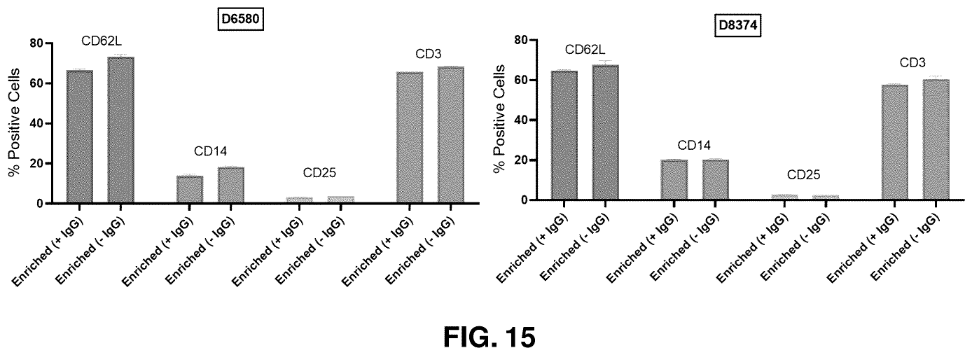

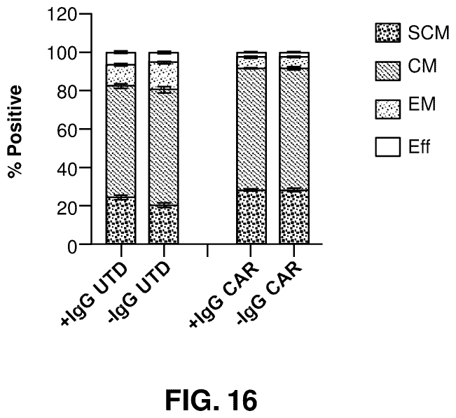

CD62L+ enrichment step. In two separate PD runs, it was investigated whether inclusion of IgG as a blocking reagent in the depletion step was helpful during the CliniMACS™ CD62L-selection step. Prior processes employed human IgG (Gammagard, Takeda) during CD62L enrichment to potentially increase the purity of the enriched cells. The intention was to decrease unwanted antibody binding (of the murine anti-CD62L monoclonal antibodies) to Fe receptors present on immune cells (such as B lymphocytes, dendritic cells, monocytes, macrophages, NKs, etc.). Side-by-side comparison of the enrichment process performed with and without IgG (Gammagard and Human IgG Affinity Purified Low Endotoxin, Innovative Research) in two different healthy donors (6580 and 8374, respectively), showed identical CD62L+ enrichment (FIG. 15). Additional markers were analyzed to determine the impact of the IgG on various cell populations. The percentage of CD3+, CD14+ and CD25+ cells is essentially the same after enrichment regardless of whether IgG is included (FIG. 6A and FIG. 6B, respectively). Cells selected with and without IgG were activated, transduced with CD 19/20 CAR lentiviral vector and expanded following the manufacturing process to explore potential impact on T cell Immunophenotype. As shown in FIG. 16, no differences were observed with or without IgG during CD62L+ selection. Results showed that IgG does not have impact on the purity of the enriched cells and therefore will not be used as a reagent in the manufacture process.

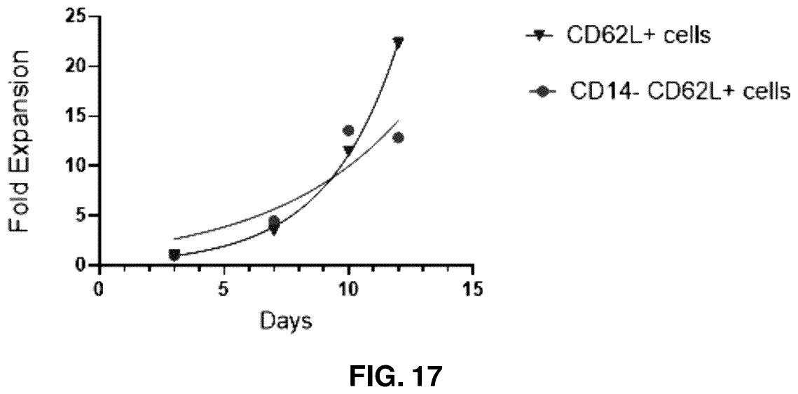

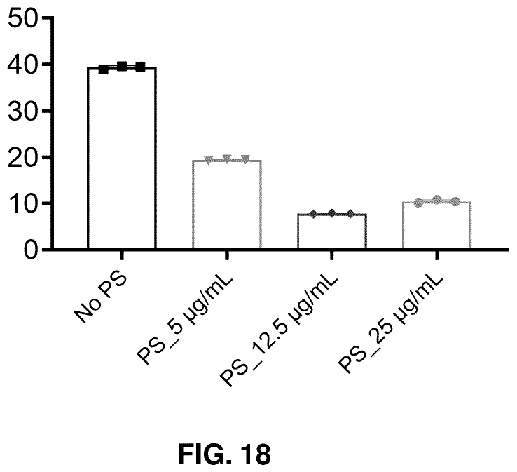

CD14+/CD25+ depletion step. As noted herein, removal of CD14/CD25-positive cells was an optional step, and in 3 out of 8 clinical material manufactured, this step was not included though clinical outcomes were favorable. It was therefore evaluated if the step to deplete CD 14+ and CD25+ cells after CD62L+ enrichment was critical for successful transduction and expansion of CART cells. Others have shown that a high monocytes content can inhibit the activation and expansion of T cells, likely because of the immunosuppressive functions of the CD14+ cell, in addition to the unspecific sequestration of the CD3/CD28 activation beads by the monocytes (Stoncek, 2016; Wang 2021). To explore this potential immunosuppressive effect, a side-by-side manufacturing process was performed comparing the expansion and transduction efficiency of the T cells with or without the depletion step. Similar level of T cells fold expansion, following TransAct activation, was observed in CD62L+ enriched cells and CD62L+ enriched/CD14+ depleted cells at different timepoints after transduction (FIG. 17). Moreover, transduction efficiency has been assessed between the two processes. No difference in CAR expression level was observed between the cells processed with and without the depletion step (30.5% and 27.3%, respectively). The percentage of monocytes during T cell culture was also assessed in PD runs using T cells from 3 different donors. Results showed that after the initial CD62L+ enrichment step, the percentage of CD14+ cells in the total population at day 3 (day of transduction) had dropped to ≤3%), and did not interfere with activation and transduction of the T cells.

TABLE 12
Percentage of CD14+ cells at day 1 and day 3 in 3 different donors.

| Product | | | | | |
|---|---|---|---|---|---|
| Metric | Method | | D0597 | D6580 | D1114 |
| Fold | Calculated based on cell number | | 22 | 19 | 31 |
| Expansion | seeded at Day 3 | | | | |
| Viability | NucleoCounter | | 88% | 93% | 98% |
| CAR⁺ | CAR⁺ by F(ab)₂ detection Flow | | 31% | 40% | 57% |
| expression | Cytometry | | | | |
| Tumor | IFN-γ | CD19⁺ Raji | 2456 pg/mL | 339 pg/mL | 2852 pg/mL |
| reactivity | secretion by | CD20⁺ Raji | 1872 pg/mL | 431 pg/mL | 4154 pg/mL |
| | ELISA | | | | |
| | (10:1) | | | | |
| VCN | ddPCR | | 2.0 | 1.9 | 2.1 |
| CD3% of Live | Flow Cytometry | | 97% | 94% | 98% |
| CD4% of | Flow Cytometry | | 45 | 40 | 61 |
| CD3 | | | | | |
| CD8 of CD3 | Flow Cytometry | | 53 | 54 | 37 |
| CD45RA⁺ | Flow Cytometry | | 33 | 43 | 33 |
| CD62L⁺ | | | | | |
| (Naive) | | | | | |
| CD45RA | Flow Cytometry | | 61 | 51 | 63 |
| CD62L⁺ (CM) | | | | | |
| CD45RA | Flow Cytometry | | 5 | 4 | 3 |
| CD62L (EM) | | | | | |

In total, these data support that the CD14 depletion step is not required to allow T cell transduction and expansion, and ImmPACT Bio will not perform the CD14+/CD25+ depletion step for clinical manufacturing.

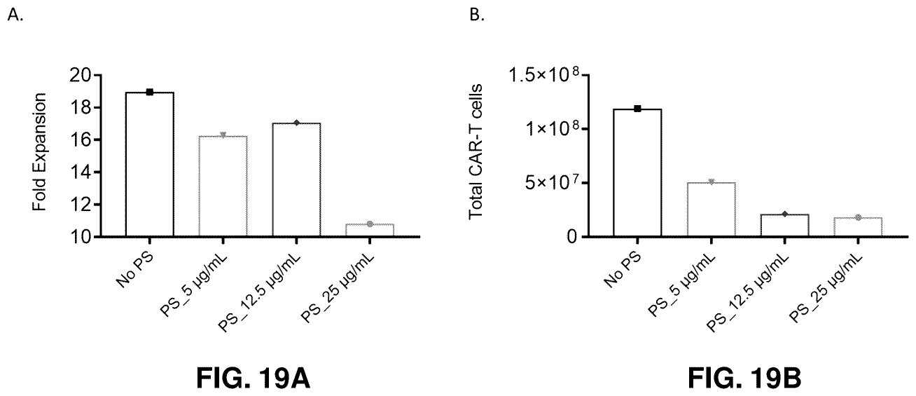

Transduction. It was then tested whether the addition of protamine sulfate, used as a transduction enhancer is necessary to achieve a consistent T cell transduction. T cells from donor 6580 (D6580) were enriched and activated as described herein. At day 3, T cells were transduced with or without protamine sulfate. Cells were expanded and collected for testing at day 12. Flow Cytometry results showed that the percentage of CAR+ cells is higher without protamine sulfate than in the groups transduced with protamine sulfate. In addition, protamine sulfate had a detrimental effect on cell expansion and therefore on the final yield of CAR+ cells (collected at day 12) (FIG. 19A and FIG. 19B, respectively).

Process Development Runs. To test the manufacturing changes adopted by the sponsor, leukapheresis material of 3 healthy donors was used to manufacture 3 lots of IMPT-314B. The parameters assessed by the studies were: a) Cell expansion; b) transduction efficiency measured by flow cytometry and vector copy number; (c) Potency of the CD19/20 CAR T cells; and d) immunophenotyping. Results from these studies, compiled from 3 healthy donors, are described in the sections below.

Fold-expansion and viability of 3 different IMPT-314 lots. Transduced T cells were sampled on days 7, 10, and 12 of the manufacturing process for assessment of CD3 and CAR expression using flow cytometry. Cell count and viability was assessed using a Nucleocounter™ on days 1, 3, 7, 10, and 12 of the process. During the manufacturing process days 3 to 12, transduced T cells expanded on average 5- to 12-fold. Cell viability remained greater than 80% throughout the entire process.

IMPT-314 showed stable and high transduction efficiency. Transduced T cells were collected to assess CD19/20 CAR expression. On days 7, 10, and 12 of the PD runs, CAR expression was measured using anti-F(ab)2 antibody. High level of CAR expression was detected (55-70%) and remained stable from day 7 to day 12 of the process.

Vector copy number. Transduction efficiency was further assessed by measurement of VCN per transduced cell. T cells were harvested on day 12 at the end of the manufacturing process and total number of copies of vector integrated into the host genome was quantified by droplet digital PCR and normalized to percentage of CAR positive cells reported by flow cytometry. VCN ranged from 2 to 3 per transduced cell in all 3 PD runs.

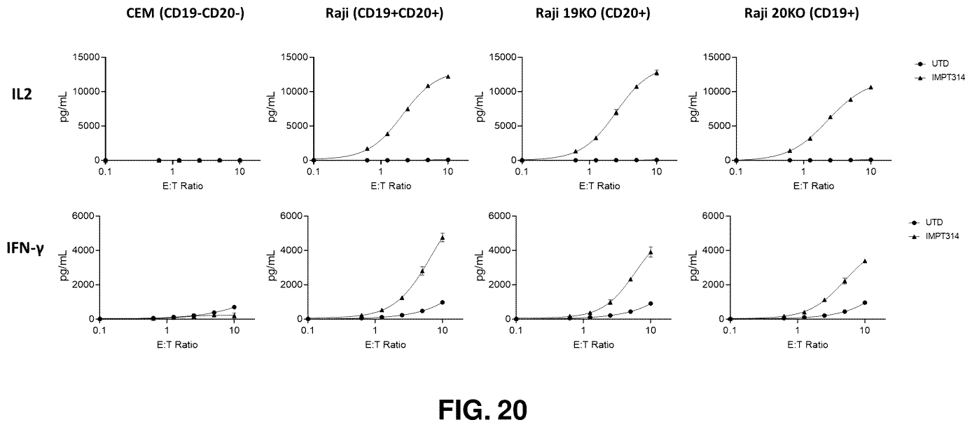

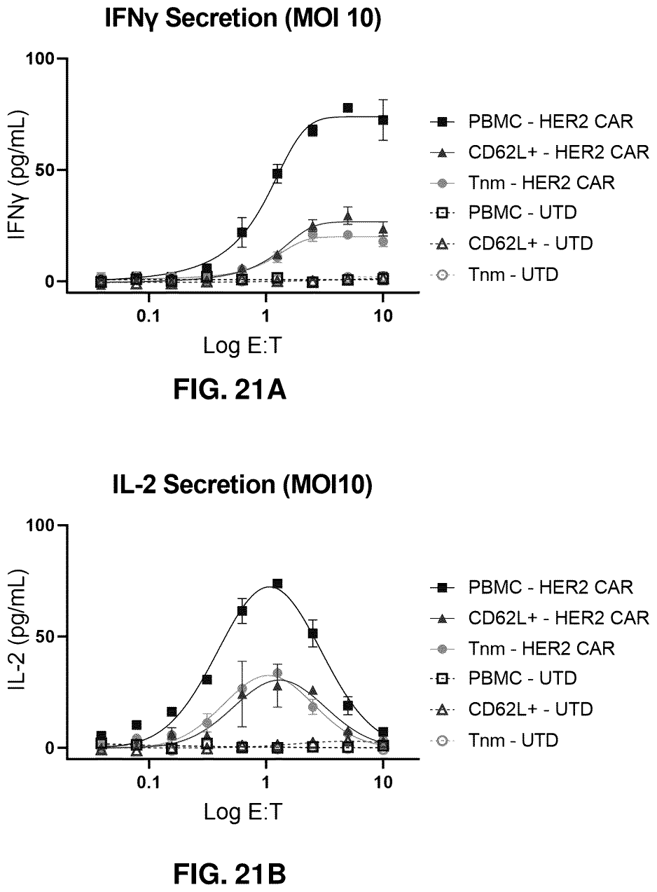

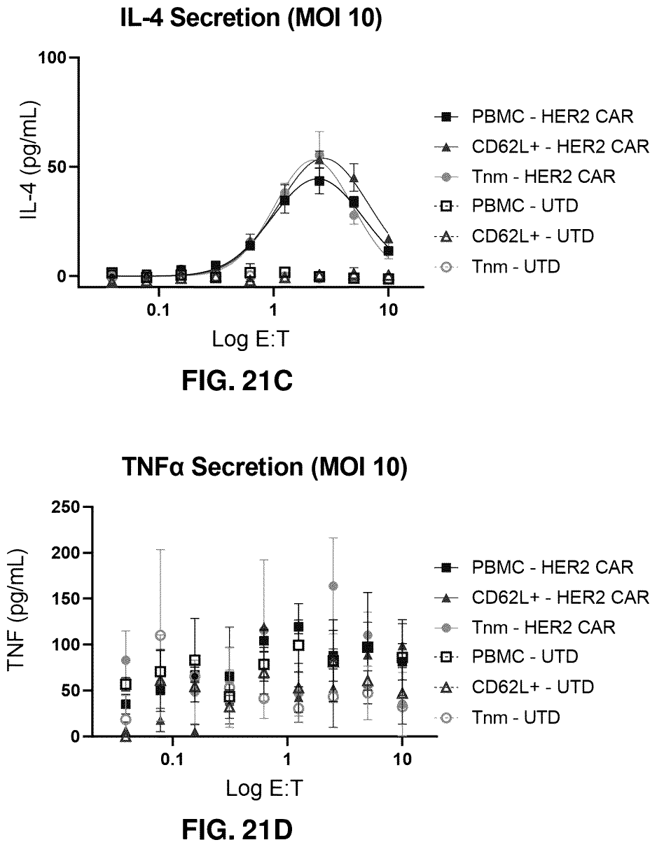

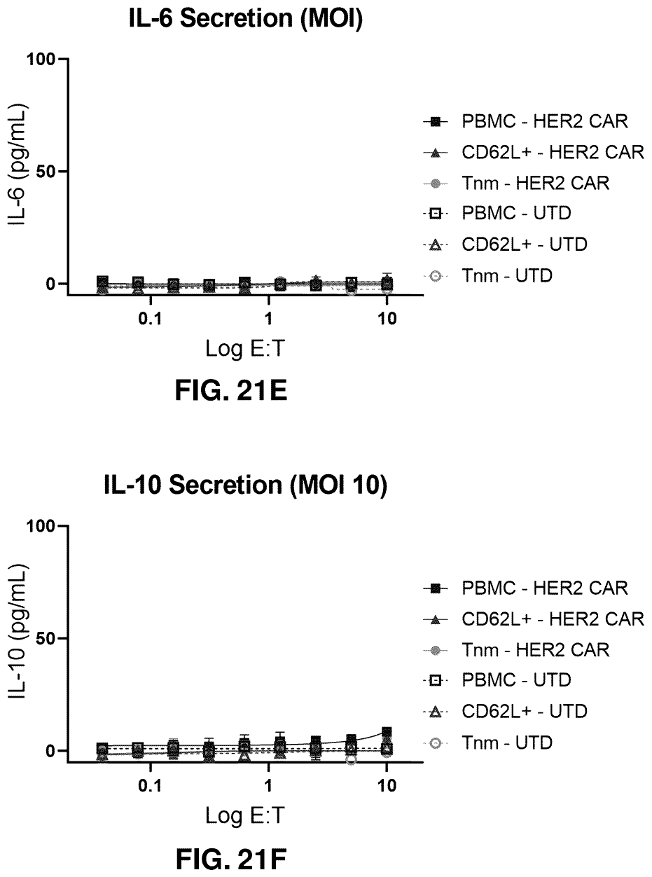

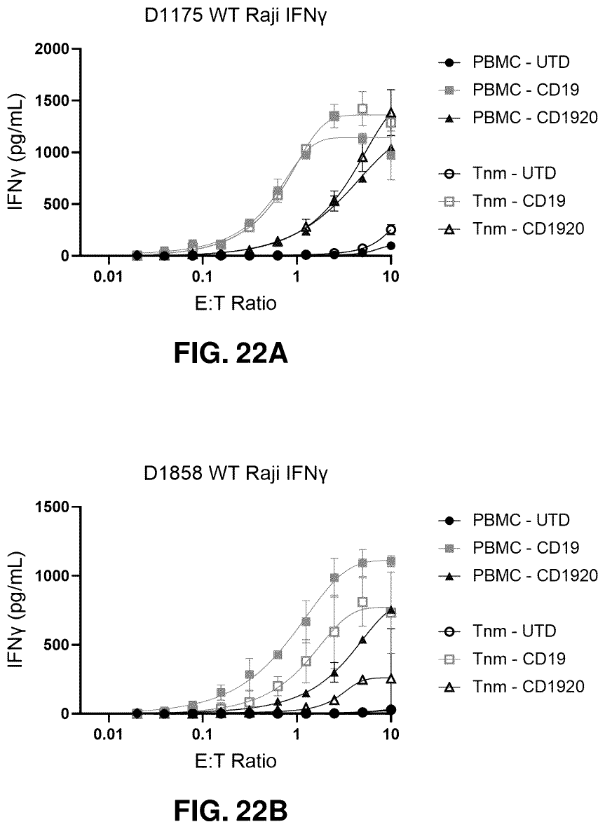

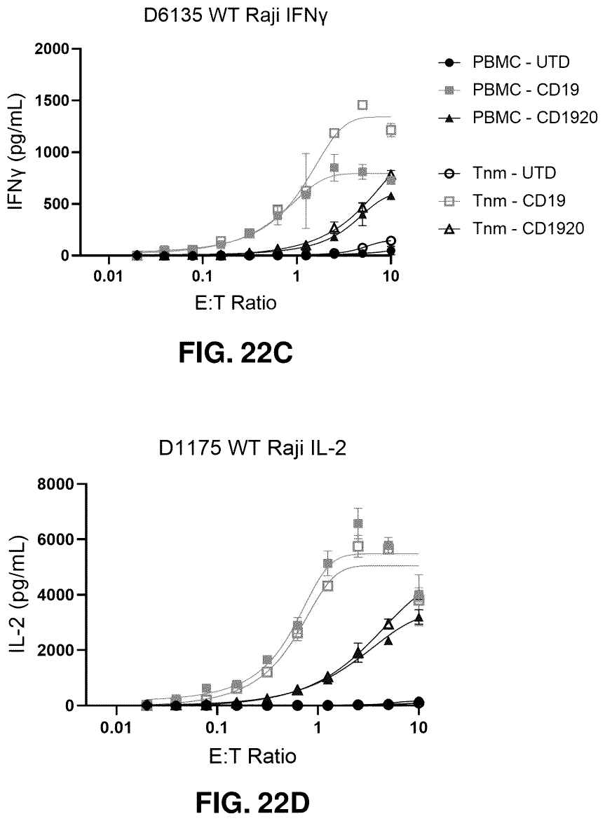

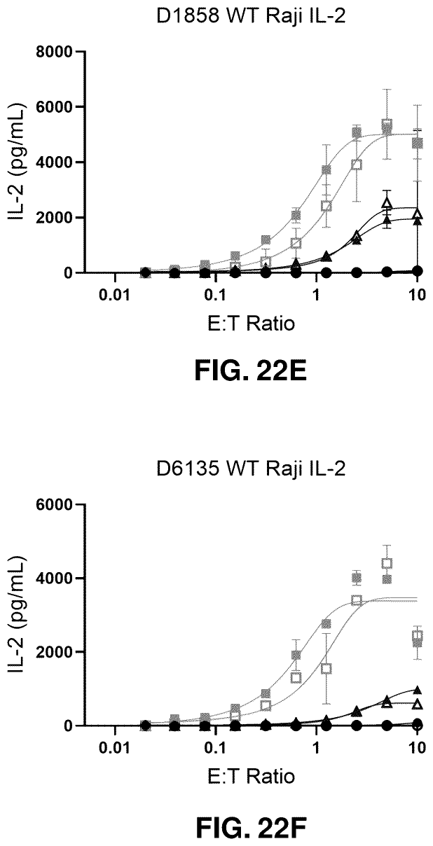

Potency of the CD19/20 CAR drug product. After characterization, potency of the CD19/20 CART cells was evaluated using cryopreserved CART cells at the end of the process run to evaluate the ability of CART cells to secrete inflammatory cytokines when co-cultured with antigen positive target cells. To examine efficacy of each scFv domain of the bispecific CD19/20 CAR, Raji cells were engineered to express 1) CD19 antigen only, and 2) CD20 antigen only. CEM cells, which do not express CD 19 or CD20, were used as a negative control. Freshly thawed CD19/20 CART cells and target cells were co-cultured at varying ratios of effector to target (E:T) in the absence of exogenous cytokines and incubated for 16-18 hours. After incubation, supernatants were analyzed for secretion of IL-2 and IFN-γ using R&D Systems ELISA kits. FIG. 20 demonstrates CAR T cell secretion of both proinflammatory cytokines after co-culture with CD19 and/or CD20 positive Raji cells. In comparison, untransduced T cells did not secrete IFN-gamma or IL-2 when co-culture with Raji cells. IMPT-314 showed robust cytokine secretion against target cell expressing CD19/C20 and single antigen (both CD19 only and CD20 only).

Immunophenotyping. On day 12, untransduced and CD19/20 transduced cells were immunophenotyped with a panel of antibodies to detect CD62L, CCR7, CD45RA, CD45RO, CD3, CD4, and CD8. At the end of the manufacturing process, >80% of untransduced and CAR T cells remained stem cell memory/naive and central memory, consistent with initial selection for CD62L positive cell population.

Flow Cytometry panel also showed a high percentage of CD3+ cells, above 96% and a similar percentage of CD8 and CD4 potency assays.

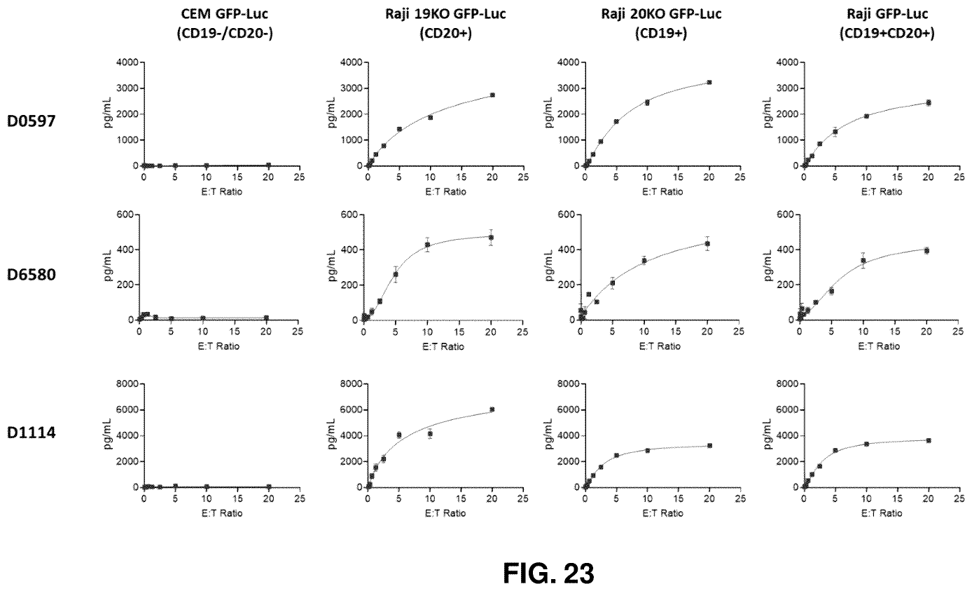

Potency of the CD 19/20 CAR T cells was evaluated using cryopreserved CAR T cells to evaluate the ability of CAR T cells to secrete inflammatory cytokines when co-cultured with antigen positive target cells. Briefly, freshly thawed IMPT-314 and target cells were co-cultured at varying ratios of E:T in the absence of exogenous cytokines and incubated for 16 to 18 hours. After incubation, supernatants were analyzed for secretion of IFN-γ using R&D Systems ELISA kit. FIG. 23 shows increased secretion of IFN-γ by CAR T cells after co-culture with CD19 and/or CD20 positive Raji cells at increasing E:T ratios, demonstrating specific activity of both the anti-CD19 and anti-CD20 element of IMPT-314.

The data from runs using this closed process demonstrated that the manufactured drug products met all of the release criteria set for the future GMP IMPT-314. The Drug Products showed excellent expansion and viability. The expression of the CAR was within range and stable, and the VCN was in the expected range. In each case, the IMPT-314 products had a high level of purity, with CD3+ cell content above 95%, with desirable central memory and naive content. Finally, the products specifically target tumor cells expressing CD19 and/or CD20, meeting the potency requirement.

Stability Studies. The studies to determine the stability of IMPT-314 final cell product are summarized in Table 13 for short-term and Table 14 for long-term stability studies, respectively. The long-term stability of the drug product is assessed in 3 healthy donors. Aliquots (10-20 ml) from Drug Product are collected into several (6-8) representative containers (CryoMACS Freezing bags).

TABLE 13
Planned Drug Product Short-Term Stability Studies
Drug Product Stability Study - Short Term

Time Points

| | | 1 | 2 | 4 |
|---|---|---|---|---|
| Test | Method | hour | hours | hours |
| Viability | NucleoCounter | [symbol] | [symbol] | [symbol] |
| | Acridine Orange/DAPI | | | |
| Recovery | NucleoCounter | [symbol] | [symbol] | [symbol] |
| | Acridine Orange/DAPI | | | |
| % CAR+ | Flow Cytometry | [symbol] | [symbol] | [symbol] |

TABLE 14
Planned Drug Product Long-Term Stability Studies
Drug Product Stability Study - Long Term

| | | Time Points | | | |
|---|---|---|---|---|---|
| | | 1 | 2 | 6 | 12 |
| Test | Method | month | months | months | months |
| Viability | NucleoCounter | [symbol] | [symbol] | [symbol] | [symbol] |
| | Acridine Orange/DAPI | | | | |
| VCN | ddPCR | [symbol] | [symbol] | [symbol] | [symbol] |
| % CAR+ | Flow Cytometry | [symbol] | [symbol] | [symbol] | [symbol] |
| IFN-γ | ELISA | [symbol] | [symbol] | [symbol] | [symbol] |
| secretion | | | | | |
| CTL | Flow Cytometry | [symbol] | [symbol] | [symbol] | [symbol] |
| Sterility | Gram Stain and | | | | [symbol] |
| | Bacterial/Fungal | | | | |
| | culture | | | | |

### Example 16: Anti-CD19/CD20 Bispecific CARs Containing Variable Hinge Regions

To ensure efficacy and safety of CAR-T cell therapy, it is important to understand CAR's structure-activity relationship. To clarify the role of hinge domain in CAR and CAR-T cell function, different CD19/CD20 bispecific CAR constructs were generated. Expression levels and antigen-specific activity on CAR-T cells was analyzed.

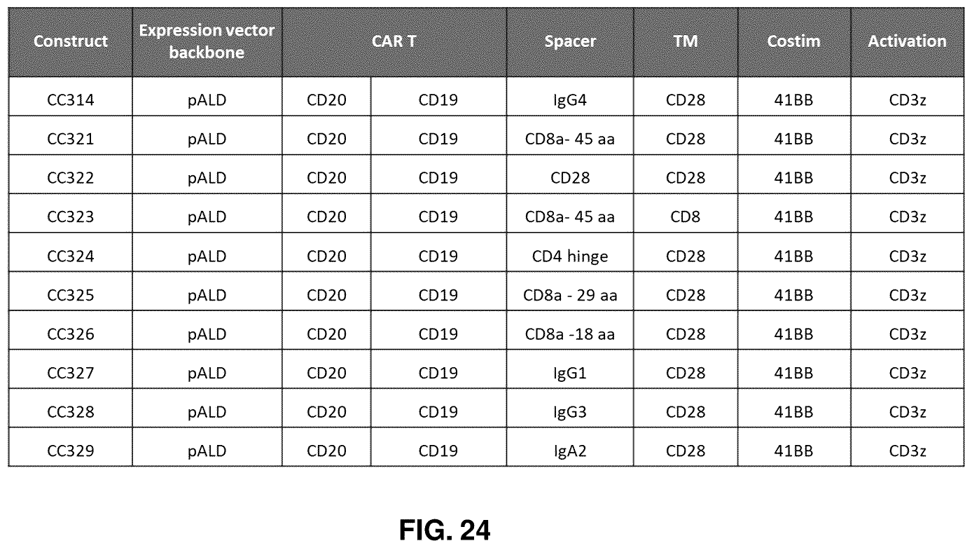

CC314B is a bi-specific lentiviral vector that expresses the antigen receptor that binds CD20 and CD19 antigens. In CC314b the scFVs FMC63 and Leu16 provide the targeting function by specifically recognizing the CD20 and CD19 tumor antigens. The hinge is derived from the human immunoglobulin IgG4 and the transmembrane domain is derived from the CD28. The IgG4 hinge provides a flexible link between the scFvs and the transmembrane domain. The hinge allows the antigen-binding domain to accommodate different orientations to facilitate antigen recognition. We have modified the hinge domain (IgG4) in CC314B, to generate nine CAR variants whose hinge domains originated from CD8α, CD28, CD4, IgG1, IgG3 and IgGA2. The intracellular signaling domains are derived from human 4-1BB and CD3ζ and are shared between all ten constructs (FIG. 24).

Flow Cytometry Analysis of CAR Expression. T cells from two healthy donors were transduced with CC314B- to CC329B, as described in FIG. 24. On Day 12 post transduction cells were harvested to assess the percentage of CD19/20 CAR+ T cells by flow cytometry. Briefly, 100,000 cells (both CAR transduced and untransduced controls) were harvested into 96 well v bottom plates and washed 3 times with staining buffer containing 0.2% BSA. Cells were resuspended in 100 μL of staining buffer and antibodies were added to the cell suspension and incubated at 4° C. for 30 minutes. After incubation, cells were washed 3 times with staining buffer, and secondary antibodies (Table 5) were added at a dilution of 1:200, and cells incubated at 4° C. for 30 minutes in the dark. After incubation, cells were washed 3 times with staining buffer and cells were resuspended in 200 μL of staining buffer and analyzed using the Attune N×T flow cytometer (Thermo Fisher). For analysis, cells are gated on forward scatter area (FSC-A) and side scatter area (SSC-A) to gate the cell population, then FSC-A and forward scatter height (FSC-H) to gate the singlets. Negative control includes staining of untransduced T cells (CAR-negative).

TABLE 15
Detection antibodies used in Flow cytometry for T cell phenotype analysis:
Antibody Panel

| Ab | Fluorophore | Clone | Manufacturer/Catalog # |
|---|---|---|---|
| CD3 | PerCP-Cy5.5 | SK7 | BioLegend: 344808 |
| CD4 | APCCy7 | RPA-T4 | BD: BDB557871 |
| CD8 | PE/Cy7 | SK1 | BioLegend: 344712 |
| CD45RA | FITC | HI100 | BioLegend: 304106 |
| CD62L | e450 | DREG-56 | eBioscience: 50-245-999 |

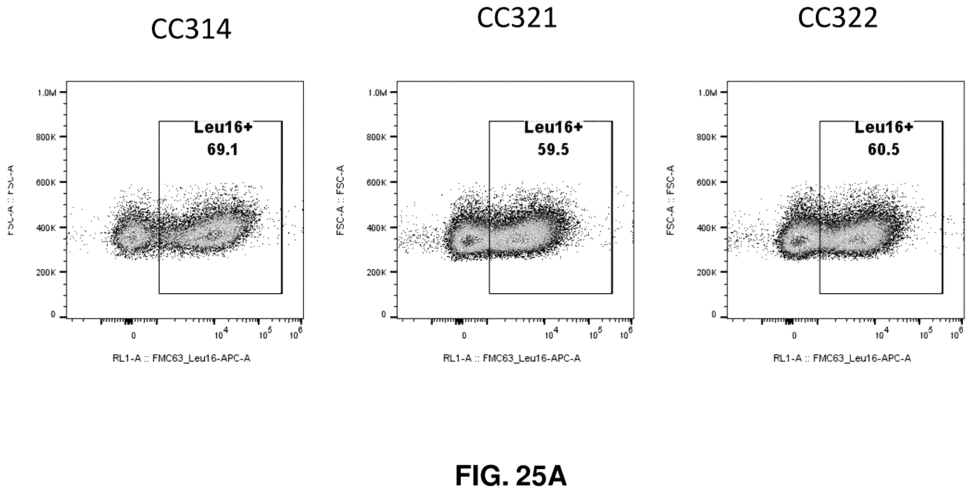

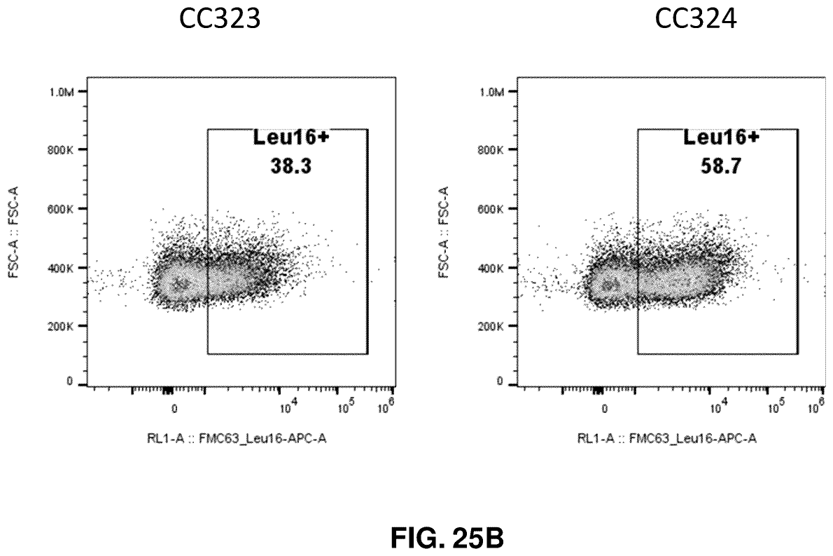

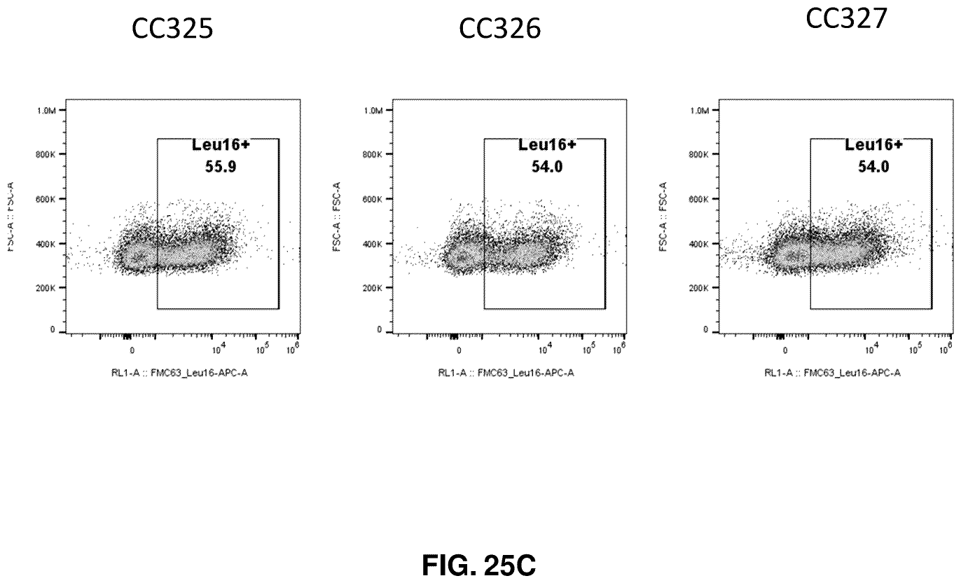

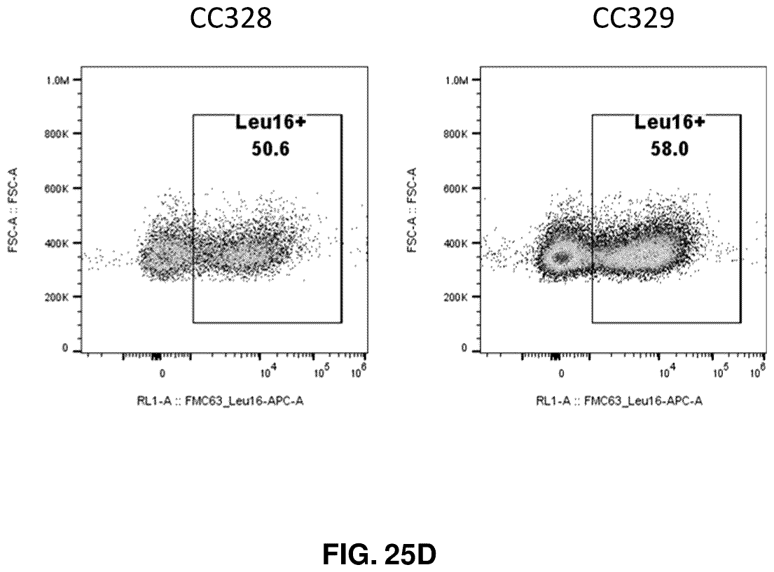

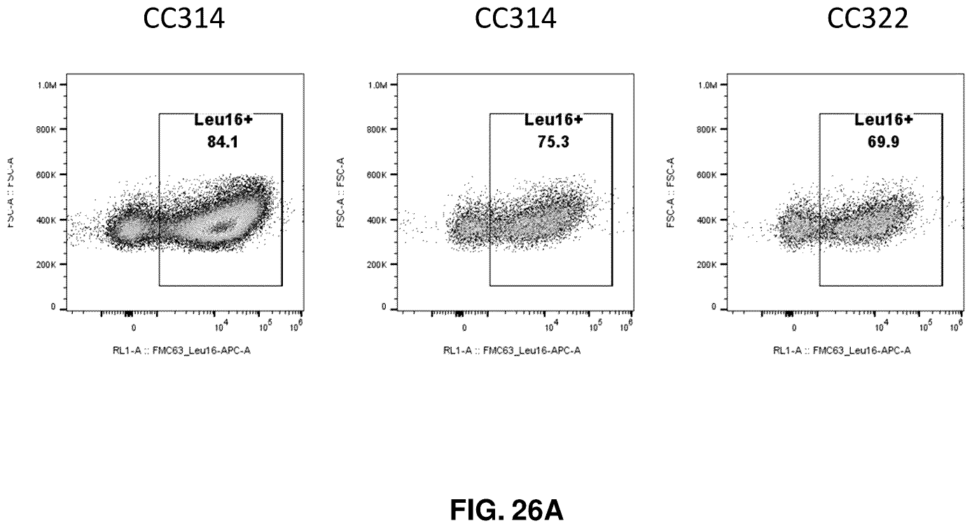

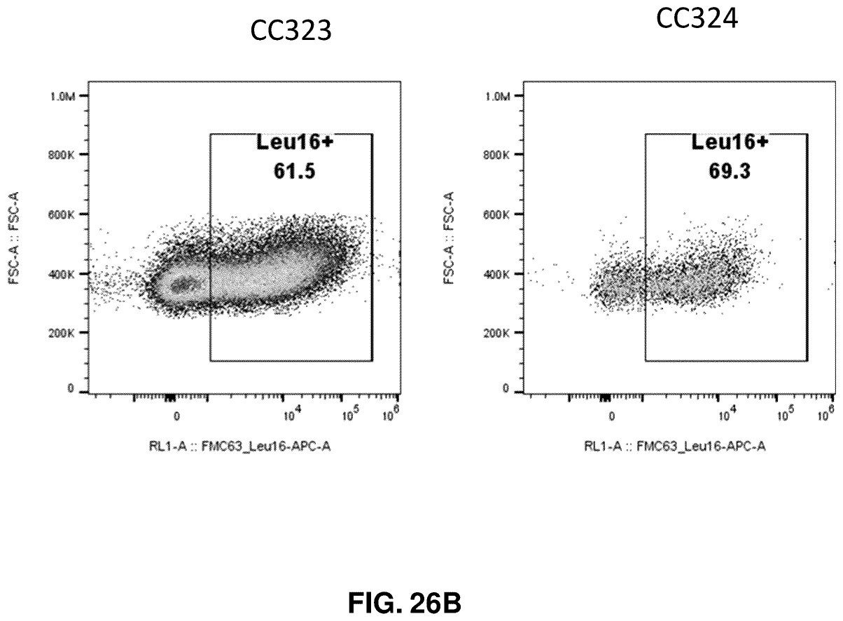

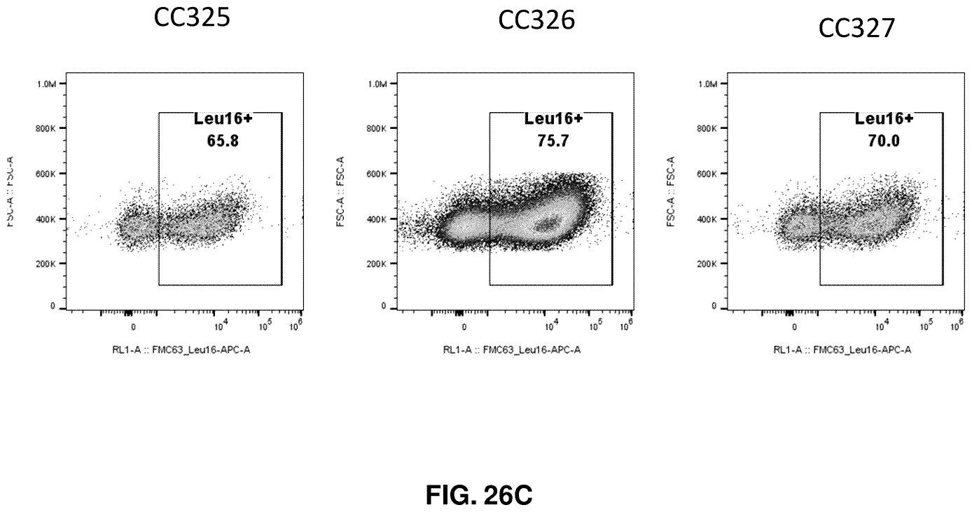

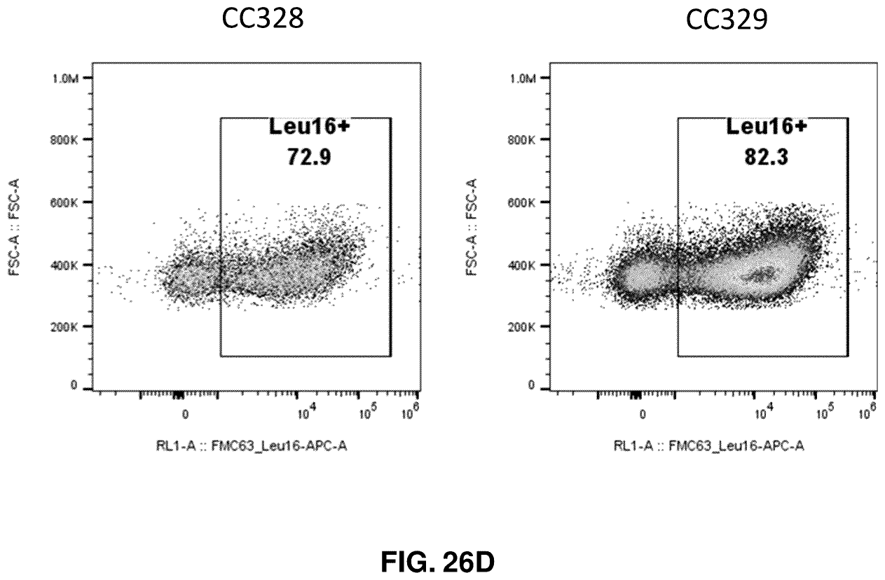

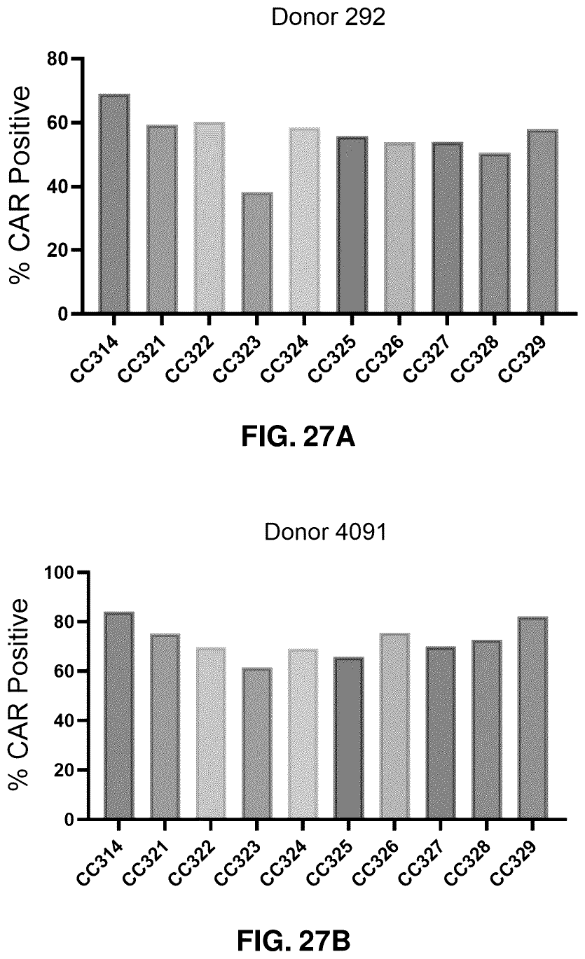

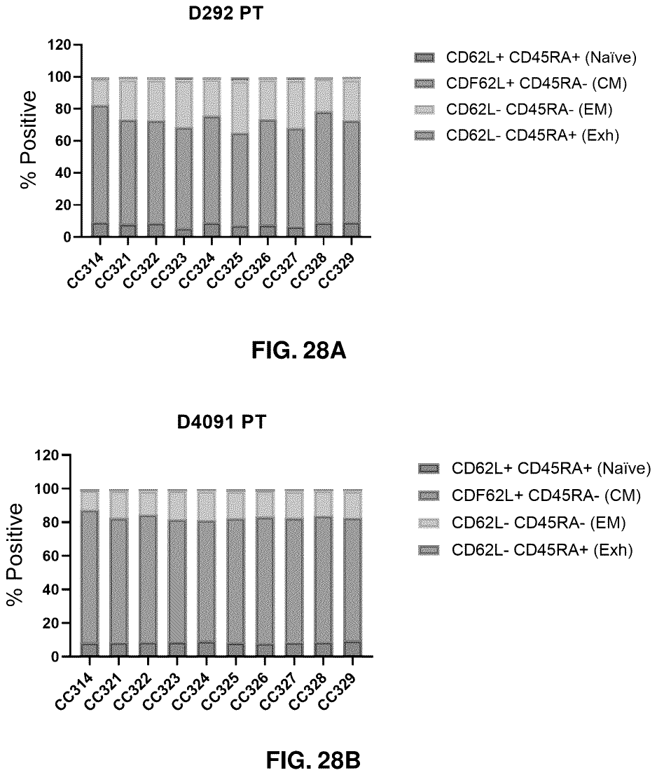

FIG. 25A-FIG. 25D, FIG. 26A-FIG. 26D, and FIG. 27A-FIG. 27B show expression levels on the surface of T cells as measured by anti-mouse Fab staining (Goat anti-ms IgG (H+L)-AF647Invitrogen). Results were obtained from two different donors.

### Flow Cytometry Analysis of T-Cell Phenotype.

T cell phenotyping was performed on the day of harvest. Cells were pelleted and stained using antibodies listed in TABLE 15. Post-staining, cells were analyzed using Attune N×T flow cytometer (Thermo Fisher). Single color controls and isotype controls were also collected for each fluorophore. On day 12 of culture, T cell phenotype was assessed by flow cytometry (FIG. 28). There were not any statistically significant difference between T cells expressing either by-specific CAR of different spacers for the following markers: CD62L and CD45RA. There was also not a statistically significant difference between T cells expressing the different constructs in the percentage of naive, effector memory, or central memory T cells as defined by CD45RA. T cells were composed of −70-85% central memory cells and 15%-20% effector memory cells.

### T Cells Cytotoxicity Assay

On Day 12, T cells transduced with LLV constructs CC341B to CC329B and untraduced (UTD) cells were plated with target WT Raji cell line which express Firefly Luciferase green fluorescent protein (FFluc GFP) and both CD19 and CD20 cell surface receptors, and engineered Raji lines which are either CD19 deleted, CD19−/CD20+(CD19KO) or CD20 deleted, CD19+/CD20− (CD20KO). Co-culture was performed in a 96-well plate. Luciferase expressing Raji WT, Raji CD19KO, Raji CD20KO or CEM cells were plated at 10,000 cells per well in a 96-well plate. Chimeric antigen receptor positive T cells or untransduced control were added at a starting effector-to-target ratio of 2.5:1 with a serial 2-fold dilution for a total of 7 dilutions. After 72 hours of coculture, CAR T cell cytotoxicity was measured by luciferase signal using Bio-Glo™ Luciferase Assay system (Promega) and quantified by Varioskan (ThermoFisher).

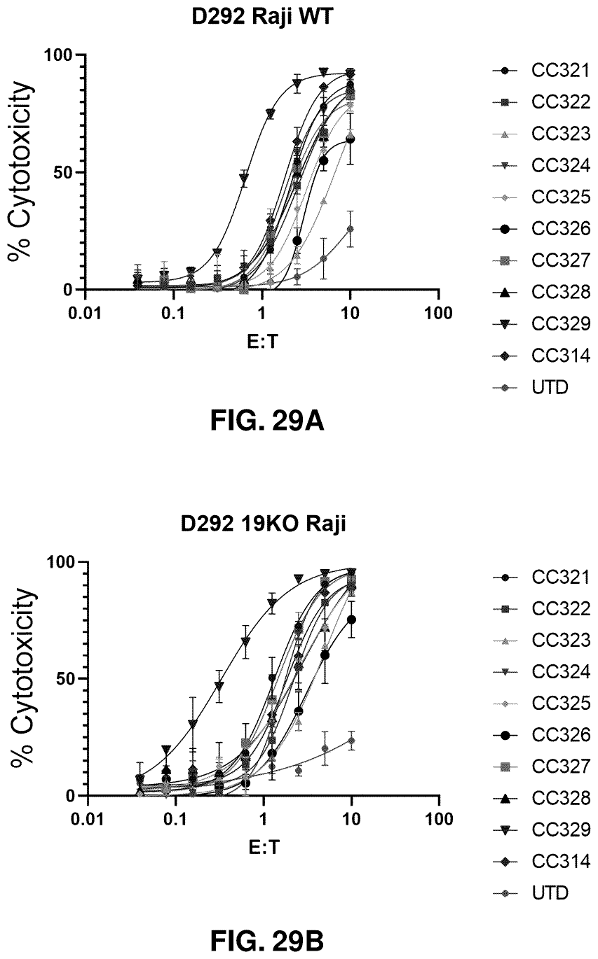

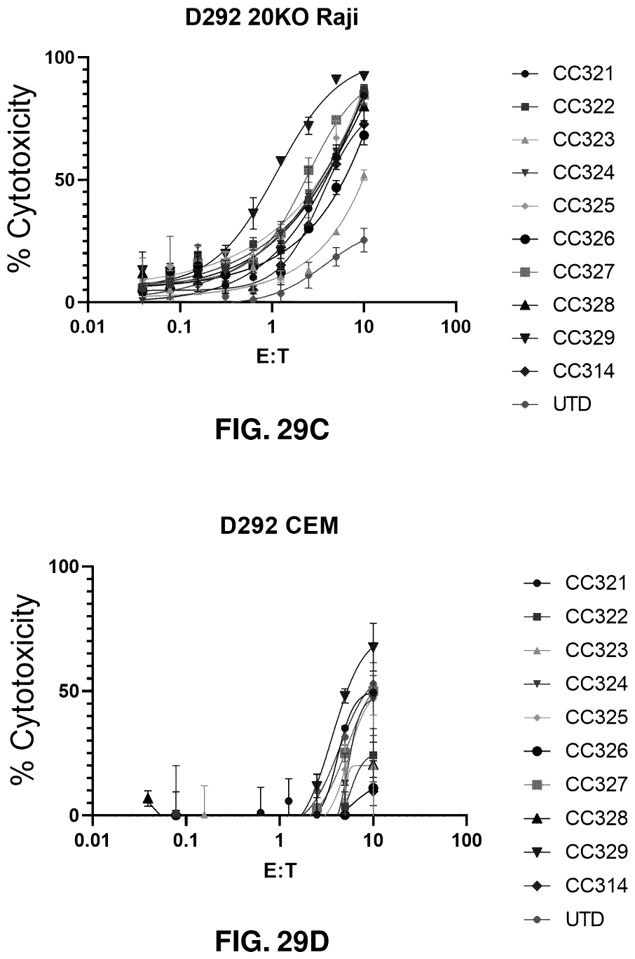

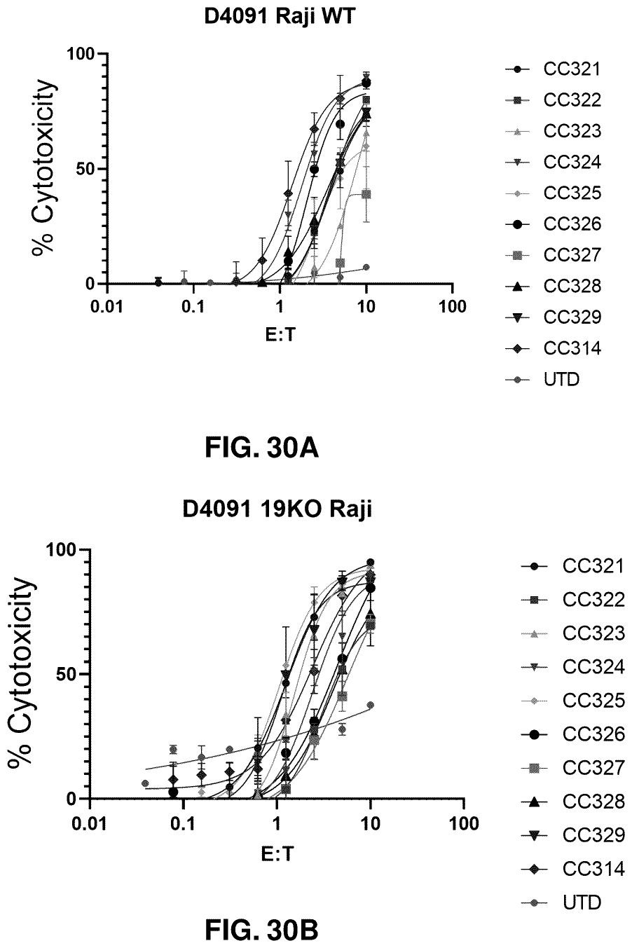

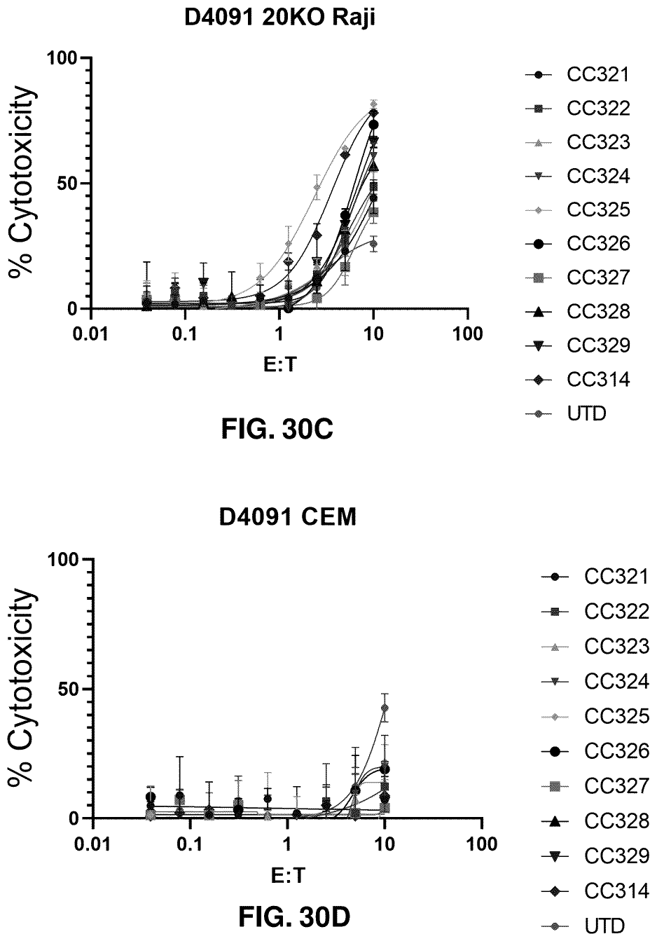

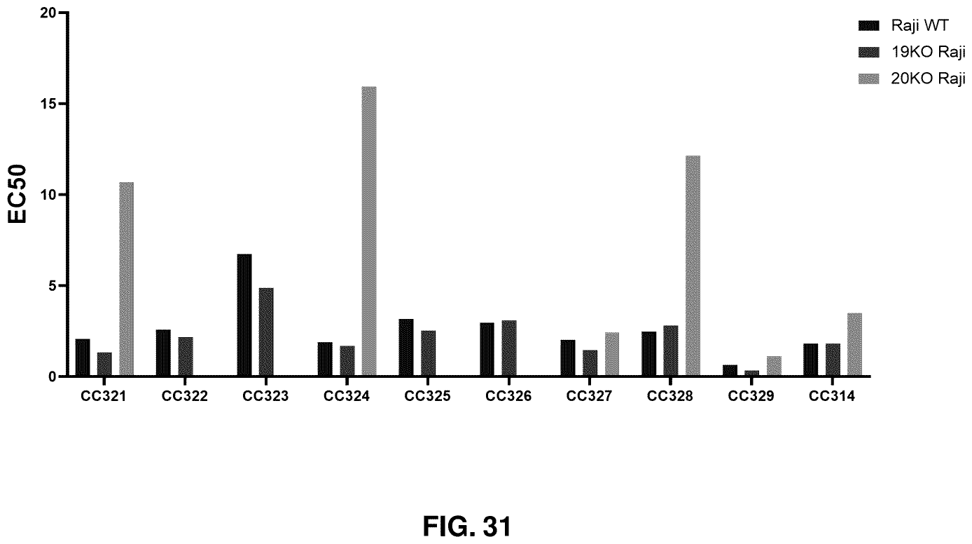

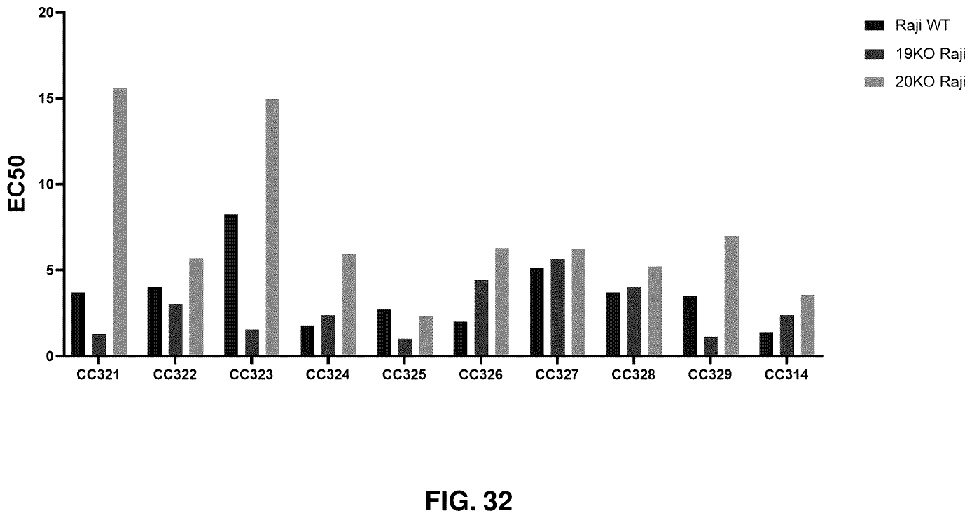

The cytotoxicity potential was measured by luciferase signal from the remaining viable CD19+, CD20+, CD19+CD20+ or CD19−CD20− target cells remaining in co-culture. Across 2 donors, all constructs exhibited robust Raji-cell lysis compared to the UTD after a 72-hour coculture as shown in FIG. 29A-FIG. 29D, FIG. 30A-FIG. 30D, FIG. 31, and FIG. 32. When co-cultured with CD19−CD20− CEM cells, both UTD and other constructs showed minimal cytotoxicity that was suspected to be due to allogeneic effect. Statistical comparison of cytotoxicity between UTD and transduced cells were performed at each E to T ratio EC50 was also plotted.

### Cytokine Secretion—Potency Assay.

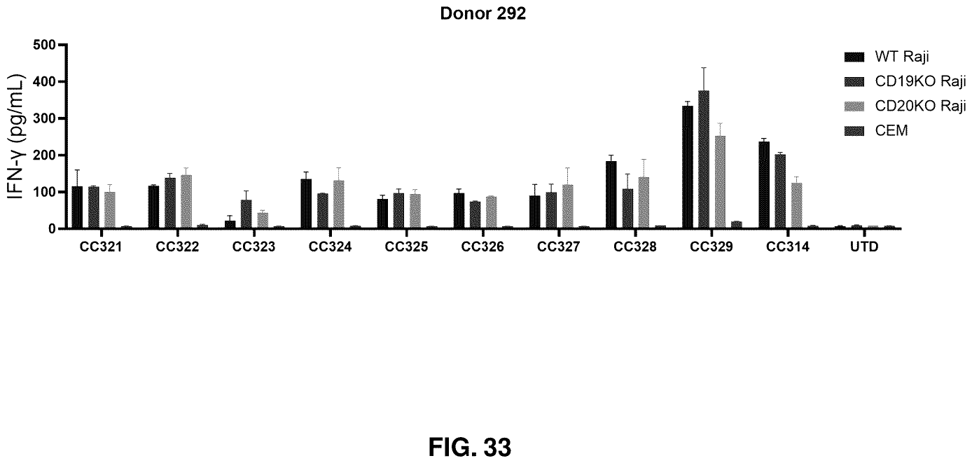

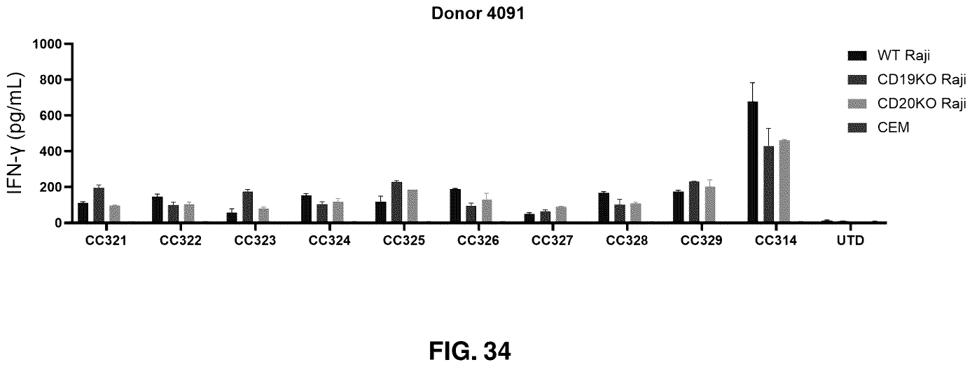

Enzyme-linked immunosorbent assays were performed on the test articles to quantify IFN-γ production to assess the response of the genetically modified TN/MEM in co-culture with antigen positive and negative tumor cells. The concentration of cytokines produced was compared with untransduced TN/MEM cells. The untransduced control produced minimal amounts IFN-γ when co-cultured with Raji, Raji CD19KO, Raji CD20KO, and CEM target cells. As expected, al bi-specific CAR constructs produced a significant amount of IFN-γ compared to the untransduced control when co-cultured with antigen-expressing cell lines: Raji, Raji CD19KO, and Raji CD20KO but not the antigen negative CEM cell line. The assay was performed in two donors as shown in FIG. 33 and FIG. 34. These results show that all constructs can recognize and respond to cells that have lost CD19 expression by producing proinflammatory cytokines and likewise, can recognize and respond to cells that have lost CD20 expression. The data further confirm that changing the hinge domain did not affect the production of IFN-γ as the response to the CD19+/CD20+ expressing target cells.

### Example 17: 8-Day Research Small-Scale Manufacturing Protocol

### Manufacturing Process

### Day 0: Leukapheresis Collection (Starting Material)

Autologous leukapheresis material was collected from healthy donors at pre-qualified apheresis centers. Whole blood was obtained from IIM donors. Whole blood was processed by diluting it in an equal volume of PBS+2% Fetal Bovine Serum and mixed. Cells were separated using Ficoll density gradient medium over a SepMate tube (Stemcell) by centrifuging at 1200×g for 30 minutes. PBMCs were collected from the buffy coat. Cell count and viability was performed on the incoming leukapheresis starting material and the extracted PBMCs.

### Day 1: Enrichment and Activation of TN/MEM Cells

The leukapheresis/PBMC product was enriched for CD62L+ cells using CD62L microbeads (Miltenyi Biotec) in LS columns with SuperMACSII Separator (Miltenyi Biotec) system. Briefly, the leukapheresis product was washed with CliniMACS buffer (phosphate buffered saline [PBS]/ethylenediaminetetraacetic acid buffer supplemented with 0.5% human serum albumin). Enriched CD62L+ cells were activated with Good Manufacturing Practice (GMP)-grade TransAct™ Human T cell expander CD3/CD28 (Miltenyi Biotec) at a 1:100 dilution, then cultured in OpTmizer media supplemented with GMP-grade recombinant human interleukin (IL)-2 (50 U/mL, Miltenyi Biotec) and IL-15 (0.5 ng/ml, Miltenyi Biotec) for 1 day at 37° C./5% carbon dioxide (CO2) in a humidified incubator.

### Day 2: Lentiviral Transduction

Activated CD62L+ cells were counted and diluted to a concentration of 1-1.5×10⁶ cells/ml. A lentivirus encoding the anti-CD19/anti-CD20 CAR was added to cells at a multiplicity of infection of 7. After adding the lentivirus, cells were incubated at 37° C. in 5% CO2 for 6 hours. Once the 6-hour incubation was completed, media containing 50 U/mL IL-2 and 0.5 ng/ml IL-15 were added to dilute lentivirus concentrations by half, lowering the final cell concentration to 0.75×10⁶ cells/mL. The cells were incubated at 37° C./5% CO2 in a humidified incubator overnight.

### Day 2, 4, 6: Media Feed

On Days 2, 4, and 6, fresh media supplemented with cytokines (Optimizer media supplemented with 50 IU/mL human IL-2 and 0.5 ng/mL human IL-15) was added to the culture and incubated at 37° C./5% CO2.

### Day 6: TransAct Wash and Cell Maintenance

On Day 6, transduced T cells were washed to remove residual TransAct and seeded with media supplemented with cytokines at a density of 0.5×106 cells/ml and incubated at 37° C./5% CO2 for an additional 2 days.

### Day 8: Harvest and Cryopreservation

On Day 8, cells were harvested and washed to remove media using PBS. The washed cells were formulated with CryoStor CS10 Freezing Solution (BioLife) and stored in vapor phase liquid nitrogen freezer.

### Patient Samples Acquired for Nonclinical Studies

All ANCA-associated vasculitis (AAV), idiopathic inflammatory myositis (JIM), systemic sclerosis (SSc), and multiple sclerosis (MS) donors were sourced from Sanguine Bio (Table 16).

TABLE 16
Summary Patient Samples Acquired for Nonclinical Studies

| Donor Status | Donor # | Disease-Relevant Current Medications |
|---|---|---|
| Healthy | D29014 | Not Relevant |
| | D127862 | |
| | D117048 | |
| ANCA-Vasculitis | D104848 | Mycophenolate |
| | D107120 | NA |
| | D107184 | Methotrexate, Mepolizumab |
| | D134939 | Methotrexate |
| | D137964 | Prednisone, Methotrexate |
| Dermatomyositis | D120323 | Hydroxychloroquine, Cetirizine |
| | D120088 | NA |
| | D110081 | Cetirizine, Montelukast |
| Anti-Synthetase | D123038 | Azathioprine, Tocilizumab |
| Syndrome | D89505 | Methotrexate |
| Systemic Sclerosis | D120771 | Hydroxychloroquine |
| | D135550 | Prednisone, Fluticasone |
| | D134319 | NA |
| Secondary- | D125933 | NA |
| Progressive | D132012 | Glatiramer acetate |
| Multiple Sclerosis | D136055 | Glatiramer acetate |
| | D128206 | Gileyna |
| | D100099 | Natalizumab |
| Relapsing-Remitting | D125980 | NA |
| Multiple Sclerosis | D125204 | Diroximel fumurate |
| | D96146 | Gileyna |
| | D138998 | NA |

Manufacturing of IMPT-514 at Research-Scale from Donors

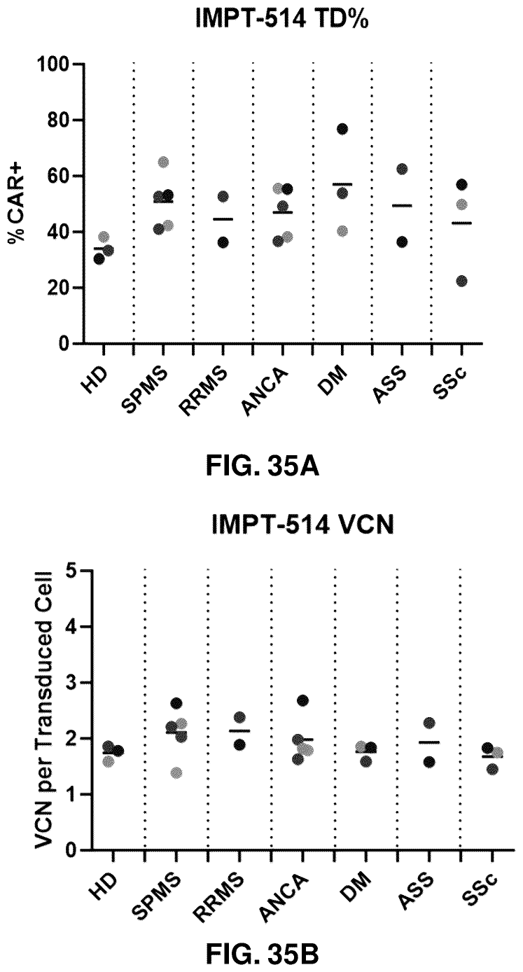

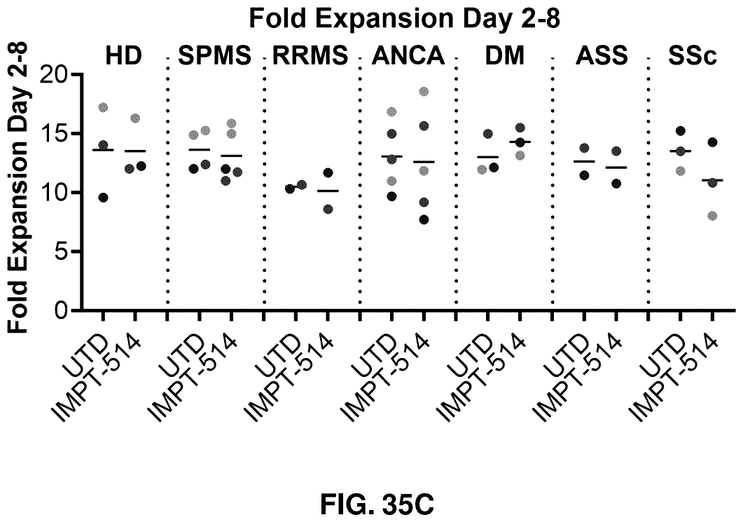

Many patients are treated with immunosuppressive medications, including glucocorticoids, methotrexate, mycophenolate, and azathioprine. To ensure that the manufacturing IMPT-514 from donors was feasible at research-scale, product derived from whole blood was used due to the limited availability of apheresis from these donors. IMPT-514 manufactured from donors were compared with 3 healthy donors, and the difference in their phenotype, expansion, and transgene integration was evaluated. The percentage of CAR positive cells was measured using an antibody against G4S linker (FIG. 35A and FIG. 35B). The expansion of IMPT-514 was comparable to healthy donors as shown in (FIG. 35C). Abbreviations: CAR=chimeric antigen receptor; HD=healthy donor; SPMS=secondary progressive multiple sclerosis; RRMS=relapsing remitting multiple sclerosis; ANCA=ANCA-associated vasculitis; DM=dermatomyositis; ASyS=anti-synthetase syndrome; SSc=systemic sclerosis. Note A: T-cells from autoimmune donors were harvested on Day 8 of the process and stained with anti-G4S linker antibody to assess the percentage of CAR-positive cells by flow cytometry. Untransduced T cells from the same donors were used as negative controls to show specificity of the staining. The values of CAR positivity were plotted in the figure above. Note B: Cell count was performed using LUNA FX7 automated cell counter (Logos Biosystems) and fold change from Day 2 to Day 8 were shown in the graph. B. Fold change from Day 3 to Day 12 were shown in the graph.

In total, IMPT-514 product was manufactured at research-scale using blood from 3 healthy donors and autoimmune donors. The research-scale IMPT-514 product manufactured using CD62L+ cells as starting material, isolated from autoimmune donors had similar CAR expression and cell fold expansion to that observed in product derived from healthy controls.

### Immunophenotyping and VCN

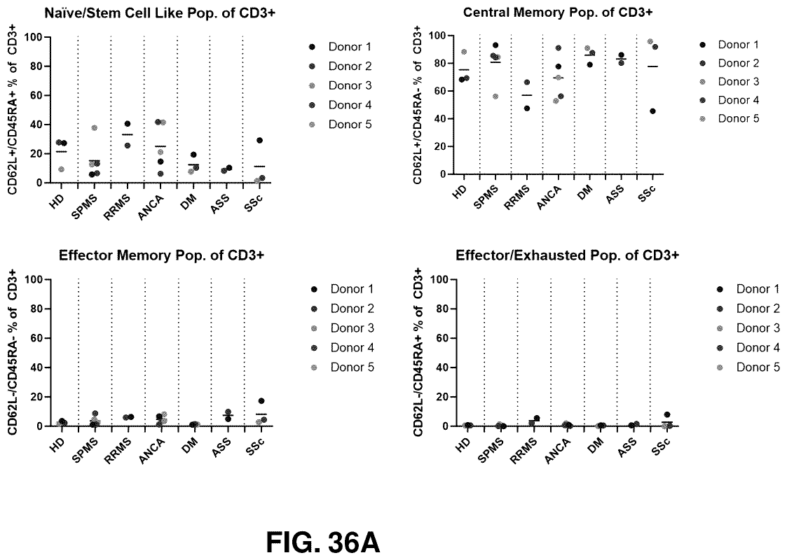

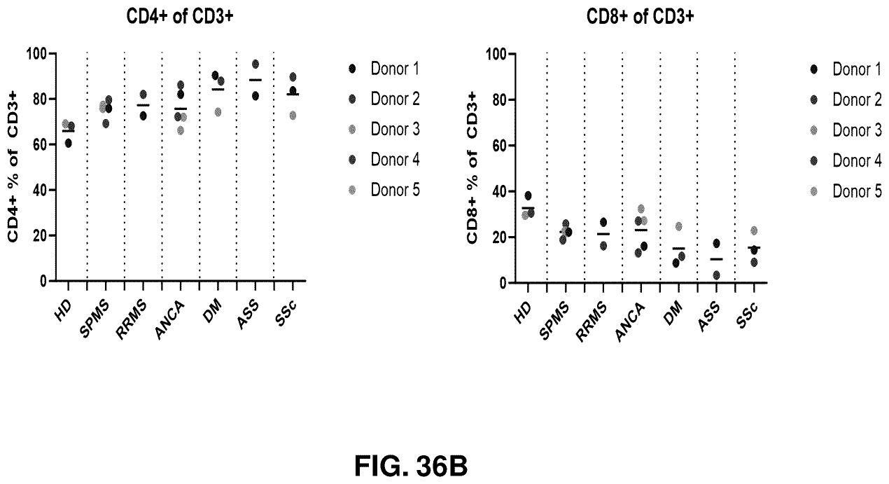

It was important to establish that the IMPT-514 product derived from autoimmune donors has similar characteristics to the IMPT-314 product, particularly the immunophenotype and vector copy number (VCN). IMPT-514 product manufactured as described above (8-Day research small-scale manufacturing protocol) was stained using a phenotyping panel including antibodies against CD45RA, CD62L, CD4, and CD8. IMPT-514 products were >85% positive for CD62L-expressing cells. In particular, the population was predominately central memory (CD45RA⁻/CD62L⁺). This finding is consistent with the initial selection of CD62L⁺ positive cells and dependably, across many donors, illustrates that the manufacturing process maintains the largely naive/memory population for cells isolated from IIM donors, and healthy donors (FIG. 36A And FIG. 36B). Abbreviations: CAR=chimeric antigen receptor; HD=healthy donor; SPMS=secondary progressive multiple sclerosis; RRMS=relapsing remitting multiple sclerosis; ANCA=ANCA-associated vasculitis; DM=dermatomyositis; ASyS=anti-synthetase syndrome; SSc=systemic sclerosis. Note: T-cells were harvested on Day 8, at the end of the process and stained for CD45RA and CD62L. The predominant population was of the naive or central memory phenotype (>85%), which is consistent with selection of the central (CD62L) phenotype on Day 1.

Transgene integration analysis by ddPCR showed that T cells from autoimmune donors had comparable VCN than healthy donors' T cells, with VCN ranging from 1.58 to 2.28 (FIG. 35A-FIG. 35C). IMPT-514 manufactured using autoimmune donors' T cells showed VCN<5, in compliance with FDA guidelines.

### Potency Assessment

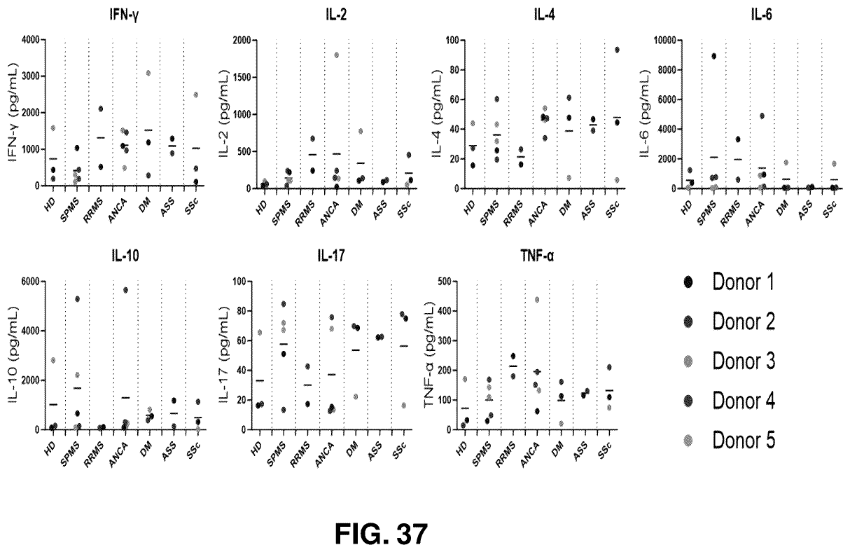

The potency of the IMPT-514 CAR T cells manufactured from CD62L+ cells isolated from autoimmune donors or healthy donors was measured by cytokine secretion assays and cytotoxicity after incubation with autologous B (CD19+/CD20+) cells. CAR T cell expansion was also evaluated in these assays. Assays were initially used to evaluate a comprehensive group of cytokines including interferon-gamma (IFN-γ), interleukin-2 (IL-2), IL-4, IL-6, IL-10, IL-17 and tumor necrosis factor-alpha (TNF-α) production. To achieve this a human Th1/Th2/Th17 CBA kit was employed to evaluate cytokine levels in supernatants 24 hours after IMPT-514 product was co-cultured at a 3:1 effector: target (E:T) cell ratio with autologous B cells. The observations demonstrate that the IMPT-514 products produce proinflammatory cytokines when incubated with target autologous B cells (FIG. 37). The secretion of proinflammatory cytokines 24 hours after co-culture of IMPT-514 and autologous B cells showed no significant differences in the levels of IFN-γ, IL-2, and TNF-α between autoimmune and healthy donors. Abbreviations: CAR=chimeric antigen receptor; HD=healthy donor; SPMS=secondary progressive multiple sclerosis; RRMS=relapsing remitting multiple sclerosis; ANCA=ANCA-associated vasculitis; DM=dermatomyositis; ASyS=anti-synthetase syndrome; SSc=systemic sclerosis.

IMPT-514 was incubated with autologous B cells at 3:1 E:T ratio for 24 hrs as cytokine secretion detected by CBA. Values represent means of technical repeats. Mean and standard deviation are plotted.

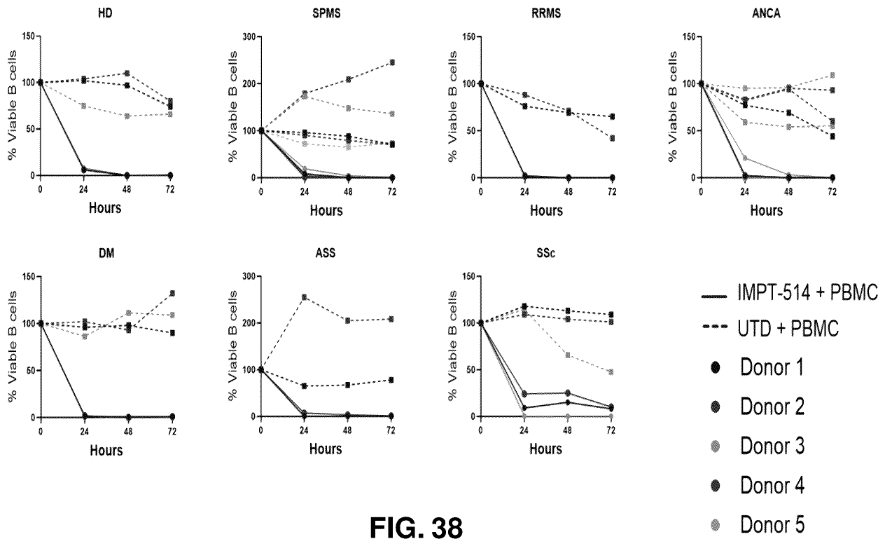

The ability of IMPT-514 to eliminate autologous B cells was assessed using IMPT-514 product and target PBMC cells from 3 healthy donors and autoimmune donors (FIG. 38). B cell lysis was assessed using flow cytometry to quantify CD19+ B cells and CAR T cells in co-culture. We observed a marked decrease in B cell counts one day after co-culture of IMPT-514 with autologous B cells within the target PBMC and complete ablation of B cells by day 3. CAR T cell cytotoxicity was comparable among all donors (FIG. 38). As expected, untransduced T cells did not impact the viability of autologous B cells (FIG. 38). Abbreviations: CAR=chimeric antigen receptor; HD=healthy donor; SPMS=secondary progressive multiple sclerosis; RRMS=relapsing remitting multiple sclerosis; ANCA=ANCA-associated vasculitis; DM=dermatomyositis; ASyS=anti-synthetase syndrome; SSc=systemic sclerosis. Note: Cytotoxicity of IMPT-514 CAR T cells transduced from healthy and IIM donors when co-cultured with B cell containing PBMC. CAR T cells were generated by lentiviral transduction with the CC314B vector at an MOI of 5. CAR T cells were co-incubated with autologous B cell containing PBMC at 3:1 E:T (CAR T cell:B cell) in a 96-well plate for 3 days.

These observations show that IMPT-514 manufactured from autoimmune donors can eliminate autologous B cells and proliferate in a similar manner to that observed with IMPT-514 T cell products derived from healthy donors. The above observations demonstrated cytokine production, T-cell proliferation, and anti-autologous B cell cytotoxicity of the IMPT-514.

### Elimination of Antibody-Producing Cells

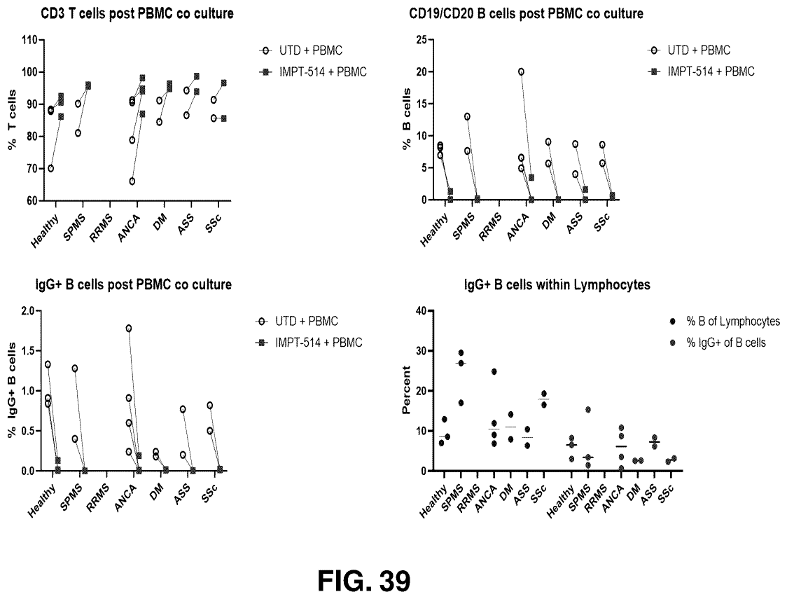

IMPT-514 or untransduced (UTD) T cells generated from healthy or autoimmune donors were incubated with autologous PBMC. After 3 days samples were stained for CD3+ T cells, B cells (CD19+ or CD20+), and intracellular IgG. Compared to co-cultures with UTD, co-cultures with IMPT-514 had significant decreases in % B cells and % IgG⁺ B cells for healthy and autoimmune donors. There were no significant differences in the post-co-culture % CD3 cells, % B cells or % IgG⁺ B cells between healthy or autoimmune donors. In summary, IMPT-514-specific elimination of both B cells and IgG⁺ B cells was observed in autologous co-cultures regardless of condition (FIG. 39). Abbreviations: CAR=chimeric antigen receptor; HD=healthy donor; SPMS=secondary progressive multiple sclerosis; RRMS=relapsing remitting multiple sclerosis; ANCA=ANCA-associated vasculitis; DM=dermatomyositis; ASyS=anti-synthetase syndrome; SSc=systemic sclerosis. Notes: Donors were co-cultured with autologous PBMC for 3 days. A) Following incubation, cells were harvested and stained for flow cytometry. CD3⁺ cells were excluded prior to gating B cells, which are defined as CD19⁺ or CD20⁺, and presented percentage relative to live lymphocytes. Paired ratio t-tests were employed to determine significance of the reductions observed (*: p<0.05, **:p<0.005). B) Percent reduction of B cells and IgG⁺ B cells in PBMC co-cultures were observed with IMPT-514 regardless of donor.

Though IgG serum levels are known to be elevated in autoimmune patients, the relative percentage of IgG⁺ B cells within peripheral blood does not account for the specificity of the IgG, tissue resident IgG-producing B cells and plasmablasts. Additionally, the patient samples tested do not represent baseline levels as these patients undergo intense immunosuppressive therapies which are known to reduce IgG levels in many patients. Relative B cell elimination was not significantly different between healthy, and autoimmune donors. These results indicate that the potentially more pathogenic IgG⁺ B cells within the peripheral blood are effectively eliminated by IMPT-514.

### Example 18: Evaluation of CAR T Activity of Humanized Leu16− Anti CD20-41BBz CAR T Cells

### Humanization of Anti CD20 Leu16 Monoclonal Antibody

Relapse due to loss of the CD19 targeted epitope presents a therapeutic challenge of CD19 CAR-T therapy. These patients universally have a poor outcome, and the unmet medical need is high. CD20 is an attractive immunotherapy target for B-cell non-Hodgkin lymphomas, and adoptive transfer of T cells genetically modified to express a chimeric antigen receptor (CAR) targeting CD20 is a promising strategy. CD20 is a proven therapeutic target for B-NHL, supported by previously approved naked and radiolabeled anti-CD20 antibodies and promising results from bispecific antibodies. CD20-targeted CAR-T is another potential adoptive immunotherapy option that could be utilized in combination or in sequence with CD19 CAR-T.

mAb Leu16 is a murine monoclonal antibody that was demonstrated to recognize B-lymphocyte surface antigen CD20. The CD20-specific Leu16-IgG4-BB-z vector was constructed by amplifying the Leu16 single-chain variable-region fragment (scFv), and 12 humanized versions of the Leu16 scFV were screen side by side with the murine Leu16 CAR.

The goal of antibody humanization was to engineer a monoclonal antibody (MAb) raised in a nonhuman species into one that is less immunogenic when administered to humans.

mAb Leu16 anti human CD20 was humanized using complementarity determining region (CDR) grafting on human Ig framework regions. The transfer of antigen binding loops from a non-human antibody donor to a human framework must be performed so that the native loop conformations are retained for binding.

The complementarity determining regions (CDRs) are the hypervariable ‘ends’ of an antibody which are responsible for where antibodies bind to a specific antigen. CDR grafting is a humanization technique whereby humanized antibody sequences are generated by carefully selecting the CDRs of the parental antibody (in this case murine) and grafting them into a human framework.

The identification of critical framework residues that contribute to CDR conformation represents the most difficult and unpredictable step in the antibody humanization process. Human frameworks for heavy and light chains were selected based on sequence and structure similarity with respect to functional human germline genes. In this regard, structural similarity was evaluated by comparing the mouse canonical CDR structure to human candidates with the same canonical structures.

Anti CD20 was humanized using a computer-aided CDR-grafting method (Abysis Database, UCL Business Plc.) and standard molecular engineering techniques to provide 18 HzLeu16 constructs. The human framework regions of the variable regions were selected based on their highest sequence homology to the subject mouse framework sequence and its canonical structure. For the purposes of the humanization analysis the assignment of amino acids to each of the CDR domains is in accordance with Kabat annotation numbering.

Molecular engineering procedures were conducted. To that end accurate determination of the murine antibody variable region DNA sequences, amplified by RT-PCR, and homology modelling of its heavy and light chains protein translation were the starting points.
Hz. Leu16 (a) VH was generated by grafting mouse CDRs into human VH1-46 Framework and used in constructs CC374, CC380.
Hz. Leu16 (b) VH was generated by grafting mouse CDRs into human VH1-46 Framework with back mutations at positions H73; used in constructs CC375, CC381.
Hz. Leu16 (c) VH was generated by grafting mouse CDRs into human VH3-23 Framework; used in constructs CC376, CC382 Hz. Leu16 (d) VH was generated by grafting mouse CDRs into human VH3-23 Framework with back mutations at positions H30; used in constructs CC377, CC383.
Hz. Leu16 (e) VH was generated by grafting mouse CDRs into human VH3-23 Framework with back mutations at positions H30T; H49G; used in constructs CC378, CC384.
Hz. Leu16 (f) VH was by generated by grafting mouse CDRs into human VH3-23 Framework with back mutations at positions H30T; H49G;H71A.
Hz. Leu16 (a) VL was by generated by grafting mouse CDRs into human VK-O2 Framework; used in constructs: CC374, CC375, CC376, CC377, CC378 and CC379.
Hz. Leu16 (b) VL was by grafting mouse CDRs into human VK-A11 Framework; CC3, CC31, CC382, CC383, CC384 and CC385.

From the nucleotide sequence information, data regarding V, D and J gene segments of the heavy and light chains of Leu16 murine antibody were obtained. Subsequently the V-(D)-J sequences were aligned with mouse Ig germ line sequences. The resulting genetic arrangements for two humanized constructs are shown in sequence alignment in TABLE 17.

TABLE 17
Summary of Humanized constructs

| | human | | FW | human | human | | FW |
|---|---|---|---|---|---|---|---|
| mAb | VK | JK | changes | VH | DH | JH | changes |
| Hz Lue16-CC374 | O2 | IGKJ4 | None | IGHV1-46 | IGHD2-2*03 | IGHJ2 | None |
| Hz Lue16-CC375 | O2 | IGKJ4 | None | IGHV1-46 | IGHD2-2*03 | IGHJ2 | T73K |
| Hz Lue16-CC376 | O2 | IGKJ4 | None | IGHV3-23 | IGHD2-2*03 | IGHJ2 | None |
| Hz Lue16-CC377 | O2 | IGKJ4 | None | IGHV3-23 | IGHD2-2*03 | IGHJ2 | S30T |
| Hz Lue16-CC378 | O2 | IGKJ4 | None | IGHV3-23 | IGHD2-2*03 | IGHJ2 | S30T + S49G |
| Hz Lue16-CC379 | O2 | IGKJ4 | None | IGHV3-23 | IGHD2-2*03 | IGHJ2 | S30T + S49G + |
| | | | | | | | R71A |
| Hz Lue16-CC380 | A11 | IGKJ4 | None | IGHV1-46 | IGHD2-2*03 | IGHJ2 | None |
| Hz Lue16-CC381 | A11 | IGKJ4 | None | IGHV1-46 | IGHD2-2*03 | IGHJ2 | T73K |
| Hz Lue16-CC382 | A11 | IGKJ4 | None | IGHV3-23 | IGHD2-2*03 | IGHJ2 | None |
| Hz Lue16-CC383 | A11 | IGKJ4 | None | IGHV3-23 | IGHD2-2*03 | IGHJ2 | S30T |
| Hz Lue16-CC384 | A11 | IGKJ4 | None | IGHV3-23 | IGHD2-2*03 | IGHJ2 | S30T + S49G |
| Hz Lue16-CC385 | A11 | IGKJ4 | None | IGHV3-23 | IGHD2-2*03 | IGHJ2 | S30T + S49G + |
| | | | | | | | R71A |

Following humanization of all selected antibodies by CDR grafting, the resulting light and heavy chain variable region amino acid sequences were analyzed to determine their homology with regard to the murine donor and human acceptor light and heavy chain variable regions. The results, shown in TABLE 18 immediately below, reveal that the humanized constructs consistently exhibited a higher homology with respect to the human acceptor sequences than with the murine donor sequences. More particularly, the murine heavy and light chain variable regions show a similar overall percentage homology to a closest match of human germline genes (86%-91%) compared with the homology of the humanized antibodies and the donor hybridoma protein sequences (72%-83%).

TABLE 18

| | Homology to | Homology to Murine |
|---|---|---|
| | Human mAb | Parent (CDR |
| Mab | (CDR acceptor) | donor) |
| Hz Leu16 VH1-46 | 91.8% | 83.6% |
| Hz Leu16 VH3-23 | 86.7% | 72.1% |
| Hz Leu16 VKO2 | 86.3% | 78.3% |
| Hz Leu16 VKA11 | 83.3% | 78.3% |

Humanized Leu16 CAR constructs were cloned into lentivirus expression system, scFvs were generated in a VL-VH configuration using a 4GS-18 linker, and a mIgK leader sequence was cloned upstream. To generate 41BBz CARs, IgG4 hinge, CD28 transmembrane, and 41BB signaling domains together with the CD3z domain were fused to Humanized Leue16 scFV by gene synthesis, following the design used for murine CD20-Leu16-41BBz CAR.

T-cells were isolated from two healthy donors, expanded in vitro, and transduced with the described set of murine and 12 humanized Leu16 (CD3ζ and 4-1BB) anti CD20 Lentivirus constructs. The anti-CD20 CAR T-cells were characterized by CAR expression level, expansion capabilities, T cell phenotype by expression of differentiation and exhaustion markers, and vitro cytotoxic activity. The newly Humanized Leu16 scFv-expressing CAR-T cells had better proliferation capacity and durable phenotypes, enabling strong reactivity against Raji cell line in vitro in comparison with the murine CAR-T cells.

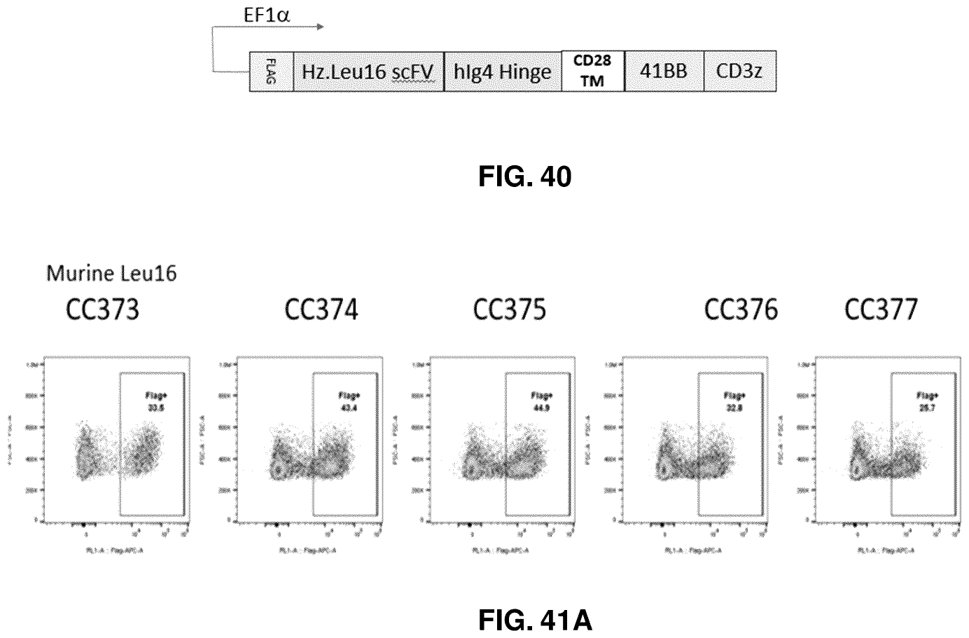

Humanized Leu16 Lenti viral CAR vectors were constructed and packaged at Vector Builder according to FIG. 40. Second generation CARs are composed of five parts: (a) the humanized Leu16 scFv domain in the VL-VH orientation using a 4GS-18 linker, cloned upstream to the mIgK leader sequence and a FLAG tag (b) a human IgG4 (Hinge-CH2-CH3) region (c) CD28 transmembrane region, (d) costimulatory domain (4-1BB), and (e) CD3ζ signaling domain. The FLAG tag was placed at the 5′ end for CAR detection (FIG. 40)

### Transduction Efficiency and CART Expression of Humanized Leu16− Anti CD20− 41BBz CAR Lentivirus Constructs

Naive/memory T cells were isolated, transduced, and cultured. Briefly, on day 1 the cells were activated with TransAct Human T cell expander CD3/CD28 (Miltenyi Biotec), supplemented with 50 IU/mL IL-2 (Miltenyi Biotec) and 0.5 ng/mL IL-15 (Miltenyi Biotec). Two days after activation, cells were transduced with lentiviral vectors constructs 373 to 385 at a multiplicity of infection (MOI) of 3, calculated from functional titer provided by Vector Builder. Cells were collected after 9 days of culture.

### Flow Cytometry Analysis of CAR+ T Cells

On day 12, cells were harvested to assess the percentage of positive Hz. Leu16 CAR positive T cells by anti-FLAG flow cytometry. Samples were analyzed using the Attune N×T flow cytometer (Thermo Fisher).

For analysis, cells are gated on forward scatter area (FSC-A) and side scatter area (SSC-A) to gate the cell population, then FSC-A and forward scatter height (FSC-H) to gate the singlets, then live cells (DAPI), then flag+ cells. Controls included single color controls (SCCs) and isotype controls for each fluorophore.

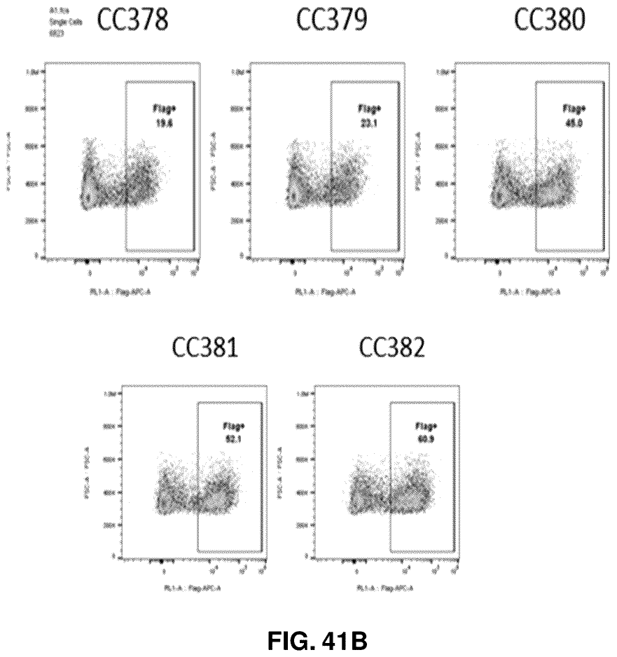

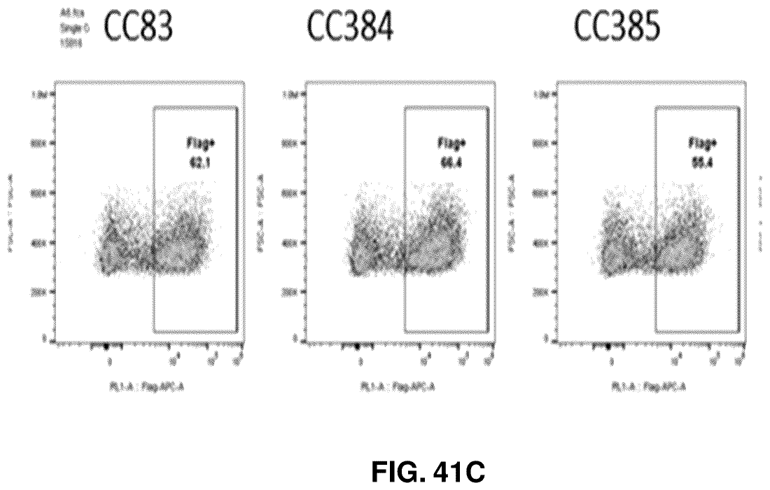

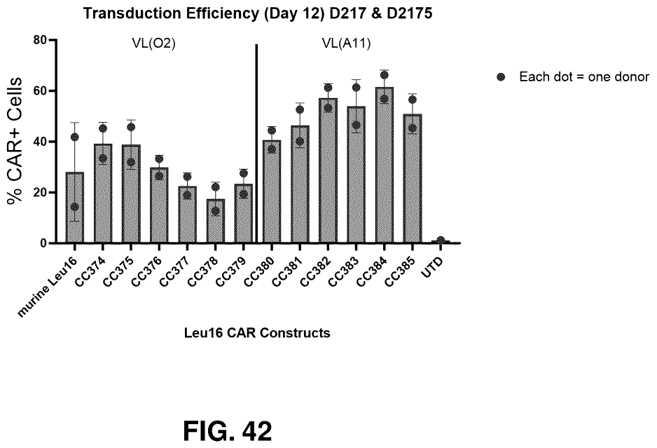

To determine the expression of the transgene, gating to distinguish between positive and negative populations for transgene expression was performed on un-transduced cells. Representative gating for transgene expression using anti FLAG tag antibody (FIG. 41A, FIG. 41B, FIG. 41C and FIG. 42). FIG. 41A, FIG. 41B, FIG. 41C: Representative flow cytometry plot from donor 217; Note: Representative gating plots to discriminate between CAR-negative (un-transduced cells) and CAR-positive cells from a single donor (217). T cells were harvested on day 12 of the process and stained with anti-FLAG to assess the percentage of CAR-positive cells by flow cytometry.

Transduction efficiency of Hz. Leu16 CAR T cells showed between 25% to 60%, CAR positive cells suggesting that it was at or above the parental murine Leu16 CAR T expression level, humanization has not affected scFv stability and CAR expression level and CAR T cells with clinical relevant showed variable expression of the CAR construct.

FIG. 42 shows a summary of transduction efficiency in two donors 217 and 2175. Transduction efficiency was tested on day 12 using T cells isolated and transduced from two donors. Each dot represents one donor. Transduction efficiency and percentage of CAR positive cell population is shown by the bar graph of CAR expression in total CD3+ T cells, at culture day 12 as measured by flow cytometry. For most constructs CAR expression levels were higher than the murine Leu16 CAR T. Back mutations to mouse residues in VH1-46 framework at position H-30, H49 and H71 reduced CAR T expression when paired to the VK-O2 light chain (CC377, CC378, and CC379. Humanized Leu16 heavy chains show better compatibility and higher expression level when paired with the human VK-A11 framework derived light chain.

### Humanized Leu16 CAR T Cell Expansion Capabilities In Vitro

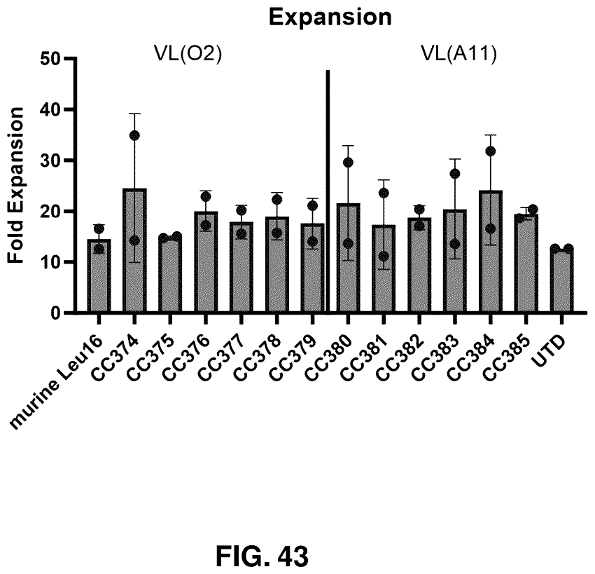

FIG. 43 shows T cell proliferation represented by a fold expansion graph. Population doublings were calculated by measuring the total cell number in culture at day 12 using a cell counter. Thirteen distinct CAR T cell products were generated from two donors by transducing αCD3/CD28 bead-activated T cells with lentiviruses constructs of murine and humanized anti Cd20 Leu16-specific CAR. The calculated fold expansion from Transduction Day (day 3) to harvest day (day 12) using the cell density values was measured by the Countess (Beckman Coulter Cell Counter). Over the course of a 12-day in vitro cell expansion period, the observed fold expansion was between 15-30 on average, with the highest fold expansion exhibited by CAR T cells transduced with humanized Leu16 construct.

### Immunophenotype Characterization of Humanized Leu16 CAR T Cells on Day 12 Post Transduction.

Naive/memory T cells (TN/MEM) were isolated, transduced, and cultured. Two days after activation, cells were transduced with lentiviral vectors CC373 to CC385 at a multiplicity of infection (MOI) of 5.

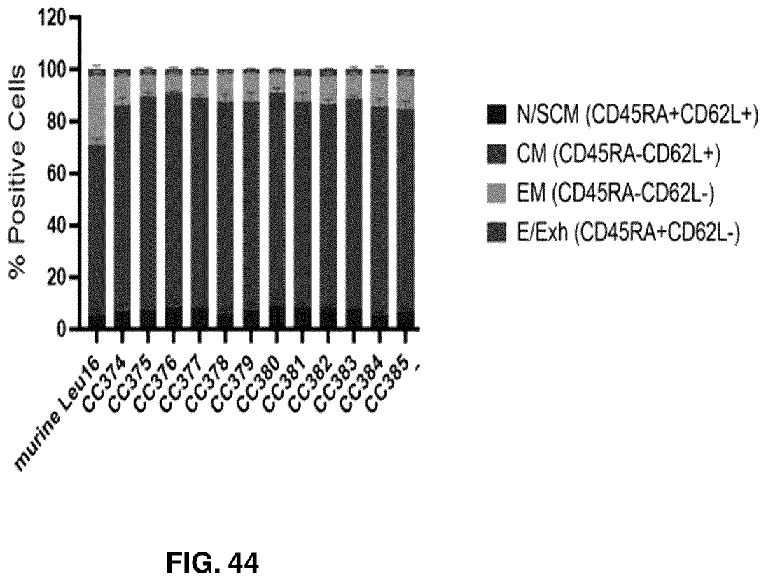

On Day 12, cells were washed with staining buffer containing 0.2% bovine serum albumin (BSA) (BD) and stained with antibody panel to detect: CD4, CD8, CD45RA and CD62L in 50 μL of staining buffer and incubated at 4° C. for 30 minutes. After staining, cells were washed with staining buffer and phenotype analysis was performed with the Attune N×T flow cytometer. Live lymphocyte population was gated on FSC-A vs side scatter-area and single cell populations were gated based on forward scatter-height vs FSC-A. Controls included single color controls (SCCs) and isotype controls for each fluorophore. Greater than 80% of the cells were either central memory (predominant population) or naive/stem cell memory cells. Cells Transduced with Humanized Leu16 has higher CM Cells Compared to Murine Leu16 as shown in FIG. 44. Abbreviations: N/SCS=naive/stem cell memory; CM=central memory; EM=effector memory; E/Exh=effector/exhausted. Note: T-cells were harvested on day 12, at the end of the process and stained for CD45RA and CD62L. Data is from average of 2 donors.

### Cytotoxicity Functional Activity of CAR T Cells.

CD20 CAR T cell-mediated killing study was performed by isolation of T enriched CD62L+ phenotype population. The cells were either un-transduced or transduced with lentiviral vector expressing a humanized Leu16 chimeric antigen receptor (CAR) directed against CD20 (Cc373-CC38), and incubated with the target CD20+ Raji Cell line.

Naive/memory T cells were isolated, transduced and cultured from two healthy donors (Donor 217 and donor 2175) to generate Hz·Leu16 CART cells. CD62L+ T cells were isolated on Day 1 and activated with TransAct Human T cell expander CD3/28, supplemented with 50 IU/mL of interleukin (IL)-2 (Miltenyi Biotec) and 0.5 ng/mL IL-15 (Miltenyi Biotec). Two days after activation, cells were transduced with 13 lentivirus samples from CC373 to CC385. Cells were collected after 12 days of culture, with media and cytokine additions on days 5, 7, and 10.

CAR T cells were collected from each culture on Day 12. Raji expressing CD20 and CD19 cell line was purchased from ATCC and were transduced to stably express Firefly Luciferase for detection of target-cell lysis in co-culture.

On Day 12, CC373 to CC385, un-transduced (UTD) and target Raji cells co-culture in a 96-well plate. Luciferase expressing Raji cells were plated at 10,000 cells per well in a 96-well plate. Chimeric antigen receptor positive T cells or untransduced control were added at a starting effector-to-target ratio of 2.5:1 with a serial 2-fold dilution for a total of 7 dilutions. After 72 hours of coculture, CAR T cell cytotoxicity.

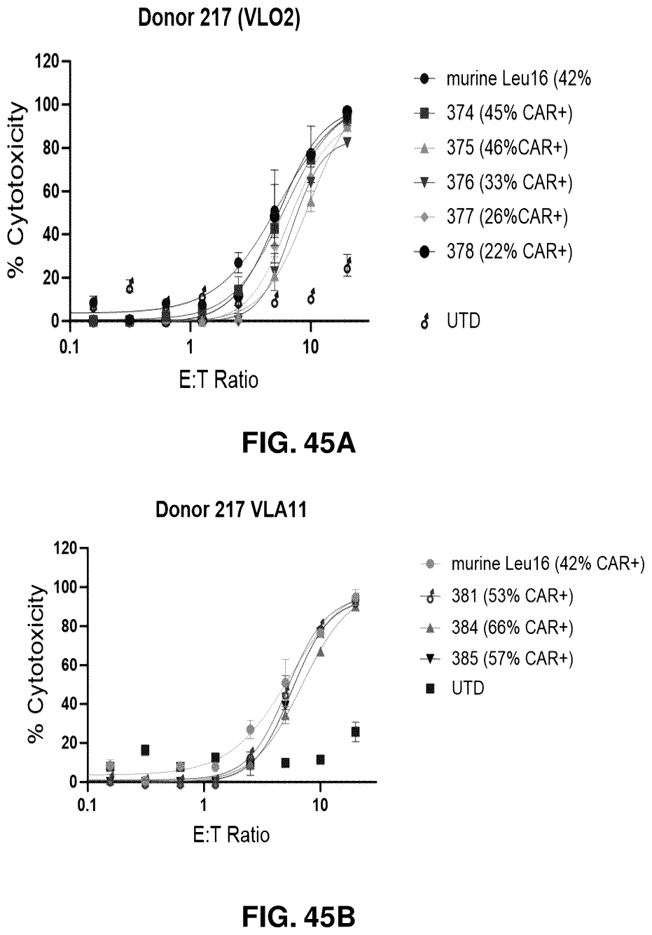

The cytotoxicity potential was measured by luciferase signal from the remaining viable, Raji cell line expressing CD20, target cells remaining in co-culture. Across 2 donors, constructs exhibited robust Raji-cell lysis compared to the UTD after a 72-hour coculture (FIG. 45A and FIG. 45B). Statistical comparison of cytotoxicity between UTD and Leu16 CAR T transduced cells were performed at each E to T ratio starting from 20:1 and titrated down serially. Abbreviations: CAR=chimeric antigen receptor; E:T=effector to target ratio

Leu16 CAR T products from two healthy donors (D217 (VLO2) and D217 (VLA11)) were generated by lentiviral transduction with the CC373-CC385 vector at an MOI of 5. CAR T cells were co-incubated with Raji cells at 2.5:1 E:T ratio with a serial 2-fold dilution at a total of 7 dilutions in a 96-well plate for 72 hours. Cytotoxicity was detected by measurement of the luciferase signal from the remaining target cells. The values shown are the means of technical replicates. Mean and standard deviation (error bar) are plotted. Values shown are the mean of triplicates, with error bars indicating one SD. P values were calculated by two-tailed Student t test; *, P<0.05; **, P<0.01; ***, P<0.001. Only comparisons with statistical significance are shown.

The ability of Hz·Leu16 CAR T cells to lyse antigen-expressing Raji target cells in response to antigen stimulation was confirmed by measurement of luciferase signal to assess the remaining target-cell after 72-hours coculture. The result suggested robust potency of Hz·Leu16 CAR T constructs against CD20 surface antigen.

### Cytokine (IFN-γ) Secretion

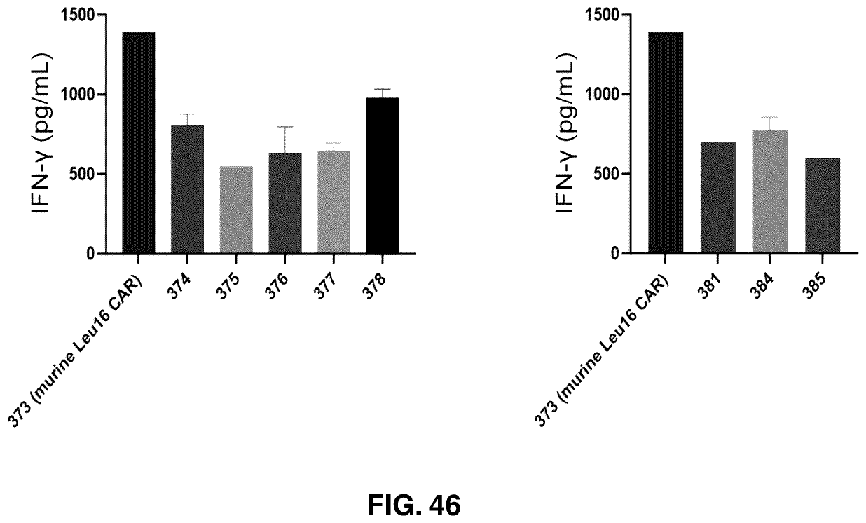

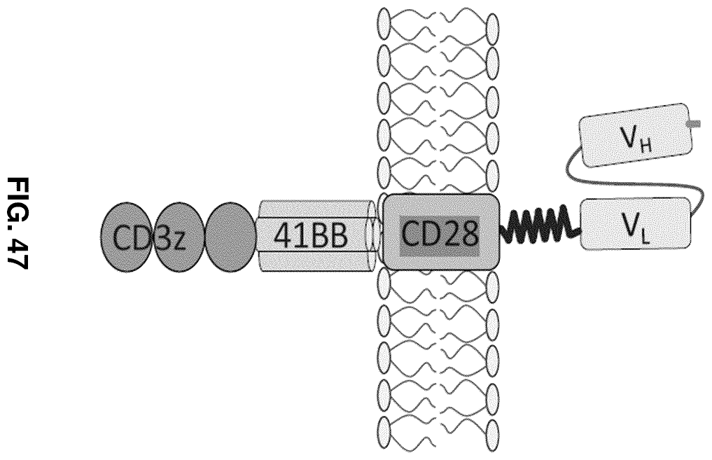

T cells enriched for a CD62L+ phenotype were transduced with lentivirus CC373-CC385 vectors. Evaluate IFN-γ secretion upon activation with target Raji CD20 positive cell line. Enzyme-linked immunosorbent assays was used quantify IFN-γ production to assess the response of the CAR T cells in co-culture with antigen positive tumor cells. The concentration of cytokines produced was compared with untransduced cells. The untransduced control produced minimal amounts and IFN-γ when co-cultured with Raji. The quantification of IFN-γ confirmed the ability of Humanized Leu16 CAR T cells to recognize and produce cytokines in response to the antigen-expressing Raji tumor cells, in comparable levels to the murine Leu16 as shown in FIG. 46.

### Example 19: Humanization of Anti CD19 FMC63 Monoclonal Antibody

FMC63 is a murine monoclonal antibody that is demonstrated to recognize B-lymphocyte surface antigen CD19. As explained above, the identification of critical framework residues that contribute to CDR conformation represents the most difficult and unpredictable step in the antibody humanization process.

Human frameworks for heavy and light chains were selected based on sequence and structure similarity with respect to functional human germline genes. In this regard, structural similarity was evaluated by comparing the mouse canonical CDR structure to human candidates with the same canonical structures.

Anti-CD19 was humanized using a computer-aided CDR-grafting method (Abysis Database, UCL Business Pk.) and molecular engineering techniques to provide several HzFMC63 constructs.

The human framework regions of the variable regions were selected based on their highest sequence homology to the subject mouse framework sequence and its canonical structure.

Accurate determination of the murine antibody variable region DNA sequences, amplified by RT-PCR, and homology modelling of its heavy and light chains protein translation were chosen as starting points.

From the nucleotide sequence information, data regarding V, D and J gene segments of the heavy and light chains of HLA2 murine antibody were obtained. Subsequently the V-(D)-J sequences were aligned with mouse Ig germ line sequences.

The resulting genetic arrangements for two humanized constructs are shown in Table 19 below.

TABLE 19

| | human | human | | FW | human | | FW |
|---|---|---|---|---|---|---|---|
| mAb | VH | DH | JH | changes | VK | JK | changes |
| Hz FMC63 | IGHV4-59 | IGHD3-22 | JH4 | none | O2 | JK2 | F71Y |
| VH | | | | | | | |
| Hz FMC63 | IGHV4-59 | IGHD3-22 | JH4 | I29L, S30P | O2 | JK2 | F71Y |
| VH | | | | | | | |
| hz FMC63 | IGHV3-23 | IGHD3-22 | JH4 | none | O2 | JK1 | F71Y |
| VH | | | | | | | |
| hz FMC63 | IGHV3-23 | IGHD3-22 | JH4 | R71K, L78V | O2 | JK1 | F71Y |
| VH | | | | | | | |
| hz FMC63 | IGHV3-23 | IGHD3-22 | JH4 | none | O2 | JK1 | F71Y/Y87F |
| VH | | | | | | | |
| hz FMC63 | IGHV3-23 | IGHD3-22 | JH4 | R71K, L78V | O2 | JK1 | T22S/F71Y |
| VH | | | | | | | |
| hz FMC63 | IGHV3-23 | IGHD3-22 | JH4 | R71K, L78V | O2 | JK1 | F71Y/F82I |
| VH | | | | | | | |
| hz FMC63 | IGHV3-23 | IGHD3-22 | JH4 | R71K, L78V | O2 | JK1 | F71Y/Y87F |
| VH | | | | | | | |
| hz FMC63 | IGHV3-23 | IGHD3-22 | JH4 | R71K, L78V | O2 | JK1 | T22S/F71Y |
| VH | | | | | | | |
| hz FMC63 | IGHV3-23 | IGHD3-22 | JH4 | R71K, L78V | O2 | JK1 | F71Y/F82I |
| VH | | | | | | | |

Following humanization of all selected antibodies by CDR grafting, the resulting light and heavy chain variable region amino acid sequences were analyzed to determine their homology with respect to the murine donor and human acceptor light and heavy chain variable regions.

The results, shown in Table 20 below, show that the humanized constructs consistently exhibited a higher homology with respect to the human acceptor sequences than with the murine donor sequences.

More particularly, the murine heavy and light chain variable regions show a similar overall percentage homology to a closest match of human germline genes (88%-92%) compared with the homology of the humanized antibodies and the donor hybridoma protein sequences (78%-89%).

TABLE 20

| | Homology to | Homology to |
|---|---|---|
| | Human mAb | Murine Parent (CDR |
| Mab | (CDR acceptor) | donor) |
| Hz FMC63 VH4- | HC 88% | HC 78% |
| 59 | | |
| Hz FMC63 VH3-23 | HC.87% | LC 72% |
| Hz FMC63 VKO2 | LC 92% | HC 85% |

### Example 20: Humanization of FMC63−anti CD19−41BBz

The CD19-specific FMC63-IgG4-BB-z vector was constructed by cloning the FMC63 single-chain variable-region fragment (scFv), and nine humanized versions of the FMC63 scFV were screen side by side with the murine FMC63 CAR.

This mAb FMC63 anti human CD19 was humanized using complementarity determining region (CDR) and contact residues grafting on human Ig framework regions. The transfer of antigen binding loops from a non-human antibody donor to a human framework must be performed so that the native loop conformations are retained for binding.

The complementarity determining regions (CDRs) are the hypervariable ‘ends’ of an antibody which are responsible for where antibodies bind to a specific antigen. CDR grafting is a humanization technique whereby humanized antibody sequences are generated by carefully selecting the CDRs of the parental antibody (in this case murine) and grafting them into a human framework.

The identification of critical framework residues that contribute to CDR conformation represents the most difficult and unpredictable step in the antibody humanization process. Human frameworks for heavy and light chains were selected based on sequence and structure similarity with respect to functional human germline genes. In this regard, structural similarity was evaluated by comparing the mouse canonical CDR structure to human candidates with the same canonical structures.

Anti CD19 was humanized using a computer-aided CDR-grafting method (Abysis Database, UCL Business Plc.) and standard molecular engineering techniques to provide 18 HzFMC63 constructs. The human framework regions of the variable regions were selected based on their highest sequence homology to the subject mouse framework sequence and its canonical structure. For the purposes of the humanization analysis the assignment of amino acids to each of the CDR domains is in accordance with Kabat annotation numbering.

Molecular engineering procedures were conducted using art-recognized techniques. To that end accurate determination of the murine antibody variable region DNA sequences, amplified by RT-PCR, and homology modelling of its heavy and light chains protein translation were the starting points.

From the nucleotide sequence information, data regarding V, D and J gene segments of the heavy and light chains of FMC63 murine antibody were obtained. Subsequently the V-(D)-J sequences were aligned with mouse Ig germ line sequences. The resulting genetic arrangements for two humanized constructs are shown in sequence alignment and the Table below.

Humanized FMC63 CAR constructs were cloned into lentivirus expression system, scFvs were generated in a VL-VH configuration using whitlow Linker, and a mIgK leader sequence was cloned upstream. To generate 41BBz CARs, IgG4 hinge, CD28 transmembrane, and 41BB signaling domains together with the CD3z domain were fused to Humanized FMC63 scFV by gene synthesis, following the design used for murine CD19-FMC63-41BBz CAR as shown in Table 21.

TABLE 21

| | human | | FW | human | human | | FW |
|---|---|---|---|---|---|---|---|
| mAb | VK | JK | changes | VH | DH | JH | changes |
| FMC63-9 | VK-O2 | IGKJ2 | L71 | VH4-59 | IGHD3-22 | JH4 | none |
| FMC63-10 | VK-O2 | IGKJ2 | L71 | VH4-59 | IGHD3-22 | JH4 | H29, H30 |
| FMC63-11 | VK-O2 | IGKJ2 | L71 | VH4-59 | IGHD3-22 | JH4 | H67 |
| FMC63-12 | VK-O2 | IGKJ2 | L71 | VH3-23 | IGHD2-2 | JH6 | none |
| FMC63-13 | VK-O2 | IGKJ2 | none | VH3-23 | IGHD2-2 | JH6 | H71, H87 |
| Hz.FMC63-16 | VK-O2 | IGKJ2 | none | VH3-23 | IGHD2-2 | JH6 | none |
| Hz.FMC63-17 | VK-O2 | IGKJ2 | L87 | VH3-23 | IGHD2-2 | JH6 | none |
| Hz.FMC63-18 | VK-O2 | IGKJ2 | L22 | VH3-23 | IGHD2-2 | JH6 | none |
| Hz.FMC63-19 | VK-O2 | IGKJ2 | L82 | VH3-23 | IGHD2-2 | JH6 | none |
| Hz.FMC63-20 | VK-O2 | IGKJ2 | none | VH3-23 | IGHD2-2 | JH6 | H71, H87 |
| Hz.FMC63-21 | VK-O2 | IGKJ2 | L87 | VH3-23 | IGHD2-2 | JH6 | H71, H87 |
| Hz.FMC63-22 | VK-O2 | IGKJ2 | L22 | VH3-23 | IGHD2-2 | JH6 | H71, H87 |
| Hz.FMC63-23 | VK-O2 | IGKJ2 | L82 | VH3-23 | IGHD2-2 | JH6 | H71, H87 |

### Evaluation of CART Activity of Humanized FMC63−Anti CD19−41BBz CAR T Cells

T-cells were isolated from two healthy donors, expanded in vitro, and transduced with the described set of murine and 9 of the humanized MFC63 (CD3ζ and 4-1BB) anti CD19 Lentivirus constructs. The anti-CD19 CAR T-cells were characterized by CAR expression level, expansion capabilities, T cell phenotype by expression of differentiation and exhaustion markers, and vitro cytotoxic activity. The newly Humanized FMC63 scFv-expressing CAR-T cells had better proliferation capacity and durable phenotypes, enabling strong reactivity against Raji cell line in vitro in comparison with the murine CAR-T cells.

### Methodology

Humanized FMC63 Lenti viral CAR vectors were constructed and packaged at Vector Builder according to the scheme below.

Second generation CARs are composed of five parts: (a) the humanized FMC63 scFv domain in the VH-VL orientation using a whitlow linker, cloned upstream to the mIgK leader sequence (b) a human IgG4 (Hinge) region (c) CD28 transmembrane region, (d) costimulatory domain (4-1BB), and (e) CD3ζ signaling domain. (FIG. 47)

### Transduction Efficiency and CAR T Expression of Humanized FMC63−Anti CD19−41BBz CAR Lentivirus Constructs

Naive/memory T cells were isolated, transduced, and cultured. Briefly, on day 1 the cells were activated with TransAct Human T cell expander CD3/CD28 (Miltenyi Biotec), supplemented with 50 IU/mL IL-2 (Miltenyi Biotec) and 0.5 ng/mL IL-15 (Miltenyi Biotec). Two days after activation, cells were transduced with lentiviral vectors constructs 373 to 385 at a multiplicity of infection (MOI) of 3, calculated from functional titer provided by Vector Builder. Cells were collected after 9 days of culture.

### Flow Cytometry Analysis of CAR+ T Cells

On day 12, cells were harvested to assess the percentage of positive Hz. FMC63 CAR positive T cells by anti-FLAG flow cytometry. Samples were analyzed using the Attune N×T flow cytometer (Thermo Fisher).

For analysis, cells are gated on forward scatter ara (FSC-A) and side scatter area (SSC-A) to gate the cell population, then FSC-A and forward scatter height (FSC-H) to gate the singlets, then live cells (DAPI), then flag+ cells. Controls included single color controls (SCCs) and isotype controls for each fluorophore.

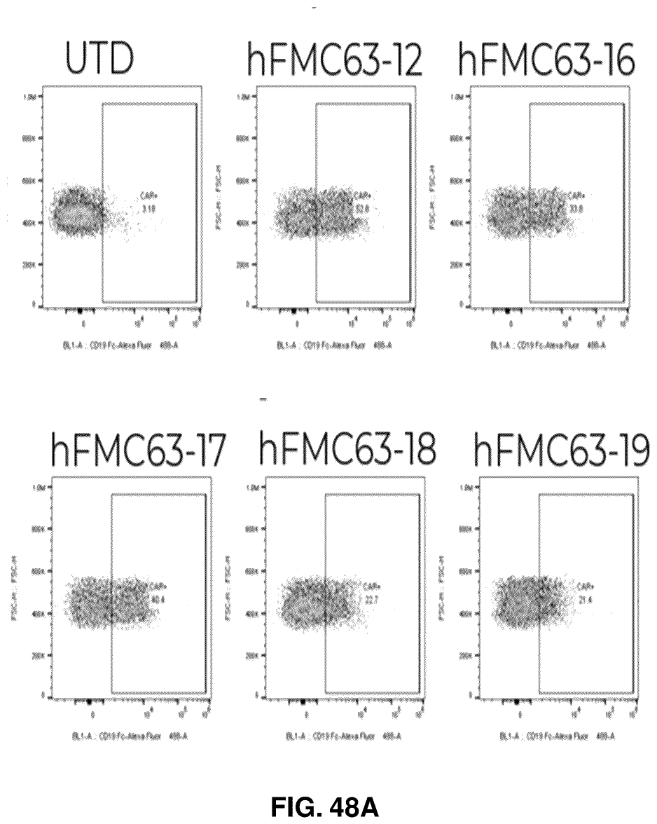

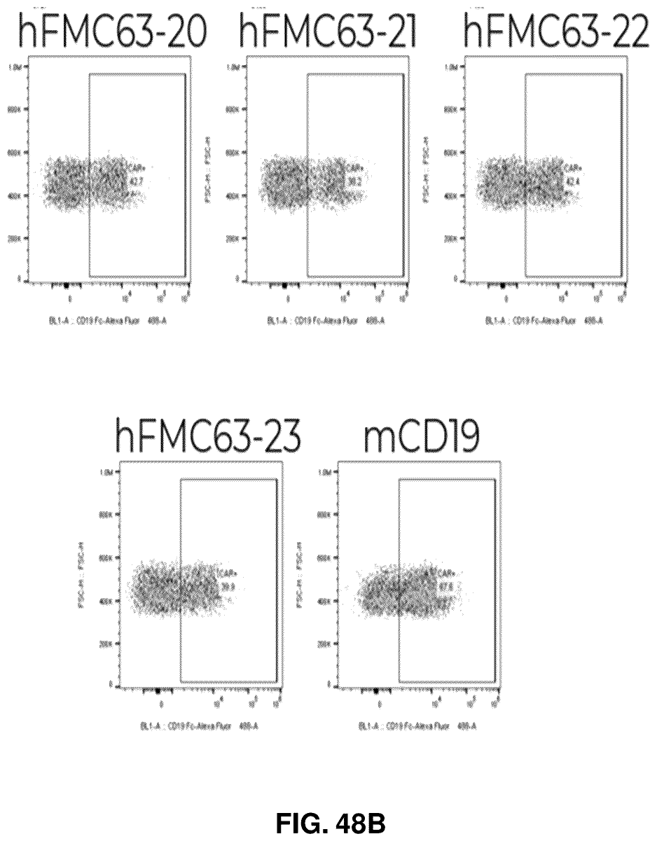

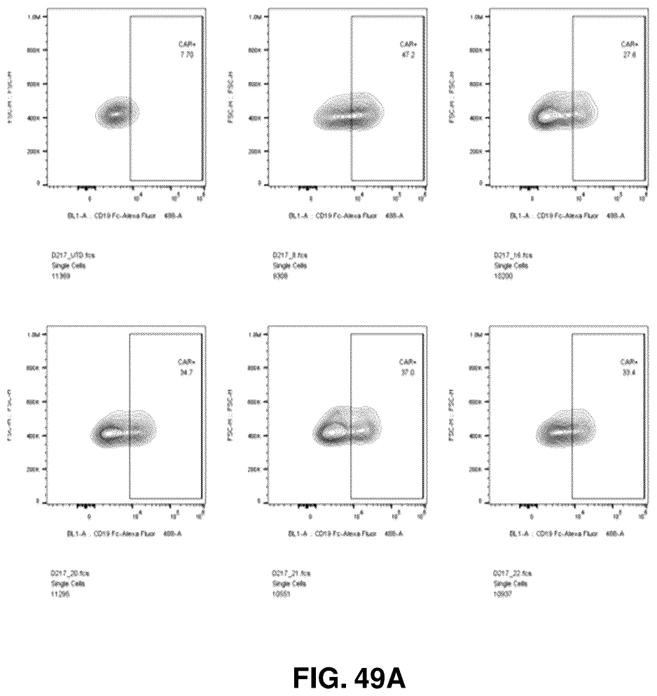

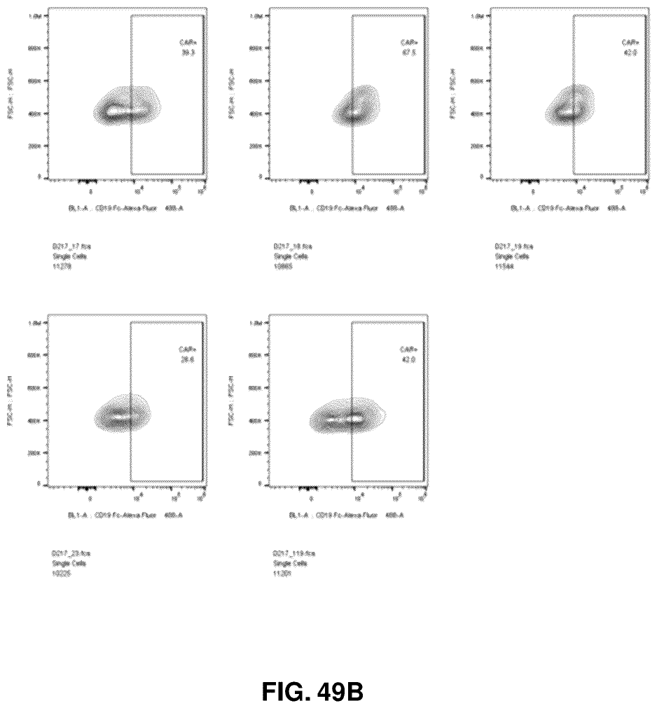

To determine the expression of the transgene, gating to distinguish between positive and negative populations for transgene expression was performed on un-transduced cells. Representative gating for transgene expression using anti FLAG tag antibody (FIG. 48 and FIG. 49)

Transduction efficiency of Hz. FMC63 CART cells showed between 25% to 60%, CAR positive cells suggesting that it was at or above the parental murine FMC63 CAR T expression level, humanization has not affected scFv stability and CAR expression level and CAR T cells with clinical relevant showed variable expression of the CAR construct.

### Summary of Transduction Efficiency in Two Donors 292:

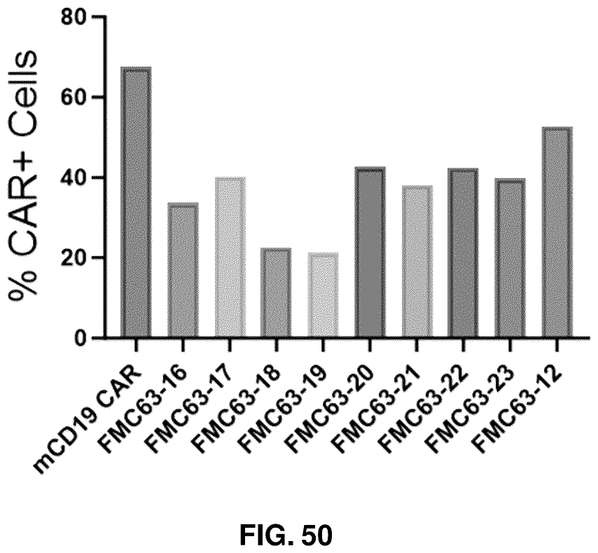

Transduction efficiency was tested on day 12 using T cells isolated and transduced from two donors. (FIG. 50)

There was an overall good CAR Expression of all humanized constructs and humanized FMC63 heavy chains using VH23 show better compatibility and higher expression level when paired with the human VK-O2 framework derived light chain.

### Cytotoxicity Functional Activity of CAR T Cells.

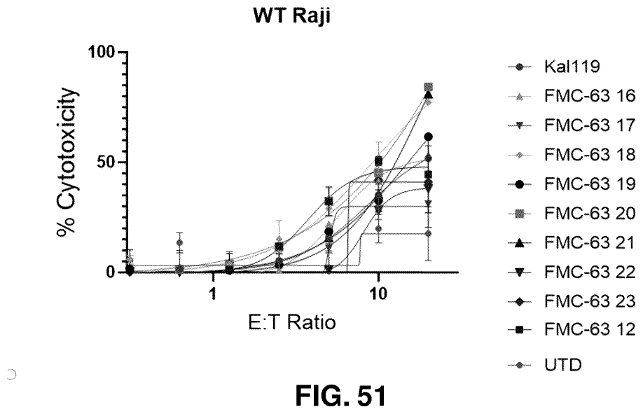

CD19 CAR T cell-mediated killing study was performed by isolation of T enriched CD62L+ phenotype population. The cells were either un-transduced or transduced with lentiviral vector expressing a humanized FMC63 chimeric antigen receptor (CAR) directed against CD19 (Cc373-CC38), and incubated with the target CD19+ Raji Cell line. (FIG. 51)

Naive/memory T cells were isolated, transduced and cultured from two healthy donors (Donor 217 and donor 2175) to generate Hz·FMC63 CART cells. CD62L+ T cells were isolated on Day 1 and activated with TransAct Human T cell expander CD3/28, supplemented with 50 IU/mL of interleukin (IL)-2 (Miltenyi Biotec) and 0.5 ng/mL IL-15 (Miltenyi Biotec). Two days after activation, cells were transduced with 13 lentivirus samples from CC373 to CC385. Cells were collected after 12 days of culture, with media and cytokine additions on days 5, 7, and 10.

CAR T cells were collected from each culture on Day 12. Raji expressing CD19 and CD19 cell line was purchased from ATCC and were transduced to stably express Firefly Luciferase for detection of target-cell lysis in co-culture.

On Day 12, CC373 to CC385, un-transduced (UTD) and target Raji cells co-culture in a 96-well plate. Luciferase expressing Raji cells were plated at 10,000 cells per well in a 96-well plate. Chimeric antigen receptor positive T cells or untransduced control were added at a starting effector-to-target ratio of 2.5:1 with a serial 2-fold dilution for a total of 7 dilutions. After 72 hours of coculture, CAR T cell cytotoxicity was measured by luciferase signal using Bio-Glo™ Luciferase Assay system (Promega) and quantified by Varioskan (ThermoFisher).

The cytotoxicity potential was measured by luciferase signal from the remaining viable, Raji cell line expressing CD19, target cells remaining in co-culture. Across 2 donors, constructs exhibited robust Raji-cell lysis compared to the UTD after a 72-hour coculture (FIG. 51). Statistical comparison of cytotoxicity between UTD and FMC63 CAR T transduced cells were performed at each E to T ratio starting from 20:1 and titrated down serially.

FMC63 CAR T products from two healthy donors (D217, and D2175) were generated by lentiviral transduction with the CC373-CC385 vector at an MOI of 5. CAR T cells were co-incubated with Raji cells at 2.5:1 E:T ratio with a serial 2-fold dilution at a total of 7 dilutions in a 96-well plate for 72 hours. Cytotoxicity was detected by measurement of the luciferase signal from the remaining target cells. The values shown are the means of technical replicates. Mean and standard deviation (error bar) are plotted. Values shown are the mean of triplicates, with error bars indicating one SD. P values were calculated by two-tailed Student t test; *, P<0.05; **, P<0.01; ***, P<0.001. Only comparisons with statistical significance are shown.

The ability of Hz·FMC63 CAR T cells to lyse antigen-expressing Raji target cells in response to antigen stimulation was confirmed by measurement of luciferase signal to assess the remaining target-cell after 72-hours coculture. The result suggested robust potency of Hz·FMC63 CAR T constructs against CD19 surface antigen.

### Cytokine (IFN-γ) Secretion

T cells enriched for a CD62L+ phenotype were, were transduced with lentivirus CC373-CC385 vectors to evaluate IFN-γ secretion upon activation with target Raji CD19 positive cell line.

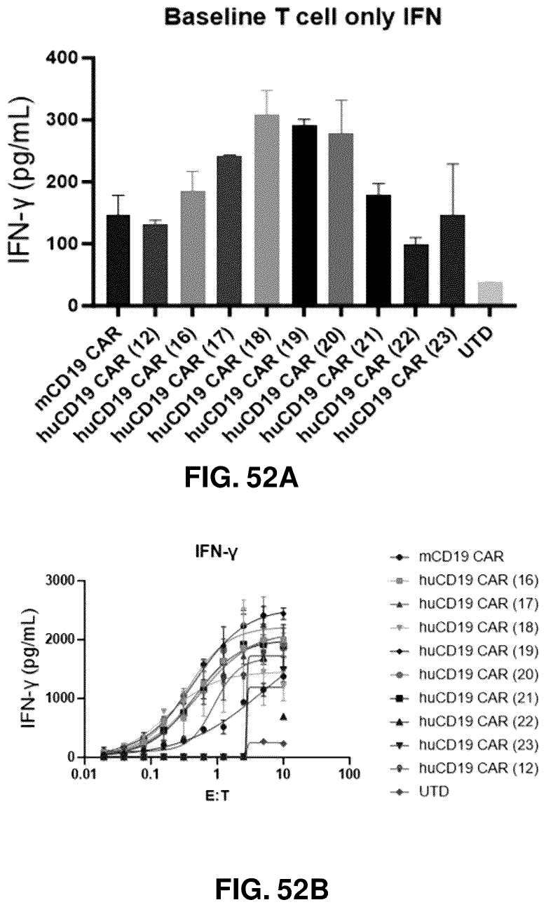

Enzyme-linked immunosorbent assays was used quantify IFN-γ production to assess the response of the CAR T cells in co-culture with antigen positive tumor cells. The concentration of cytokines produced was compared with untransduced cells. The untransduced control produced minimal amounts and IFN-γ when co-cultured with Raji. (FIG. 52).

The quantification of IFN-γ confirmed the ability of Humanized FMC63 CAR T cells to recognize and produce cytokines in response to the antigen-expressing Raji tumor cells, in comparable levels to the murine FMC63.

### REFERENCES

Abramson J S, Palomba M L, Gordon U, et al. Lisocabtagene maraleucel for patients with relapsed or refractory large B-cell lyphomas (TRANSCEND NHL 001): a multicentre seamless design study. Lancet. 2020; 396(10254):839--852. doi:10.1016/S0140-6736(20)31366-0.
Food and Drug Administration. Guidance for Industry: Considerations for the Development of Chimeric Antigen Receptor (CAR) T Cell Products Draft Guidance for Industry. 2022.
Fry T J, Shah N N, Orentas R J, Stetler-Stevenson M, Yuan C M, Rarnakrishna S, Wolters P, Martin S, Delbrook C, Yates B, Shalabi H, Fountaine T J, Shern J F, Majzner R G, Stroncek D F, Sabatino M, Feng Y, Dimitrov D S, Zhang L, Nguyen S, Qin H, Dropulic B, Lee D V, Mackall CL CD22-targeted CART cells induce remission in B-ALL that is naive or resistant to CD 19-targeted CAR immunotherapy. Nat Med. 2018 January:24(1):20-28. doi: 10.1038/nm.4441. Epub 2017 Nov. 20. PMID: 29155426: PMCID: PMC5774642.
Gardner, R. A., Finney, 0., Annesley, C., Brakke, H., Summers, C., Leger, K., Jensen,_M. C. (2017). Intent-to-treat leukemia remission by CD19 CART cells of defined formulation and dose in children and young adults. Blood, 129(25), 3322-3331._doi: 10.1182/blood2017-02-769208.
Ghorashian S, Kramer A M, Onuoha S, Wright G, Bartram J, Richardson R, Alban S J, Casanovas-Company J, Castro F, Popova B, Villanueva K, Yeung J, Vetharoy W, Guvenel A, Wawrzyniecka P A, Mekkaoui L, Cheung G W, Pinner D, Chu J, Lucchini G, Silva J, Ciocarlie 0, Lazareva A, Inglott S, Gilmour K C, Ahsan G, Ferrari M, Manzoor S, Champion K, Brooks T, Lopes A, Hackshaw A, Farzaneh F, Chiesa R, Rao K, Bonney D, Samarasinghe S, Goulden N, Vora A, Veys P, Hough R, Wynn R, Pule M A, Amrolia P J. Enhanced CAR T cell expansion and prolonged persistence in pediatric patients with ALL treated with a low-affinity CD19 CAR. Nat Med. 2019 September; 25(9):1408-1414. doi: 10.1038/s41591-019-0549-5. Epub 2019 Sep. 2. PMID: 31477906.
Jalbert, Jessica J., et al. “Real-World Treatment Patterns After CD19-Directed CART Cell Therapy Among Patients with Diffuse Large B Cell Lymphoma.” Advances in Therapy (2022): 1-11.
Jensen, M. C, Popplewell, L, Cooper, L J, et al. Antitransgene rejection responses contribute to attenuated persistence of adoptively transferred CD20/CD 19-specific chimeric antigen receptor redirected T cells in humans. Biol Blood Marrow Transplant. 2010; 16(9), 1245-1256.
Kawalekar, Omkar U., et al. “Distinct signaling of coreceptors regulates specific metabolism pathways and impacts memory development in CAR T cells.” Immunity 44.2 (2016): 380-390.
Leonie et al., (2021) AT cell redirection platform for co-targeting dual antigens on solid tumors, mAbs, 13:1, e1933690
Liang, A., Ye, S., LL P., Huang, J., Zhu, S., Yao, X., . . . & Yao, Y. (2021). Safety and efficacy of a novel anti-CD20 chimeric antigen receptor (CAR)-T cell therapy in relapsed/refractory (r/r) B-cell non-Hodgkin lymphoma (B-NHL) patients after failing CD 19 CAR-T therapy.
Neelapu S S, Locke F L, Bartlett N L, et al. Axicabtagene Ciloleucel CART-Cell Therapy in Refractory Large B-Cell Lymphoma. N Engl J Med. 2017; 377(26):2531-2544._doi: I 0.1056/NEJMoal 707 44 7.
National Cancer Institute. Cancer Stat Facts: Non-Hodgkin Lymphoma. Surveillance, Epidemiology, and End Results (SEER) Database. Accessed Jun. 9, 2022._https://seer.cancer.gov I statfacts/html/nhl.html.
Sadelain, M., Riviere, I., & Riddell, S. (2017). Therapeutic T cell engineering. Nature, 545(7655), 423-431. doi: 10.1038/nature22395
Sato Y, Takata H, Kobayashi N, Nagata S, Nakagata N, Ueno T, Takiguchi M. Failure of effector function of human CD8+ T Cells in NOD/SCID/JAK3-J− immunodeficient mice transplanted with human CD34+ hematopoietic stem cells. PLoS One. 2010 Oct_1; 5(10):e13109. doi: 10.1371/journal.pone.0013109. PMID: 20957041; PMCID: PMC2948507.
Schneider, D., Xiong, Y., Wu, D., Nlle, V., Schmitz, S., Haso, W., . . . Orentas, R. J._(2017). A tandem CD19/CD20 CAR lentiviral vector drives on-target and off-target antigen modulation in leukemia cell lines. J Immunother Cancer, 5, 42.doi: 10.1186/s40425-017-0246-1
Schroeder B A, Jess J, Sankaran H, Shah N N. Clinical trials for chimeric antigen receptor T-cell therapy: lessons learned and future directions. Curr Opin Hematol. 2022 Jul_1; 29(4):225-232. doi: 10.1097/MOH.0000000000000723. PMID: 35787551; PMCID: PMC9354650.
Schuster S J, Bishop M R, Tam C S, et al. Tisagenlecleucel in Adult Relapsed or Refractory Diffuse Large B-Cell Lymphoma. N Engl J Med. 2019; 380(1):45-56._doi: 10.1056/NEJMoal 804980
Shah, Nirav N., et al. “Bispecific anti-CD20, anti-CD19 CART cells for relapsed B cell malignancies: a phase 1 dose escalation and expansion trial.” Nature medicine 26.10_(2020): 1569-1575.
Shalabi H, Kraft I L, Wang H W, Yuan C M, Yates B, Delbrook C, Zimbelman J D, Giller R, Stetler-Stevenson M, Jaffe E S, Lee D W, Shem J F, Fry T J, Shah N N. Sequential loss of tumor surface antigens following chimeric antigen receptor T-cell therapies in diffuse large B-cell lymphoma. Haematologica. 2018 May; 103(5):e215-e218. doi:_10.3324/haematol.2017.183459. Epub 2018 Feb. 1. PMID: 29419431; PMCID: PMC5927974.
Simon S, Riddell S R. Dual Targeting with CART Cells to Limit Antigen Escape in Multiple Myeloma. Blood Cancer Discov. 2020 Aug. 3; 1(2):130-133
Spiegel, J. Y., et al., CART cells with dual targeting of CD19 and CD22 in adult patients with recurrent or refractory B cell malignancies: a phase 1 trial, Nature Medicine, Vol. 27, Issue No. 8, pp. 1419-1431.
Stroncek D. F., et al., Elutriated lymphocytes for manufacturing chimeric antigen receptor T cells. J Transl Med (2017 Mar. 16), Vol. 15, Issue No. 1. doi: 10.1186/s12967-017-1160-5.
Till, B. G., et al., CD20-specific adoptive immunotherapy for lymphoma using a chimeric antigen receptor with both CD28 and 4-1BB domains: pilot clinical trial results. Blood_(2012), Vol. 119, Issue No. 17, pp. 3940-3950. doi:10.1182/blood-2011-10-387969.
Tsui, C., et al., MYB orchestrates T cell exhaustion and response to checkpoint inhibition, Nature, Vol. 609, Issue No. 7926, pp. 354-360. doi: 10.1038/s41586-022-05105-1. Epub 2022 Aug. 17. PMID: 35978192.
Wang, X., et al., Phenotypic and functional attributes of lentivirus-modified CD19-specific human CD8+ central memory T cells manufactured at clinical scale, J Immunother, (2012), Vol. 35, Issue No. 9, pp. 689-701 doi: 10.1097/CJI.0b013e318270dec7.
Wang, M., et al, KTE-X 19 CART-cell therapy in relapsed or refractory mantle-cell lymphoma, New England Journal of 1\lledicine, Vol. 382, Issue No. 14 (2020): 1331-1342.
Wang. X. et al., Depletion of high-content CD14+ cells from apheresis products is critical for successful transduction and expansion of CART cells during large-scale cGMP manufacturing, Mal Ther Methods Clin Dev., Vol. 22, pp. 377-387. Published 2021 Jul_16.doi: 10.1016/j.orntrn.2021.06.014.
Zah, E. et. al., T Cells Expressing CD19/CD20 Bispecific Chimeric Antigen Receptors Prevent Antigen Escape by Malignant B Cells. Cancer Immunol Res. 2016 June; 4(6):498-508. doi: 10.1158/2326-6066. CIR-15-0231. Epub 2016 Apr. 8.
Zhang, X. et al., Feasibility, and Efficacy of Donor-Derived cdl9-Targeted Car t-Cell Therapy in Refractory/Relapsed(r/r)b-Cell Acute Lymphoblastic Leukemia (b-all) Patients, Blood, Volume 136, Supplement 1, 2020, Pages 4-6, ISSN 0006-4971, https://doi.org/10.1182/blood-2020-137299.
Zhang, Y. et al., Long-term activity of tandem CD19/CD20 CAR therapy in_refractory/relapsed B-cell lymphoma: a single-arm, phase 1-2 trial, Leukemia, Vol. 36, Issue No. 1, pp. 189-196, doi: 10.1038/s41375-021-01345-8 (2022).

Other embodiments of the disclosure are described in the items below:
1. A polypeptide comprising a CD19/CD20 chimeric antigen receptor (CAR), wherein the CAR comprises an amino acid sequence that is at least 70%, 80%, 85%, 86%, 87%, 88%, 89%, 90%, 91%, 92%, 93%, 94%, 95%, 96%, 97%, 98%, or 99% identical to any one of SEQ ID NOs: 2, 4, 6, 8, 10, 12, 14, 16, 18, or 20.
2. A polypeptide comprising a CD19/CD20 chimeric antigen receptor (CAR), wherein the CAR comprises an amino acid sequence that is at least 70%, 80%, 85%, 86%, 87%, 88%, 89%, 90%, 91%, 92%, 93%, 94%, 95%, 96%, 97%, 98%, or 99% identical to any one of SEQ ID NOs: 1, 3, 5, 7, 9, 11, 13, 15, 17, 19, or 52-72.
3. A polypeptide comprising a CD19/CD20 chimeric antigen receptor (CAR), wherein the CAR comprises an amino acid sequence that is at least 70%, 80%, 85%, 86%, 87%, 88%, 89%, 90%, 91%, 92%, 93%, 94%, 95%, 96%, 97%, 98%, or 99% identical to any one of SEQ ID NOs: 182-217.
4. A polypeptide comprising a CD19/CD20 chimeric antigen receptor (CAR), wherein the CAR comprises an amino acid sequence that is at least 70%, 80%, 85%, 86%, 87%, 88%, 89%, 90%, 91%, 92%, 93%, 94%, 95%, 96%, 97%, 98%, or 99% identical to any one of SEQ ID NOs: 218-229.
5. A polypeptide comprising a CD20 chimeric antigen receptor (CAR), the CAR comprising a humanized anti-CD20 scFV comprising a heavy chain variable region at least 70%, 80%, 85%, 86%, 87%, 88%, 89%, 90%, 91%, 92%, 93%, 94%, 95%, 96%, 97%, 98%, or 99% identical to any one of SEQ ID NOs: 79, 83, 87, 91, 95 or 99.
6. A polypeptide of item 5 comprising a CD20 chimeric antigen receptor (CAR), the CAR comprising a humanized anti-CD20 scFV comprising a light chain variable region at least 70%, 80%, 85%, 86%, 87%, 88%, 89%, 90%, 91%, 92%, 93%, 94%, 95%, 96%, 97%, 98%, or 99% identical to any one of SEQ ID NOs: 104 or 108.
7. A polypeptide comprising a CD20 chimeric antigen receptor (CAR), wherein the CAR comprises an amino acid sequence at least 70%, 80%, 85%, 86%, 87%, 88%, 89%, 90%, 91%, 92%, 93%, 94%, 95%, 96%, 97%, 98%, 99%, or 100% identical any one of SEQ ID NOs: 80-82, 84-86, 88-90, 92-94, 96-98, 100-103, 105-107, or 109-141.
8. A polypeptide of any one of items 5-7, wherein the polypeptide further comprises a CD19 CAR.
9. A polypeptide comprising a CD19 chimeric antigen receptor (CAR), the CAR comprising an amino acid sequence 70%, 80%, 85%, 86%, 87%, 88%, 89%, 90%, 91%, 92%, 93%, 94%, 95%, 96%, 97%, 98%, or 99% identical to any one of SEQ ID NOs: 142-181.
10. A method of treating Lupus in a patient comprising administering to said patient an effective amount of an anti-CD19/CD20 engineered immune cell, said engineered immune cell comprising the chimeric antigen receptor set forth in SEQ ID NO. 2.
11. The method according to item 10, wherein said immune cell is a T cell, NK cell, or Treg cell.
12. The method according to item 10 or item 11, wherein said Lupus is SLE.
13. The method according to item 11 or item 12 wherein the dose of said T cells is in the range of 1×10⁶ to 2×10⁸ cells.
14. The method according to any one of item 11-13 wherein the dose of said T cells is 5×10⁷ CAR-T cells.
15. A method of treating Lupus in a patient comprising administering to said patient an effective amount of an anti-CD19/CD20 engineered immune cell, said engineered immune cell comprising a chimeric antigen receptor comprising at least one scFv contained in SEQ ID NO 2; a costimulatory domain; and an activating domain comprising CD3z.
16. A method for treating Systemic Lupus Erythematosus (SLE) comprising: Obtaining immune cells from a patient; Engineering said immune cells to contain a chimeric antigen receptor, wherein said chimeric antigen receptor comprises a CD19/CD20 binding domain, a transmembrane domain, and an intracellular domain; Expanding said immune cells; and. Administering said immune cells to said patient.
17. The method according to item 15 or claim 16, wherein said immune cell is a T cell.
18. The method according to any one of claims 15-17, wherein said binding domain comprises one or more scFvs.
19. The method according to any one of items 15-18, wherein the intracellular domain comprises one or more intracellular costimulatory domain, and one or more intracellular activating domain.
20. The method according to any one of items 15-19, wherein the intracellular activating domain is CD3z.
21. The method according to any one of items 15-19, wherein the intracellular costimulatory domain is 4-IBB.
22. The method according to any one of items 15 to 19, wherein the intracellular costimulatory domain is CD28.
23. The method according to any one of items 17 to 22, wherein the dose of said T cells is in the range of 1×10⁶ to 2×10⁸ cells.
24. The method according to item 23, wherein the dose of said T cells is 5×10⁷ CAR-T cells.
25. A method of treating autoimmune disease in a patient comprising administering to said patient an effective amount of an anti-CD19/CD20 engineered immune cell, said engineered immune cell comprising a chimeric antigen receptor set forth in SEQ ID NO. 2.
26. The method according to item 24 wherein the autoimmune disease is at least one of Lupus, Rheumatoid Arthritis, IBD, and psoriasis.
27. A polypeptide comprising a CD19 chimeric antigen receptor (CAR), comprising: a) an anti-CD19 scFv, said scFv having at least one CDR sequence isolated or derived from a nonhuman subject, and wherein said scFv comprises a heavy chain variable region and light chain variable region; b) a spacer domain; c) a transmembrane domain; d) a 4-1BB cytoplasmic signaling domain; and e) a CD3 zeta signaling domain.
28. The polypeptide of item 27, wherein the at least one CDR sequences isolated or derived from a nonhuman subject.
29. A polypeptide comprising a CD19/CD20 chimeric antigen receptor (CAR), comprising an anti-CD20 scFv comprising a light chain variable region and heavy chain variable region; a (G4S)4 linker; an anti-CD19 scFv comprising a heavy chain variable region and light chain variable region; a spacer domain; a transmembrane domain; a 4-1BB cytoplasmic signaling domain; and a CD3 zeta signaling domain wherein at least one of the anti-CD20 scFv or anti-CD19 scFv comprises at least one CDR sequence isolated or derived from a nonhuman subject.
30. The polypeptide of any one of items 27 to 29, wherein the at least one CDR sequences may optionally contain one or more point mutations.
31. A nucleic acid encoding a polypeptide according to any one of items 27 to 30.
32. A cell comprising the polypeptide of any one of items 27 to 30.
33. The cell of item 32, wherein the cell is a T cell.
34. A plasmid comprising the nucleic acid of item 31.
35. A vector comprising the nucleic acid of item 31.
36. A cell comprising the nucleic acid of item 31.
37. A method of treating a disease or disorder comprising administering to a patient an effective amount of a population of cells according to item 32 or item 33.
38. The method according to item 37, wherein said disease or disorder is at least one of lupus, lymphoma, leukemia, glioma, and/or glioblastoma.
39. The method according to item 38, wherein said lymphoma is NHL.
40. The method according to item 38, wherein said lymphoma is DLBCL, follicular lymphoma, MALT, CTCL, or MCL.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

It will be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A polypeptide comprising a CD19/CD20 chimeric antigen receptor (CAR), the CAR comprising:
an anti-CD20 scFv comprising (i) a light chain variable region having the amino acid sequence of SEQ ID NO: 34, and (ii) a heavy chain variable region having the amino acid sequences of SEQ ID NO: 35;
a (G₄S)ₙ linker, wherein n is 1, 2, 3, or 4;
an anti-CD19 scFv comprising (i) a heavy chain variable region having the amino acid sequence of SEQ ID NO: 36, and (ii) a light chain variable region having the amino acid sequences of SEQ ID NO: 37;
a spacer domain comprising the amino acid sequence of any one selected from SEQ ID NOs: 21-29;
a transmembrane domain comprising the amino acid sequence of SEQ ID NO: 30 or 31;
a 4-1BB cytoplasmic signaling domain comprising the amino acid sequence of SEQ ID NO: 32; and
a CD3 zeta signaling domain comprising the amino acid sequence of SEQ ID NO: 33.

2. The polypeptide of claim 1, wherein at least one of the anti-CD20 scFv or anti-CD19 scFv comprises at least one CDR sequence isolated or derived from a nonhuman subject.

3. The polypeptide of claim 1, wherein the CAR comprises the formula from N terminus to C terminus:
[anti-CD20 scFv]-[(G₄S)ₙ linker]-[anti-CD19 scFv]-[spacer domain]-[transmembrane domain]-[4-lBB cytoplasmic signaling domain]-[CD3 zeta signaling domain].

4. The polypeptide of claim 1, wherein the (G₄S)ₙ linker is (G₄S)₁ comprising the amino acid sequence of SEQ ID NO: 48.

5. (canceled)

6. The polypeptide of claim 1, wherein the (G₄S)ₙ linker is (G₄S)₂ comprising the amino acid sequence of SEQ ID NO: 49.

7. (canceled)

8. The polypeptide of claim 1 3, wherein the (G₄S)ₙ linker is (G₄S)₃ comprising the amino acid sequence of SEQ ID NO: 50.

9. (canceled)

10. The polypeptide of a claim 1, wherein the (G₄S)ₙ linker is (G₄S)₄ comprising the amino acid sequence of SEQ ID NO: 51.

11. (canceled)

12. A polypeptide comprising a CD19/CD20 chimeric antigen receptor (CAR), wherein the CAR comprises the amino acid sequence selected from the group consisting of SEQ ID NOs: 2, 4, 6, 8, 10, 12, 14, 16, 18, and 20.

13. A nucleic acid encoding a polypeptide of claim 1.

14. A plasmid, a vector, and/or cell comprising the nucleic acid of claim 13.

15. A nucleic acid encoding a polypeptide of claim 12.

16. A plasmid, a vector, and/or a cell comprising the nucleic acid of claim 15.

17. A cell comprising the polypeptide of claim 1.

18. The cell of claim 17, wherein the cell is an immune cell.

19. The cells of claim 18, wherein the immune cell is selected from the group consisting of T cell, NK cell, and Treg cell.

20. A method of treating an autoimmune disease in a patient in need thereof, comprising administering to the patient an effective amount of a population of cells according to claim 17.

21. The method of claim 20, wherein the population of cells are immune cells.

22. A method of treating an autoimmune disease in a patient in need thereof, the method comprising:
obtaining immune cells from a patient;
engineering the immune cells, wherein the engineered immune cells comprise a polypeptide of claim 1;
expanding the engineered immune cells; and
administering an effective amount of the expanded engineered immune cells to the patient.

23. A method of treating an autoimmune disease in a patient comprising administering to said patient an effective amount of an anti-CD19/CD20 engineered immune cells, wherein said engineered immune cells comprises the chimeric antigen receptor set forth in SEQ ID NO. 2.

24. The method of claim 21, wherein the immune cells are selected from the group consisting of T cells, NK cells, and Treg cells.

25. A method of treating an autoimmune disease in a patient in need thereof, comprising administering to the patient an effective amount of an engineered T cell, the engineered T cell comprising a polypeptide comprising a CD19/CD20 chimeric antigen receptor (CAR), the CAR comprising:
an anti-CD20 scFv comprising (i) a light chain variable region having the amino acid sequence of SEQ ID NO: 34, and (ii) a heavy chain variable region having the amino acid sequences of SEQ ID NO: 35;
a (G₄S)ₙ linker, wherein n is 1, 2, 3, or 4;
an anti-CD19 scFv comprising (i) a heavy chain variable region having the amino acid sequence of SEQ ID NO: 36, and (ii) a light chain variable region having the amino acid sequences of SEQ ID NO: 37;
a spacer domain comprising the amino acid sequence of any one selected from SEQ ID NOs: 21-29;
a transmembrane domain comprising the amino acid sequence of SEQ ID NO: 30 or 31;
a 4-1BB cytoplasmic signaling domain comprising the amino acid sequence of SEQ ID NO: 32; and
a CD3 zeta signaling domain comprising the amino acid sequence of SEQ ID NO: 33.

26. The method of claim 24, wherein at least one of the anti-CD20 scFv or anti-CD19 scFv comprises at least one CDR sequence isolated or derived from a nonhuman subject.

27. The method of claim 24, wherein the CAR comprises the formula from N terminus to C terminus:
[anti-CD20 scFv]-[(G₄S)ₙ linker]-[anti-CD19 scFv]-[spacer domain]-[transmembrane domain]-[4-1BB cytoplasmic signaling domain]-[CD3 zeta signaling domain].

28. The method of claim 24, wherein the (G₄S)ₙ linker is (G₄S)₁ comprising the amino acid sequence of SEQ ID NO: 48.

29. (canceled)

30. The method of claim 24, wherein the (G₄S)ₙ linker is (G₄S)₂ comprising the amino acid sequence of SEQ ID NO: 49.

31. (canceled)

32. The method of claim 24, wherein the (G₄S)ₙ linker is (G₄S)₃ comprising the amino acid sequence of SEQ ID NO: 50.

33. (canceled)

34. The method of claim 24, wherein the (G₄S)_{I} linker is (G₄S)₄ comprising the amino acid sequence of SEQ ID NO: 51.

35. (canceled)

36. The method of claim 20, wherein the autoimmune disease is selected from the group consisting of lupus, rheumatoid arthritis (RA), systemic lupus erythematosus (SLE), inflammatory bowel disease (IBD/Crohn's), Type 1 diabetes mellitus, Guillain-Barre syndrome, chronic inflammatory demyelinating polyneuropathy, psoriasis, Graves' Disease, Hashimoto's Thyroiditis, Myasthenia Gravis, scleroderma, systemic sclerosis, Multiple Sclerosis (MS), autoimmune neuropathy, transverse myelitis, optic neuritis, neuromyelitis optica, acute dissernlnated encephalomyelitis, autoimmune or paraneoplastic encephalitis, spasticity, Idiopathic inflammatory myopathies, Anca Associated Vasculitis, and Systemic Sclerosis.

37. The method of claim 36, wherein the autoimmune disease is lupus, SLE, Idiopathic inflammatory myopathies, Anca Associated Vasculitis, Systemic Sclerosis and MS.

38. The method of claim 36, wherein the autoimmune disease is RA, IBD, or psoriasis.

39. The method of claim 20, wherein the effective amount is between about 1×10⁶ and about 2×10⁸ cells.

40. (canceled)

41. A polypeptide comprising a CD19 chimeric antigen receptor (CAR), comprising:
an anti-CD19 scFv comprising (i) a heavy chain variable region having the amino acid sequence of SEQ ID NO: 36, and (ii) a light chain variable region having the amino acid sequences of SEQ ID NO: 37;
a spacer domain comprising the amino acid sequence of any one selected from SEQ ID NOs: 21-29;
a transmembrane domain comprising the amino acid sequence of SEQ ID NO: 30 or 31;
a 4-1BB cytoplasmic signaling domain comprising the amino acid sequence of SEQ ID NO: 32; and
a CD3 zeta signaling domain comprising the amino acid sequence of SEQ ID NO: 33.

42. The polypeptide of claim 41, wherein the anti-CD19 scFv comprises at least one CDR sequence isolated or derived from a nonhuman subject.

43. The polypeptide of claim 41, wherein the CAR comprises the formula from N terminus to C terminus:
[anti-CD19 scFv]-[spacer domain]-[transmembrane domain]-[4-1BB cytoplasmic signaling domain]-[CD3 zeta signaling domain].

44. A nucleic acid encoding a polypeptide according to claim 41.

45. A plasmid, vector, and/or cell comprising the nucleic acid of claim 44.

46. (canceled)

47. (canceled)

48. A cell comprising the polypeptide of claim 41.

49. The cell of claim 48, wherein the cell is an immune cell.

50. The cells of claim 49, wherein the immune cell is selected from the group consisting of T cell, NK cell, and Treg cell.

51. A method of treating an autoimmune disease in a patient in need thereof, comprising administering to the patient an effective amount of a population of cells according to claim 48.

52. The method of claim 51, wherein the population of cells are immune cells.

53. A method of treating an autoimmune disease in a patient in need thereof, the method comprising:
obtaining immune cells from a patient;
engineering the immune cells, wherein the engineered immune cells comprise a polypeptide of claim 41;
expanding the engineered immune cells; and
administering an effective amount of the expanded engineered immune cells to the patient.

54. The method of claim 53, wherein the immune cells is selected from the group consisting of T cells, NK cells, and Treg cells.

55. A method of treating an autoimmune disease in a patient comprising administering to the patient an effective amount of an engineered T cell, the engineered T cell comprising a polypeptide comprising a CD19-OR-CD20 chimeric antigen receptor (CAR), wherein the CAR comprises from N-terminus to C-terminus:
an anti-CD20 scFv with a variable heavy domain and a variable light domain obtained and/or derived from ofatumumab;
a (G4S)n linker, wherein n is 1, 3, or 4;
an anti-CD19 scFv comprising a variable heavy domain and a variable light domain;
a spacer;
a transmembrane domain;
a co-stimulatory domain; and
a CD3-zeta cytoplasmic signaling domain.

56. The method of claim 55, wherein the (G₄S)ₙ linker is (G₄S)₁.

57. The method of claim 55, wherein the (G₄S)ₙ linker is (G₄S)₃.

58. The method of claim 55, wherein the (G₄S)ₙ linker is (G₄S)₄.

59. The method of claim 51, wherein the autoimmune disease is selected from the group consisting of lupus, rheumatoid arthritis (RA), systemic lupus erythematosus (SLE), inflammatory bowel disease (IBD/Crohn's), Type 1 diabetes mellitus, Guillain-Barre syndrome, chronic inflammatory demyelinating polyneuropathy, psoriasis, Graves' Disease, Hashimoto's Thyroiditis, Myasthenia Gravis, scleroderma, systemic sclerosis, Multiple Sclerosis (MS), autoimmune neuropathy, transverse myelitis, optic neuritis, neuromyelitis optica, acute dissernlnated encephalomyelitis, autoimmune or paraneoplastic encephalitis, spasticity, Idiopathic inflammatory myopathies, Anca Associated Vasculitis, and Systemic Sclerosis.

60. The method of claim 59, wherein the autoimmune disease is lupus, SLE, Idiopathic inflammatory myopathies, Anca Associated Vasculitis, Systemic Sclerosis and MS.

61. The method of claim 59, wherein the autoimmune disease is RA, IBD or psoriasis.

62. A cell comprising the polypeptide of claim 12.

63. The cell of claim 63, wherein the cell is an immune cell.

64. The cells of claim 64, wherein the immune cell is selected from the group consisting of T cell, NK cell, and Treg cell.

65. A method of treating an autoimmune disease in a patient in need thereof, comprising administering to the patient an effective amount of a population of cells according to claim 62.
